(19) United States
(12) Patent Application Publication
(10) Pub. No.: US 2026/0333993 A1
(43) Pub. Date: Oct. 22, 2026

(54) HETEROCYCLIC COMPOUND, ORGANIC LIGHT-EMITTING DEVICE COMPRISING SAME, AND COMPOSITION FOR ORGANIC LAYER

(71) Applicant: LT MATERIALS CO., LTD., Yongin-si, Gyeonggi-do (KR)

(72) Inventors: Yu Jin HEO, Yongin-si (KR); Jun Tae MO, Yongin-si (KR); Dong Jun KIM, Yongin-si (KR); Dae Hyuk CHOI, Yongin-si (KR)

(73) Assignee: LT MATERIALS CO., LTD., Yongin-si, Gyeonggi-do (KR)

(21) Appl. No.: 18/880,747

(22) PCT Filed: Jun. 12, 2023
(86) PCT No.: PCT/KR2023/008044

§ 371 Date: Jan. 2, 2025

(30) Foreign Application Priority Data
Jul. 13, 2022 (KR) 10-2022-0086421

(51) Int. Cl. H10K 50/11; C07B 59/00; C07D 405/04; H10K 85/60

(52) U.S. Cl. CPC H10K 50/11; C07B 59/002; C07D 405/04; H10K 85/626; H10K 85/633; H10K 85/636; H10K 85/654; H10K 85/6572; H10K 85/6574; H10K 85/6576; C07B 2200/05

(57) ABSTRACT

The present specification relates to a heterocyclic compound represented by Chemical Formula 1, an organic light emitting device including the same, and a composition for an organic material layer.

### TECHNICAL FIELD

This application claims priority to and the benefits of Korean Patent Application No. 10-2022-0086421, filed with the Korean Intellectual Property Office on Jul. 13, 2022, the entire contents of which are incorporated herein by reference.

The present disclosure relates to a heterocyclic compound, an organic light emitting device including the same, and a composition for an organic material layer.

### BACKGROUND ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to the organic light emitting device having such a structure, electrons and holes injected from the two electrodes bind and pair in the organic thin film, and then light is emitted as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of performing a role of a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### DISCLOSURE

### Technical Problem

The present disclosure is directed to providing a heterocyclic compound, an organic light emitting device including the same, and a composition for an organic material layer.

### Technical Solution

One embodiment of the present application provides a heterocyclic compound represented by the following Chemical Formula 1.

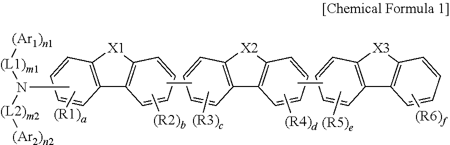

In Chemical Formula 1,
X1 to X3 are the same as or different from each other, and each independently O or S,
Ar₁ and Ar₂ are the same as or different from each other, and each independently a substituted or unsubstituted C6 to C60 aryl group; or a substituted or unsubstituted C2 to C60 heteroaryl group,
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
L1 and L2 are the same as or different from each other, and each independently a direct bond; a substituted or unsubstituted arylene group having 6 to 60 carbon atoms; or a substituted or unsubstituted heteroarylene group having 2 to 60 carbon atoms,
a to e are the same as or different from each other, and each independently an integer of 0 to 3,
f is an integer of 0 to 4,
m1 and m2 are the same as or different from each other, and each independently an integer of 0 to 4, and
n1 and n2 are the same as or different from each other, and each independently an integer of 1 to 5.

In addition, one embodiment of the present application provides an organic light emitting device including: a first electrode; a second electrode provided opposite to the first electrode; and one or more organic material layers provided between the first electrode and the second electrode, wherein the one or more organic material layers include the heterocyclic compound represented by Chemical Formula 1.

In addition, one embodiment of the present application provides an organic light emitting device, wherein the one or more organic material layers including the heterocyclic compound of Chemical Formula 1 further include a heterocyclic compound represented by the following Chemical Formula A.

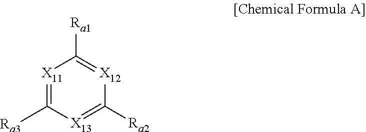

In Chemical Formula A,
X₁₁ to X₁₃ are the same as or different from each other, and each independently N or CR′,
at least one of X₁ to X₁₃ is N, and
Rₐ₁ to Rₐ₃ and R′ are the same as or different from each other, and each independently selected from the group consisting of hydrogen; deuterium; a halogen group; a cyano group; substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

In addition, another embodiment of the present application provides a composition organic material layer, the composition including: the heterocyclic compound represented by Chemical Formula 1, and the heterocyclic compound represented by Chemical Formula A.

### Advantageous Effects

A heterocyclic compound according to one embodiment can be used as an organic material layer material of an organic light emitting device. The compound is capable of performing roles of a hole injection layer material, an electron blocking layer material, a hole transport layer material, a light emitting layer material, an electron transport layer material, a hole blocking layer material, an electron injection layer material and the like in an organic light emitting device. Particularly, the compound can be used as a light emitting layer material of an organic light emitting device.

The heterocyclic compound can be used as a light emitting material either alone or as a mixture with a P-type host, and may be used as a host material or a dopant material of a light emitting layer.

Particularly, since the heterocyclic compound represented by Chemical Formula 1 includes a heteroaryl-based substituent in the dibenzofuran or dibenzothiophene structure that is a core structure, electronic properties are strengthened in the dibenzofuran or dibenzothiophene structure, the core structure, and a band-gap of the compound and the triplet excited state energy level (T₁) value can be adjusted. Accordingly, when using the compound represented by Chemical Formula 1 in an organic material layer, it is possible to lower a driving voltage of an organic light emitting device, enhance light emission efficiency, and enhance lifetime properties of an organic light emitting device due to thermal stability of the compound.

Specifically, when the compound represented by Chemical Formula 1 is used as a host material of a light emitting layer, the heteroatom of the amine group enhances a hole injection ability of the hole transport group, thereby increasing efficiency and lifetime. Particularly, by fixing the position of the arylamine group corresponding to a hole transporting group of Chemical Formula 1 to a specific position as in Chemical Formula 1, a steric arrangement is obtained, and since strong charge transfer is possible by spatially separating HOMO (highest occupied molecular orbital) and LUMO (lowest unoccupied molecular orbital), the compound is suitable as a red host material, and high efficiency can be expected when used as an organic material in an organic light emitting device.

### DESCRIPTION OF DRAWINGS

FIGS. 1 to 3 are diagrams each schematically illustrating a lamination structure of an organic light emitting device according to one embodiment of the present disclosure.

### MODE FOR DISCLOSURE

Hereinafter, the present disclosure will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, a term “substituted or unsubstituted” means being substituted with one or more substituents selected from the group consisting of deuterium; halogen; a cyano group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent in which two or more substituents selected from among the substituents exemplified above are linked or being unsubstituted.

In the present specification, R, R′, and R″ are the same as or different from each other, and may be each independently a substituted or unsubstituted C1 to C60 alkyl group; a substituted or unsubstituted C6 to C60 aryl group; or a substituted or unsubstituted C2 to C60 heteroaryl group.

In the present specification, “the number of protons” means the number of substituents that a specific compound may have, and specifically, the number of protons may mean the number of hydrogens. For example, unsubstituted benzene may be expressed to have the number of protons of 5, an unsubstituted naphthyl group may be expressed to have the number of protons of 7, a naphthyl group substituted with a phenyl group may be expressed to have the number of protons of 6, and an unsubstituted biphenyl group may be expressed to have the number of protons of 9.

In the present specification, the halogen may be fluorine, chlorine, bromine or iodine.

In the present specification, the alkyl group includes a linear or branched form having 1 to 60 carbon atoms, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes a linear or branched form having 2 to 60 carbon atoms, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes a linear or branched form having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes a monocyclic or polycyclic group having 3 to 60 carbon atoms, and may be further substituted with other substituents. Herein, the polycyclic group means a group in which the cycloalkyl group is directly linked to or fused with another cyclic group. Herein, the another cyclic group may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples thereof may include a cyclopropyl group, a cyclobutyl group, a a 3-methylcyclopentyl group, cyclopentyl group, a 2,3-dimethylcyclopentyl group, a cyclohexyl group, a 3-group, a 2,3-methylcyclohexyl group, a 4-methylcyclohexyl dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes a monocyclic or polycyclic group having 2 to 60 carbon atoms, and may be further substituted with other substituents. Herein, the polycyclic group means a group in which the heterocycloalkyl group is directly linked to or fused with another cyclic group. Herein, the another cyclic group may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes a monocyclic or polycyclic group having 6 to 60 carbon atoms, and may be further substituted with other substituents. Herein, the polycyclic group means a group in which the aryl group is directly linked to or fused with another cyclic group. Herein, the another cyclic group may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

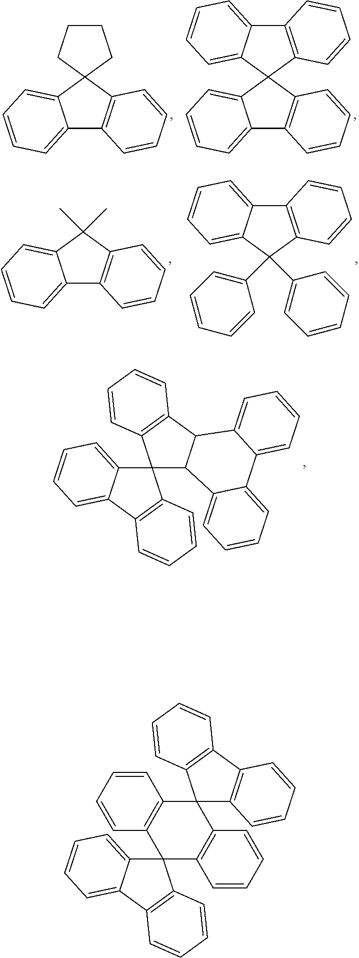

and the like may be included, however, the structure is not limited thereto.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

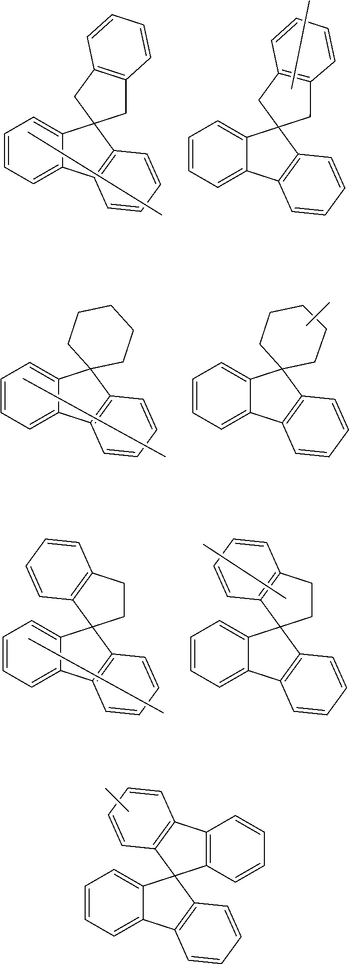

In the present specification, the heteroaryl group includes S, O, Se, N or Si as a heteroatom, includes a monocyclic or polycyclic group having 2 to 60 carbon atoms, and may be further substituted with other substituents. Herein, the polycyclic group means a group in which the heteroaryl group is directly linked to or fused with another cyclic group. Herein, the another cyclic group may be a heteroaryl group, but may also be different such as a cycloalkyl group, a types of cyclic groups heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, an imidazopyridinyl group, a diazanaphthalenyl group, a triazaindenyl group, a 2-indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl a phenazinyl group, a dibenzosilole group, a spirobi (dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group, a dimethylamine group, an ethylamine group, a diethylamine group, phenylamine group, a a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions to which substituents may come are all hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

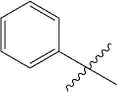 [SMILES: *c1ccccc1]

may mean that the total number of substituents that the phenyl group may have is 5 (T1 in the formula), and the number of deuterium among these is 1 (T2 in the formula). In other words, having a deuterium content of 20% in a phenyl group may be represented by the following structural formulae.

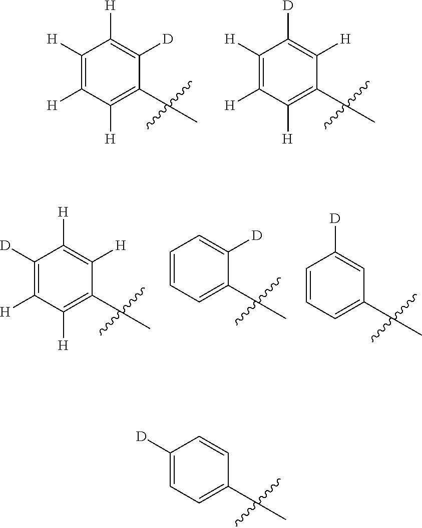 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include benzene, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

One embodiment of the present application provides a heterocyclic compound represented by the following Chemical Formula 1.

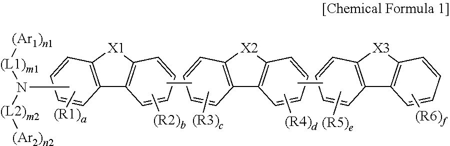

In Chemical Formula 1,
X1 to X3 are the same as or different from each other, and each independently O or S,
Ar₁ and Ar₂ are the same as or different from each other, and each independently a substituted or unsubstituted C6 to C60 aryl group; or a substituted or unsubstituted C2 to C60 heteroaryl group,
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
L1 and L2 are the same as or different from each other, and each independently a direct bond; a substituted or unsubstituted arylene group having 6 to 60 carbon atoms; or a substituted or unsubstituted heteroarylene group having 2 to 60 carbon atoms,
a to e are the same as or different from each other, and each independently an integer of 0 to 3,
f is an integer of 0 to 4,
m1 and m2 are the same as or different from each other, and each independently an integer of 0 to 4, and
n1 and n2 are the same as or different from each other, and each independently an integer of 1 to 5.

In one embodiment of the present application, Chemical Formula 1 may be represented by any one of the following Chemical Formula 1-1 to Chemical Formula 1-8.

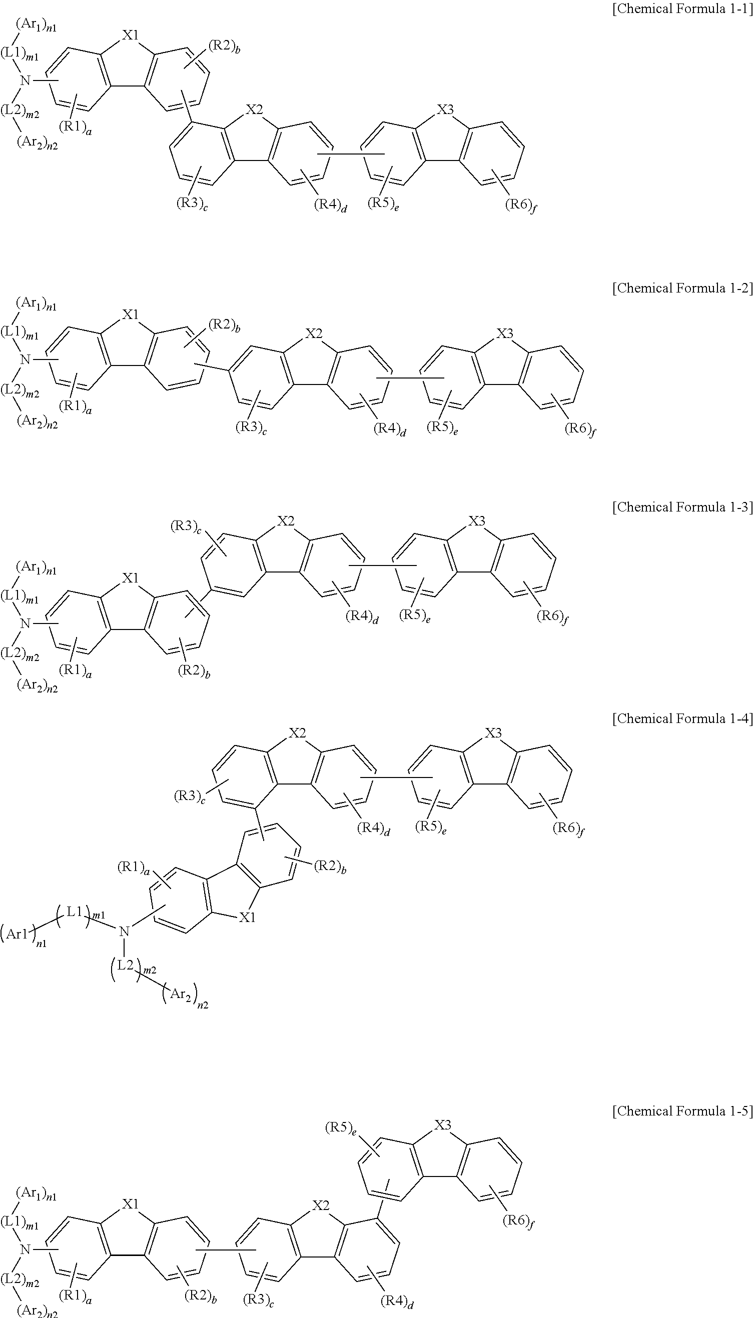

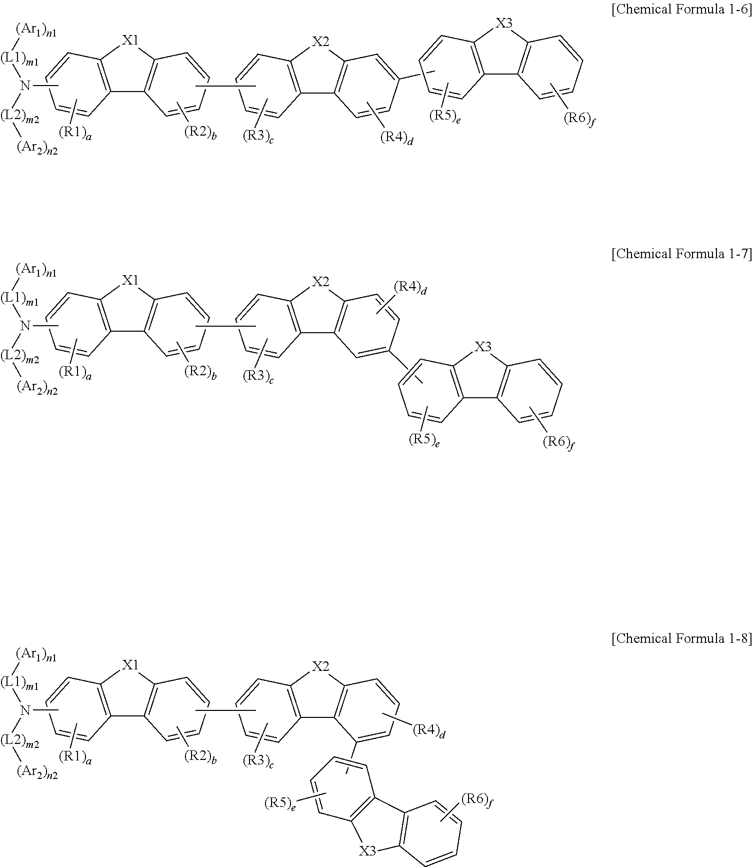

In Chemical Formula 1-1 to Chemical Formula 1-8, each substituent has the same definition as in Chemical Formula 1.

In one embodiment of the present application, X1 to X3 may be O.

In one embodiment of the present application, X1 to X3 may be S.

In one embodiment of the present application, X1 and X2 are O, and X₃ may be S.

In one embodiment of the present application, X1 and X3 are 0, and X2 may be S.

In one embodiment of the present application, X2 and X3 are 0, and X1 may be S.

In one embodiment of the present application, X1 and X2 are S, and X₃ may be O.

In one embodiment of the present application, X1 and X3 are S, and X2 may be O.

In one embodiment of the present application, X2 and X3 are S, and X1 may be O.

In one embodiment of the present application, R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring or a substituted or unsubstituted C2 to C60 heteroring, and R101, R102 and R103 are the same as or different from each other and may be each independently a substituted or unsubstituted C1 to C60 alkyl group; a substituted or unsubstituted C6 to C60 aryl group; or a substituted or unsubstituted C2 to C60 heteroaryl group.

In one embodiment of the present application, R1 to R6 are the same as or different from each other, and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102 and —SiR101R102R103, or two or more groups adjacent to each other bond to each other to form a substituted or unsubstituted C6 to C30 aromatic hydrocarbon ring or a substituted or unsubstituted C2 to C30 heteroring, and R101, R102 and R103 are the same as or different from each other and may be each independently a substituted or unsubstituted C1 to C60 alkyl group; a substituted or unsubstituted C6 to C60 aryl group; or a substituted or unsubstituted C2 to C60 heteroaryl group.

In one embodiment of the present application, R1 to R6 are the same as or different from each other, and each independently selected from the group consisting of hydrogen; deuterium; halogen; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring or a substituted or unsubstituted C2 to C20 heteroring.

In one embodiment of the present application, R1 to R6 are the same as or different from each other, and may be each independently hydrogen; deuterium; halogen; a substituted or unsubstituted C1 to C60 alkyl group; a substituted or unsubstituted C6 to C60 aryl group; or a substituted or unsubstituted C2 to C60 heteroaryl group.

In one embodiment of the present application, R1 to R6 are the same as or different from each other, and may be each independently hydrogen; deuterium; halogen; a substituted or unsubstituted C1 to C30 alkyl group; a substituted or unsubstituted C6 to C30 aryl group; or a substituted or unsubstituted C2 to C30 heteroaryl group.

In one embodiment of the present application, R1 to R6 are the same as or different from each other, and may be each independently hydrogen; deuterium; halogen; a substituted or unsubstituted C1 to C20 alkyl group; a substituted or unsubstituted C6 to C20 aryl group; or a substituted or unsubstituted C2 to C20 heteroaryl group.

In one embodiment of the present application, R1 to R6 are the same as or different from each other, and may be each independently hydrogen; deuterium; halogen; or a substituted or unsubstituted C6 to C20 aryl group.

In one embodiment of the present application, R1 to R6 are the same as or different from each other, and may be each independently hydrogen; deuterium; halogen; a substituted or unsubstituted monocyclic C6 to C10 aryl group; or a substituted or unsubstituted polycyclic C10 to C20 aryl group.

In one embodiment of the present application, R1 to R6 are the same as or different from each other, and may be each independently hydrogen; deuterium; halogen; a substituted or unsubstituted phenyl group; or a substituted or unsubstituted biphenyl group.

In one embodiment of the present application, R1 to R6 are the same as or different from each other, and may be each independently hydrogen; deuterium; or halogen.

In one embodiment of the present application, R1 to R6 are the same as or different from each other, and may be each independently hydrogen; deuterium; or fluorine.

In one embodiment of the present application, Ar₁ and Ar₂ are the same as or different from each other, and may be each independently a substituted or unsubstituted C6 to C60 aryl group; or a substituted or unsubstituted C2 to C60 heteroaryl group.

In one embodiment of the present application, Ar₁ and Ar₂ are the same as or different from each other, and may be each independently a substituted or unsubstituted C6 to C30 aryl group; or a substituted or unsubstituted C2 to C30 heteroaryl group.

In one embodiment of the present application, Ar₁ and Ar₂ are the same as or different from each other, and may be each independently a substituted or unsubstituted C6 to C60 aryl group.

In one embodiment of the present application, Ar₁ and Ar₂ are the same as or different from each other, and may be each independently a substituted or unsubstituted C6 to C40 aryl group.

In one embodiment of the present application, Ar₁ and Ar₂ are the same as or different from each other, and may be each independently a substituted or unsubstituted C6 to C30 aryl group.

In one embodiment of the present application, Ar₁ and Ar₂ are the same as or different from each other, and may be each independently a substituted or unsubstituted C6 to C20 aryl group.

In one embodiment of the present application, Ar₁ and Ar₂ are the same as or different from each other, and may be each independently a substituted or unsubstituted monocyclic C6 to C10 aryl group; or a substituted or unsubstituted polycyclic C10 to C20 aryl group.

In one embodiment of the present application, Ar₁ and Ar₂ are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted anthracenyl group; a substituted or unsubstituted pyrenyl group; a substituted or unsubstituted chrysenyl group; or a substituted or unsubstituted phenanthrenyl group.

In one embodiment of the present application, Ar₁ and Ar₂ are the same as or different from each other, and each independently a phenyl group unsubstituted or substituted with deuterium, fluorine, a butyl group or a naphthyl group; a biphenyl group unsubstituted or substituted with deuterium or fluorine; a terphenyl group unsubstituted or substituted with deuterium; a naphthyl group unsubstituted or substituted with deuterium or fluorine; a phenanthrenyl group unsubstituted or substituted with deuterium; or a pyrenyl group unsubstituted or substituted with deuterium.

In one embodiment of the present application, the heterocyclic compound represented by Chemical Formula 1 may not include deuterium, or may have a deuterium content of, for example, 1% or greater, 5% or greater, 10% or greater, 15% or greater, 20% or greater, 25% or greater, 30% or greater, 35% or greater, 40% or greater, 45% or greater or 50% or greater, and 100% or less, 95% or less, 90% or less, 85% or less, 80% or less, 75% or less, 70% or less, 65% or less or 60% or less based on the total number of hydrogen atoms and deuterium atoms in the heterocyclic compound represented by Chemical Formula 1.

In one embodiment of the present application, the heterocyclic compound represented by Chemical Formula 1 may not include deuterium, or may have a deuterium content of 1% to 100% based on the total number of hydrogen atoms and deuterium atoms.

In one embodiment of the present application, the heterocyclic compound represented by Chemical Formula 1 may not include deuterium, or may have a deuterium content of 10% to 100% based on the total number of hydrogen atoms and deuterium atoms.

In one embodiment of the present application, the heterocyclic compound represented by Chemical Formula 1 may not include deuterium, or may have a deuterium content of 20% to 90% based on the total number of hydrogen atoms and deuterium atoms.

In one embodiment of the present application, the heterocyclic compound represented by Chemical Formula 1 may not include deuterium, or may have a deuterium content of 30% to 80% based on the total number of hydrogen atoms and deuterium atoms.

In one embodiment of the present application, the heterocyclic compound represented by Chemical Formula 1 may not include deuterium, or may have a deuterium content of 40% to 70% based on the total number of hydrogen atoms and deuterium atoms.

In one embodiment of the present application, the heterocyclic compound represented by Chemical Formula 1 may not include deuterium, or may have a deuterium content of 50% to 60% based on the total number of hydrogen atoms and deuterium atoms.

In one embodiment of the present application, L1, L2, Ar1, Ar2 and R1 to R6 may all include H that is not deuterated.

In one embodiment of the present application, at least one of L1, L2, Ar1, Ar2 and R1 to R6 includes D, and at least one of L1, L2, Ar1, Ar2 and R1 to R6 may include at least one H that is not deuterated.

In one embodiment of the present application, L1, L2, Ar1, Ar2 and R1 to R6 may all include D.

One embodiment of the present application provides a heterocyclic compound, wherein Chemical Formula 1 is represented by any one of the following compounds. In addition, in one embodiment of the present application, the following compounds are just one example, and the present application is not limited thereto and may include other compounds included in Chemical Formula 1 including additional substituents.

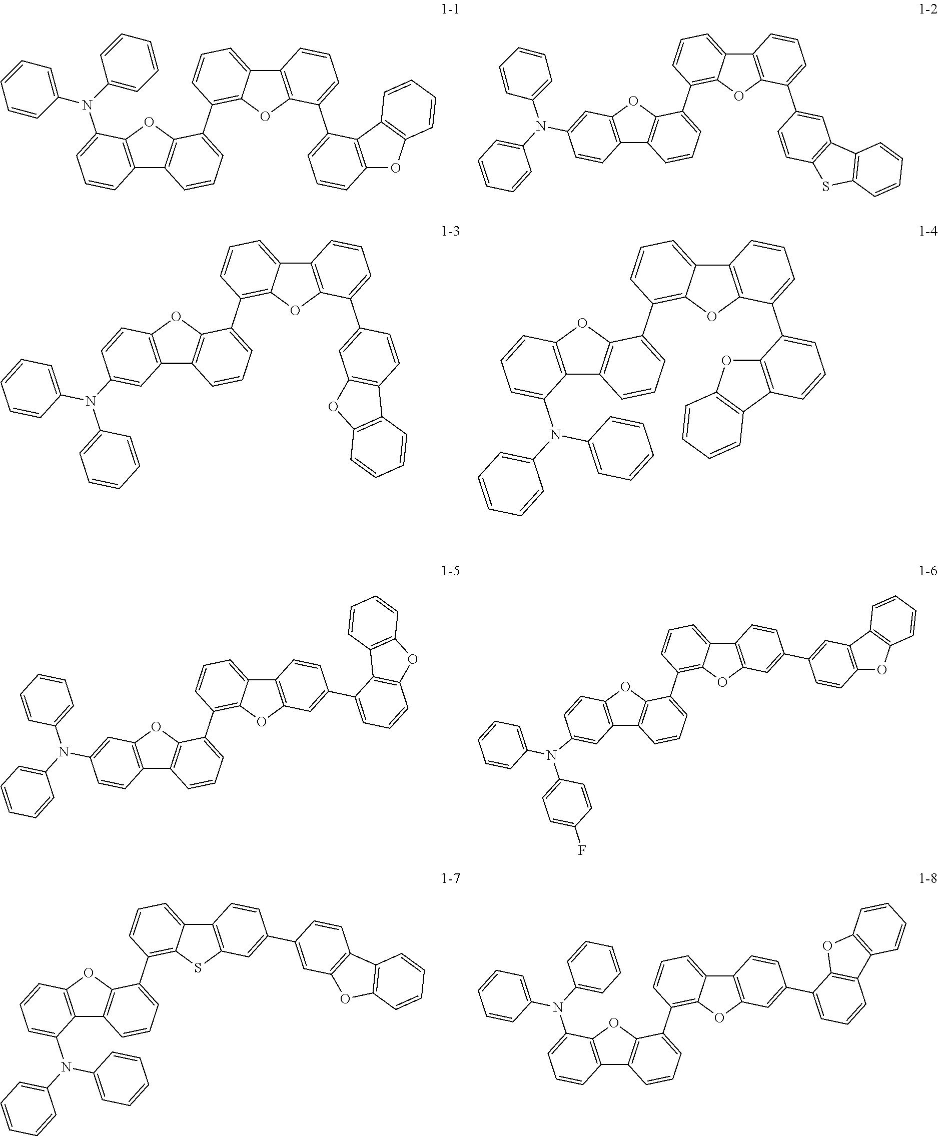

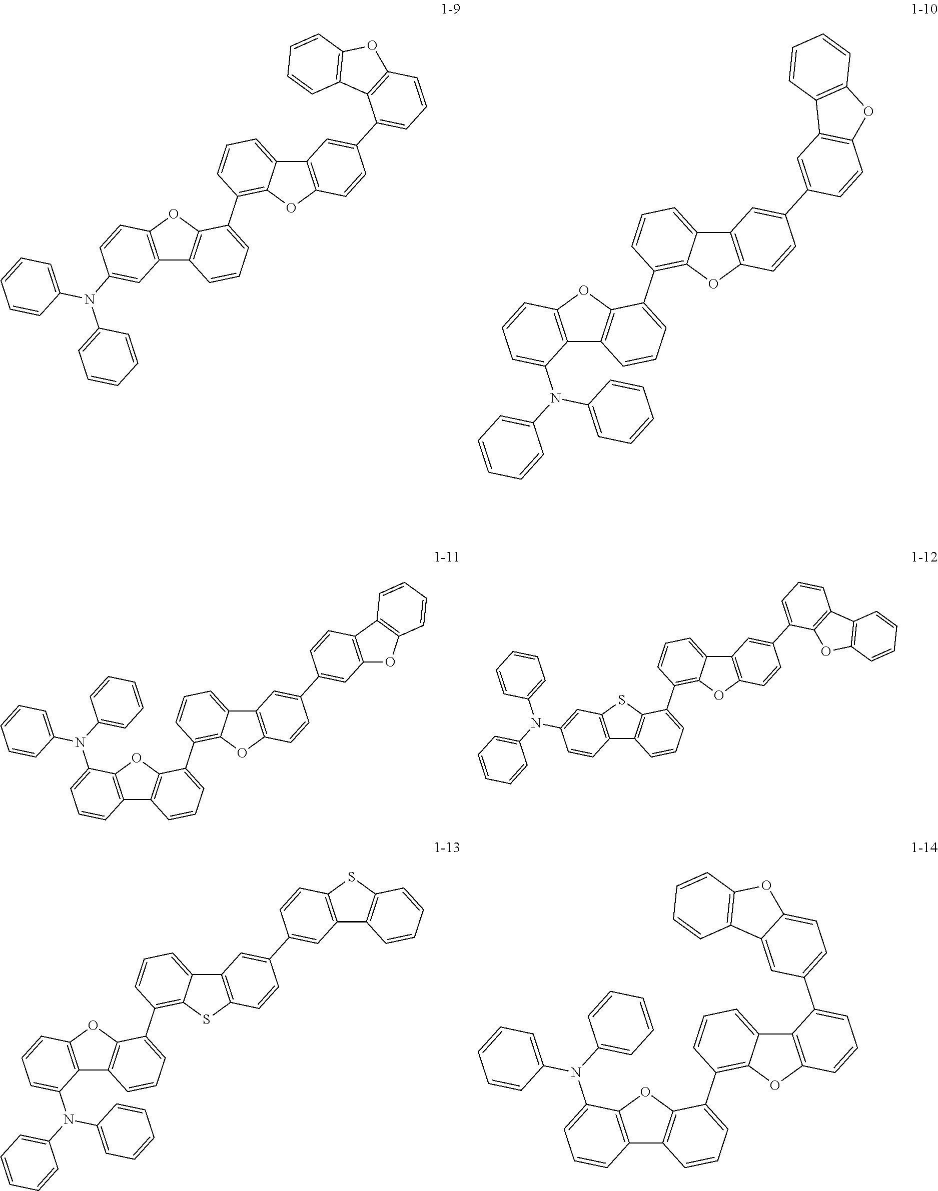

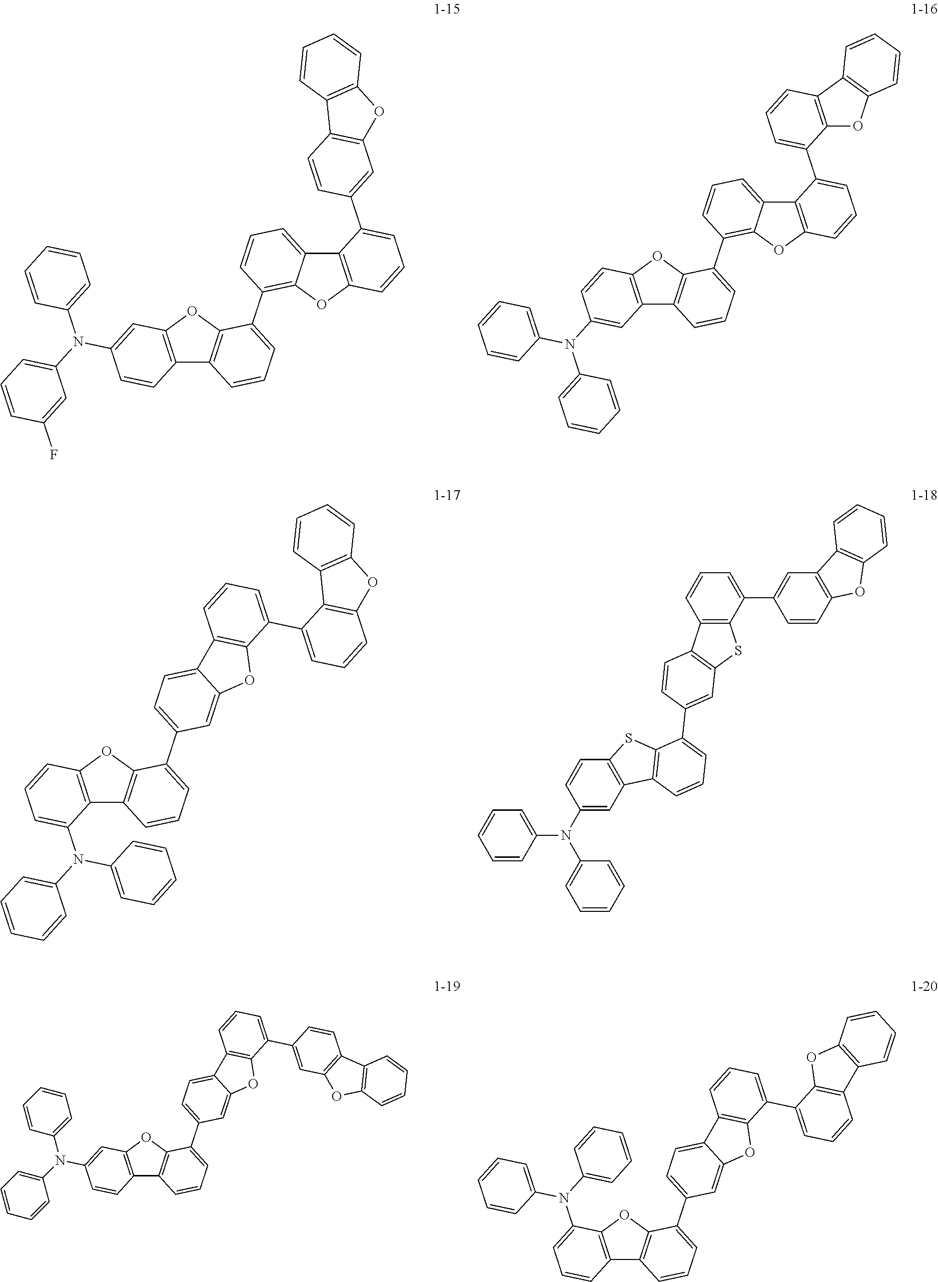

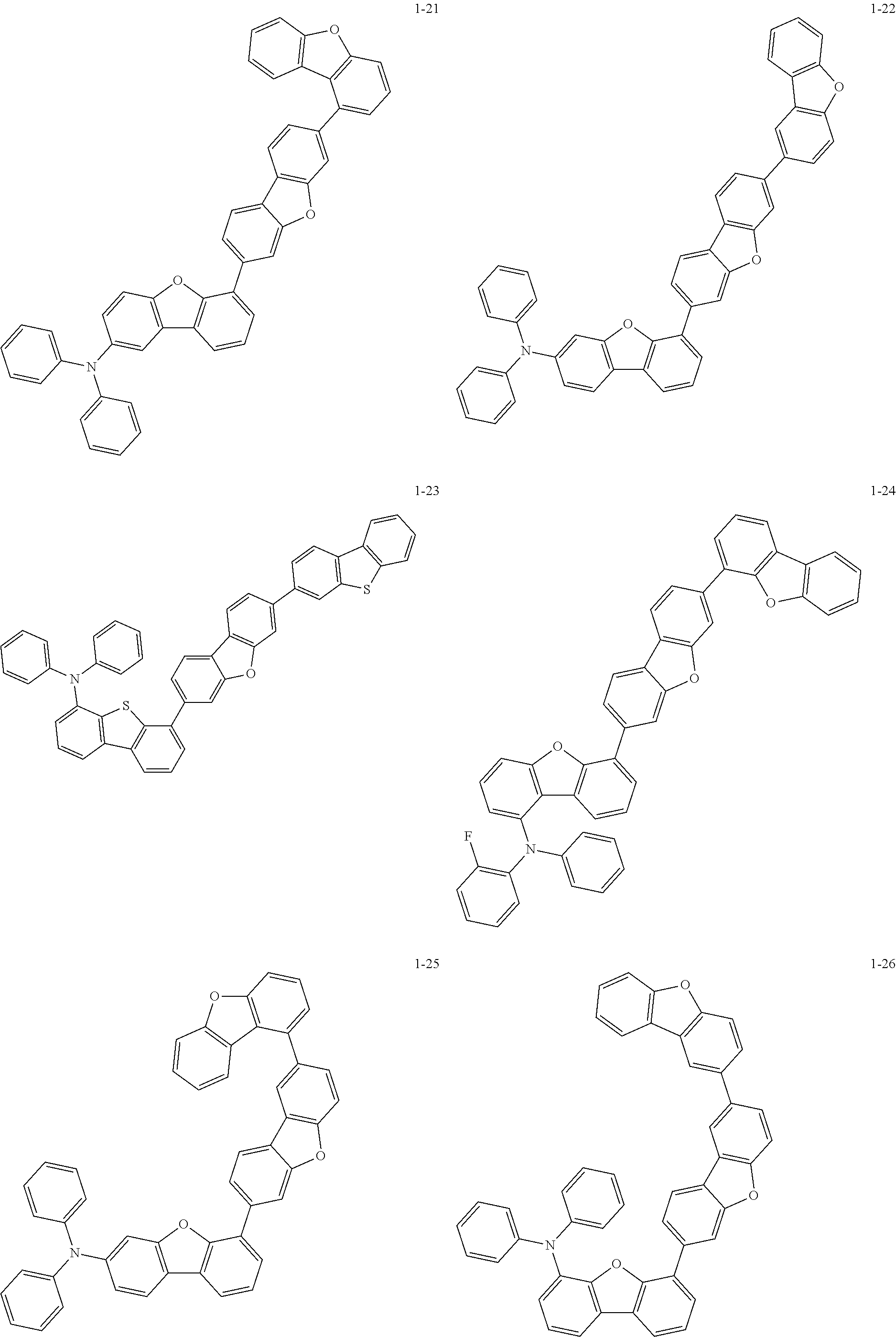

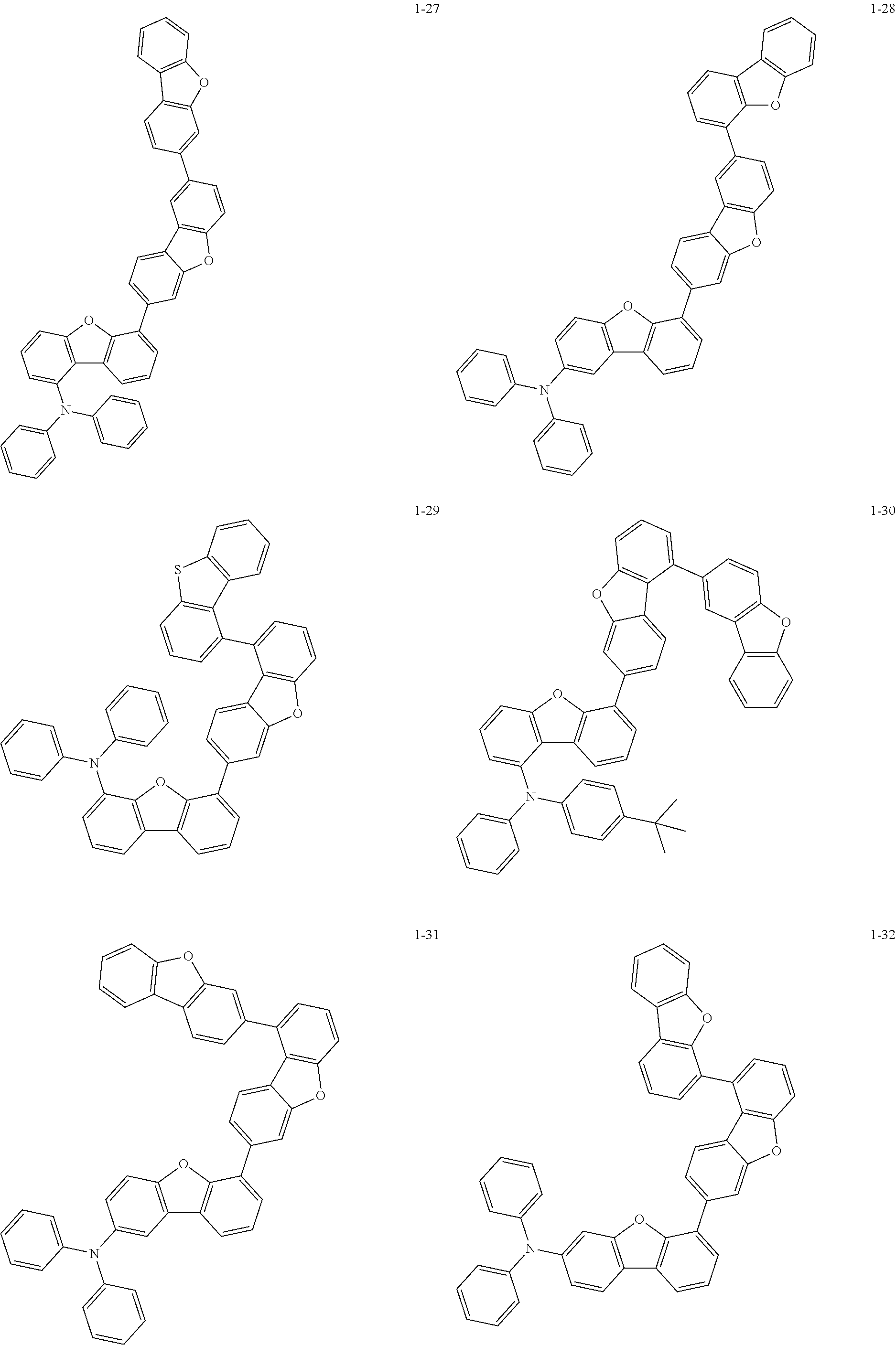

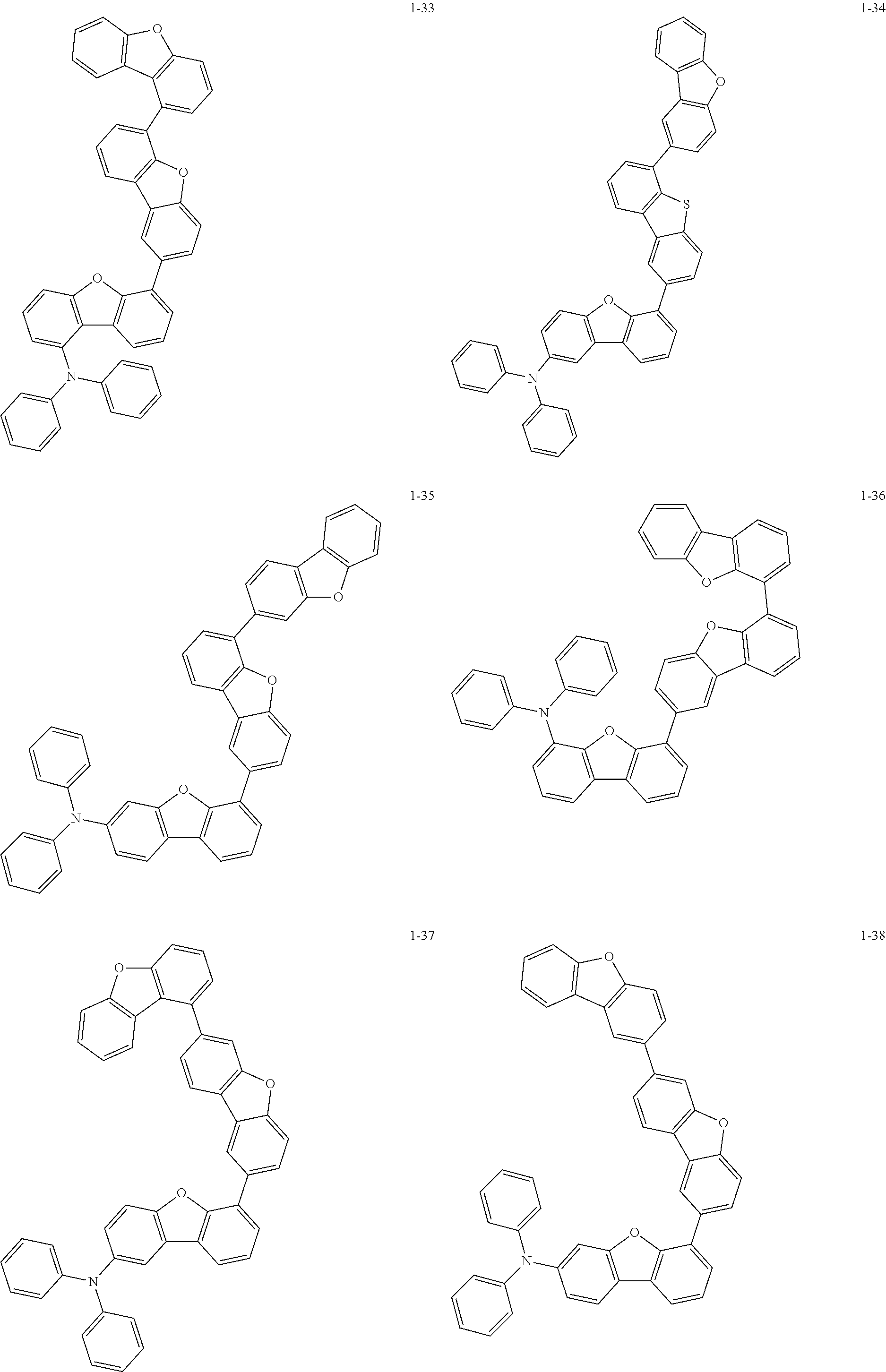

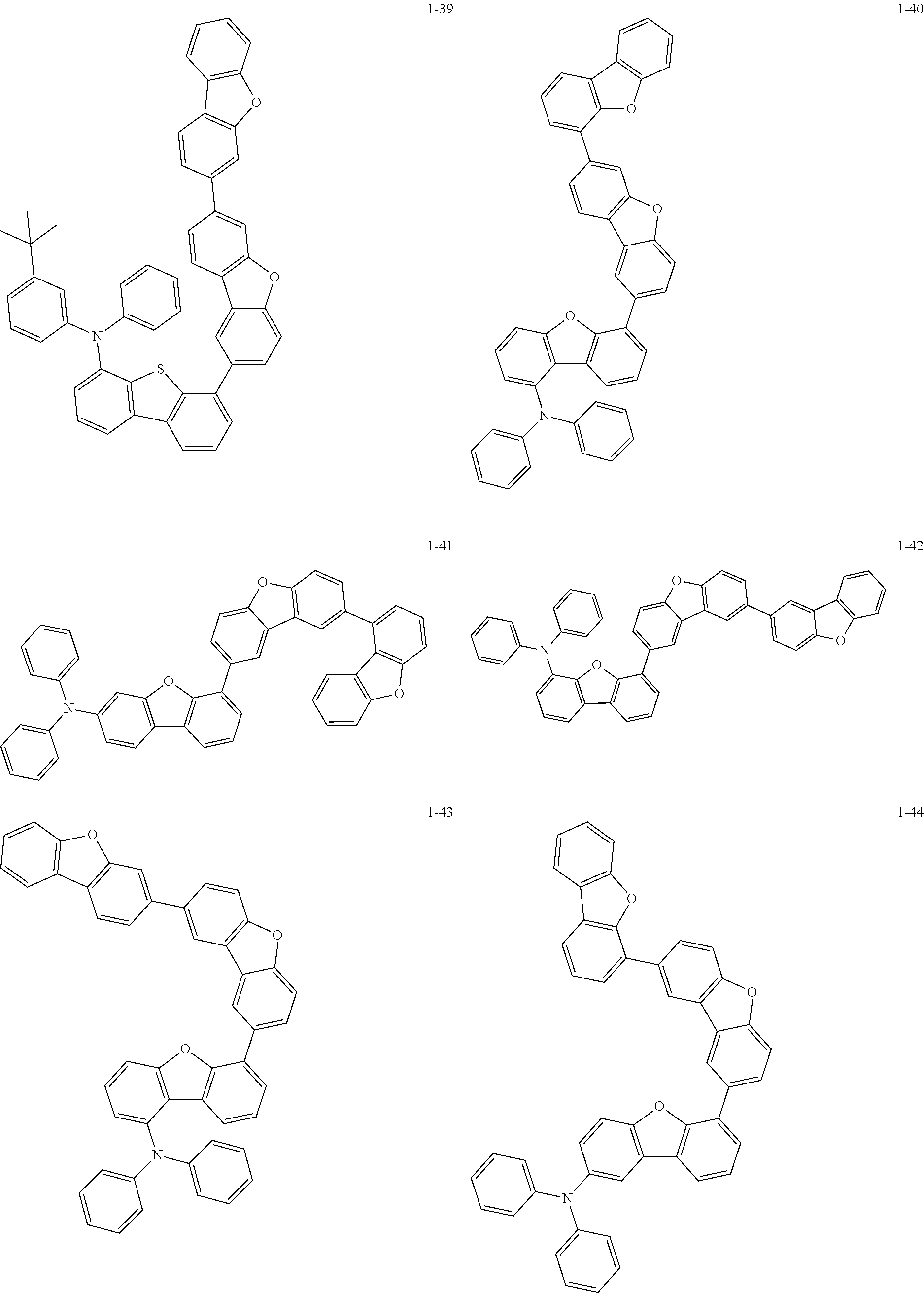

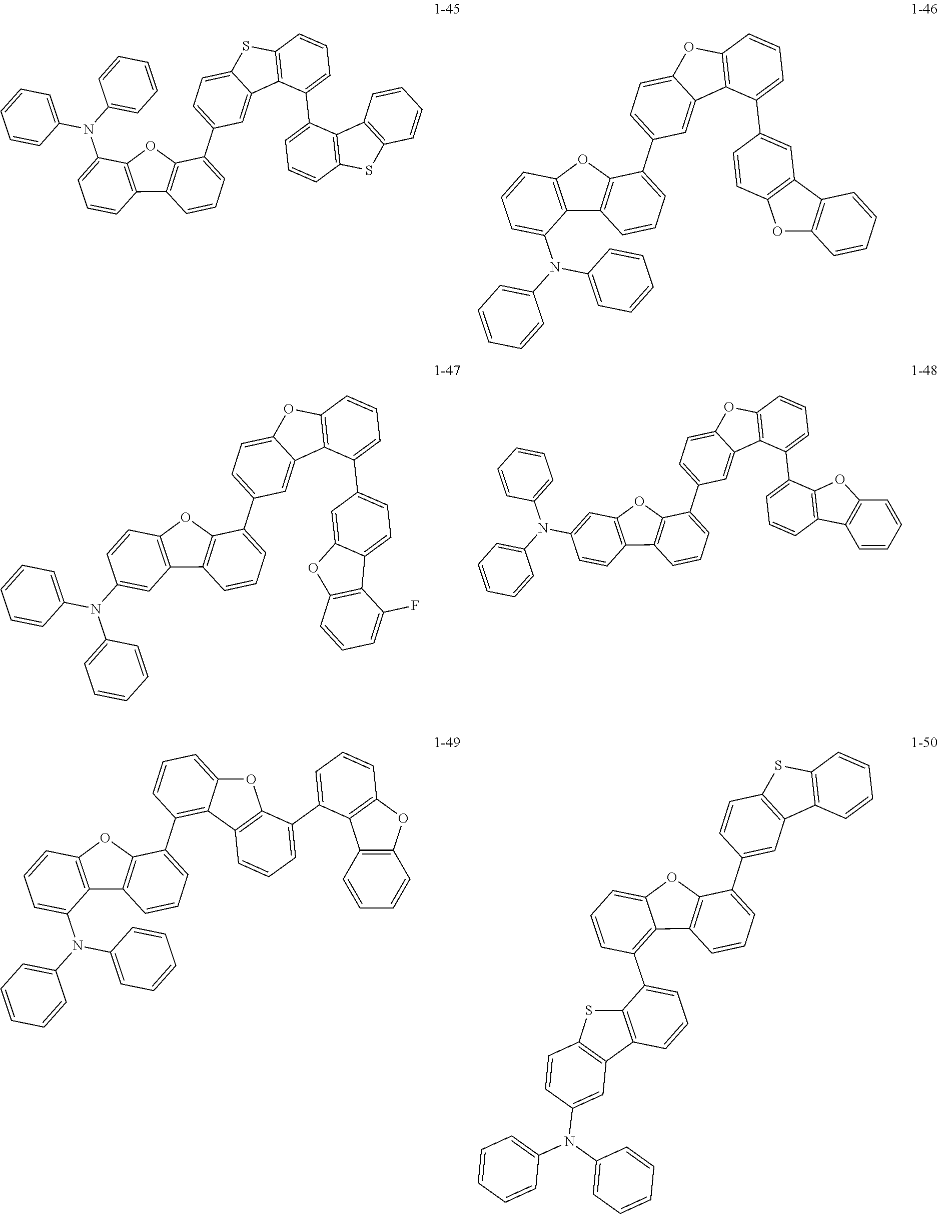

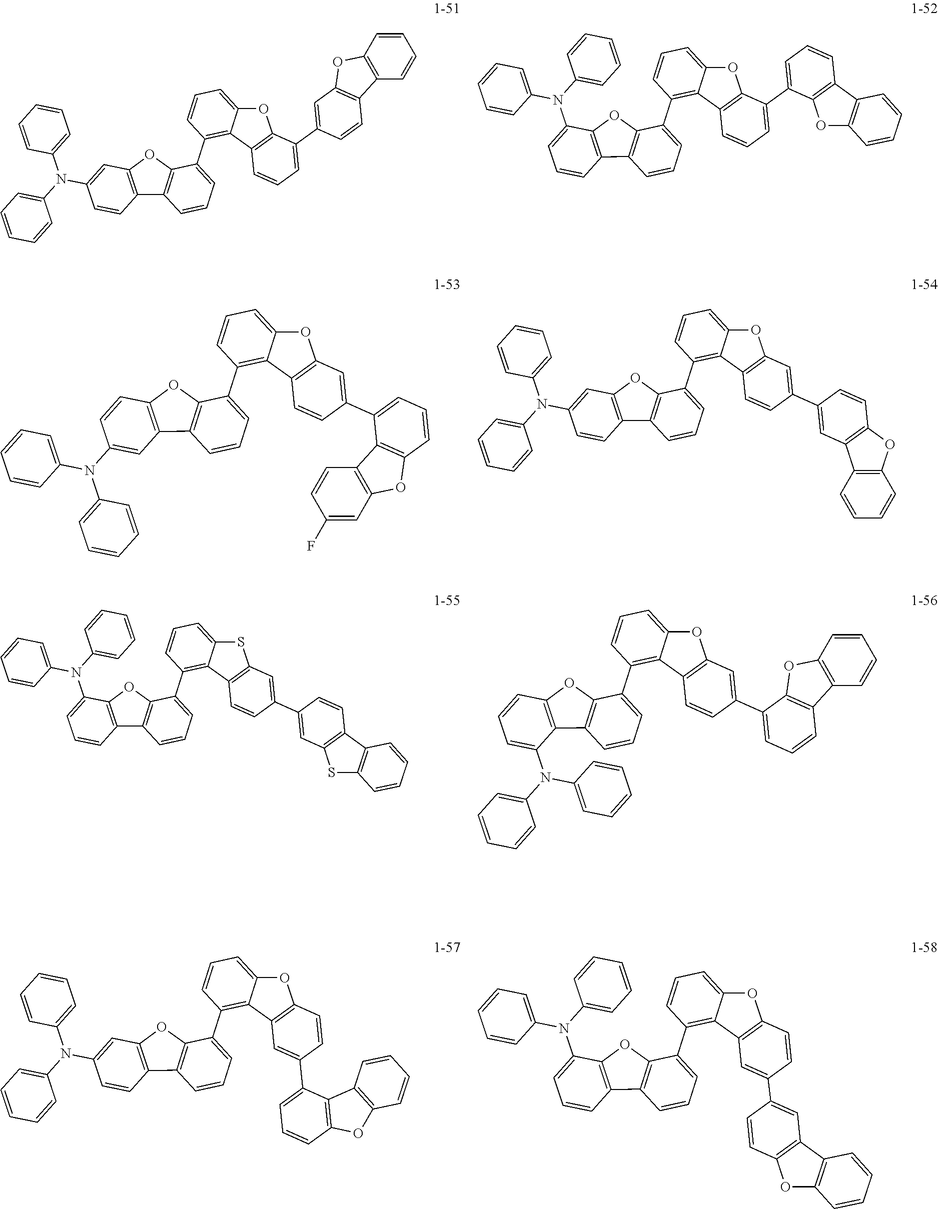

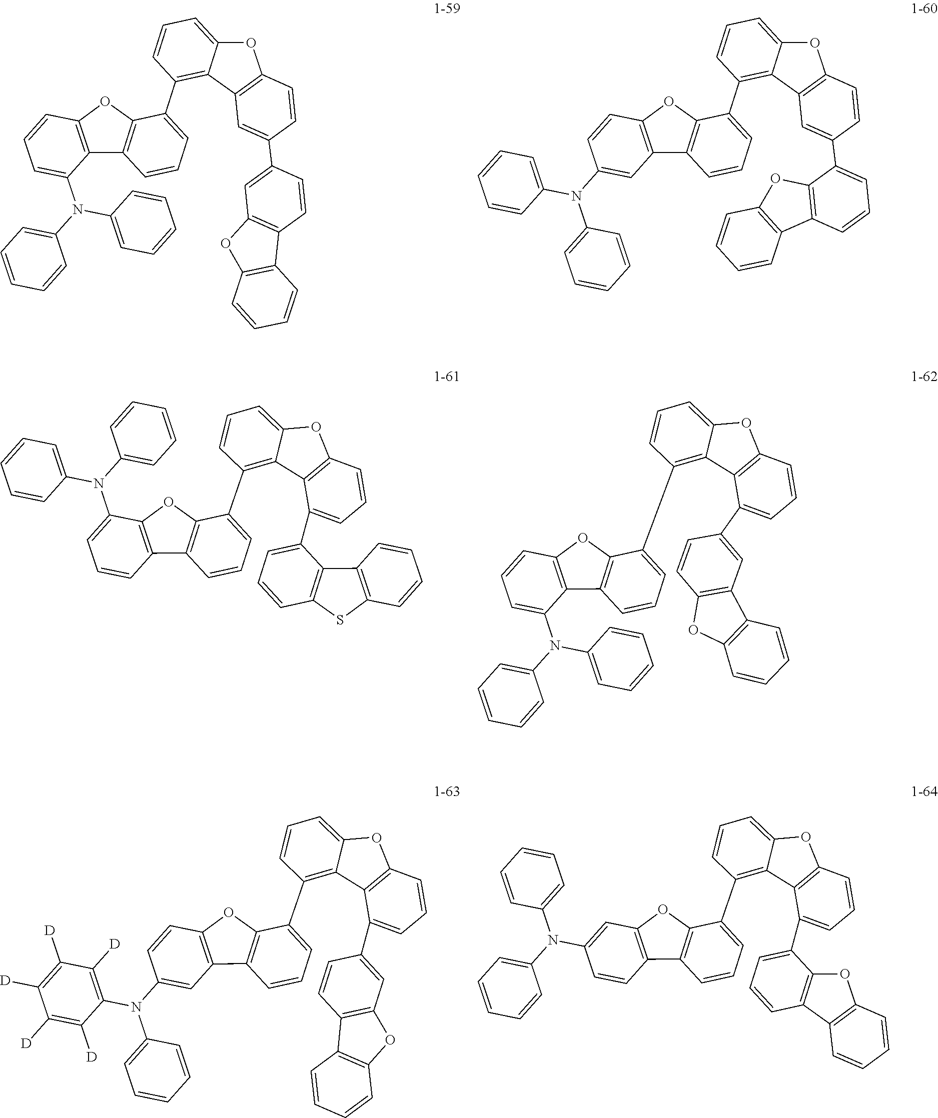

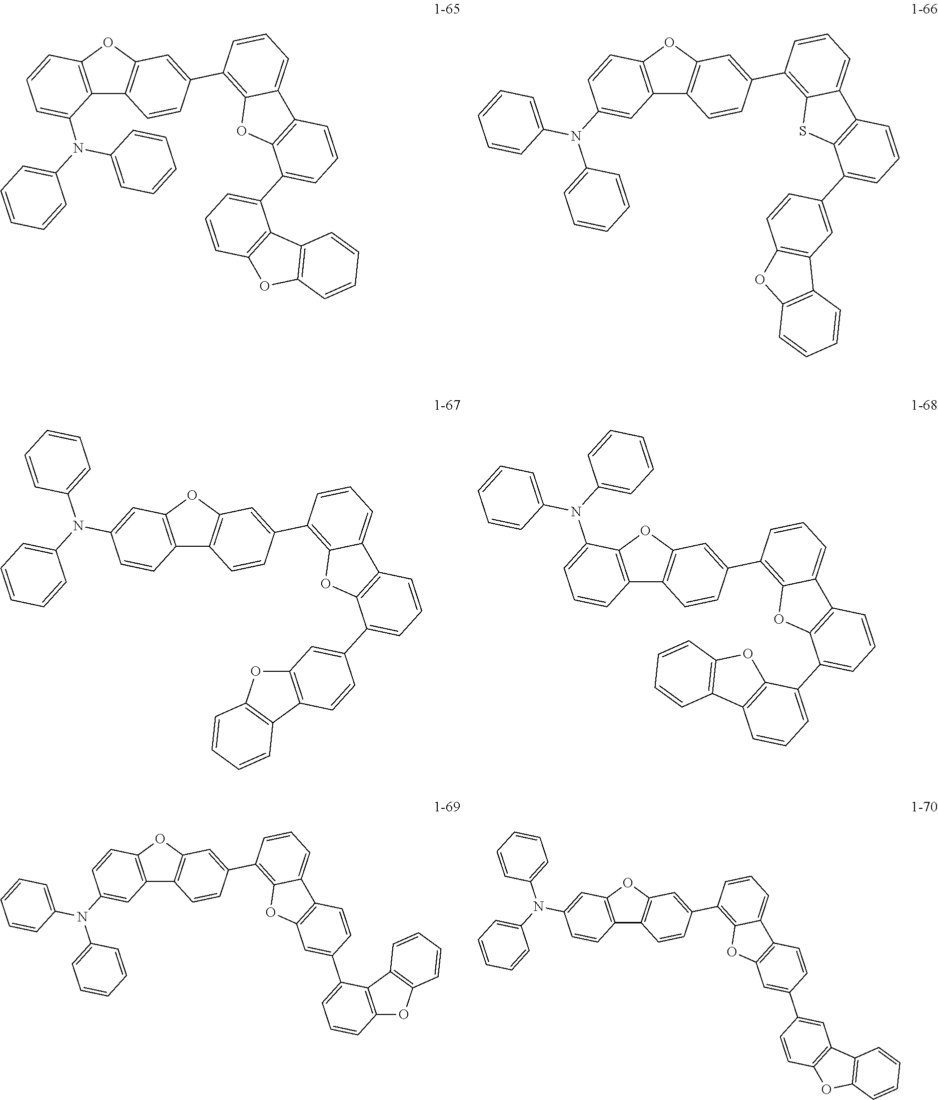

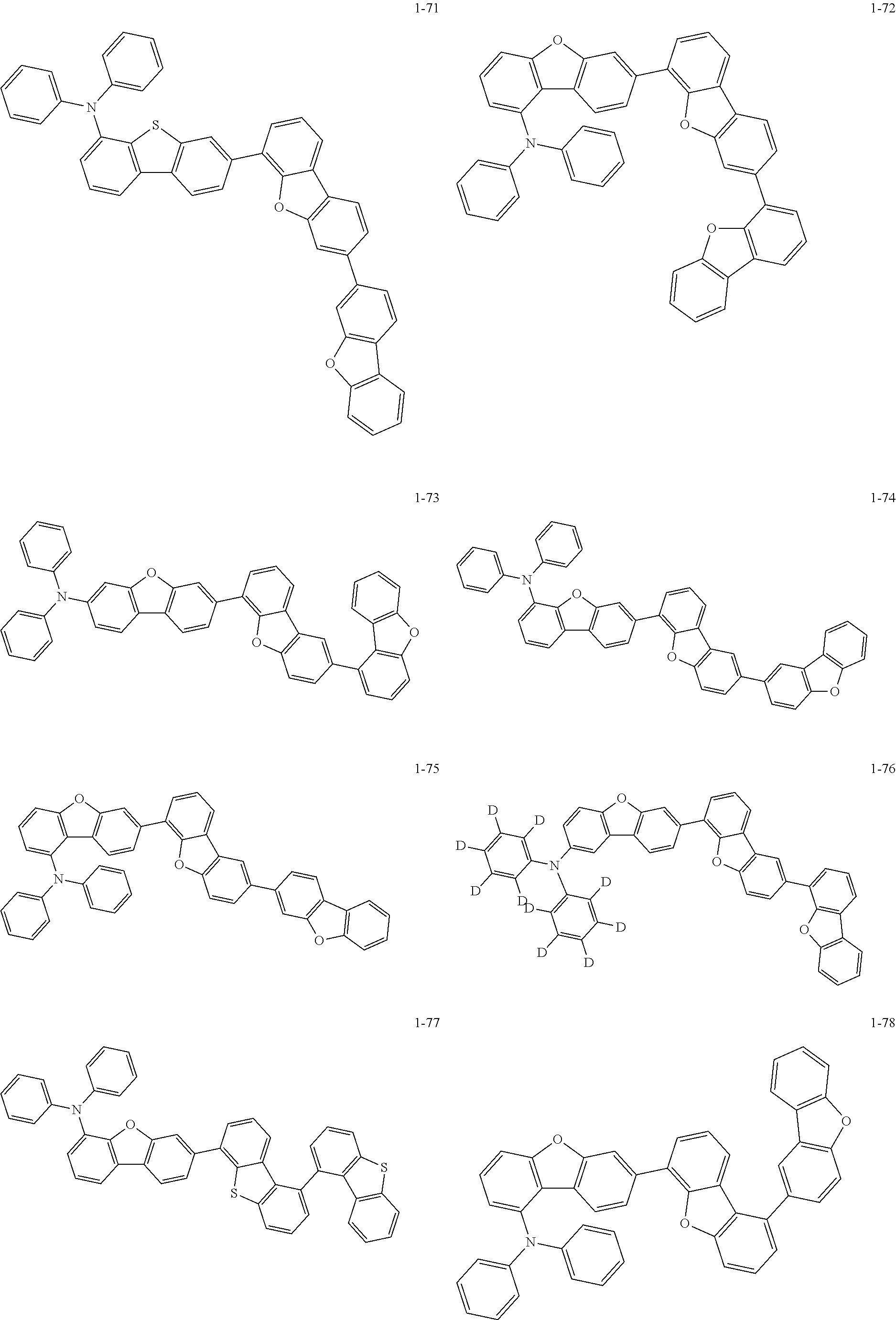

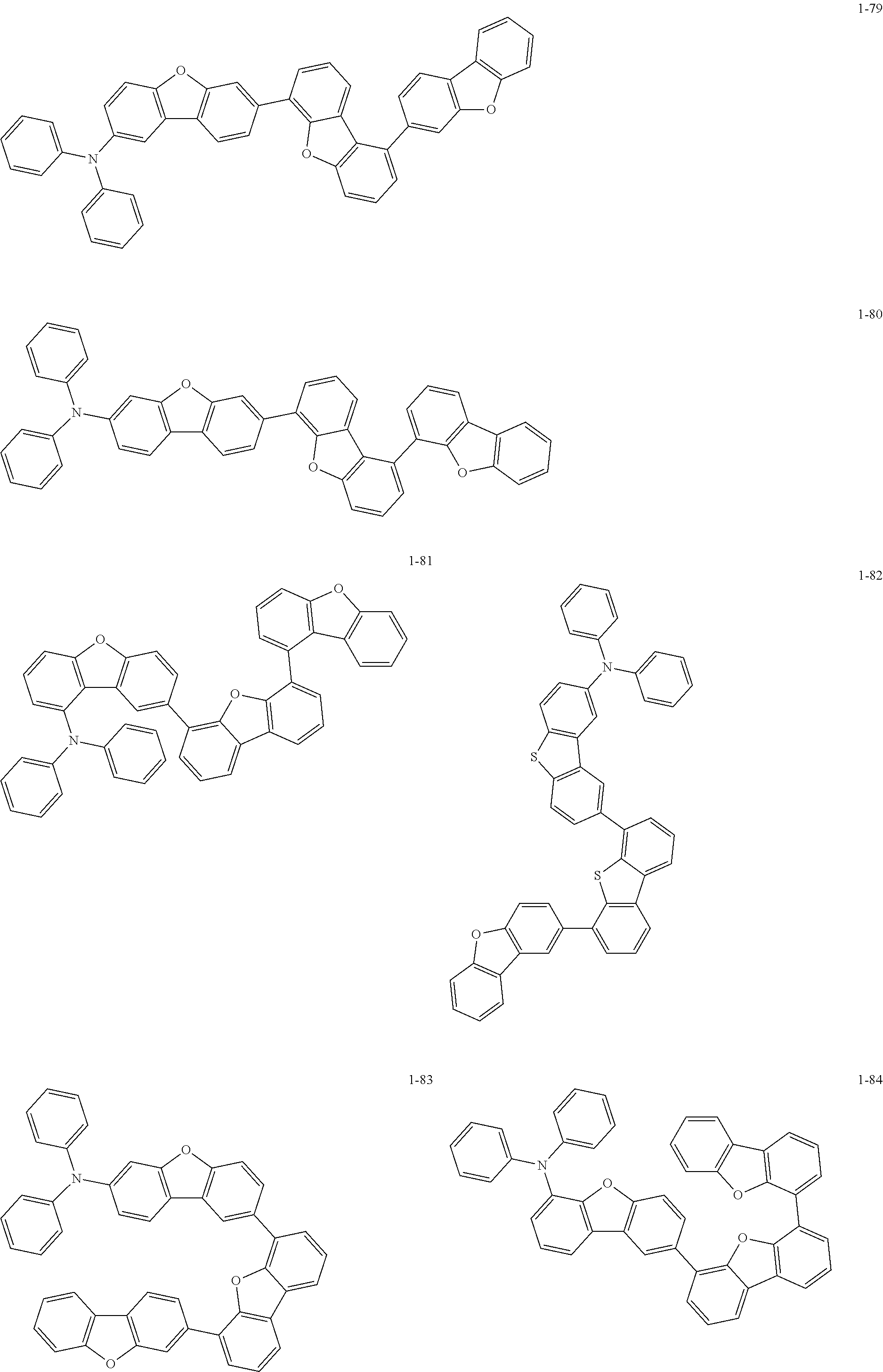

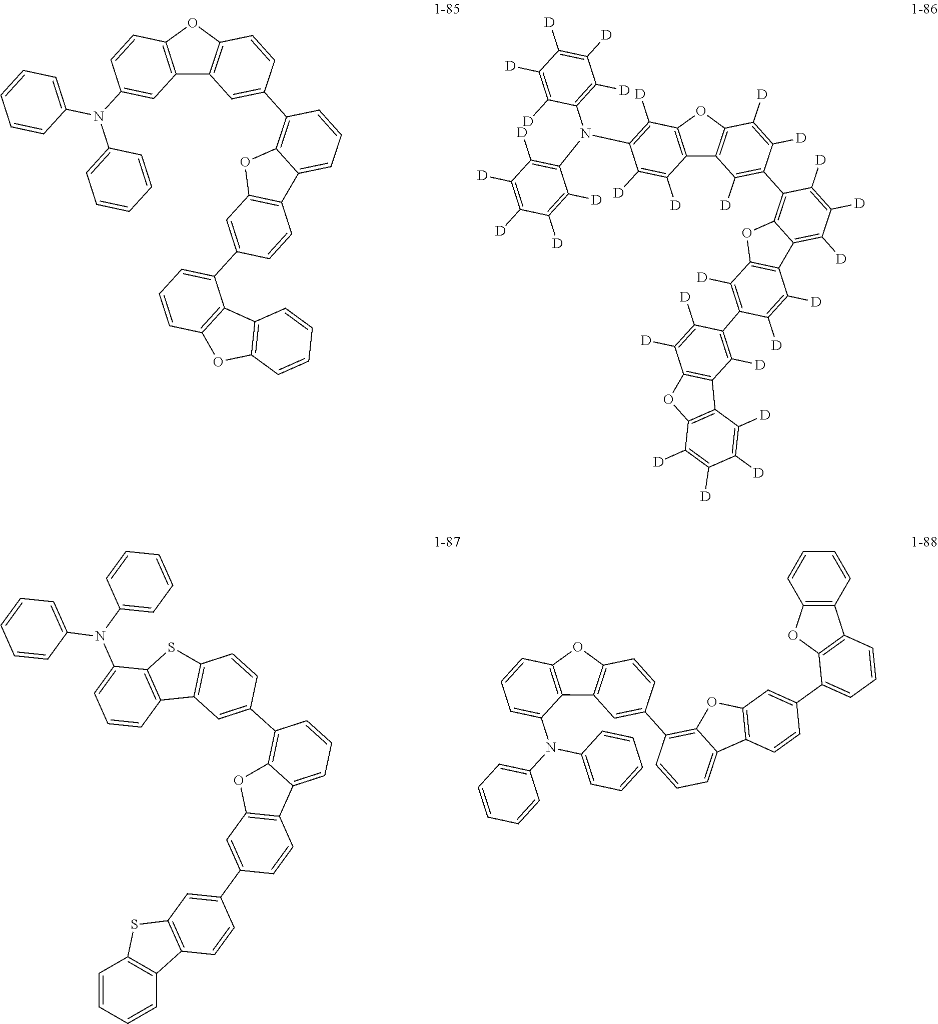

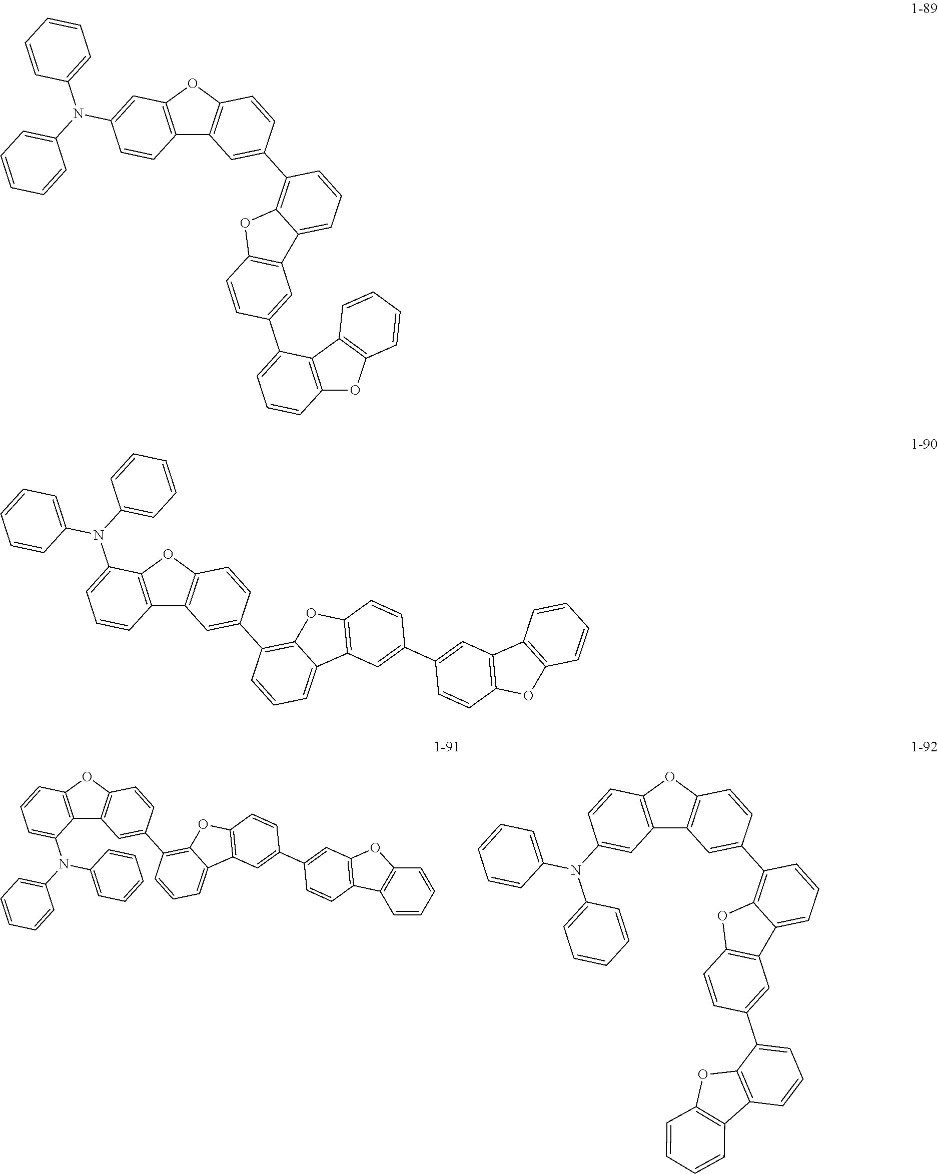 [SMILES: c1ccc(N(c2ccccc2)c2ccc3c(c2)oc2ccc(-c4cccc5c4oc4ccc(-c6cccc7oc8ccccc8c67)cc45)cc23)cc1 ; c1ccc(N(c2ccccc2)c2cccc3c2oc2ccc(-c4cccc5c4oc4ccc(-c6ccc7oc8ccccc8c7c6)cc45)cc23)cc1 ; c1ccc(N(c2ccccc2)c2cccc3oc4ccc(-c5cccc6c5oc5ccc(-c7ccc8c(c7)oc7ccccc78)cc56)cc4c23)cc1 ; c1ccc(N(c2ccccc2)c2ccc3oc4ccc(-c5cccc6c5oc5ccc(-c7cccc8c7oc7ccccc78)cc56)cc4c3c2)cc1]

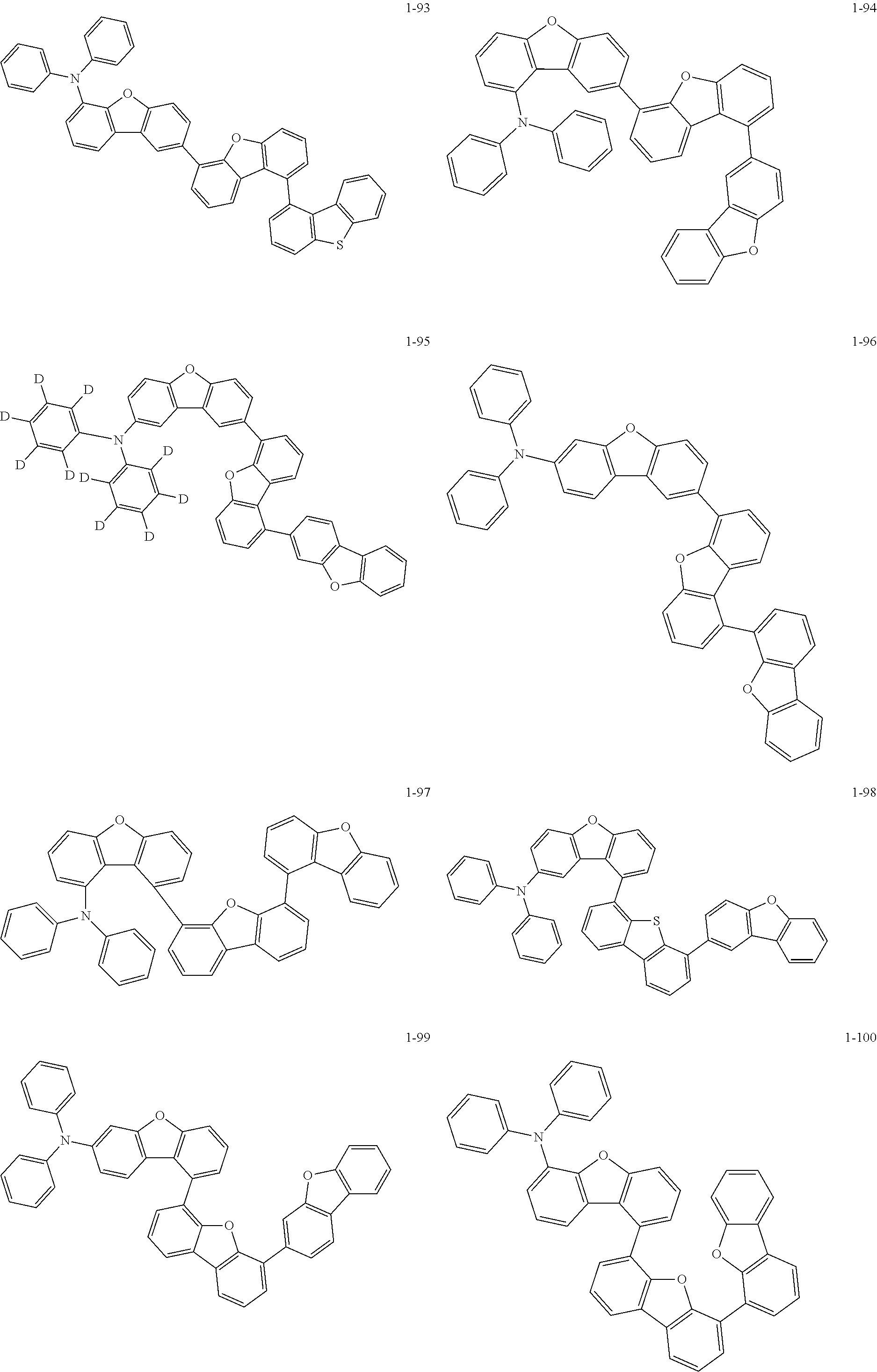

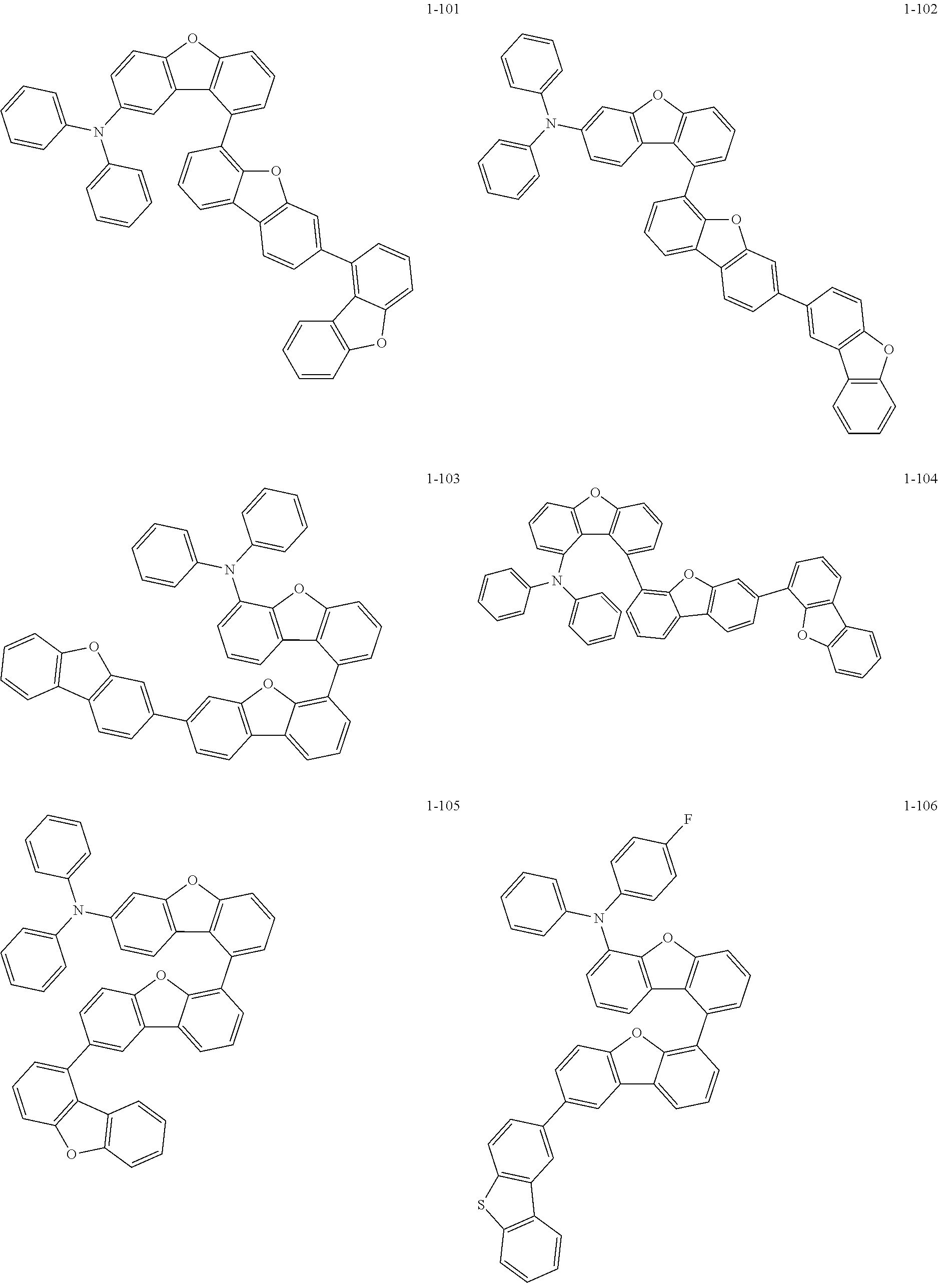

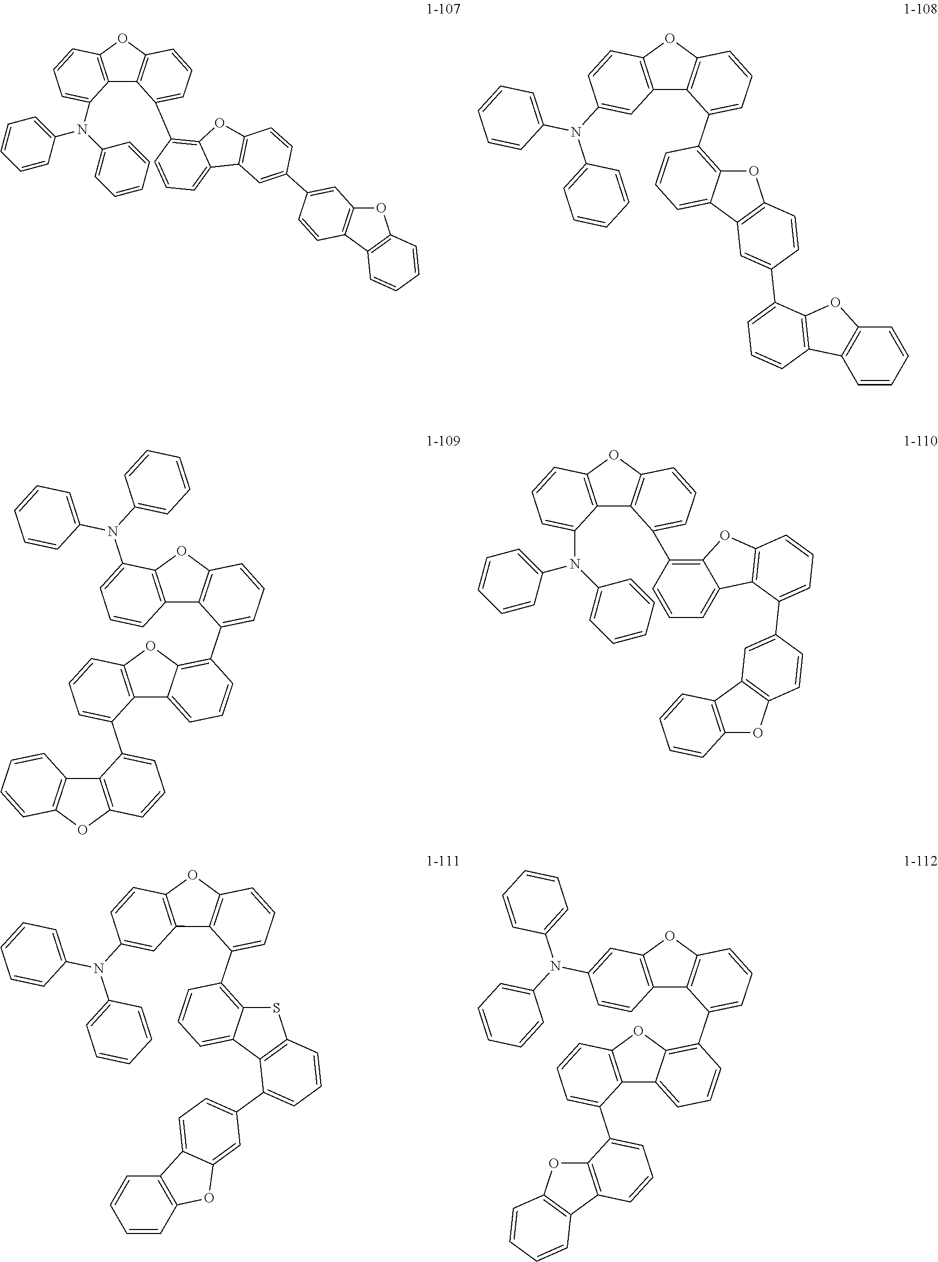

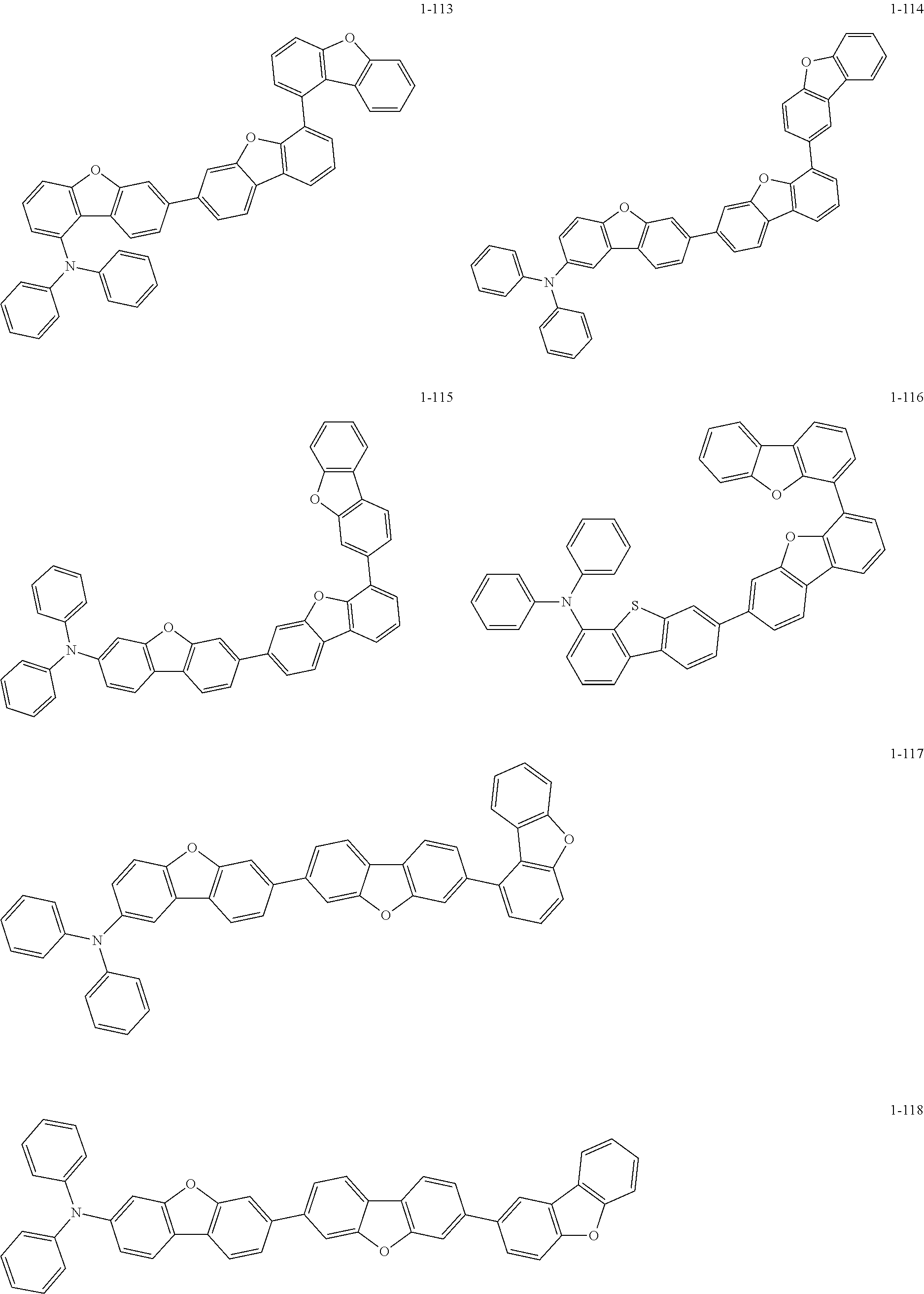

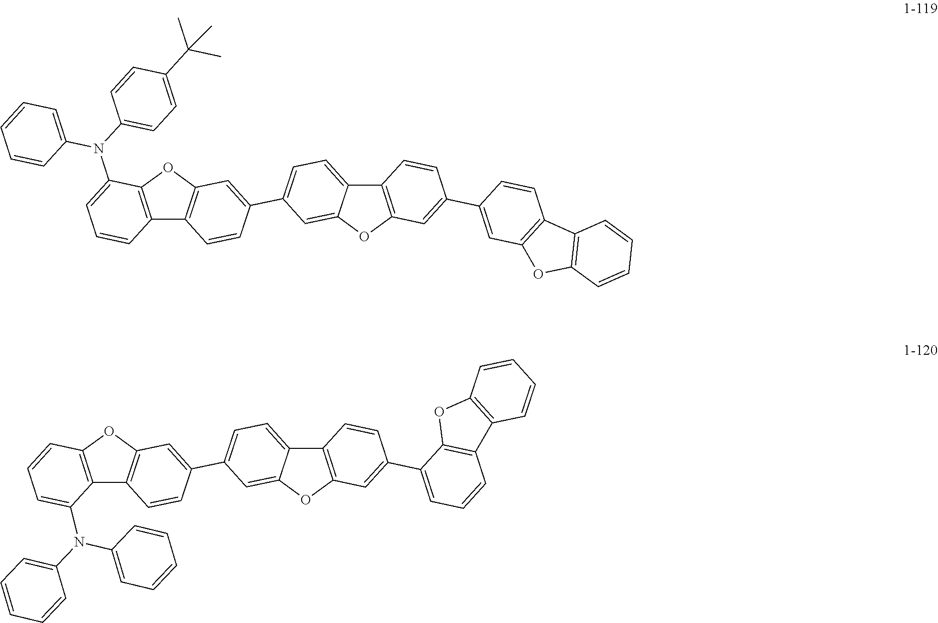 [SMILES: CC(C)(C)c1ccc(N(c2ccccc2)c2cccc3c2oc2cc(-c4ccc5c(c4)oc4cc(-c6ccc7c(c6)oc6ccccc67)ccc45)ccc23)cc1 ; c1ccc(N(c2ccccc2)c2cccc3oc4cc(-c5ccc6c(c5)oc5cc(-c7cccc8c7oc7ccccc78)ccc56)ccc4c23)cc1]

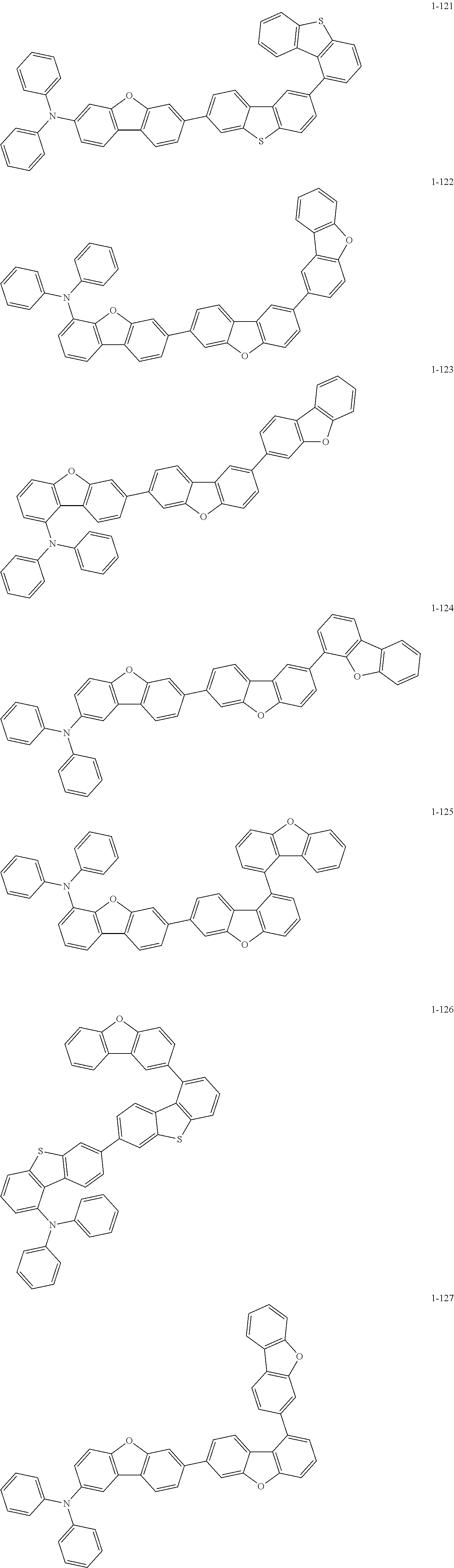

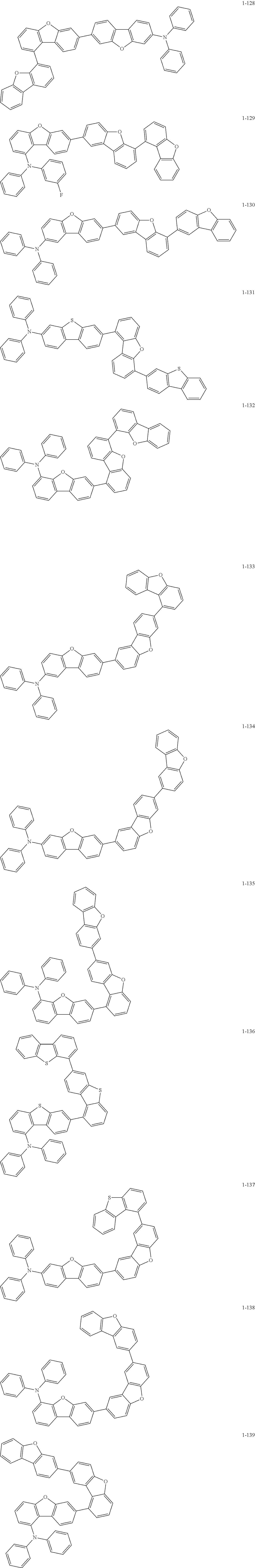

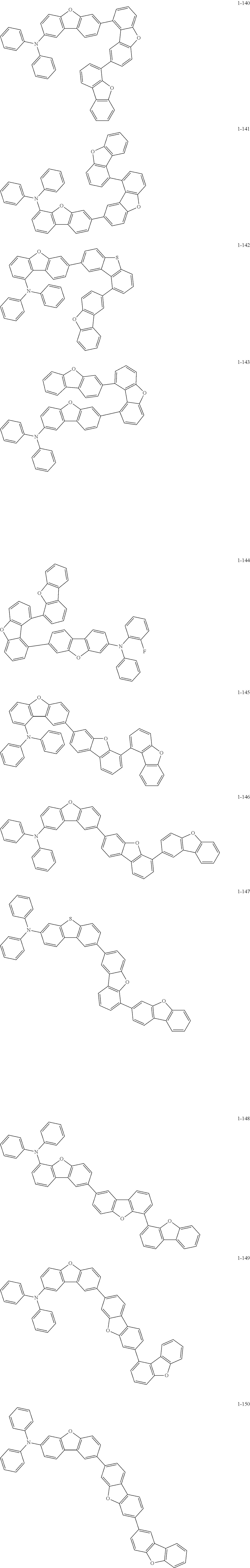

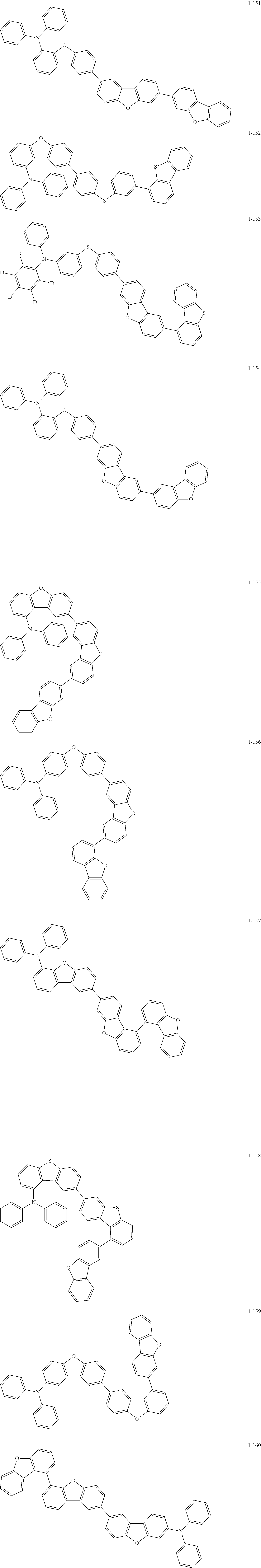

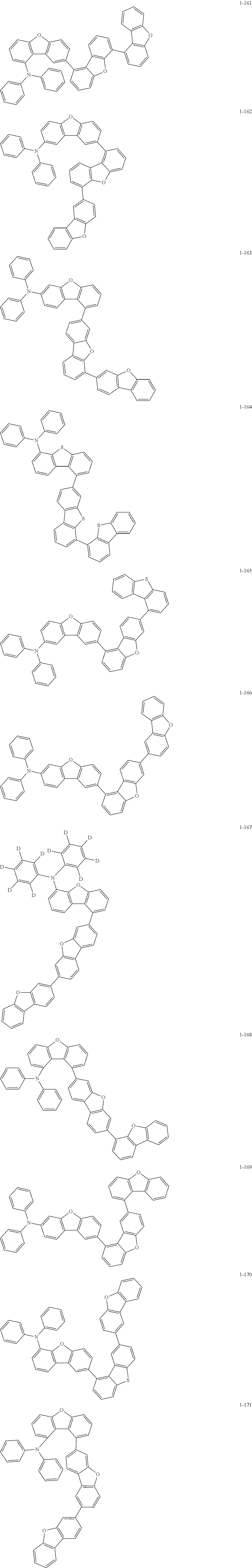

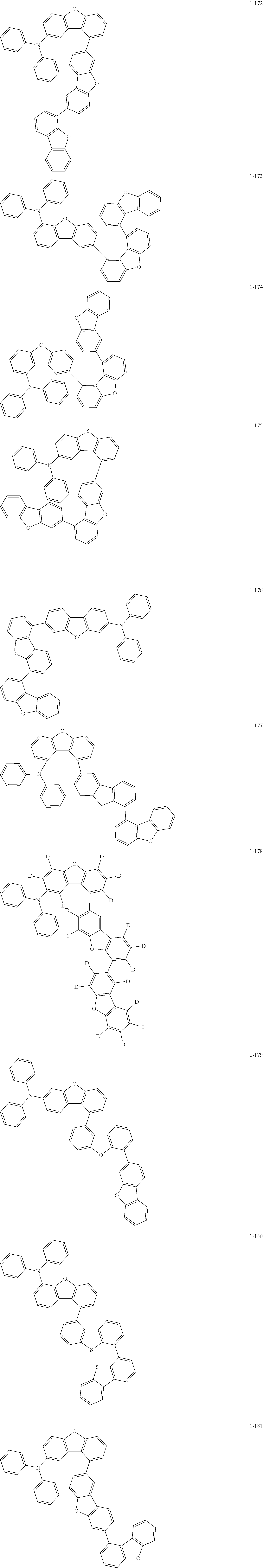

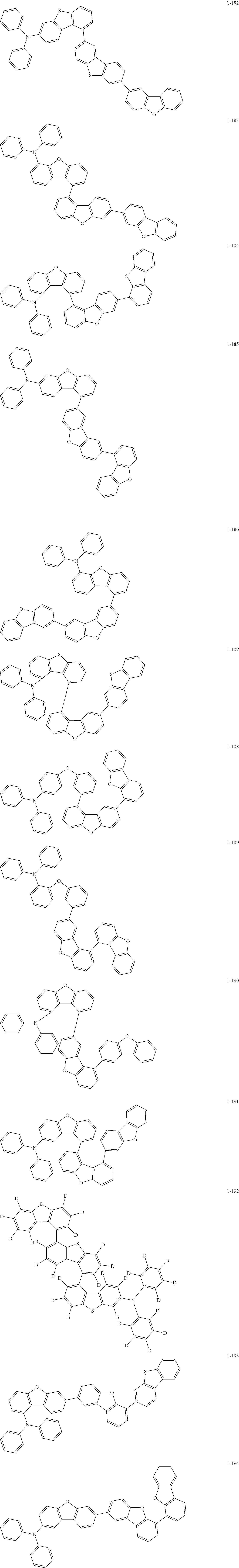

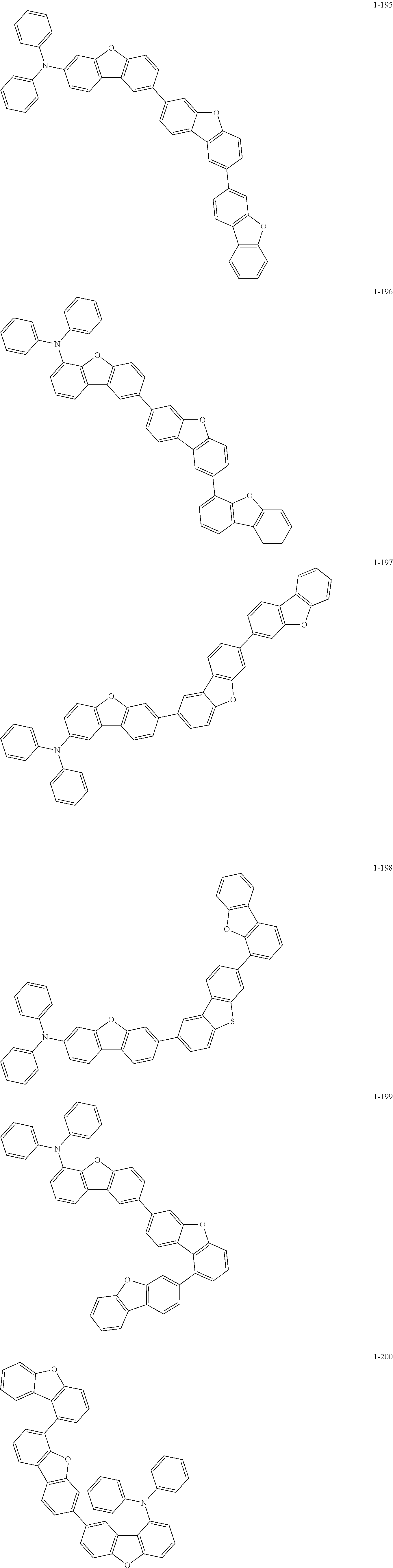

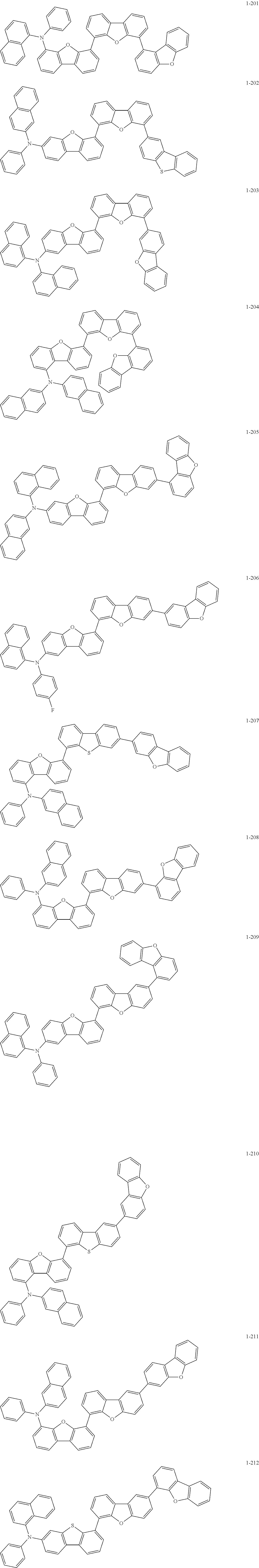

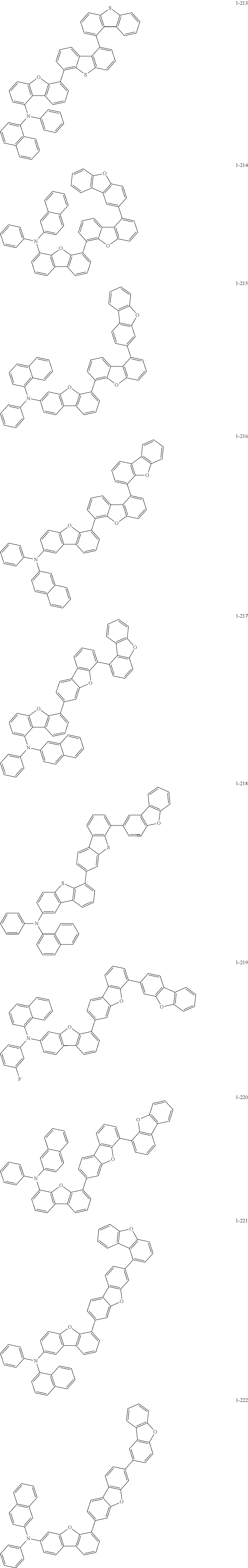

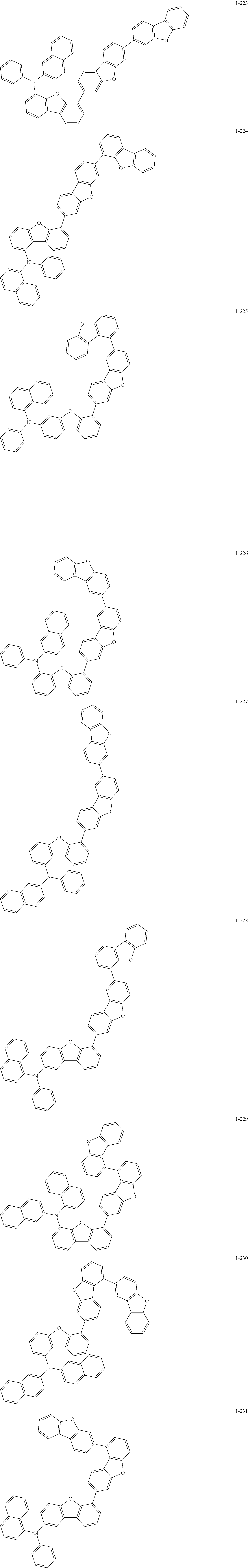

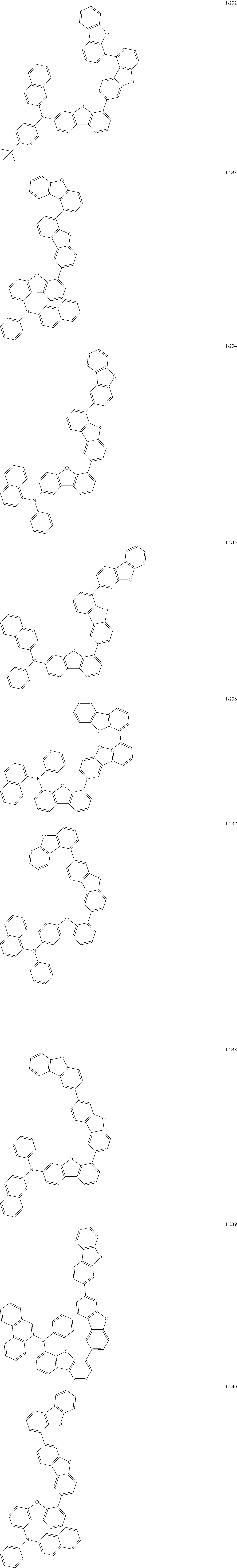

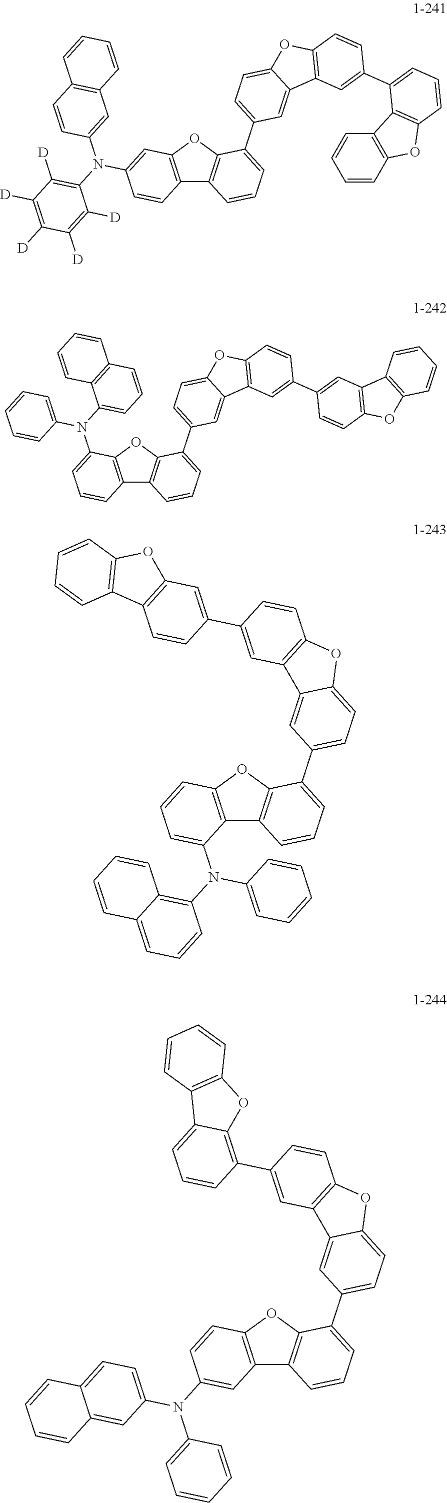

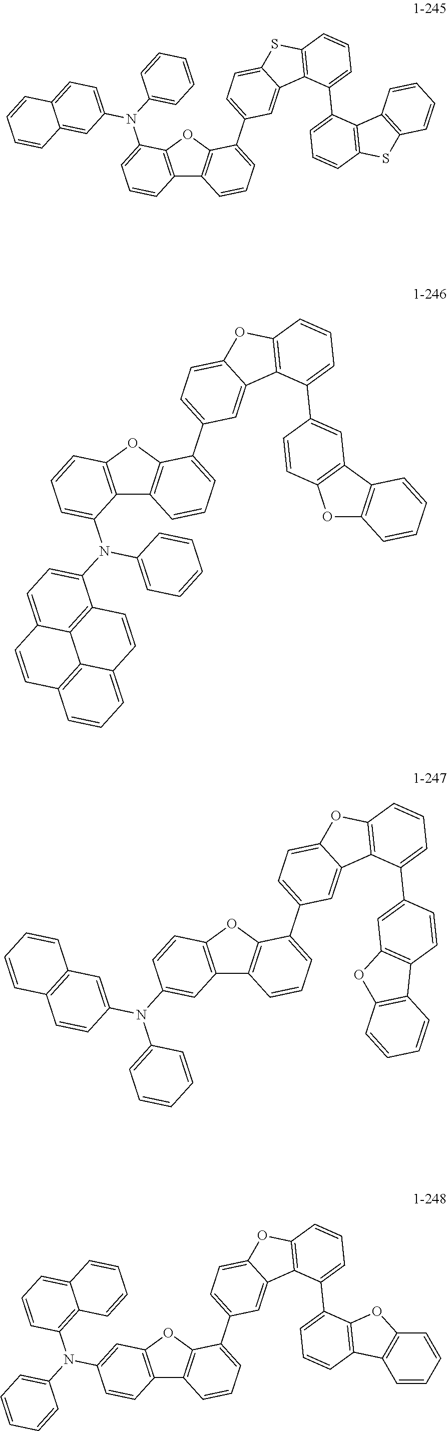 [SMILES: c1ccc(N(c2ccc3ccccc3c2)c2cccc3c2oc2c(-c4ccc5sc6cccc(-c7cccc8sc9ccccc9c78)c6c5c4)cccc23)cc1 ; c1ccc(N(c2ccc3ccc4cccc5ccc2c3c45)c2cccc3oc4c(-c5ccc6oc7cccc(-c8ccc9oc%10ccccc%10c9c8)c7c6c5)cccc4c23)cc1 ; c1ccc(N(c2ccc3ccccc3c2)c2ccc3oc4c(-c5ccc6oc7cccc(-c8ccc9c(c8)oc8ccccc89)c7c6c5)cccc4c3c2)cc1 ; c1ccc(N(c2ccc3c(c2)oc2c(-c4ccc5oc6cccc(-c7cccc8c7oc7ccccc78)c6c5c4)cccc23)c2cccc3ccccc23)cc1]

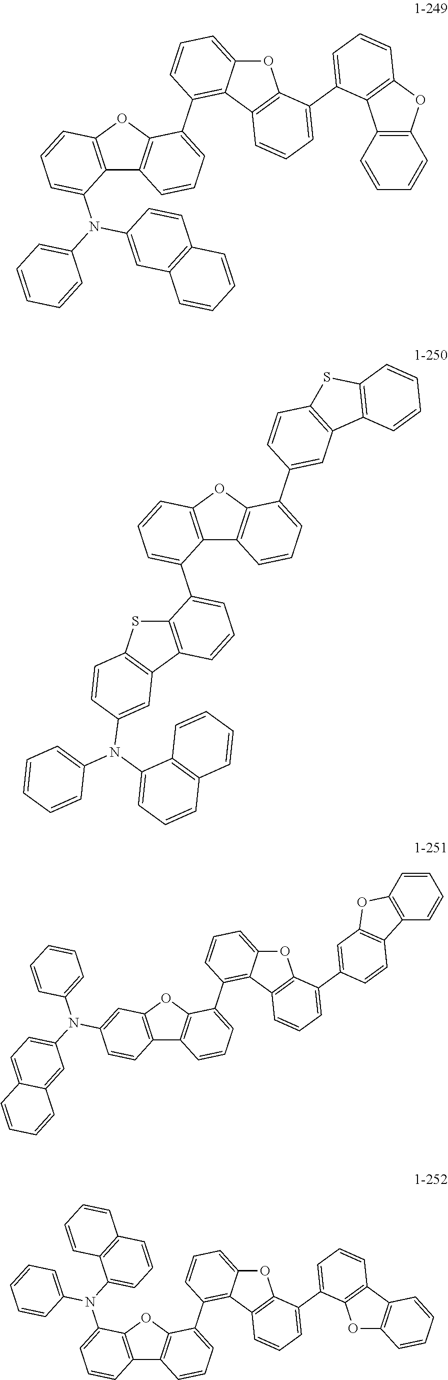 [SMILES: c1ccc(N(c2ccc3ccccc3c2)c2cccc3oc4c(-c5cccc6oc7c(-c8cccc9oc%10ccccc%10c89)cccc7c56)cccc4c23)cc1 ; c1ccc(N(c2ccc3sc4c(-c5cccc6oc7c(-c8ccc9sc%10ccccc%10c9c8)cccc7c56)cccc4c3c2)c2cccc3ccccc23)cc1 ; c1ccc(N(c2ccc3ccccc3c2)c2ccc3c(c2)oc2c(-c4cccc5oc6c(-c7ccc8c(c7)oc7ccccc78)cccc6c45)cccc23)cc1 ; c1ccc(N(c2cccc3ccccc23)c2cccc3c2oc2c(-c4cccc5oc6c(-c7cccc8c7oc7ccccc78)cccc6c45)cccc23)cc1]

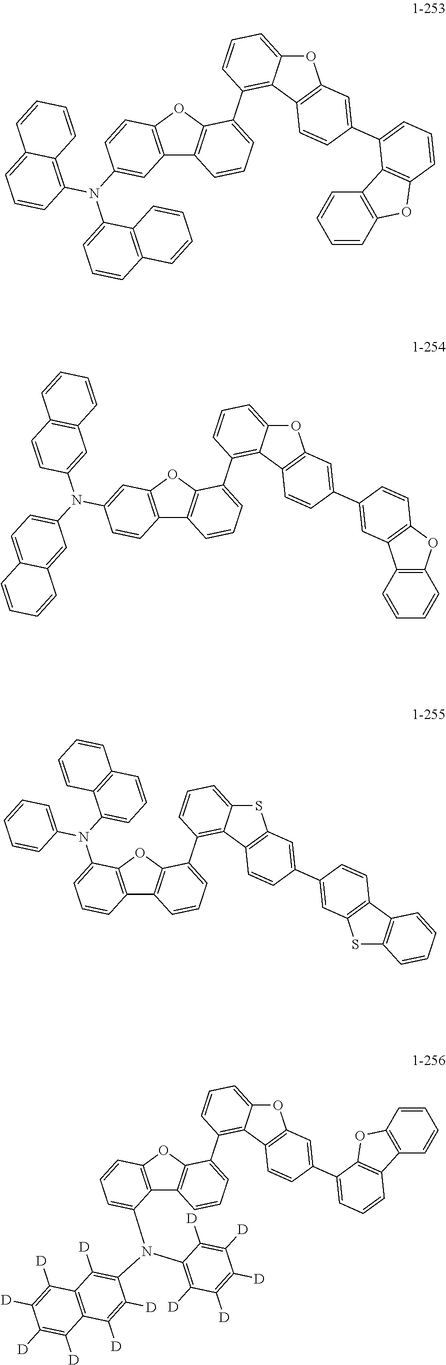

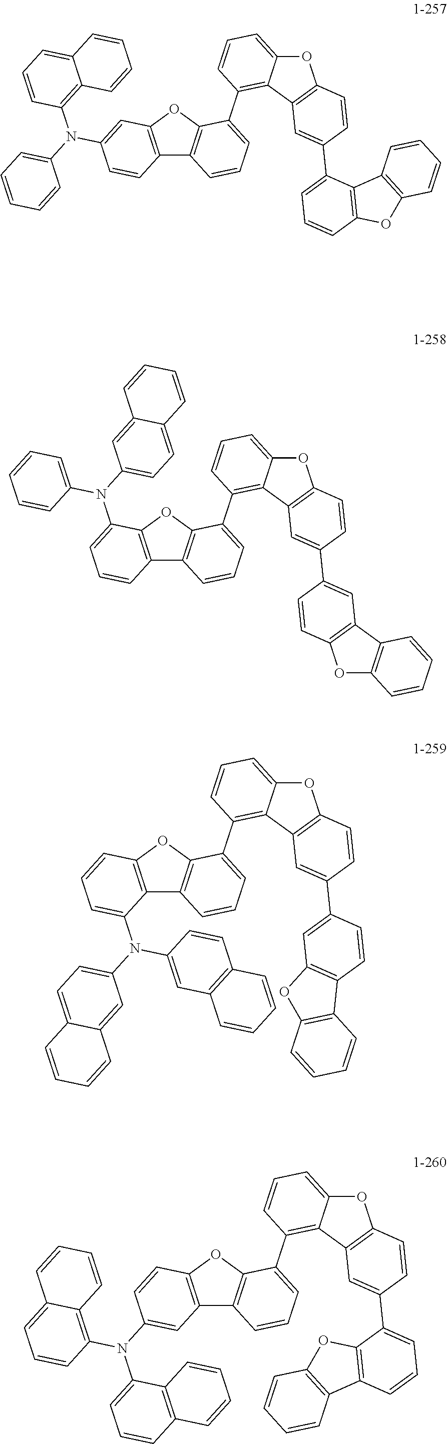 [SMILES: c1ccc(N(c2ccc3c(c2)oc2c(-c4cccc5oc6ccc(-c7cccc8oc9ccccc9c78)cc6c45)cccc23)c2cccc3ccccc23)cc1 ; c1ccc(N(c2ccc3ccccc3c2)c2cccc3c2oc2c(-c4cccc5oc6ccc(-c7ccc8oc9ccccc9c8c7)cc6c45)cccc23)cc1 ; c1ccc2cc(N(c3ccc4ccccc4c3)c3cccc4oc5c(-c6cccc7oc8ccc(-c9ccc%10c(c9)oc9ccccc9%10)cc8c67)cccc5c34)ccc2c1 ; c1ccc2c(N(c3ccc4oc5c(-c6cccc7oc8ccc(-c9cccc%10c9oc9ccccc9%10)cc8c67)cccc5c4c3)c3cccc4ccccc34)cccc2c1]

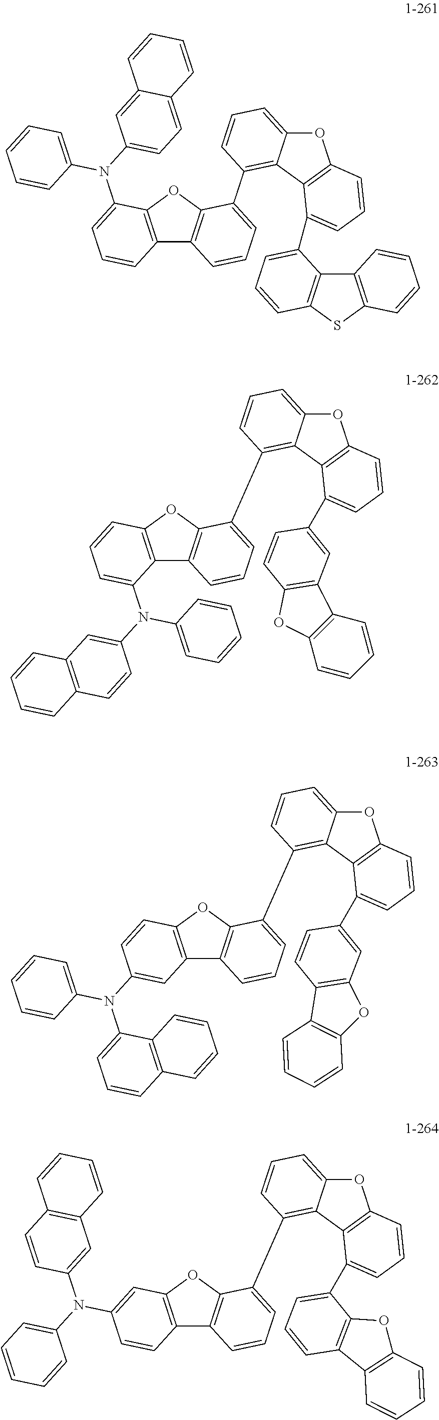 [SMILES: c1ccc(N(c2ccc3ccccc3c2)c2cccc3c2oc2c(-c4cccc5oc6cccc(-c7cccc8sc9ccccc9c78)c6c45)cccc23)cc1 ; c1ccc(N(c2ccc3ccccc3c2)c2cccc3oc4c(-c5cccc6oc7cccc(-c8ccc9oc%10ccccc%10c9c8)c7c56)cccc4c23)cc1 ; c1ccc(N(c2ccc3oc4c(-c5cccc6oc7cccc(-c8ccc9c(c8)oc8ccccc89)c7c56)cccc4c3c2)c2cccc3ccccc23)cc1 ; c1ccc(N(c2ccc3ccccc3c2)c2ccc3c(c2)oc2c(-c4cccc5oc6cccc(-c7cccc8c7oc7ccccc78)c6c45)cccc23)cc1]

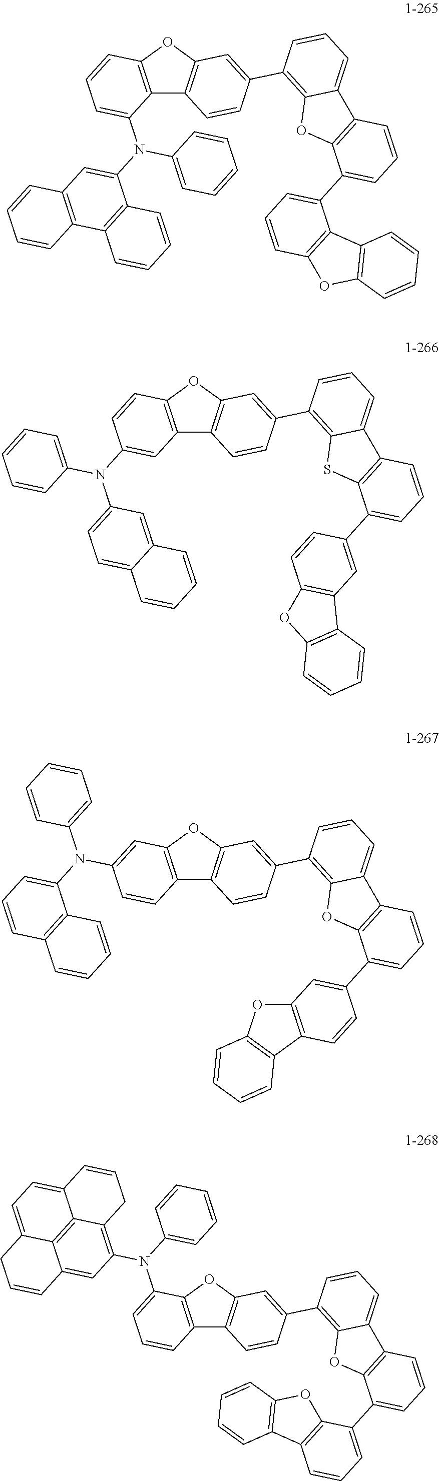 [SMILES: c1ccc(N(c2cc3ccccc3c3ccccc23)c2cccc3oc4cc(-c5cccc6c5oc5c(-c7cccc8oc9ccccc9c78)cccc56)ccc4c23)cc1 ; c1ccc(N(c2ccc3ccccc3c2)c2ccc3oc4cc(-c5cccc6c5sc5c(-c7ccc8oc9ccccc9c8c7)cccc56)ccc4c3c2)cc1 ; c1ccc(N(c2ccc3c(c2)oc2cc(-c4cccc5c4oc4c(-c6ccc7c(c6)oc6ccccc67)cccc45)ccc23)c2cccc3ccccc23)cc1 ; C1=Cc2cc(N(c3ccccc3)c3cccc4c3oc3cc(-c5cccc6c5oc5c(-c7cccc8c7oc7ccccc78)cccc56)ccc34)c3c4c(ccc(c24)C1)C=CC3]

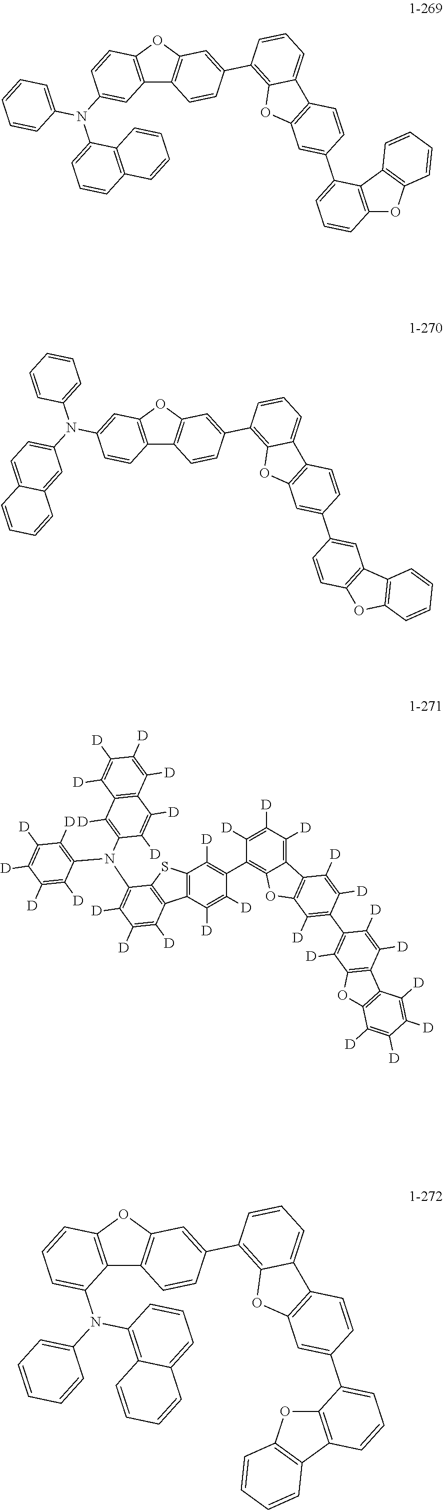

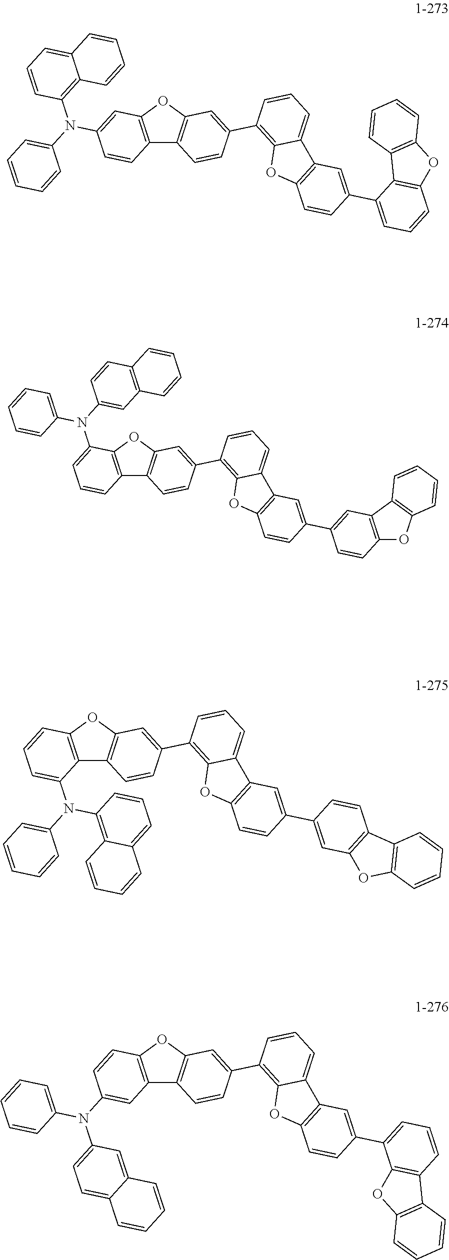 [SMILES: c1ccc(N(c2ccc3c(c2)oc2cc(-c4cccc5c4oc4ccc(-c6cccc7oc8ccccc8c67)cc45)ccc23)c2cccc3ccccc23)cc1 ; c1ccc(N(c2ccc3ccccc3c2)c2cccc3c2oc2cc(-c4cccc5c4oc4ccc(-c6ccc7oc8ccccc8c7c6)cc45)ccc23)cc1 ; c1ccc(N(c2cccc3ccccc23)c2cccc3oc4cc(-c5cccc6c5oc5ccc(-c7ccc8c(c7)oc7ccccc78)cc56)ccc4c23)cc1 ; c1ccc(N(c2ccc3ccccc3c2)c2ccc3oc4cc(-c5cccc6c5oc5ccc(-c7cccc8c7oc7ccccc78)cc56)ccc4c3c2)cc1]

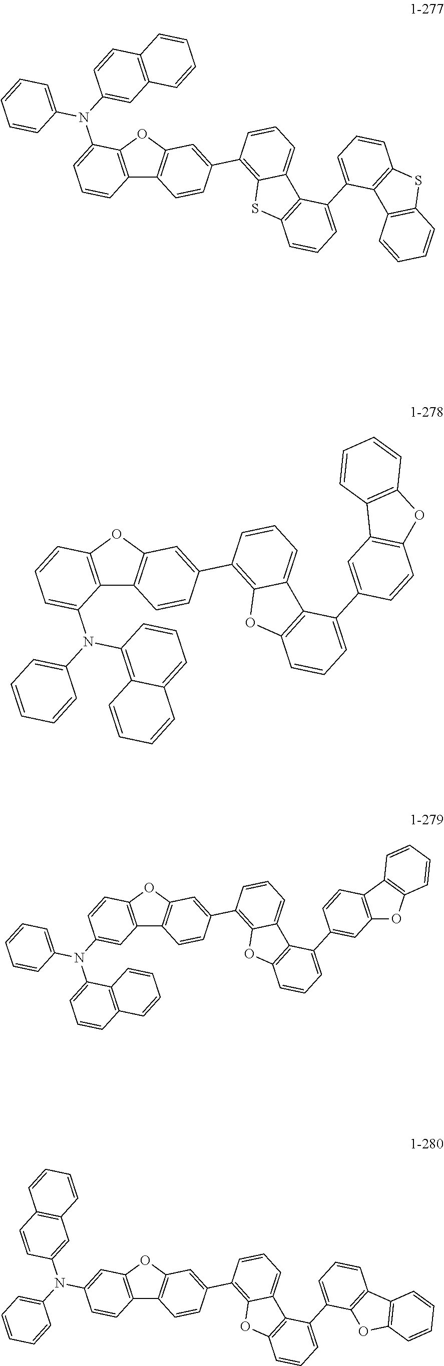 [SMILES: c1ccc(N(c2ccc3ccccc3c2)c2cccc3c2oc2cc(-c4cccc5c4sc4cccc(-c6cccc7sc8ccccc8c67)c45)ccc23)cc1 ; c1ccc(N(c2cccc3ccccc23)c2cccc3oc4cc(-c5cccc6c5oc5cccc(-c7ccc8oc9ccccc9c8c7)c56)ccc4c23)cc1 ; c1ccc(N(c2ccc3oc4cc(-c5cccc6c5oc5cccc(-c7ccc8c(c7)oc7ccccc78)c56)ccc4c3c2)c2cccc3ccccc23)cc1 ; c1ccc(N(c2ccc3ccccc3c2)c2ccc3c(c2)oc2cc(-c4cccc5c4oc4cccc(-c6cccc7c6oc6ccccc67)c45)ccc23)cc1]

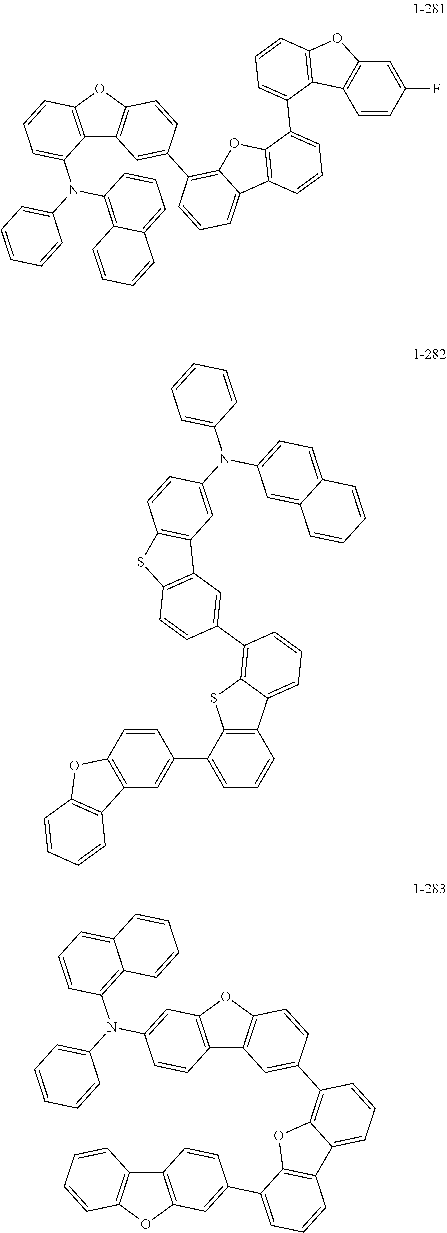 [SMILES: Fc1ccc2c(c1)oc1cccc(-c3cccc4c3oc3c(-c5ccc6oc7cccc(N(c8ccccc8)c8cccc9ccccc89)c7c6c5)cccc34)c12 ; c1ccc(N(c2ccc3ccccc3c2)c2ccc3sc4ccc(-c5cccc6c5sc5c(-c7ccc8oc9ccccc9c8c7)cccc56)cc4c3c2)cc1 ; c1ccc(N(c2ccc3c(c2)oc2ccc(-c4cccc5c4oc4c(-c6ccc7c(c6)oc6ccccc67)cccc45)cc23)c2cccc3ccccc23)cc1]

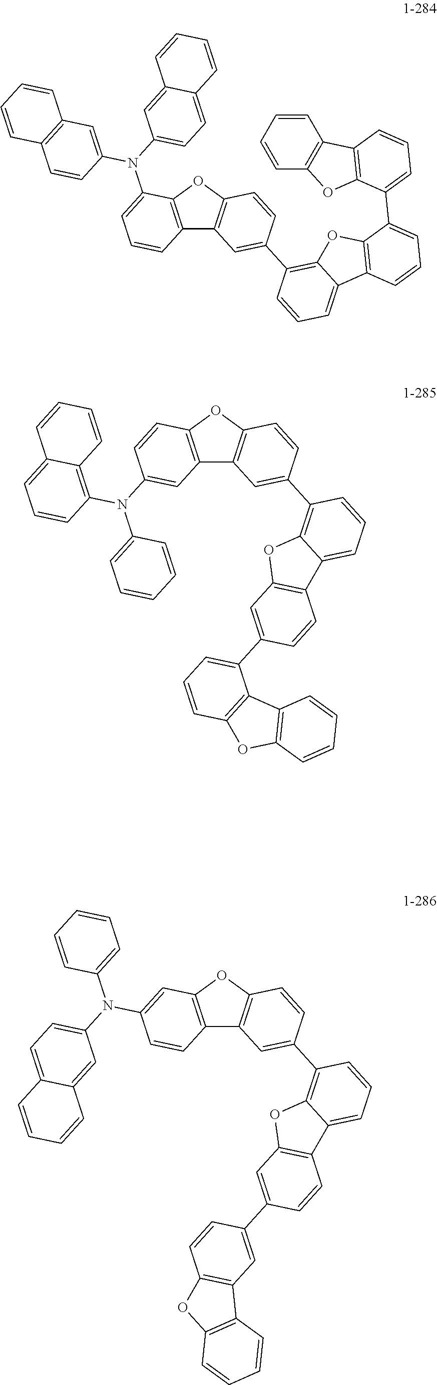 [SMILES: c1ccc2cc(N(c3ccc4ccccc4c3)c3cccc4c3oc3ccc(-c5cccc6c5oc5c(-c7cccc8c7oc7ccccc78)cccc56)cc34)ccc2c1 ; c1ccc(N(c2ccc3oc4ccc(-c5cccc6c5oc5cc(-c7cccc8oc9ccccc9c78)ccc56)cc4c3c2)c2cccc3ccccc23)cc1 ; c1ccc(N(c2ccc3ccccc3c2)c2ccc3c(c2)oc2ccc(-c4cccc5c4oc4cc(-c6ccc7oc8ccccc8c7c6)ccc45)cc23)cc1]

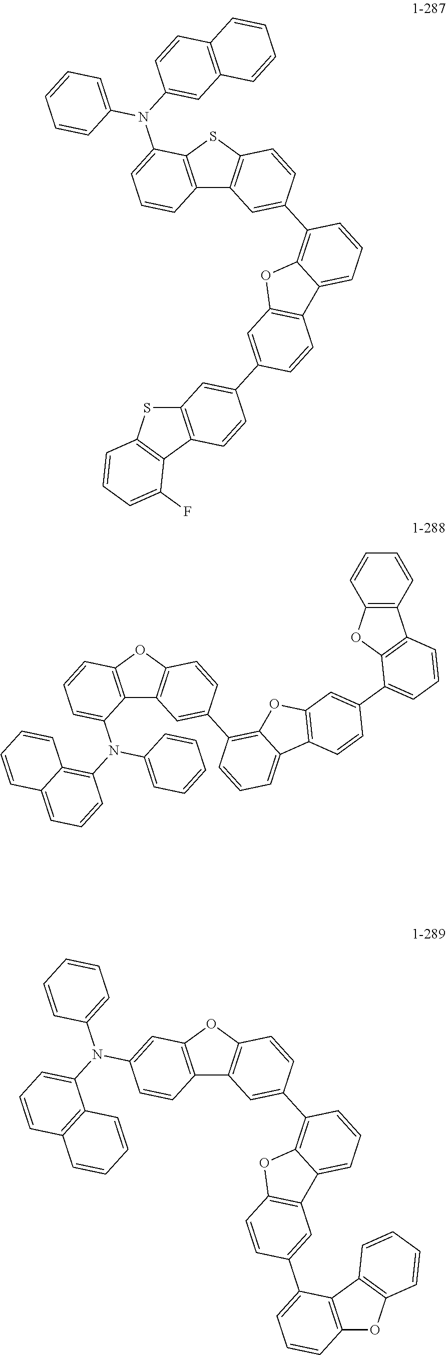

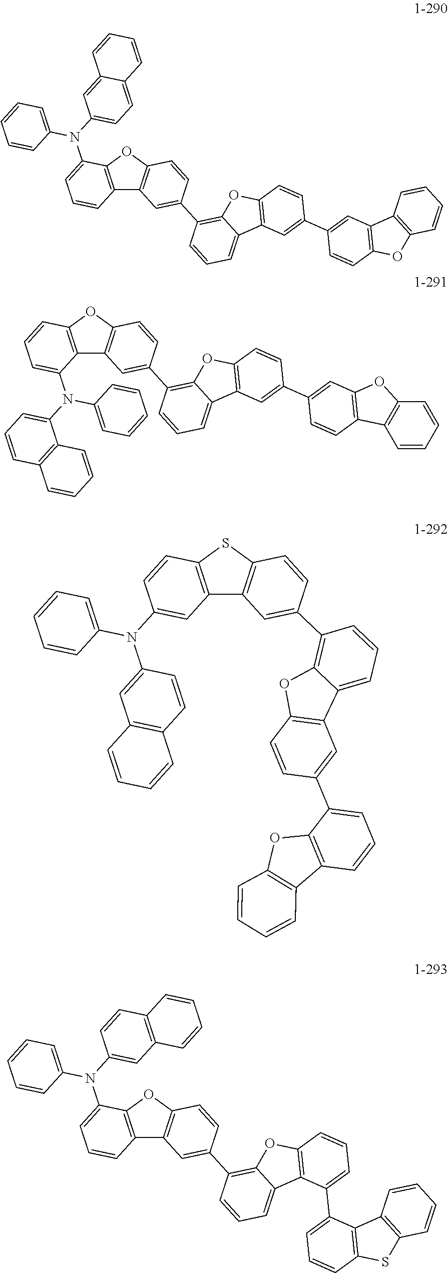 [SMILES: c1ccc(N(c2ccc3ccccc3c2)c2cccc3c2oc2ccc(-c4cccc5c4oc4ccc(-c6ccc7oc8ccccc8c7c6)cc45)cc23)cc1 ; c1ccc(N(c2cccc3ccccc23)c2cccc3oc4ccc(-c5cccc6c5oc5ccc(-c7ccc8c(c7)oc7ccccc78)cc56)cc4c23)cc1 ; c1ccc(N(c2ccc3ccccc3c2)c2ccc3sc4ccc(-c5cccc6c5oc5ccc(-c7cccc8c7oc7ccccc78)cc56)cc4c3c2)cc1 ; c1ccc(N(c2ccc3ccccc3c2)c2cccc3c2oc2ccc(-c4cccc5c4oc4cccc(-c6cccc7sc8ccccc8c67)c45)cc23)cc1]

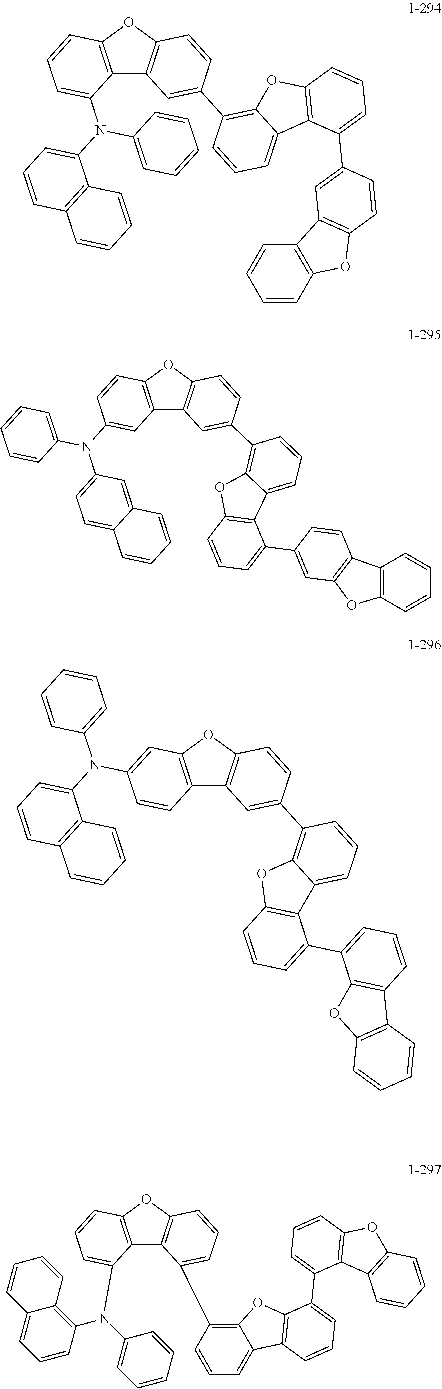 [SMILES: c1ccc(N(c2cccc3ccccc23)c2cccc3oc4ccc(-c5cccc6c5oc5cccc(-c7ccc8oc9ccccc9c8c7)c56)cc4c23)cc1 ; c1ccc(N(c2ccc3ccccc3c2)c2ccc3oc4ccc(-c5cccc6c5oc5cccc(-c7ccc8c(c7)oc7ccccc78)c56)cc4c3c2)cc1 ; c1ccc(N(c2ccc3c(c2)oc2ccc(-c4cccc5c4oc4cccc(-c6cccc7c6oc6ccccc67)c45)cc23)c2cccc3ccccc23)cc1 ; c1ccc(N(c2cccc3ccccc23)c2cccc3oc4cccc(-c5cccc6c5oc5c(-c7cccc8oc9ccccc9c78)cccc56)c4c23)cc1]

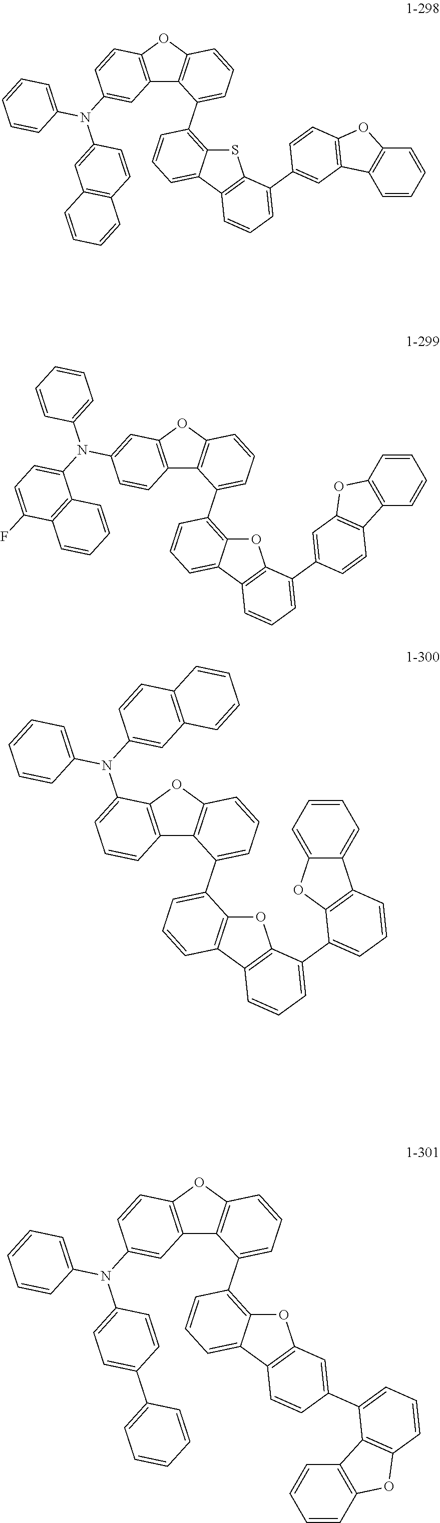

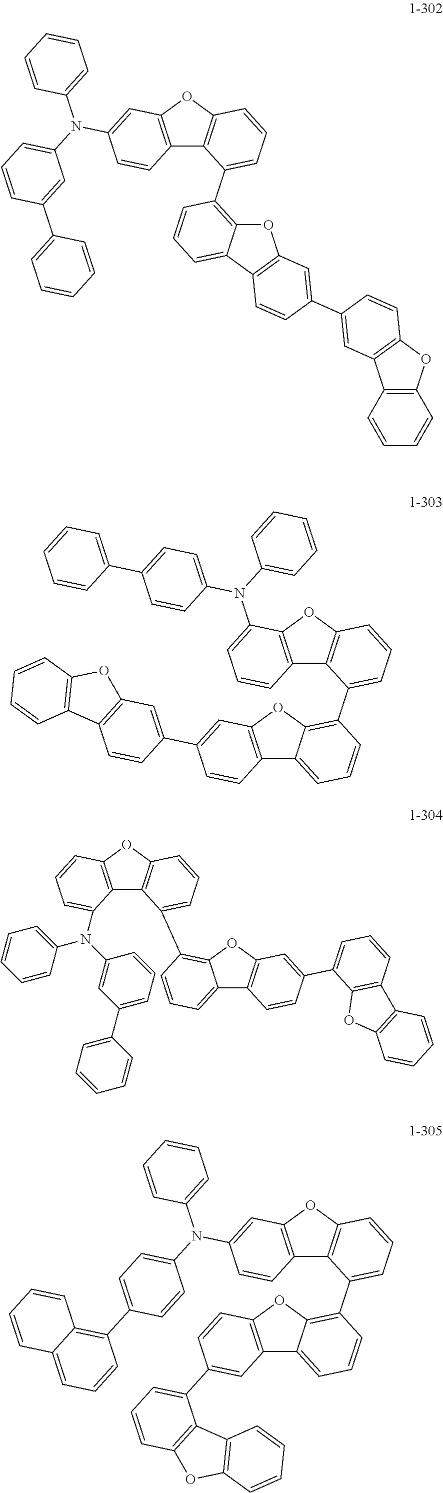

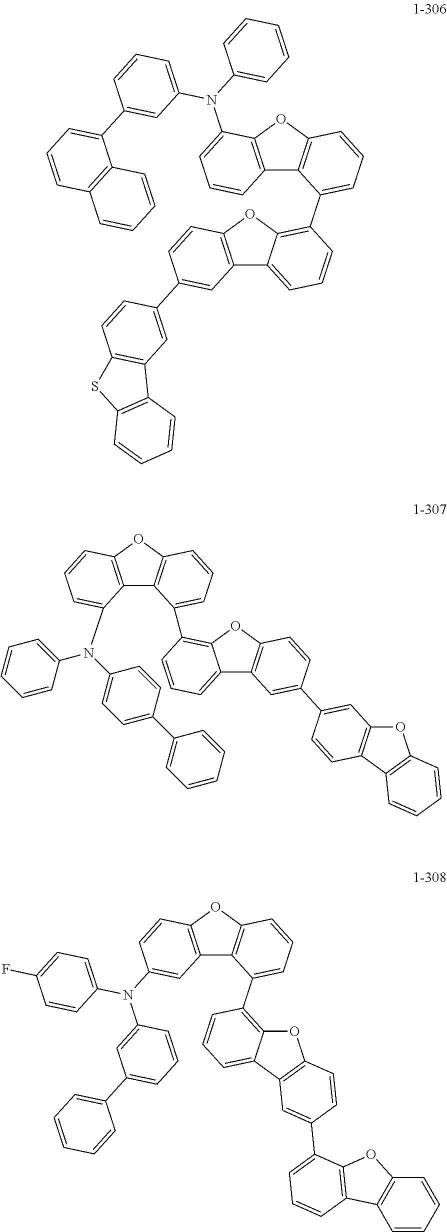 [SMILES: c1ccc(N(c2cccc(-c3cccc4ccccc34)c2)c2cccc3c2oc2cccc(-c4cccc5c4oc4ccc(-c6ccc7sc8ccccc8c7c6)cc45)c23)cc1 ; c1ccc(-c2ccc(N(c3ccccc3)c3cccc4oc5cccc(-c6cccc7c6oc6ccc(-c8ccc9c(c8)oc8ccccc89)cc67)c5c34)cc2)cc1 ; Fc1ccc(N(c2cccc(-c3ccccc3)c2)c2ccc3oc4cccc(-c5cccc6c5oc5ccc(-c7cccc8c7oc7ccccc78)cc56)c4c3c2)cc1]

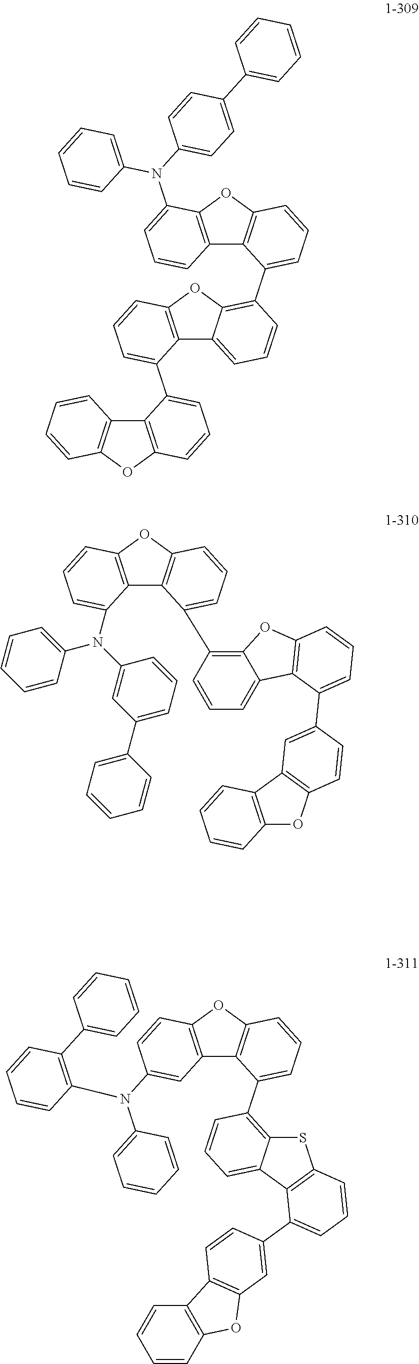 [SMILES: c1ccc(-c2ccc(N(c3ccccc3)c3cccc4c3oc3cccc(-c5cccc6c5oc5cccc(-c7cccc8oc9ccccc9c78)c56)c34)cc2)cc1 ; c1ccc(-c2cccc(N(c3ccccc3)c3cccc4oc5cccc(-c6cccc7c6oc6cccc(-c8ccc9oc%10ccccc%10c9c8)c67)c5c34)c2)cc1 ; c1ccc(-c2ccccc2N(c2ccccc2)c2ccc3oc4cccc(-c5cccc6c5sc5cccc(-c7ccc8c(c7)oc7ccccc78)c56)c4c3c2)cc1]

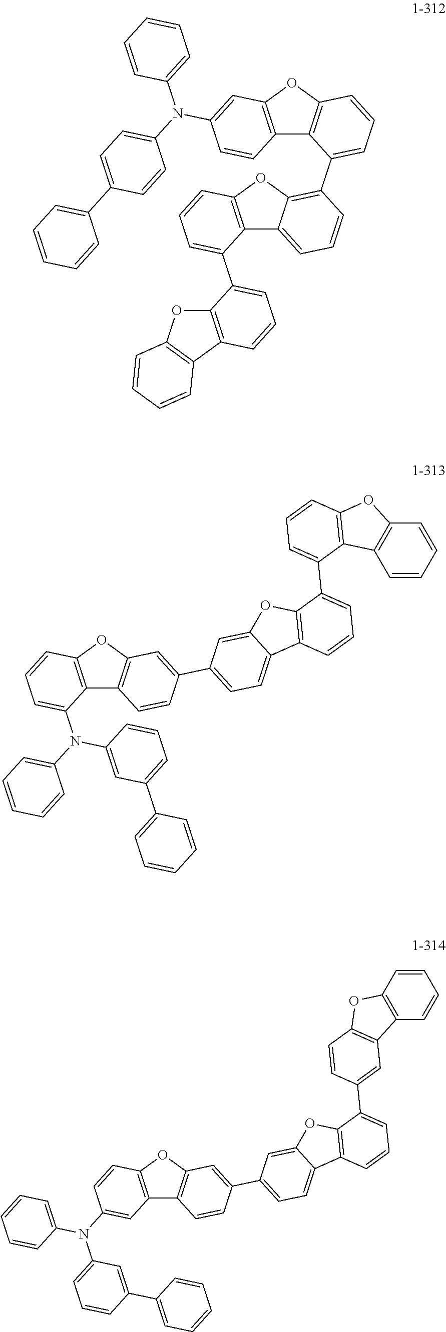 [SMILES: c1ccc(-c2ccc(N(c3ccccc3)c3ccc4c(c3)oc3cccc(-c5cccc6c5oc5cccc(-c7cccc8c7oc7ccccc78)c56)c34)cc2)cc1 ; c1ccc(-c2cccc(N(c3ccccc3)c3cccc4oc5cc(-c6ccc7c(c6)oc6c(-c8cccc9oc%10ccccc%10c89)cccc67)ccc5c34)c2)cc1 ; c1ccc(-c2cccc(N(c3ccccc3)c3ccc4oc5cc(-c6ccc7c(c6)oc6c(-c8ccc9oc%10ccccc%10c9c8)cccc67)ccc5c4c3)c2)cc1]

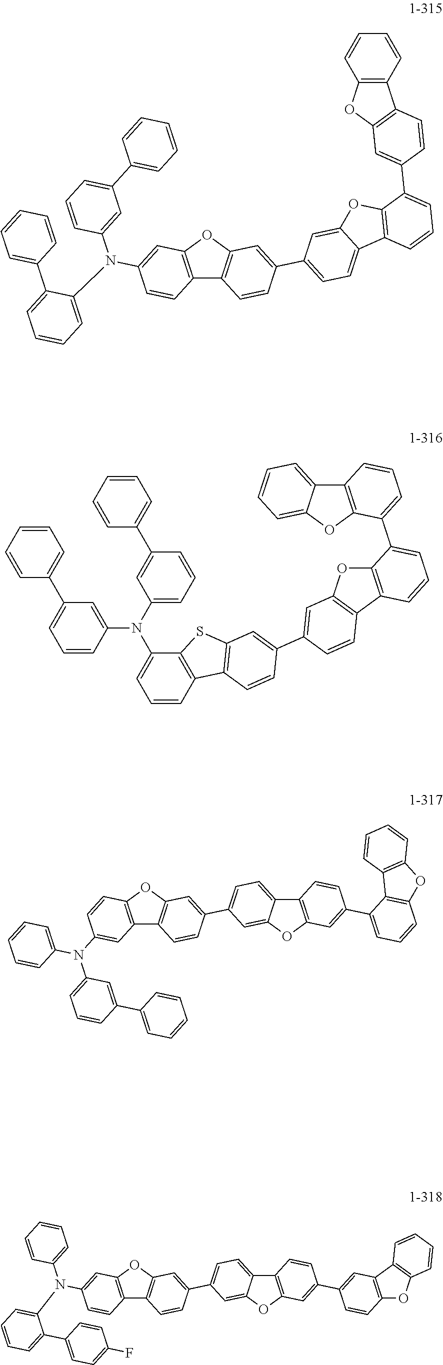 [SMILES: c1ccc(-c2cccc(N(c3ccc4c(c3)oc3cc(-c5ccc6c(c5)oc5c(-c7ccc8c(c7)oc7ccccc78)cccc56)ccc34)c3ccccc3-c3ccccc3)c2)cc1 ; c1ccc(-c2cccc(N(c3cccc(-c4ccccc4)c3)c3cccc4c3sc3cc(-c5ccc6c(c5)oc5c(-c7cccc8c7oc7ccccc78)cccc56)ccc34)c2)cc1 ; c1ccc(-c2cccc(N(c3ccccc3)c3ccc4oc5cc(-c6ccc7c(c6)oc6cc(-c8cccc9oc%10ccccc%10c89)ccc67)ccc5c4c3)c2)cc1 ; Fc1ccc(-c2ccccc2N(c2ccccc2)c2ccc3c(c2)oc2cc(-c4ccc5c(c4)oc4cc(-c6ccc7oc8ccccc8c7c6)ccc45)ccc23)cc1]

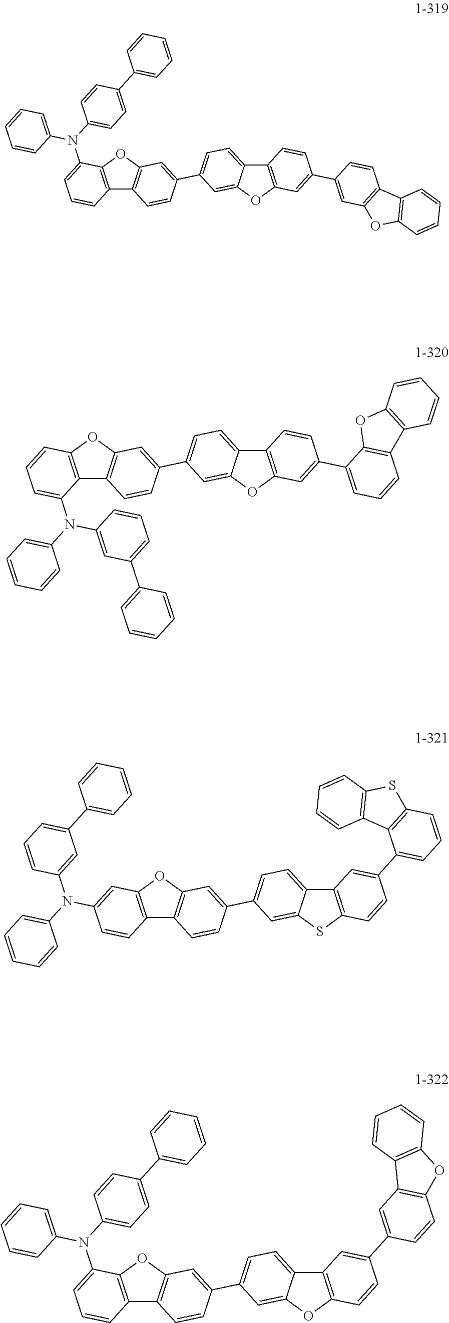 [SMILES: c1ccc(-c2ccc(N(c3ccccc3)c3cccc4c3oc3cc(-c5ccc6c(c5)oc5cc(-c7ccc8c(c7)oc7ccccc78)ccc56)ccc34)cc2)cc1 ; c1ccc(-c2cccc(N(c3ccccc3)c3cccc4oc5cc(-c6ccc7c(c6)oc6cc(-c8cccc9c8oc8ccccc89)ccc67)ccc5c34)c2)cc1 ; c1ccc(-c2cccc(N(c3ccccc3)c3ccc4c(c3)oc3cc(-c5ccc6c(c5)sc5ccc(-c7cccc8sc9ccccc9c78)cc56)ccc34)c2)cc1 ; c1ccc(-c2ccc(N(c3ccccc3)c3cccc4c3oc3cc(-c5ccc6c(c5)oc5ccc(-c7ccc8oc9ccccc9c8c7)cc56)ccc34)cc2)cc1]

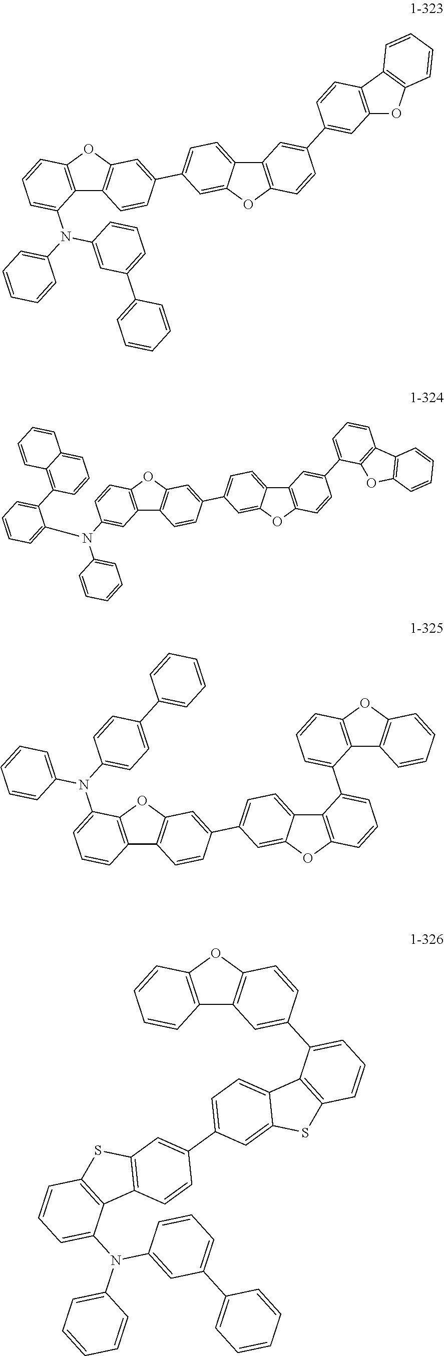 [SMILES: c1ccc(-c2cccc(N(c3ccccc3)c3cccc4oc5cc(-c6ccc7c(c6)oc6ccc(-c8ccc9c(c8)oc8ccccc89)cc67)ccc5c34)c2)cc1 ; c1ccc(N(c2ccc3oc4cc(-c5ccc6c(c5)oc5ccc(-c7cccc8c7oc7ccccc78)cc56)ccc4c3c2)c2ccccc2-c2cccc3ccccc23)cc1 ; c1ccc(-c2ccc(N(c3ccccc3)c3cccc4c3oc3cc(-c5ccc6c(c5)oc5cccc(-c7cccc8oc9ccccc9c78)c56)ccc34)cc2)cc1 ; c1ccc(-c2cccc(N(c3ccccc3)c3cccc4sc5cc(-c6ccc7c(c6)sc6cccc(-c8ccc9oc%10ccccc%10c9c8)c67)ccc5c34)c2)cc1]

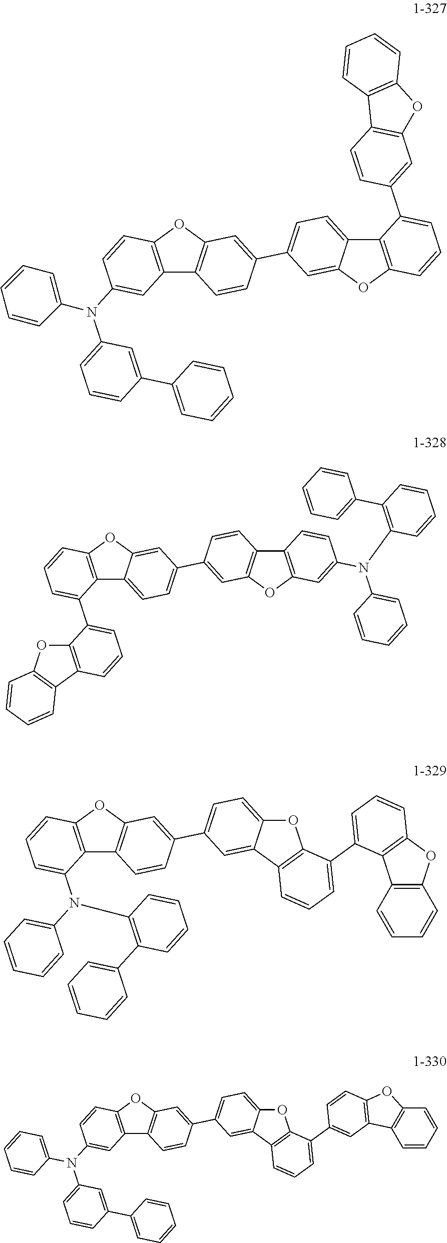 [SMILES: c1ccc(-c2cccc(N(c3ccccc3)c3ccc4oc5cc(-c6ccc7c(c6)oc6cccc(-c8ccc9c(c8)oc8ccccc89)c67)ccc5c4c3)c2)cc1 ; c1ccc(-c2ccccc2N(c2ccccc2)c2ccc3c(c2)oc2cc(-c4ccc5c(c4)oc4cccc(-c6cccc7c6oc6ccccc67)c45)ccc23)cc1 ; c1ccc(-c2ccccc2N(c2ccccc2)c2cccc3oc4cc(-c5ccc6oc7c(-c8cccc9oc%10ccccc%10c89)cccc7c6c5)ccc4c23)cc1 ; c1ccc(-c2cccc(N(c3ccccc3)c3ccc4oc5cc(-c6ccc7oc8c(-c9ccc%10oc%11ccccc%11c%10c9)cccc8c7c6)ccc5c4c3)c2)cc1]

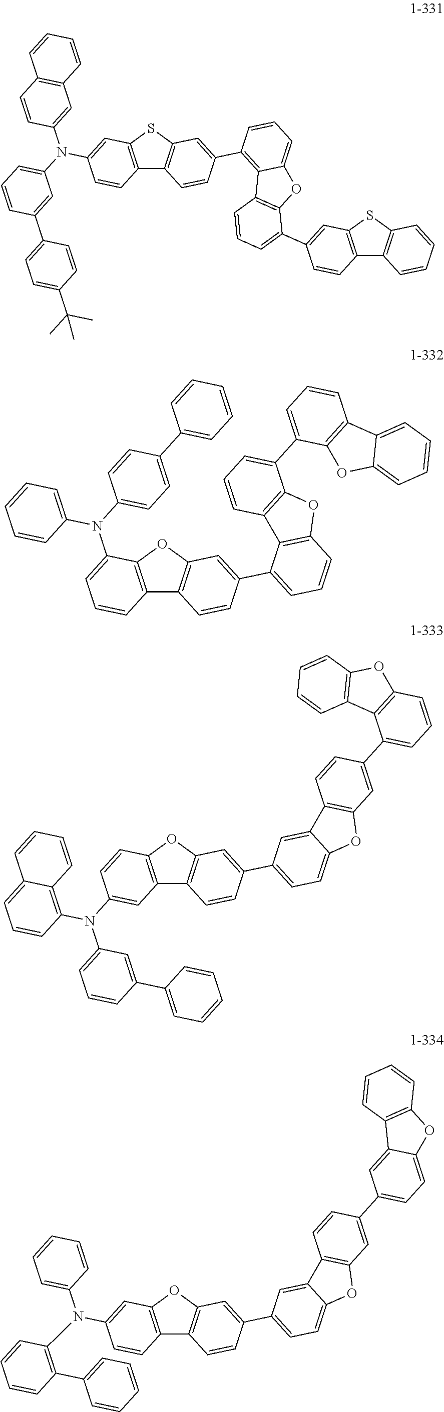 [SMILES: CC(C)(C)c1ccc(-c2cccc(N(c3ccc4ccccc4c3)c3ccc4c(c3)sc3cc(-c5cccc6oc7c(-c8ccc9c(c8)sc8ccccc89)cccc7c56)ccc34)c2)cc1 ; c1ccc(-c2ccc(N(c3ccccc3)c3cccc4c3oc3cc(-c5cccc6oc7c(-c8cccc9c8oc8ccccc89)cccc7c56)ccc34)cc2)cc1 ; c1ccc(-c2cccc(N(c3ccc4oc5cc(-c6ccc7oc8cc(-c9cccc%10oc%11ccccc%11c9%10)ccc8c7c6)ccc5c4c3)c3cccc4ccccc34)c2)cc1 ; c1ccc(-c2ccccc2N(c2ccccc2)c2ccc3c(c2)oc2cc(-c4ccc5oc6cc(-c7ccc8oc9ccccc9c8c7)ccc6c5c4)ccc23)cc1]

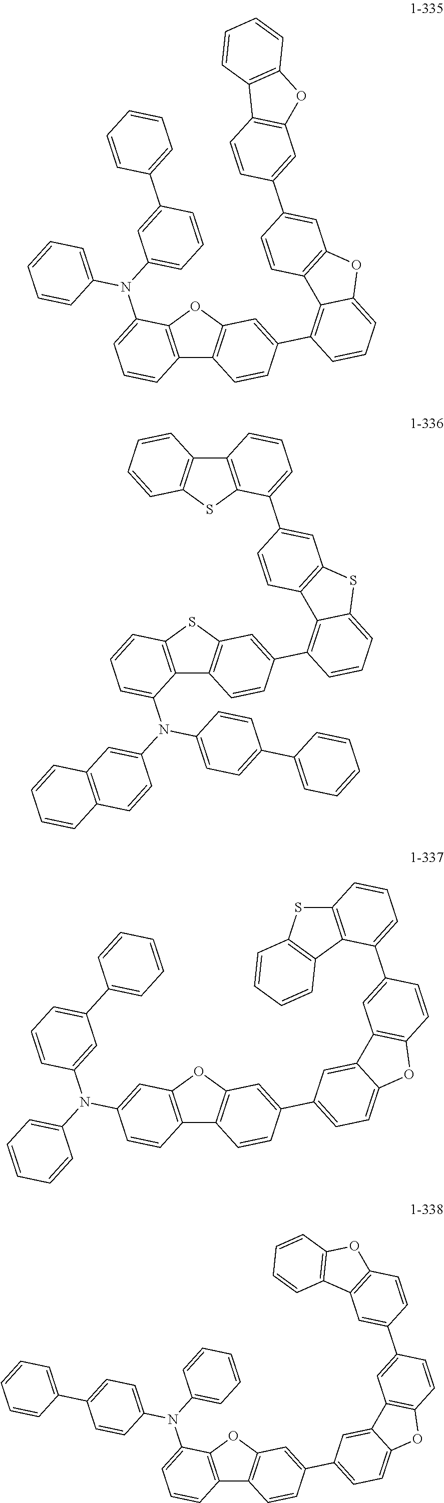 [SMILES: c1ccc(-c2cccc(N(c3ccccc3)c3cccc4c3oc3cc(-c5cccc6oc7cc(-c8ccc9c(c8)oc8ccccc89)ccc7c56)ccc34)c2)cc1 ; c1ccc(-c2ccc(N(c3ccc4ccccc4c3)c3cccc4sc5cc(-c6cccc7sc8cc(-c9cccc%10c9sc9ccccc9%10)ccc8c67)ccc5c34)cc2)cc1 ; c1ccc(-c2cccc(N(c3ccccc3)c3ccc4c(c3)oc3cc(-c5ccc6oc7ccc(-c8cccc9sc%10ccccc%10c89)cc7c6c5)ccc34)c2)cc1 ; c1ccc(-c2ccc(N(c3ccccc3)c3cccc4c3oc3cc(-c5ccc6oc7ccc(-c8ccc9oc%10ccccc%10c9c8)cc7c6c5)ccc34)cc2)cc1]

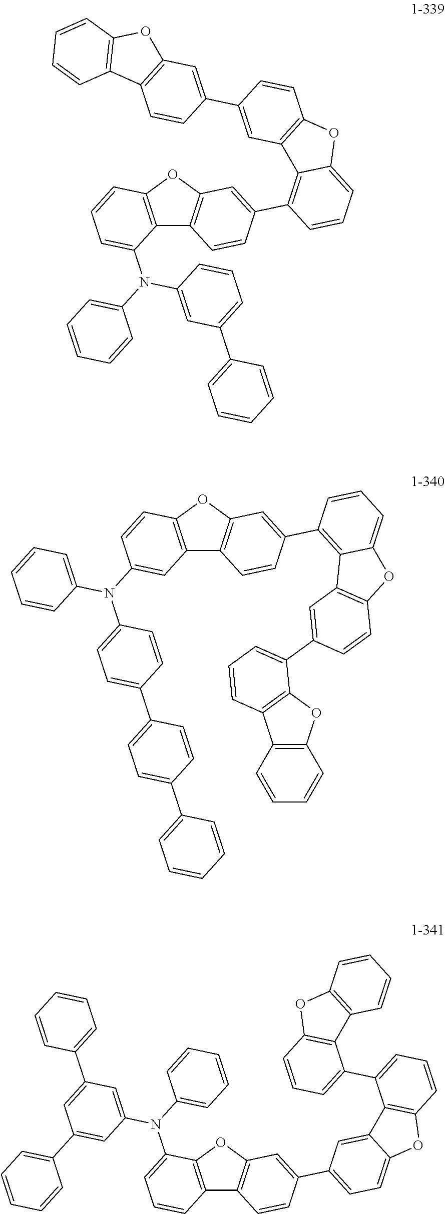 [SMILES: c1ccc(-c2cccc(N(c3ccccc3)c3cccc4oc5cc(-c6cccc7oc8ccc(-c9ccc%10c(c9)oc9ccccc9%10)cc8c67)ccc5c34)c2)cc1 ; c1ccc(-c2ccc(-c3ccc(N(c4ccccc4)c4ccc5oc6cc(-c7cccc8oc9ccc(-c%10cccc%11c%10oc%10ccccc%10%11)cc9c78)ccc6c5c4)cc3)cc2)cc1 ; c1ccc(-c2cc(-c3ccccc3)cc(N(c3ccccc3)c3cccc4c3oc3cc(-c5ccc6oc7cccc(-c8cccc9oc%10ccccc%10c89)c7c6c5)ccc34)c2)cc1]

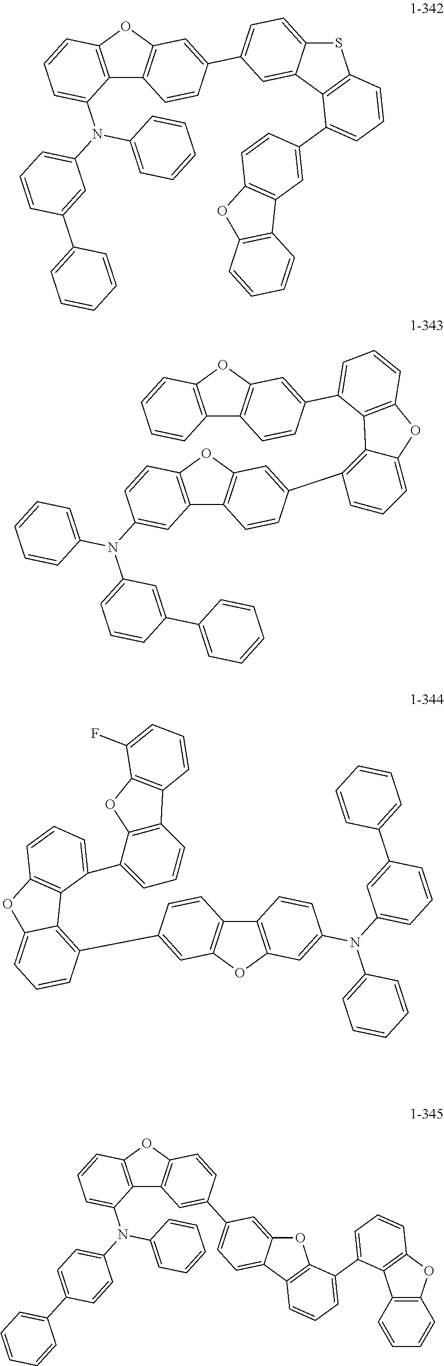 [SMILES: c1ccc(-c2cccc(N(c3ccccc3)c3cccc4oc5cc(-c6ccc7sc8cccc(-c9ccc%10oc%11ccccc%11c%10c9)c8c7c6)ccc5c34)c2)cc1 ; c1ccc(-c2cccc(N(c3ccccc3)c3ccc4oc5cc(-c6cccc7oc8cccc(-c9ccc%10c(c9)oc9ccccc9%10)c8c67)ccc5c4c3)c2)cc1 ; Fc1cccc2c1oc1c(-c3cccc4oc5cccc(-c6ccc7c(c6)oc6cc(N(c8ccccc8)c8cccc(-c9ccccc9)c8)ccc67)c5c34)cccc12 ; c1ccc(-c2ccc(N(c3ccccc3)c3cccc4oc5ccc(-c6ccc7c(c6)oc6c(-c8cccc9oc%10ccccc%10c89)cccc67)cc5c34)cc2)cc1]

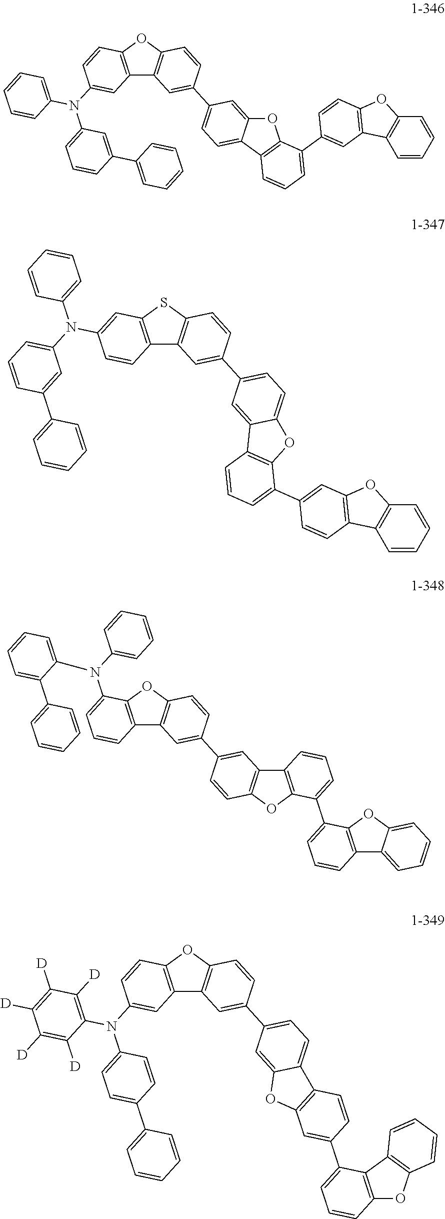 [SMILES: c1ccc(-c2cccc(N(c3ccccc3)c3ccc4oc5ccc(-c6ccc7c(c6)oc6c(-c8ccc9oc%10ccccc%10c9c8)cccc67)cc5c4c3)c2)cc1 ; c1ccc(-c2cccc(N(c3ccccc3)c3ccc4c(c3)sc3ccc(-c5ccc6oc7c(-c8ccc9c(c8)oc8ccccc89)cccc7c6c5)cc34)c2)cc1 ; c1ccc(-c2ccccc2N(c2ccccc2)c2cccc3c2oc2ccc(-c4ccc5oc6c(-c7cccc8c7oc7ccccc78)cccc6c5c4)cc23)cc1 ; [2H]c1c([2H])c([2H])c(N(c2ccc(-c3ccccc3)cc2)c2ccc3oc4ccc(-c5ccc6c(c5)oc5cc(-c7cccc8oc9ccccc9c78)ccc56)cc4c3c2)c([2H])c1[2H]]

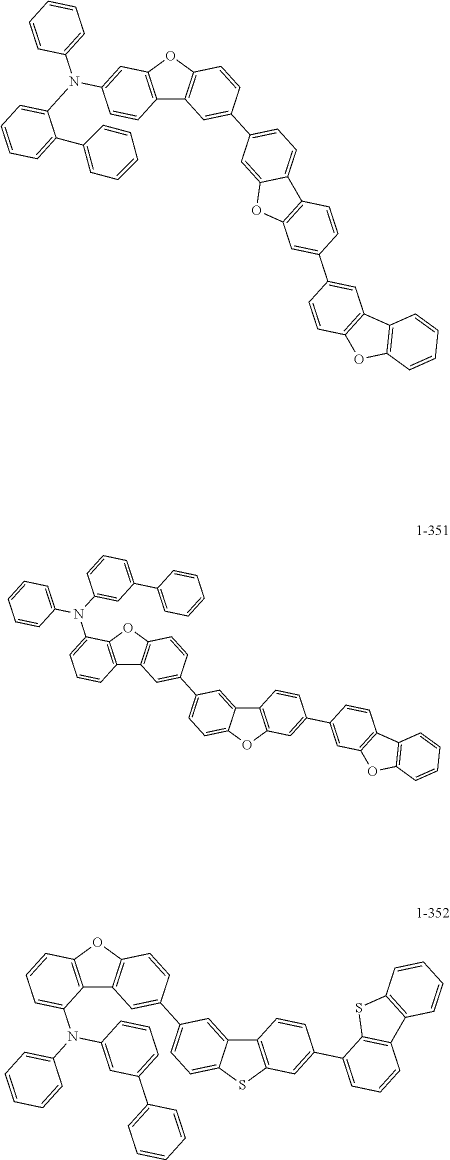 [SMILES: c1ccc(-c2ccccc2N(c2ccccc2)c2ccc3c(c2)oc2ccc(-c4ccc5c(c4)oc4cc(-c6ccc7oc8ccccc8c7c6)ccc45)cc23)cc1 ; c1ccc(-c2cccc(N(c3ccccc3)c3cccc4c3oc3ccc(-c5ccc6oc7cc(-c8ccc9c(c8)oc8ccccc89)ccc7c6c5)cc34)c2)cc1 ; c1ccc(-c2cccc(N(c3ccccc3)c3cccc4oc5ccc(-c6ccc7sc8cc(-c9cccc%10c9sc9ccccc9%10)ccc8c7c6)cc5c34)c2)cc1]

In addition, by introducing various substituents to the structure of Chemical Formula 1, compounds having unique properties of the introduced substituents may be synthesized. For example, by introducing substituents normally used for a hole injection layer material, a hole transport layer material, a light emitting layer material, an electron transport layer material and a charge generation layer material used for manufacturing an organic light emitting device to the core structure, materials satisfying conditions required for each organic material layer may be synthesized.

In addition, by introducing various substituents to the structure of Chemical Formula 1, the energy bandgap may be finely controlled, and meanwhile, properties at interfaces between organic materials may be enhanced, and material applications may become diverse.

Another embodiment of the present disclosure provides an organic light emitting device including the heterocyclic compound represented by Chemical Formula 1. The “organic light emitting device” may be expressed in terms such as an “organic light emitting diode”, an “OLED”, an “OLED device” and an “organic electroluminescent device”.

One embodiment of the present application provides an organic light emitting device including: a first electrode; a second electrode provided opposite to the first electrode; and one or more organic material layers provided between the first electrode and the second electrode, wherein the one or more organic material layers include the heterocyclic compound represented by Chemical Formula 1.

In one embodiment of the present application, the first electrode may be a positive electrode, and the second electrode may be a negative electrode.

In another embodiment, the first electrode may be a negative electrode, and the second electrode may be a positive electrode.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the heterocyclic compound represented by Chemical Formula 1 may be used as a material of the green organic light emitting device.

In one embodiment of the present application, the organic light emitting device may be a red organic light emitting device, and the heterocyclic compound represented by Chemical Formula 1 may be used as a material of the red organic light emitting device.

In one embodiment of the present application, the organic light emitting device may be a blue organic light emitting device, and the heterocyclic compound according to Chemical Formula 1 may be used as a light emitting layer material of the blue organic light emitting device.

In one embodiment of the present application, the organic light emitting device may be a green organic light emitting device, and the heterocyclic compound represented by Chemical Formula 1 may be used as a light emitting layer material of the green organic light emitting device.

In one embodiment of the present application, the organic light emitting device may be a red organic light emitting device, and the heterocyclic compound represented by Chemical Formula 1 may be used as a light emitting layer material of the red organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a light emitting layer, an electron transport layer, an electron injection layer and the like as the organic material layer. However, the structure of the organic light emitting device is not limited thereto, and may include a smaller number of organic material layers.

In the organic light emitting device of the present disclosure, the organic material layer includes a light emitting layer, and the light emitting layer may include the heterocyclic compound represented by Chemical Formula 1. When the heterocyclic compound is used in the light emitting layer, HOMO (highest occupied molecular orbital) and LUMO (lowest unoccupied molecular orbital) are spatially separated, enabling strong charge transfer, and therefore, driving efficiency and lifetime of the organic light emitting device may become superior. More specifically, since the heterocyclic compound represented by Chemical Formula 1 includes a heteroaryl-based substituent in the dibenzofuran or dibenzothiophene structure that is a core structure, electronic properties are strengthened in the dibenzofuran or dibenzothiophene structure, the core structure, and a band-gap of the compound and the triplet excited state energy level (T₁) value may be adjusted. Accordingly, when using the compound represented by Chemical Formula 1 in the organic material layer, it is possible to lower a driving voltage of the organic light emitting device, enhance light emission efficiency, and enhance lifetime properties of the organic light emitting device due to thermal stability of the compound.

In the organic light emitting device of the present disclosure, the organic material layer includes a light emitting layer, and the light emitting layer may include the heterocyclic compound of Chemical Formula 1.

In the organic light emitting device of the present disclosure, the organic material layer includes a light emitting layer, and the light emitting layer may include the heterocyclic compound of Chemical Formula 1 as a light emitting layer host.

In another embodiment of the present disclosure, the organic light emitting device may further include, one, two or more layers selected from the group consisting of a light emitting layer, a hole injection layer, a hole transport layer, a hole transport auxiliary layer, an electron injection layer, an electron transport layer, an electron blocking layer and a hole blocking layer.

In one embodiment of the present disclosure, the organic light emitting device may include one or more organic material layers, the one or more organic material layers may include a hole transport layer, and the hole transport layer may include the heterocyclic compound represented by Chemical Formula 1. In one embodiment of the present disclosure, the organic light emitting device may include one or more organic material layers, the one or more organic material layers may include a hole transport auxiliary layer, and the hole transport auxiliary layer may include the heterocyclic compound represented by Chemical Formula 1.

In one embodiment of the present disclosure, the one or more organic material layer include the heterocyclic compound represented by Chemical Formula 1, and a phosphorescent dopant may be used therewith.

As the phosphorescent dopant material, those known in the art may be used. For example, phosphorescent dopant materials represented by LL′MX′, LL′L″M, LMX′X″, L2MX′ and L3M may be used, however, the scope of the present disclosure is not limited by these examples.

M may be iridium, platinum, osmium or the like.

L is an anionic bidentate ligand coordinated to M by sp² carbon and heteroatom, and X may function to trap electrons or holes. Nonlimiting examples of L, L′ and L″ may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples:

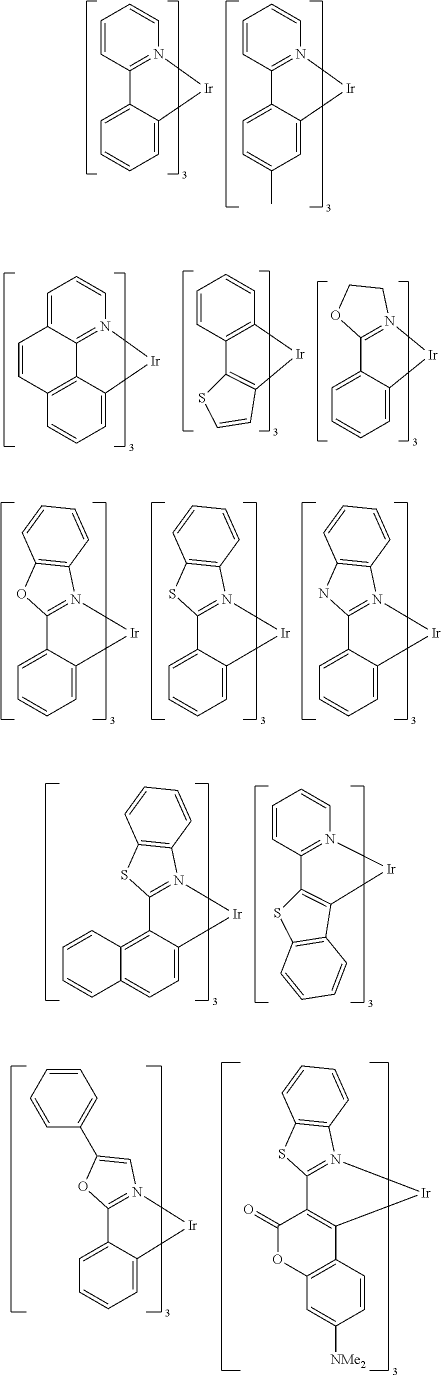

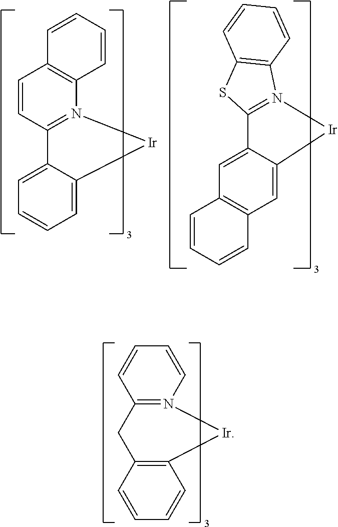

In one embodiment of the present disclosure, the one or more organic material layer include the heterocyclic compound represented by Chemical Formula 1, and an iridium-based dopant may be used therewith.

In one embodiment of the present disclosure, as the iridium-based dopant, (piq)₂(Ir) (acac), a red phosphorescent dopant, may be used.

In one embodiment of the present disclosure, as the iridium-based dopant, Ir(ppy)₃, a green phosphorescent dopant, may be used.

In one embodiment of the present disclosure, a content of the dopant may be from 1% to 15%, preferably from 2% to 10% and more preferably from 3% to 7% based on the total weight of the light emitting layer.

In the organic light emitting device according to one embodiment of the present disclosure, the one or more organic material layer include a hole transport layer or a hole transport auxiliary layer, and the hole transport layer or the hole transport auxiliary layer may include the heterocyclic compound represented by Chemical Formula 1.

In the organic light emitting device according to another embodiment of the present disclosure, the one or more organic material layer include an electron injection layer or an electron transport layer, and the electron injection layer or the electron transport layer may include the heterocyclic compound represented by Chemical Formula 1.

In the organic light emitting device according to another embodiment of the present disclosure, the one or more organic material layer include an electron blocking layer or a hole blocking layer, and the electron blocking layer or the hole blocking layer may include the heterocyclic compound represented by Chemical Formula 1.

In the organic light emitting device according to another embodiment of the present disclosure, the one or more organic material layer include an electron transport layer, a light emitting layer or a hole blocking layer, and the electron transport layer, the light emitting layer or the hole blocking layer may include the heterocyclic compound represented by Chemical Formula 1.

In the organic light emitting device according to another embodiment of the present disclosure, the one or more organic material layer include a light emitting layer, and the light emitting layer may include the heterocyclic compound represented by Chemical Formula 1.

In the organic light emitting device according to another embodiment of the present disclosure, the one or more organic material layer include a light emitting layer, the light emitting layer includes a host material, and the host material may include the heterocyclic compound represented by Chemical Formula 1. In the organic light emitting device according to still another embodiment, the light emitting layer may include two or more host materials, and at least one of the host materials may include the heterocyclic compound represented by Chemical Formula 1.

In the organic light emitting device according to still another embodiment, two or more host materials may be pre-mixed and used in the light emitting layer, and at least one of the two or more host materials may include the heterocyclic compound represented by Chemical Formula 1.

The pre-mixing means, before depositing the two or more host materials on the organic material layer, putting and mixing the materials first in one source of supply.

In the organic light emitting device according to one embodiment of the present application, an organic light emitting device in which the organic material layer including the heterocyclic compound represented by Chemical Formula 1 further includes a heterocyclic compound represented by the following Chemical Formula A is provided.

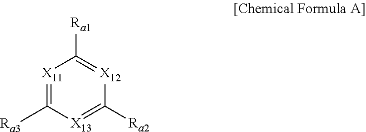

In Chemical Formula A,
X₁₁ to X₁₃ are the same as or different from each other, and each independently N or CR′,
at least one of X₁₁ to X₁₃ is N, and
Rₐ₁ to Rₐ₃ and R′ are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

In one embodiment of the present application, X₁ to X₁₃ are N; or CR′, and at least one of X₁ to X₁₃ may be N.

In one embodiment of the present application, X₁₁ to X₁₃ may be N.

In one embodiment of the present application, X₁₁ and X₁₂ are N, and X₁₃ may be CR′.

In one embodiment of the present application, X₁₁ and X₁₃ are N, and X₁₂ may be CR′.

In one embodiment of the present application, Rₐ₁ to Rₐ₃ and R′ are the same as or different from each other, and may be each independently selected from the group consisting of hydrogen; deuterium; a substituted or unsubstituted C6 to C60 aryl group; and a substituted or unsubstituted C2 to C60 heteroaryl group.

In one embodiment of the present application, Rₐ₁ to Rₐ₃ and R′ are the same as or different from each other, and may be each independently selected from the group consisting of hydrogen; deuterium; a substituted or unsubstituted C6 to C30 aryl group; and a substituted or unsubstituted C2 to C30 heteroaryl group.

In one embodiment of the present application, Rₐ₁ to Rₐ₃ and R′ are the same as or different from each other, and may be each independently selected from the group consisting of hydrogen; deuterium; a substituted or unsubstituted C6 to C20 aryl group; and a substituted or unsubstituted C2 to C20 heteroaryl group.

In one embodiment of the present application, Rₐ₁ to Rₐ₃ and R′ are the same as or different from each other, and may be each independently selected from the group consisting of a substituted or unsubstituted C6 to C20 aryl group; and a substituted or unsubstituted C2 to C20 heteroaryl group.

When the heterocyclic compound of Chemical Formula 1 and the heterocyclic compound of Chemical Formula A are included in the organic material layer of the organic light emitting device, effects of more superior efficiency and lifetime are obtained. Such a result may lead to a forecast that an exciplex phenomenon occurs when the two compounds are included at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and therefore, a driving voltage may be lowered, which resultantly helps with enhancement in the lifetime.

In one embodiment of the present application, Chemical Formula A may be represented by any one of the following Chemical Formulae A-1 to A-4.

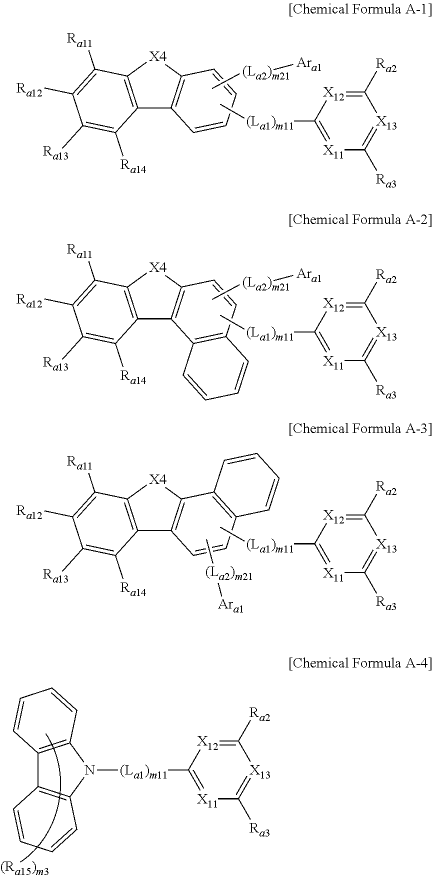

In Chemical Formula A-1 to Chemical Formula A-4,
X₁₁ to X₁₃, Rₐ₂ and Rₐ₃ have the same definitions as in Chemical Formula A,
X4 is O, S or CRₐₐR_{bb},
Rₐₐ and R_{bb} are the same as or different from each other, and may be each independently selected from the group consisting of hydrogen; deuterium; a halogen group; —CN; a substituted or unsubstituted C1 to C60 alkyl group; substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group,
Rₐ₁₁ to Rₐ₁₅ are the same as or different from each other, and each independently selected from the group consisting of hydrogen; deuterium; a halogen group; —CN; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring,
Lₐ₁ and Lₐ₂ are the same as or different from each other, and each independently a direct bond; a substituted or unsubstituted C6 to C60 arylene group; or a substituted or unsubstituted C2 to C60 heteroarylene group,
Arₐ₁ is hydrogen; deuterium; a substituted or unsubstituted C6 to C60 aryl group; or a substituted or unsubstituted C2 to C60 heteroaryl group,
m11 and m21 are an integer of 0 to 2, and when m11 and m21 are 2, substituents in the parentheses are the same as or different from each other, and
m3 is an integer of 0 to 8, and when m3 is 2 or greater, substituents in the parentheses are the same as or different from each other.

In one embodiment of the present application, Rₐ₂ and Rₐ₃ are the same as or different from each other, and may be each independently selected from the group consisting of hydrogen; deuterium; a halogen group; —CN; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

In another embodiment, Rₐ₂ and Rₐ₃ are the same as or different from each other, and may be each independently selected from the group consisting of a substituted or unsubstituted C6 to C60 aryl group; and a substituted or unsubstituted C2 to C60 heteroaryl group.

In another embodiment, Rₐ₂ and Rₐ₃ are the same as or different from each other, and may be each independently selected from the group consisting of a substituted or unsubstituted C6 to C40 aryl group; and a substituted or unsubstituted C2 to C40 heteroaryl group.

In another embodiment, Rₐ₂ and Rₐ₃ are the same as or different from each other, and may be each independently selected from the group consisting of a C6 to C40 aryl group unsubstituted or substituted with a C6 to C40 aryl group or a C2 to C40 heteroaryl group; and a C2 to C40 heteroaryl group.

In another embodiment, Rₐ₂ and Rₐ₃ are the same as or different from each other, and may be each independently selected from the group consisting of a C6 to C20 aryl group unsubstituted or substituted with a C6 to C20 aryl group or a C2 to C20 heteroaryl group; and a C2 to C20 heteroaryl group.

In another embodiment, Rₐ₂ and Rₐ₃ are the same as or different from each other, and may be each independently a substituted or unsubstituted phenyl group; a substituted or unsubstituted naphthyl group; a substituted or unsubstituted biphenyl group; a substituted or unsubstituted terphenyl group; a substituted or unsubstituted phenanthrenyl group; a substituted or unsubstituted dibenzofuran group; a substituted or unsubstituted fluorenyl group; a substituted or unsubstituted dibenzothiophene group; or a group represented by the following Chemical Formula B-1.

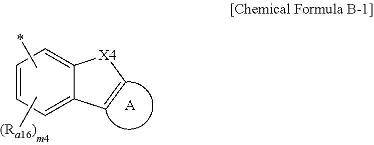

In Chemical Formula B-1,
X4 has the same definition as in Chemical Formula A-1 to Chemical Formula A-3,
Rₐ₁₆ is selected from the group consisting of hydrogen; deuterium; a halogen group; —CN; a substituted or unsubstituted C1 to C20 alkyl group; a substituted or unsubstituted C6 to C20 aryl group; and a substituted or unsubstituted C2 to C20 heteroaryl group,
m4 is an integer of 0 to 3,
* is a connection point bonding to carbon between X11 and X13 in Chemical Formula A-1 to Chemical Formula A-3, and
the ring A is represented by the following Chemical Formula B-2.

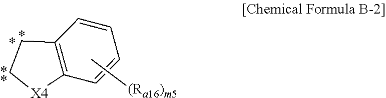

In Chemical Formula B-2,
X4 and Rₐ₁₆ have the same definitions as in Chemical Formula B-1,
m5 is an integer of 0 to 4, and
** is a connection point with Chemical Formula B-1.

In another embodiment, Rₐ₂ and Rₐ₃ are the same as or different from each other, and each independently a phenyl group unsubstituted or substituted with a phenyl group, a naphthyl group or a carbazole group; a naphthyl group; a biphenyl group; a terphenyl group; a phenanthrenyl group; a dibenzofuran group; a spirobifluorenyl group; a dibenzothiophene group; a dimethylbenzoindenothiophene group; or a dimethylbenzoindenofuran group.

In one embodiment of the present application, R′ may be hydrogen; or deuterium.

In one embodiment of the present application, Rₐ₁₁ to Rₐ₁₅ are the same as or different from each other, and each independently selected from the group consisting of hydrogen; deuterium; a halogen group; —CN; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other may bond to each other to form a substituted or unsubstituted C6 to C60 aromatic hydrocarbon ring; or a substituted or unsubstituted C2 to C60 heteroring.

In another embodiment, Rₐ₁₁ to Rₐ₁₅ are the same as or different from each other, and each independently selected from the group consisting of hydrogen; deuterium; a substituted or unsubstituted C6 to C60 aryl group; and a substituted or unsubstituted C2 to C60 heteroaryl group, or two or more groups adjacent to each other may bond to each other to form a substituted or unsubstituted C6 to C60 aromatic hydrocarbon ring.

In another embodiment, Rₐ₁₁ to Rₐ₁₅ are the same as or different from each other, and each independently selected from the group consisting of hydrogen; deuterium; a substituted or unsubstituted C6 to C60 aryl group; and a substituted or unsubstituted C2 to C60 heteroaryl group, or two or more groups adjacent to each other may bond to each other to form a substituted or unsubstituted C6 to C60 aromatic hydrocarbon ring.

In another embodiment, Rₐ₁₁ to Rₐ₁₅ are the same as or different from each other, and each independently selected from the group consisting of hydrogen; deuterium; a substituted or unsubstituted C6 to C40 aryl group; and a substituted or unsubstituted C2 to C40 heteroaryl group, or two or more groups adjacent to each other may bond to each other to form a substituted or unsubstituted C6 to C40 aromatic hydrocarbon ring.

In another embodiment, Rₐ₁₁ to Rₐ₁₅ are the same as or different from each other, and each independently selected from the group consisting of hydrogen; deuterium; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring.

In another embodiment, Rₐ₁₁ to Rₐ₁₅ are the same as or different from each other, and each independently selected from the group consisting of hydrogen; deuterium; a substituted or unsubstituted phenyl group; substituted or unsubstituted biphenyl group; a substituted or unsubstituted naphthyl group; a substituted or unsubstituted terphenyl group; a substituted or unsubstituted phenanthrenyl group; a substituted or unsubstituted dibenzofuran group; and a substituted or unsubstituted dibenzothiophene group, or two or more groups adjacent to each other may bond to each other to form a benzene ring.

In one embodiment of the present application, Lₐ₁ and Lₐ₂ are the same as or different from each other, and may be each independently a direct bond; a substituted or unsubstituted C6 to C60 arylene group; or a substituted or unsubstituted C2 to C60 heteroarylene group.

In another embodiment, Lₐ₁ and Lₐ₂ are the same as or different from each other, and may be each independently a direct bond; a substituted or unsubstituted C6 to C40 arylene group; or a substituted or unsubstituted C2 to C40 heteroarylene group.

In another embodiment, Lₐ₁ and Lₐ₂ are the same as or different from each other, and may be each independently a direct bond; a substituted or unsubstituted C6 to C20 arylene group; or a substituted or unsubstituted C2 to C20 heteroarylene group.

In another embodiment, Lₐ₁ and Lₐ₂ are the same as or different from each other, and may be each independently a direct bond; a substituted or unsubstituted phenylene group; a substituted or unsubstituted biphenylene group; or a substituted or unsubstituted naphthalene group.

In one embodiment of the present application, Arₐ₁ may be hydrogen; deuterium; a substituted or unsubstituted C6 to C60 aryl group; or a substituted or unsubstituted C2 to C60 heteroaryl group.

In another embodiment, Arai may be hydrogen; deuterium; a substituted or unsubstituted C6 to C40 aryl group; or a substituted or unsubstituted C2 to C40 heteroaryl group.

In another embodiment, Arai may be hydrogen; deuterium; a substituted or unsubstituted C6 to C20 aryl group; or a substituted or unsubstituted C2 to C20 heteroaryl group.

In another embodiment, Arai may be hydrogen; deuterium; a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; or a substituted or unsubstituted phenanthrenyl group.

In one embodiment of the present application, the heterocyclic compound of Chemical Formula A may be represented by any one of the following compounds.

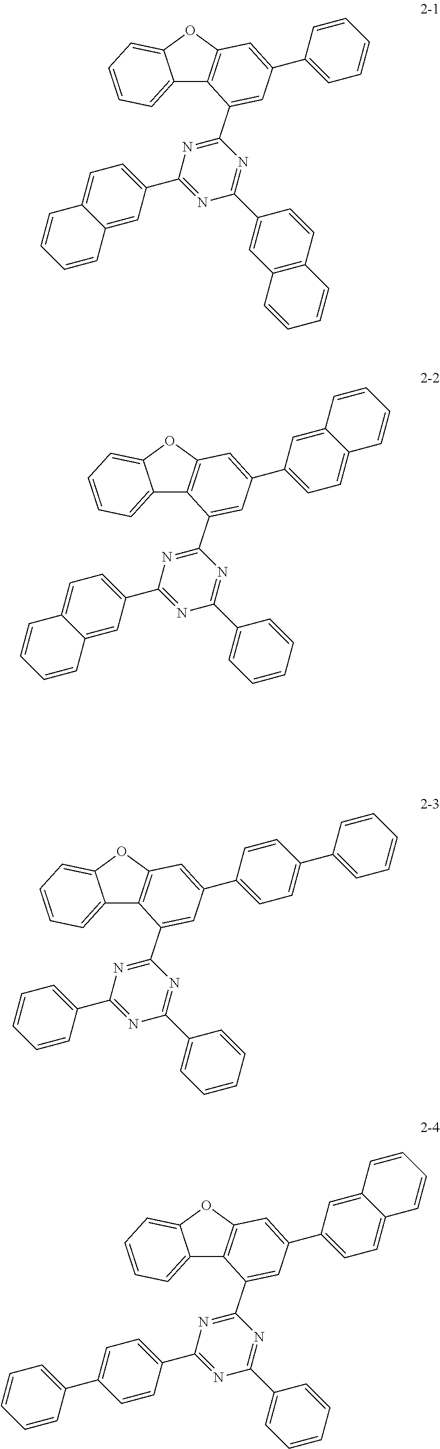 [SMILES: c1ccc(-c2cc(-c3nc(-c4ccc5ccccc5c4)nc(-c4ccc5ccccc5c4)n3)c3c(c2)oc2ccccc23)cc1 ; c1ccc(-c2nc(-c3ccc4ccccc4c3)nc(-c3cc(-c4ccc5ccccc5c4)cc4oc5ccccc5c34)n2)cc1 ; c1ccc(-c2ccc(-c3cc(-c4nc(-c5ccccc5)nc(-c5ccccc5)n4)c4c(c3)oc3ccccc34)cc2)cc1 ; c1ccc(-c2ccc(-c3nc(-c4ccccc4)nc(-c4cc(-c5ccc6ccccc6c5)cc5oc6ccccc6c45)n3)cc2)cc1]

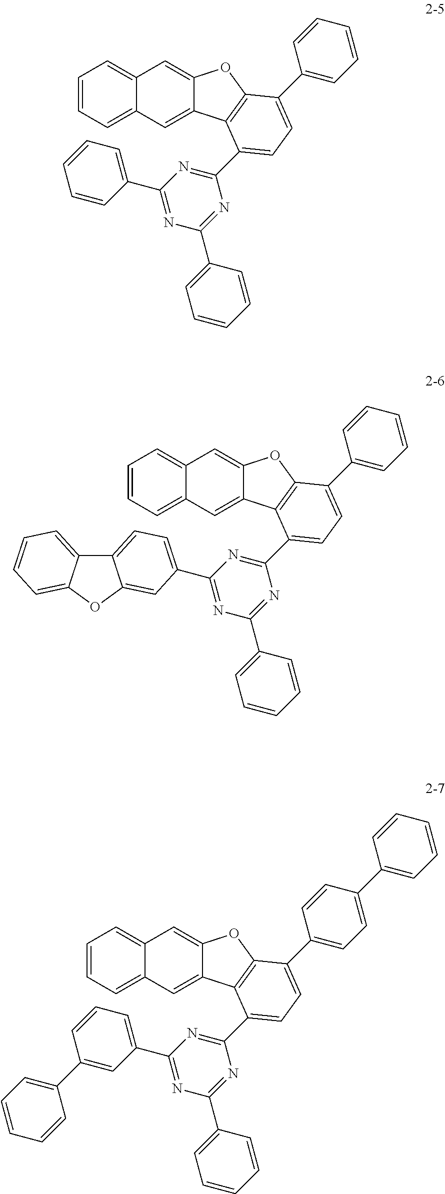 [SMILES: c1ccc(-c2nc(-c3ccccc3)nc(-c3ccc(-c4ccccc4)c4oc5cc6ccccc6cc5c34)n2)cc1 ; c1ccc(-c2nc(-c3ccc4c(c3)oc3ccccc34)nc(-c3ccc(-c4ccccc4)c4oc5cc6ccccc6cc5c34)n2)cc1 ; c1ccc(-c2ccc(-c3ccc(-c4nc(-c5ccccc5)nc(-c5cccc(-c6ccccc6)c5)n4)c4c3oc3cc5ccccc5cc34)cc2)cc1]

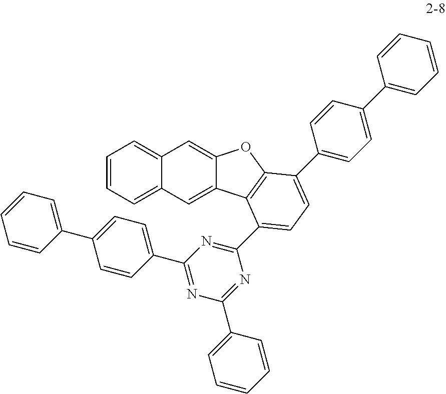 [SMILES: c1ccc(-c2ccc(-c3nc(-c4ccccc4)nc(-c4ccc(-c5ccc(-c6ccccc6)cc5)c5oc6cc7ccccc7cc6c45)n3)cc2)cc1]

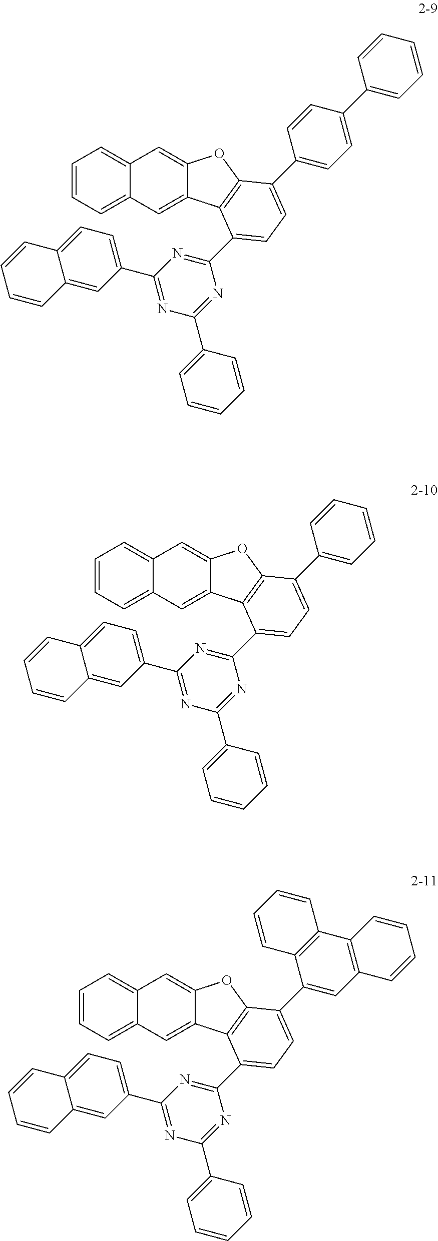 [SMILES: c1ccc(-c2ccc(-c3ccc(-c4nc(-c5ccccc5)nc(-c5ccc6ccccc6c5)n4)c4c3oc3cc5ccccc5cc34)cc2)cc1 ; c1ccc(-c2nc(-c3ccc4ccccc4c3)nc(-c3ccc(-c4ccccc4)c4oc5cc6ccccc6cc5c34)n2)cc1 ; c1ccc(-c2nc(-c3ccc4ccccc4c3)nc(-c3ccc(-c4cc5ccccc5c5ccccc45)c4oc5cc6ccccc6cc5c34)n2)cc1]

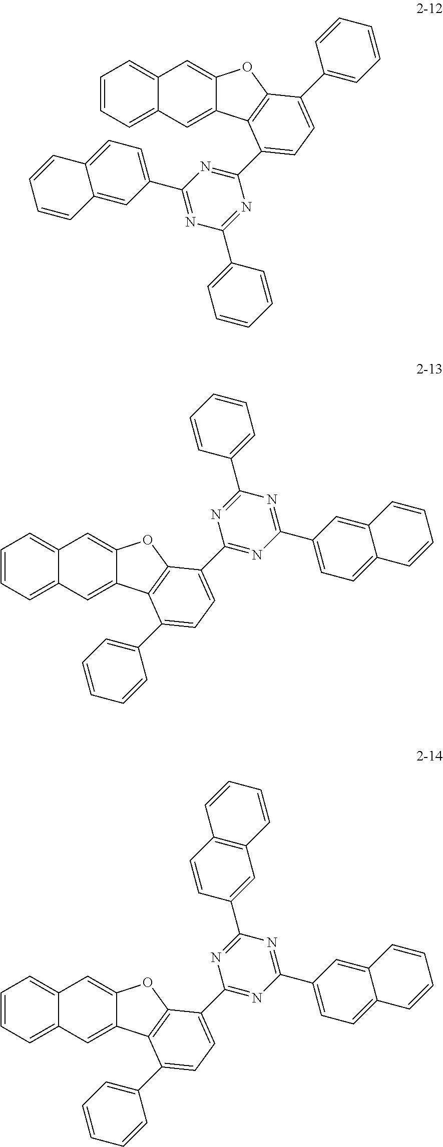 [SMILES: c1ccc(-c2nc(-c3ccc4ccccc4c3)nc(-c3ccc(-c4ccccc4)c4oc5cc6ccccc6cc5c34)n2)cc1 ; c1ccc(-c2nc(-c3ccc4ccccc4c3)nc(-c3ccc(-c4ccccc4)c4c3oc3cc5ccccc5cc34)n2)cc1 ; c1ccc(-c2ccc(-c3nc(-c4ccc5ccccc5c4)nc(-c4ccc5ccccc5c4)n3)c3oc4cc5ccccc5cc4c23)cc1]

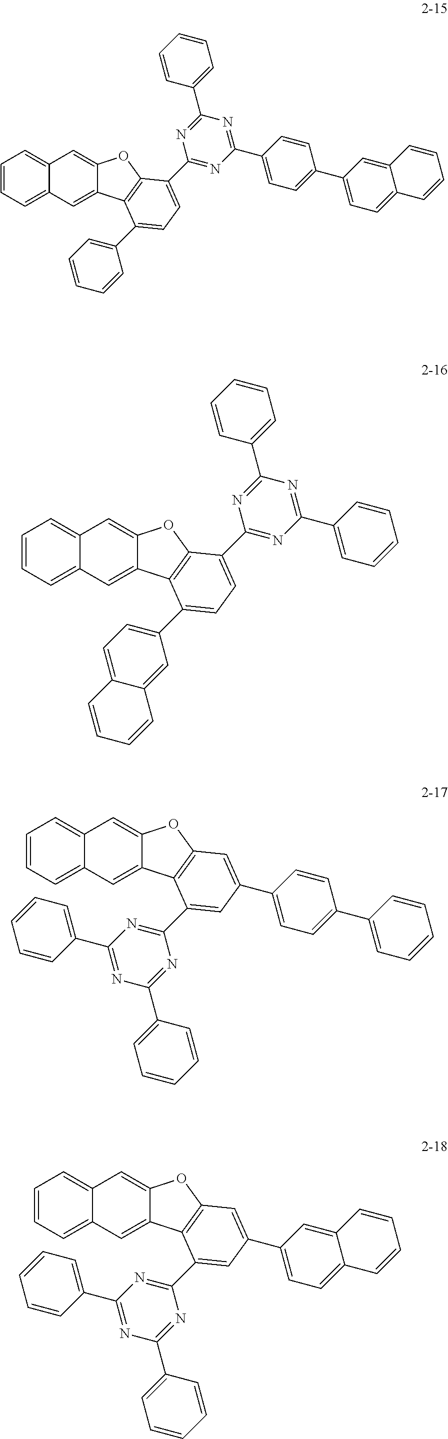 [SMILES: c1ccc(-c2nc(-c3ccc(-c4ccc5ccccc5c4)cc3)nc(-c3ccc(-c4ccccc4)c4c3oc3cc5ccccc5cc34)n2)cc1 ; c1ccc(-c2nc(-c3ccccc3)nc(-c3ccc(-c4ccc5ccccc5c4)c4c3oc3cc5ccccc5cc34)n2)cc1 ; c1ccc(-c2ccc(-c3cc(-c4nc(-c5ccccc5)nc(-c5ccccc5)n4)c4c(c3)oc3cc5ccccc5cc34)cc2)cc1 ; c1ccc(-c2nc(-c3ccccc3)nc(-c3cc(-c4ccc5ccccc5c4)cc4oc5cc6ccccc6cc5c34)n2)cc1]

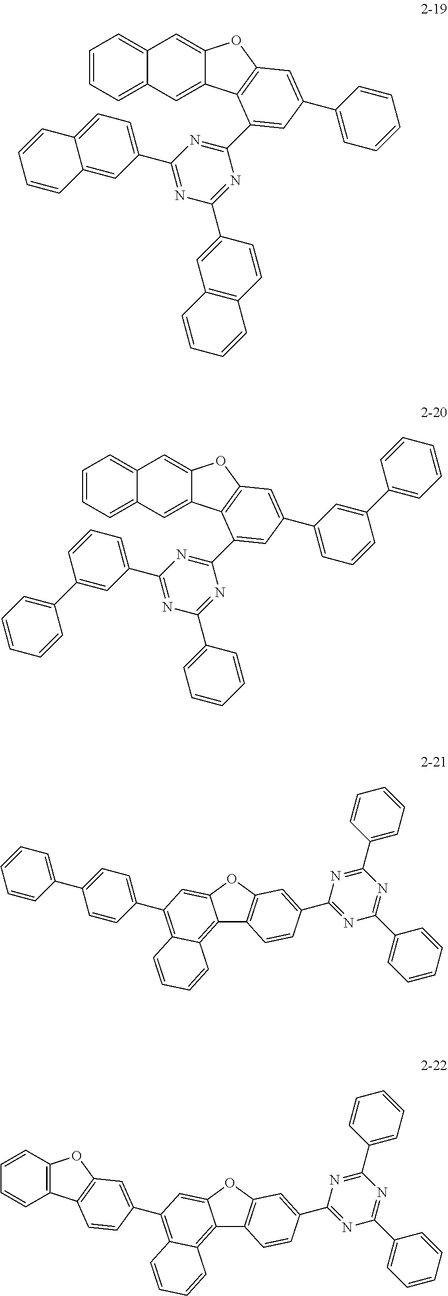 [SMILES: c1ccc(-c2cc(-c3nc(-c4ccc5ccccc5c4)nc(-c4ccc5ccccc5c4)n3)c3c(c2)oc2cc4ccccc4cc23)cc1 ; c1ccc(-c2cccc(-c3cc(-c4nc(-c5ccccc5)nc(-c5cccc(-c6ccccc6)c5)n4)c4c(c3)oc3cc5ccccc5cc34)c2)cc1 ; c1ccc(-c2ccc(-c3cc4oc5cc(-c6nc(-c7ccccc7)nc(-c7ccccc7)n6)ccc5c4c4ccccc34)cc2)cc1 ; c1ccc(-c2nc(-c3ccccc3)nc(-c3ccc4c(c3)oc3cc(-c5ccc6c(c5)oc5ccccc56)c5ccccc5c34)n2)cc1]

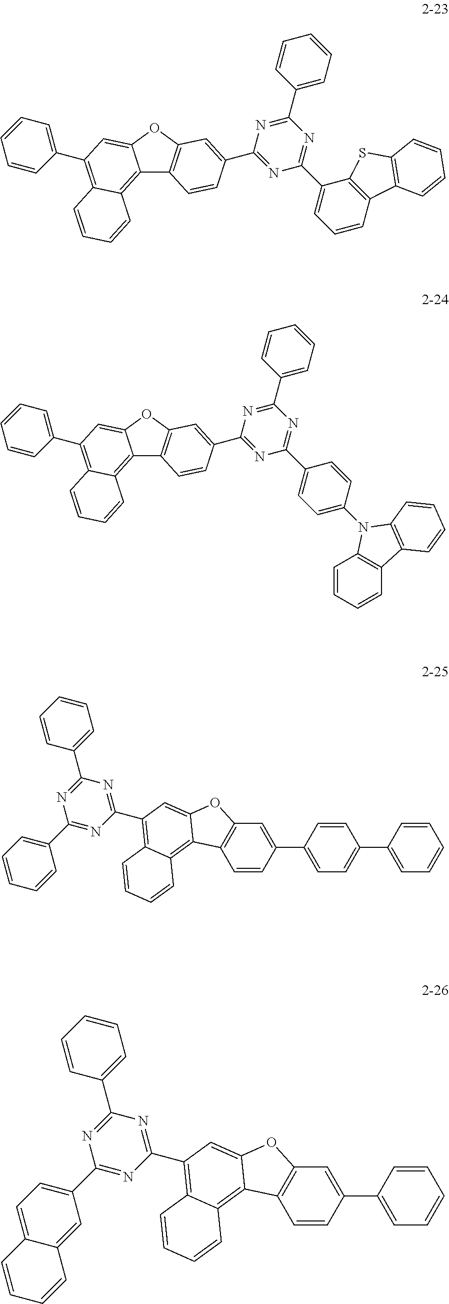 [SMILES: c1ccc(-c2nc(-c3ccc4c(c3)oc3cc(-c5ccccc5)c5ccccc5c34)nc(-c3cccc4c3sc3ccccc34)n2)cc1 ; c1ccc(-c2nc(-c3ccc(-n4c5ccccc5c5ccccc54)cc3)nc(-c3ccc4c(c3)oc3cc(-c5ccccc5)c5ccccc5c34)n2)cc1 ; c1ccc(-c2ccc(-c3ccc4c(c3)oc3cc(-c5nc(-c6ccccc6)nc(-c6ccccc6)n5)c5ccccc5c34)cc2)cc1 ; c1ccc(-c2ccc3c(c2)oc2cc(-c4nc(-c5ccccc5)nc(-c5ccc6ccccc6c5)n4)c4ccccc4c23)cc1]

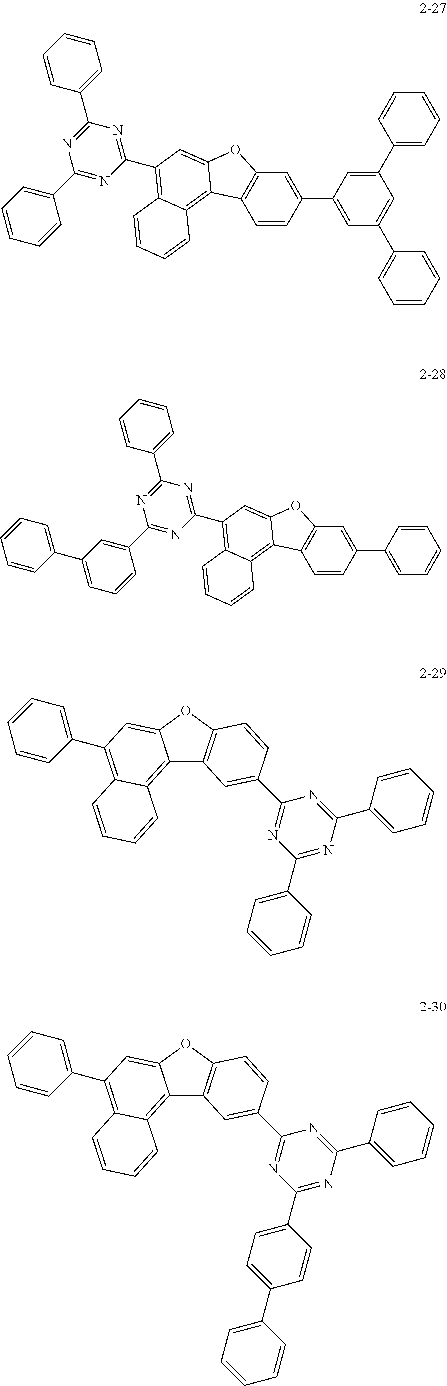 [SMILES: c1ccc(-c2cc(-c3ccccc3)cc(-c3ccc4c(c3)oc3cc(-c5nc(-c6ccccc6)nc(-c6ccccc6)n5)c5ccccc5c34)c2)cc1 ; c1ccc(-c2cccc(-c3nc(-c4ccccc4)nc(-c4cc5oc6cc(-c7ccccc7)ccc6c5c5ccccc45)n3)c2)cc1 ; c1ccc(-c2nc(-c3ccccc3)nc(-c3ccc4oc5cc(-c6ccccc6)c6ccccc6c5c4c3)n2)cc1 ; c1ccc(-c2ccc(-c3nc(-c4ccccc4)nc(-c4ccc5oc6cc(-c7ccccc7)c7ccccc7c6c5c4)n3)cc2)cc1]

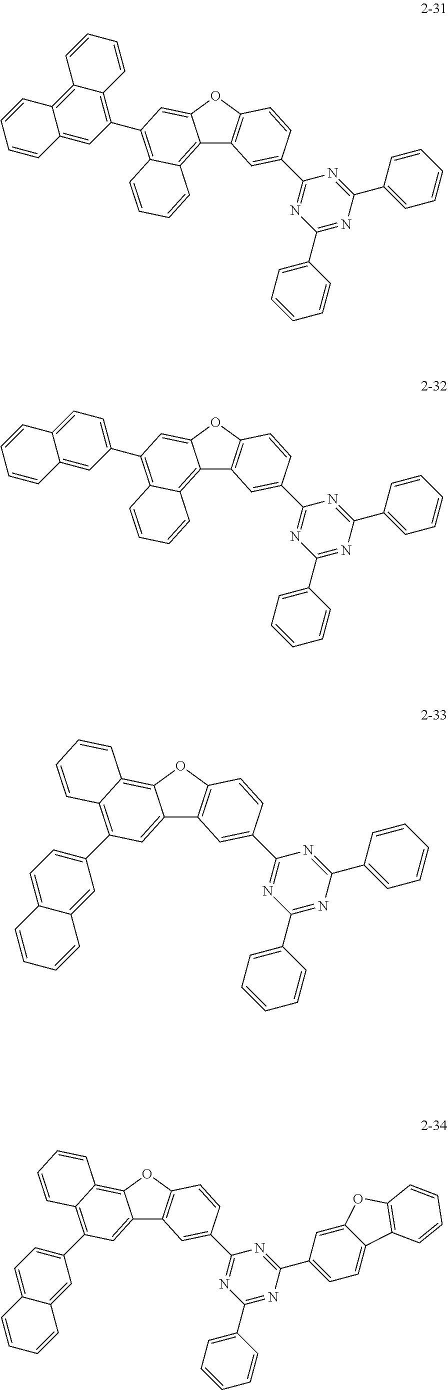 [SMILES: c1ccc(-c2nc(-c3ccccc3)nc(-c3ccc4oc5cc(-c6cc7ccccc7c7ccccc67)c6ccccc6c5c4c3)n2)cc1 ; c1ccc(-c2nc(-c3ccccc3)nc(-c3ccc4oc5cc(-c6ccc7ccccc7c6)c6ccccc6c5c4c3)n2)cc1 ; c1ccc(-c2nc(-c3ccccc3)nc(-c3ccc4oc5c6ccccc6c(-c6ccc7ccccc7c6)cc5c4c3)n2)cc1 ; c1ccc(-c2nc(-c3ccc4c(c3)oc3ccccc34)nc(-c3ccc4oc5c6ccccc6c(-c6ccc7ccccc7c6)cc5c4c3)n2)cc1]

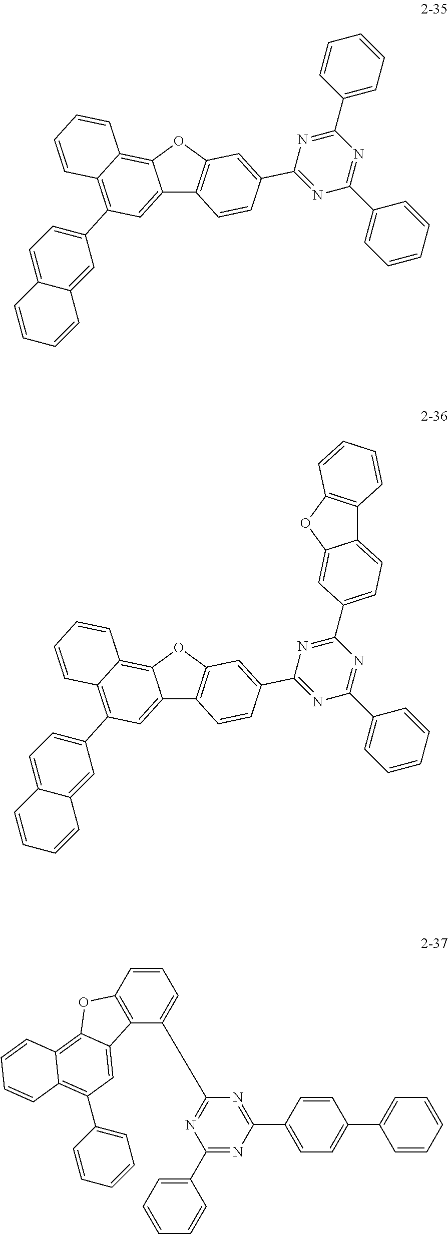 [SMILES: c1ccc(-c2nc(-c3ccccc3)nc(-c3ccc4c(c3)oc3c5ccccc5c(-c5ccc6ccccc6c5)cc43)n2)cc1 ; c1ccc(-c2nc(-c3ccc4c(c3)oc3ccccc34)nc(-c3ccc4c(c3)oc3c5ccccc5c(-c5ccc6ccccc6c5)cc43)n2)cc1 ; c1ccc(-c2ccc(-c3nc(-c4ccccc4)nc(-c4cccc5oc6c7ccccc7c(-c7ccccc7)cc6c45)n3)cc2)cc1]

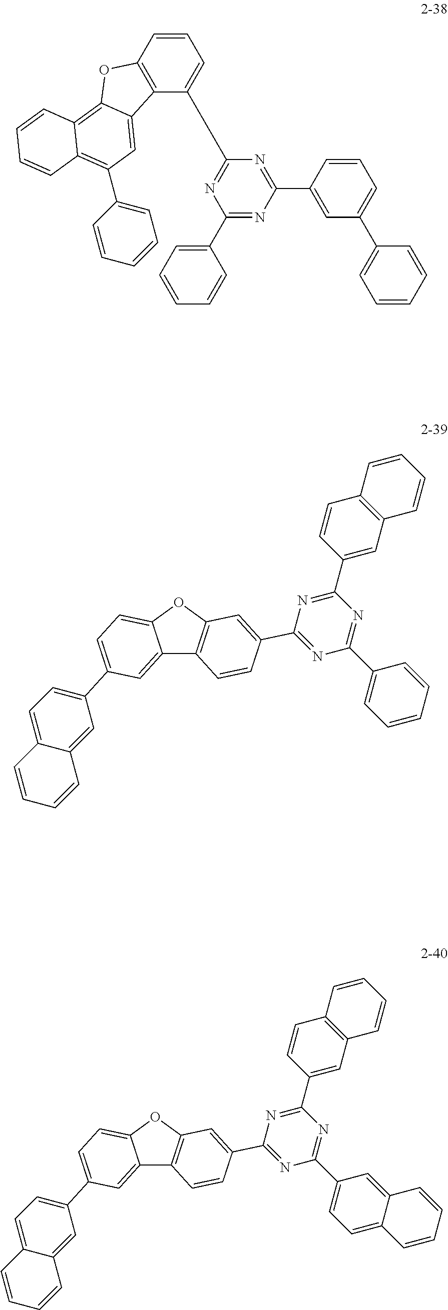 [SMILES: c1ccc(-c2cccc(-c3nc(-c4ccccc4)nc(-c4cccc5oc6c7ccccc7c(-c7ccccc7)cc6c45)n3)c2)cc1 ; c1ccc(-c2nc(-c3ccc4ccccc4c3)nc(-c3ccc4c(c3)oc3ccc(-c5ccc6ccccc6c5)cc34)n2)cc1 ; c1ccc2cc(-c3ccc4oc5cc(-c6nc(-c7ccc8ccccc8c7)nc(-c7ccc8ccccc8c7)n6)ccc5c4c3)ccc2c1]

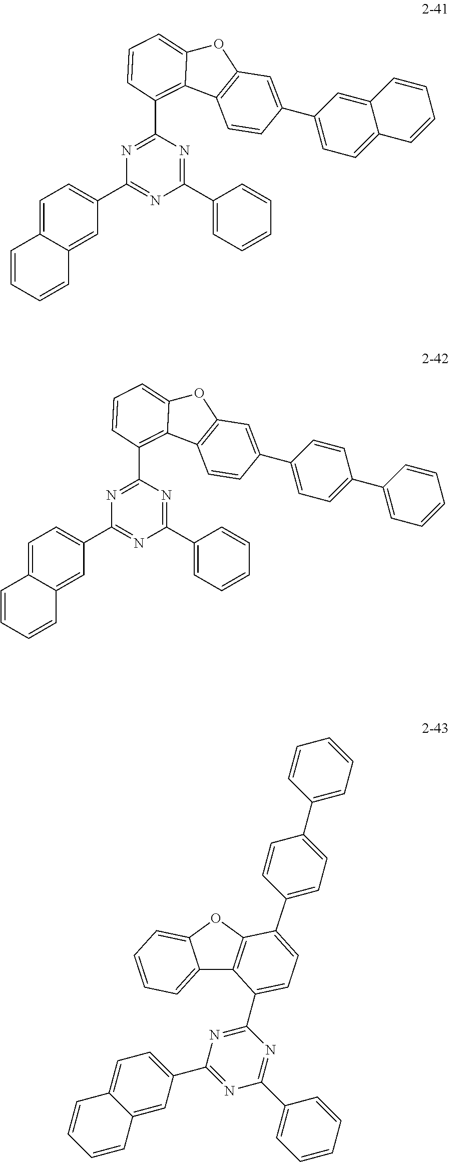 [SMILES: c1ccc(-c2nc(-c3ccc4ccccc4c3)nc(-c3cccc4oc5cc(-c6ccc7ccccc7c6)ccc5c34)n2)cc1 ; c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1]

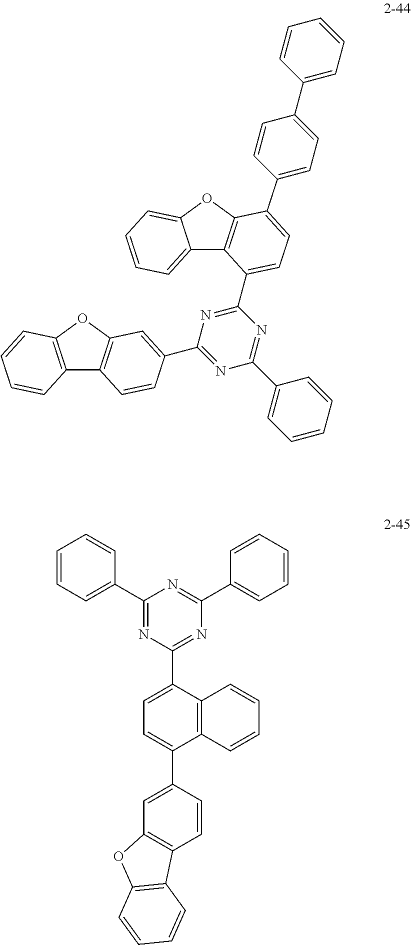 [SMILES: c1ccc(-c2ccc(-c3ccc(-c4nc(-c5ccccc5)nc(-c5ccc6c(c5)oc5ccccc56)n4)c4c3oc3ccccc34)cc2)cc1 ; c1ccc(-c2nc(-c3ccccc3)nc(-c3ccc(-c4ccc5c(c4)oc4ccccc45)c4ccccc34)n2)cc1]

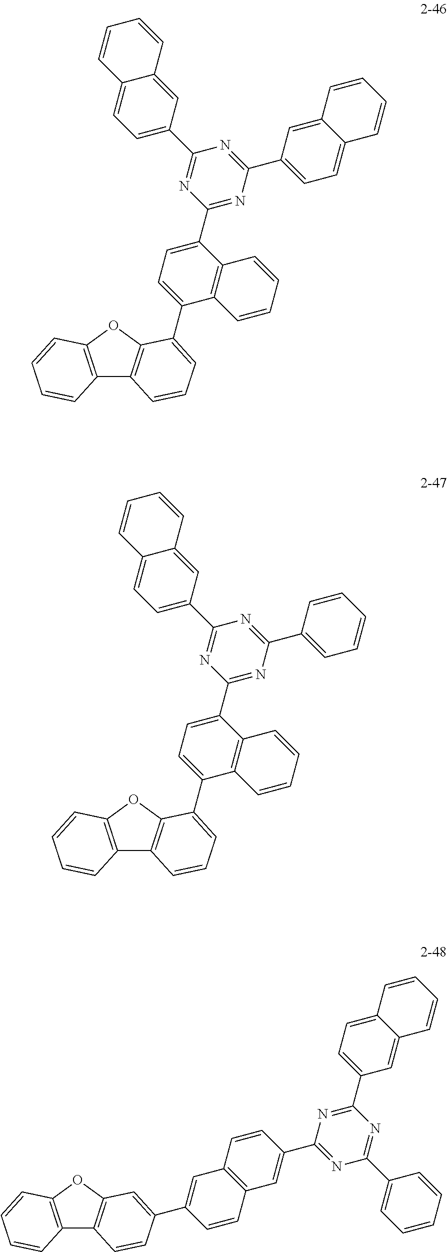 [SMILES: c1ccc2cc(-c3nc(-c4ccc5ccccc5c4)nc(-c4ccc(-c5cccc6c5oc5ccccc56)c5ccccc45)n3)ccc2c1 ; c1ccc(-c2nc(-c3ccc4ccccc4c3)nc(-c3ccc(-c4cccc5c4oc4ccccc45)c4ccccc34)n2)cc1 ; c1ccc(-c2nc(-c3ccc4ccccc4c3)nc(-c3ccc4cc(-c5ccc6c(c5)oc5ccccc56)ccc4c3)n2)cc1]

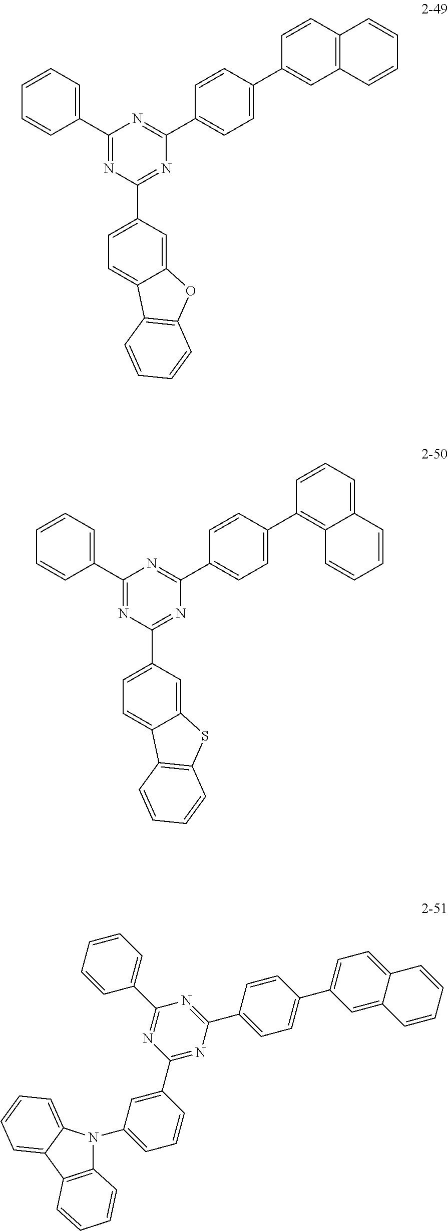 [SMILES: c1ccc(-c2nc(-c3ccc(-c4ccc5ccccc5c4)cc3)nc(-c3ccc4c(c3)oc3ccccc34)n2)cc1 ; c1ccc(-c2nc(-c3ccc(-c4cccc5ccccc45)cc3)nc(-c3ccc4c(c3)sc3ccccc34)n2)cc1 ; c1ccc(-c2nc(-c3ccc(-c4ccc5ccccc5c4)cc3)nc(-c3cccc(-n4c5ccccc5c5ccccc54)c3)n2)cc1]

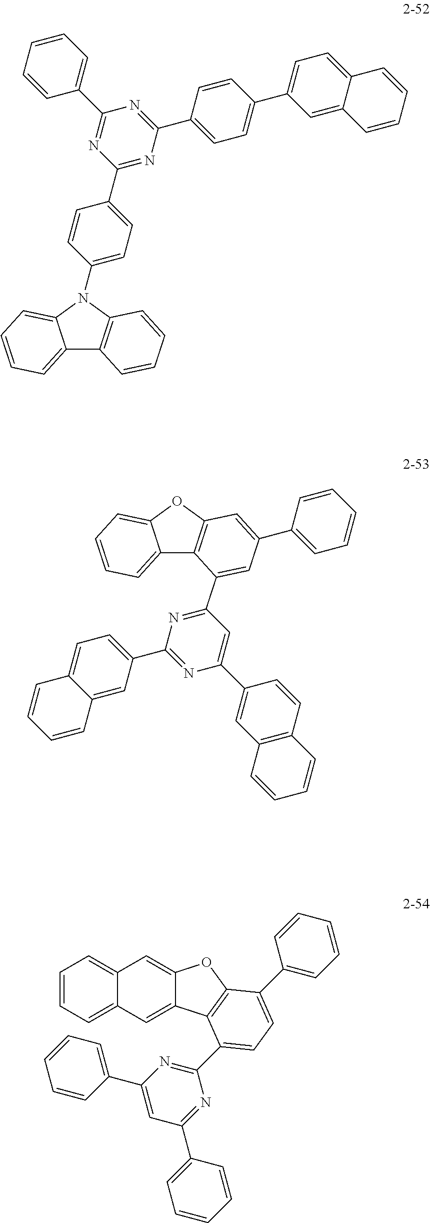 [SMILES: c1ccc(-c2nc(-c3ccc(-c4ccc5ccccc5c4)cc3)nc(-c3ccc(-n4c5ccccc5c5ccccc54)cc3)n2)cc1 ; c1ccc(-c2cc(-c3cc(-c4ccc5ccccc5c4)nc(-c4ccc5ccccc5c4)n3)c3c(c2)oc2ccccc23)cc1 ; c1ccc(-c2cc(-c3ccccc3)nc(-c3ccc(-c4ccccc4)c4oc5cc6ccccc6cc5c34)n2)cc1]

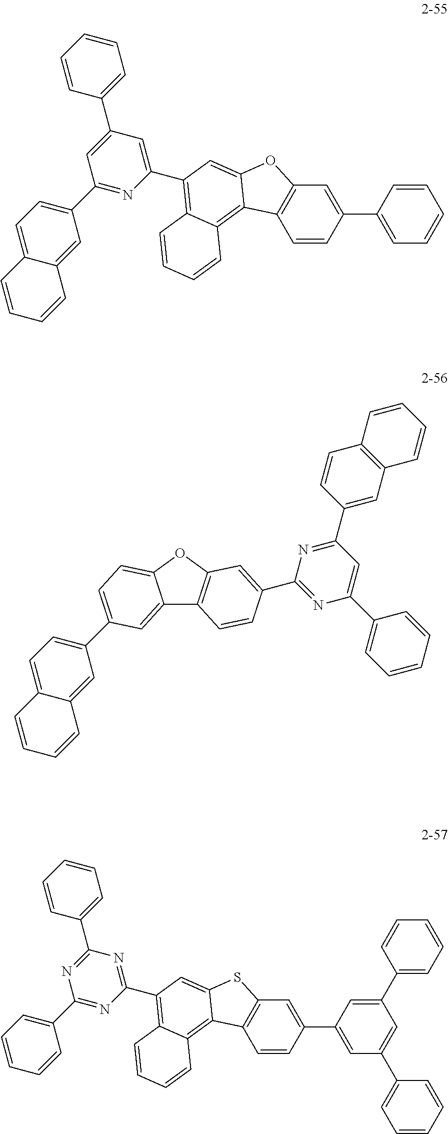 [SMILES: c1ccc(-c2cc(-c3ccc4ccccc4c3)nc(-c3cc4oc5cc(-c6ccccc6)ccc5c4c4ccccc34)c2)cc1 ; c1ccc(-c2cc(-c3ccc4ccccc4c3)nc(-c3ccc4c(c3)oc3ccc(-c5ccc6ccccc6c5)cc34)n2)cc1 ; c1ccc(-c2cc(-c3ccccc3)cc(-c3ccc4c(c3)sc3cc(-c5nc(-c6ccccc6)nc(-c6ccccc6)n5)c5ccccc5c34)c2)cc1]

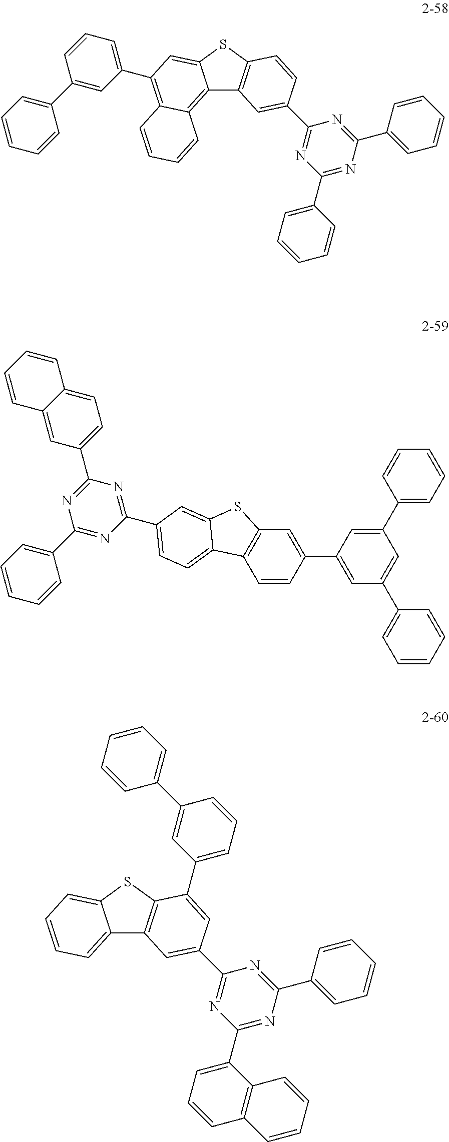 [SMILES: c1ccc(-c2cccc(-c3cc4sc5ccc(-c6nc(-c7ccccc7)nc(-c7ccccc7)n6)cc5c4c4ccccc34)c2)cc1 ; c1ccc(-c2cc(-c3ccccc3)cc(-c3ccc4c(c3)sc3cc(-c5nc(-c6ccccc6)nc(-c6ccc7ccccc7c6)n5)ccc34)c2)cc1 ; c1ccc(-c2cccc(-c3cc(-c4nc(-c5ccccc5)nc(-c5cccc6ccccc56)n4)cc4c3sc3ccccc34)c2)cc1]

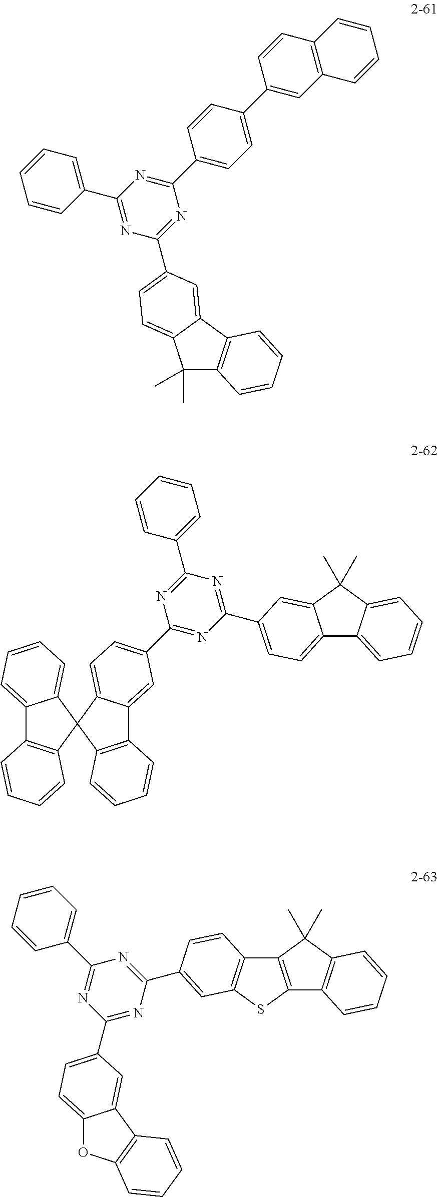 [SMILES: CC1(C)c2ccccc2-c2cc(-c3nc(-c4ccccc4)nc(-c4ccc(-c5ccc6ccccc6c5)cc4)n3)ccc21 ; CC1(C)c2ccccc2-c2ccc(-c3nc(-c4ccccc4)nc(-c4ccc5c(c4)-c4ccccc4C54c5ccccc5-c5ccccc54)n3)cc21 ; CC1(C)c2ccccc2-c2sc3cc(-c4nc(-c5ccccc5)nc(-c5ccc6oc7ccccc7c6c5)n4)ccc3c21]

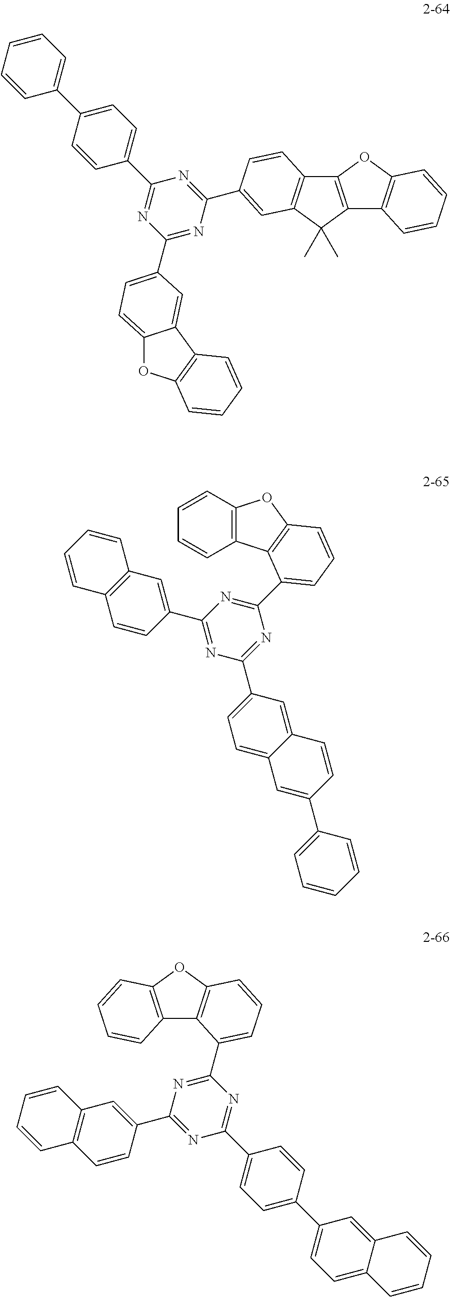 [SMILES: CC1(C)c2cc(-c3nc(-c4ccc(-c5ccccc5)cc4)nc(-c4ccc5oc6ccccc6c5c4)n3)ccc2-c2oc3ccccc3c21 ; c1ccc(-c2ccc3cc(-c4nc(-c5ccc6ccccc6c5)nc(-c5cccc6oc7ccccc7c56)n4)ccc3c2)cc1 ; c1ccc2cc(-c3ccc(-c4nc(-c5ccc6ccccc6c5)nc(-c5cccc6oc7ccccc7c56)n4)cc3)ccc2c1]

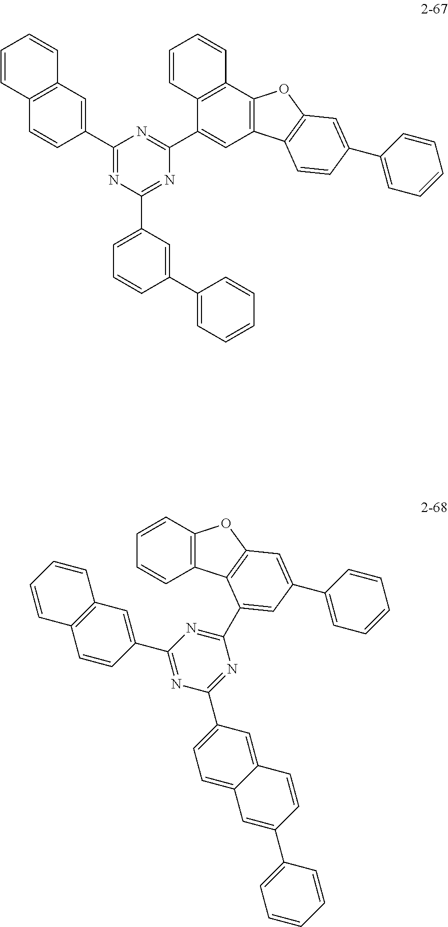 [SMILES: c1ccc(-c2cccc(-c3nc(-c4ccc5ccccc5c4)nc(-c4cc5c6ccc(-c7ccccc7)cc6oc5c5ccccc45)n3)c2)cc1 ; c1ccc(-c2ccc3cc(-c4nc(-c5ccc6ccccc6c5)nc(-c5cc(-c6ccccc6)cc6oc7ccccc7c56)n4)ccc3c2)cc1]

In addition, another embodiment of the present application provides a composition for an organic material layer, the composition including: the heterocyclic compound represented by Chemical Formula 1; and the heterocyclic compound represented by Chemical Formula A.

Specific descriptions on the heterocyclic compound represented by Chemical Formula 1 and the heterocyclic compound represented by Chemical Formula A are the same as the descriptions provided above.

The heterocyclic compound represented by Chemical Formula 1 and the heterocyclic compound represented by Chemical Formula A may have a weight ratio of 1:10 to 10:1, 1:8 to 8:1, 1:5 to 5:1 or 1:2 to 2:1 in the composition, however, the ratio is not limited thereto.

The composition may be used when forming an organic material layer of an organic light emitting device, and particularly, may be more preferably used when forming a host of a light emitting layer.

The composition has a form in which two or more compounds are simply mixed, and materials in a powder state may be mixed before forming an organic material layer of an organic light emitting device, or compounds in a liquid state at a proper temperature or higher may be mixed. The composition is in a solid state at a melting point of each material or lower, and may be kept in a liquid state when adjusting a temperature.

The composition may further include materials known in the art such as solvents and additives.

The organic light emitting device according to one embodiment of the present application may be manufactured using common organic light emitting device manufacturing methods and materials except that one or more organic material layers are formed using the heterocyclic compound represented by Chemical Formula 1 and the heterocyclic compound represented by Chemical Formula A described above.

The compound represented by Chemical Formula 1 and the heterocyclic compound represented by Chemical Formula A may be formed into the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a light emitting layer, an electron transport layer, an electron injection layer and the like as the organic material layer. However, the structure of the organic light emitting device is not limited thereto, and may include a smaller number of organic material layers.

In one embodiment of the present application, the organic light emitting device may be a blue organic light emitting device, and the heterocyclic compound according to Chemical Formula 1 and the heterocyclic compound according to Chemical Formula A may be used as a material of the blue organic light emitting device.

In one embodiment of the present application, the organic light emitting device may be a green organic light emitting device, and the compound represented by Chemical Formula 1 and the heterocyclic compound represented by Chemical Formula A may be used as a material of the green organic light emitting device.

In one embodiment of the present application, the organic light emitting device may be a red organic light emitting device, and the compound represented by Chemical Formula 1 and the heterocyclic compound represented by Chemical Formula A may be used as a material of the red organic light emitting device.

The organic light emitting device of the present disclosure may further include one, two or more layers selected from the group consisting of a light emitting layer, a hole injection layer, a hole transport layer, an electron injection layer, an electron transport layer, an electron blocking layer and a hole blocking layer.

One embodiment of the present application provides an organic light emitting device, wherein the organic material layer includes at least one of a hole blocking layer, an electron injection layer and an electron transport layer, and at least one of the hole blocking layer, the electron injection layer and the electron transport layer includes the heterocyclic compound represented by Chemical Formula 1 and the heterocyclic compound represented by Chemical Formula A.

One embodiment of the present application provides an organic light emitting device, wherein the organic material layer includes a light emitting layer, and the light emitting layer includes the heterocyclic compound represented by Chemical Formula 1 and the heterocyclic compound represented by Chemical Formula A.

One embodiment of the present application provides an organic light emitting device, wherein the organic material layer includes a light emitting layer, the light emitting layer includes a host material, and the host material includes the heterocyclic compound represented by Chemical Formula 1 and the heterocyclic compound represented by Chemical Formula A.

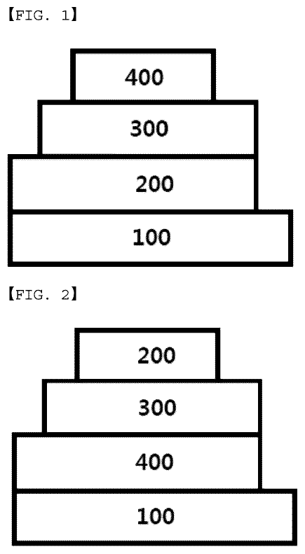

FIGS. 1 to 3 illustrate a lamination order of electrodes and organic material layers of the organic light emitting device according to one embodiment of the present disclosure. However, it is not intended that the scope of the present application be limited by these drawings, and structures of organic light emitting devices known in the art may also be applied to the present application.

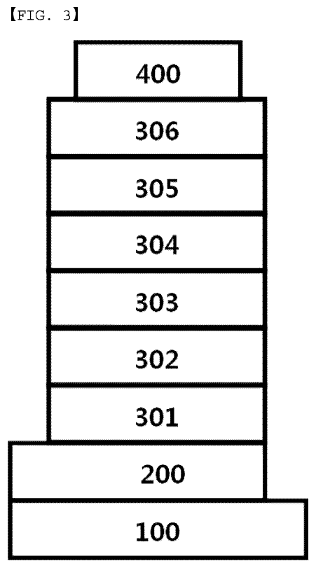

FIG. 1 illustrates an organic light emitting device in which a positive electrode 200, an organic material layer 300 and a negative electrode 400 are sequentially laminated on a substrate 100. However, the structure is not limited only to such a structure, and as illustrated in FIG. 2, an organic light emitting device in which a negative electrode, an organic material layer and a positive electrode are sequentially laminated on a substrate may also be obtained.

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

One embodiment of the present disclosure provides a method for manufacturing an organic light emitting device, the method including: preparing a substrate; forming a first electrode on the substrate; forming one or more organic material layers on the first electrode; and forming a second electrode on the organic material layers, wherein the forming of organic material layers includes forming one or more organic material layers using the composition for an organic material layer according to one embodiment of the present disclosure.

In one embodiment of the present disclosure, the forming of organic material layers may be forming organic material layers using a thermal vacuum deposition method after pre-mixing the heterocyclic compound represented by Chemical Formula 1.

The pre-mixing means, before depositing the heterocyclic compound represented by Chemical Formula 1 on the organic material layer, putting and mixing the materials first in one source of supply.

The pre-mixed material may be referred to as the composition for an organic material layer according to one embodiment of the present application.

The organic material layer including the heterocyclic compound represented by Chemical Formula 1 may further include other materials as necessary.

In the organic light emitting device according to one embodiment of the present disclosure, materials other than the heterocyclic compound represented by Chemical Formula 1 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involved in light emission together may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a principle similar to that in the organic light emitting device.

Hereinafter, preferred examples are provided to help to understand the present disclosure, however, the following examples are only provided to more readily understand the present disclosure, and the present disclosure is not limited thereto.

### PREPARATION EXAMPLE

### <Preparation Example 1> Preparation of Compound 1-1

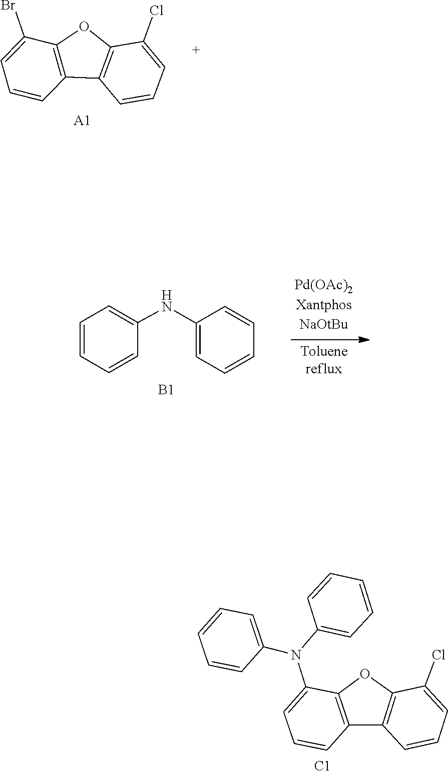

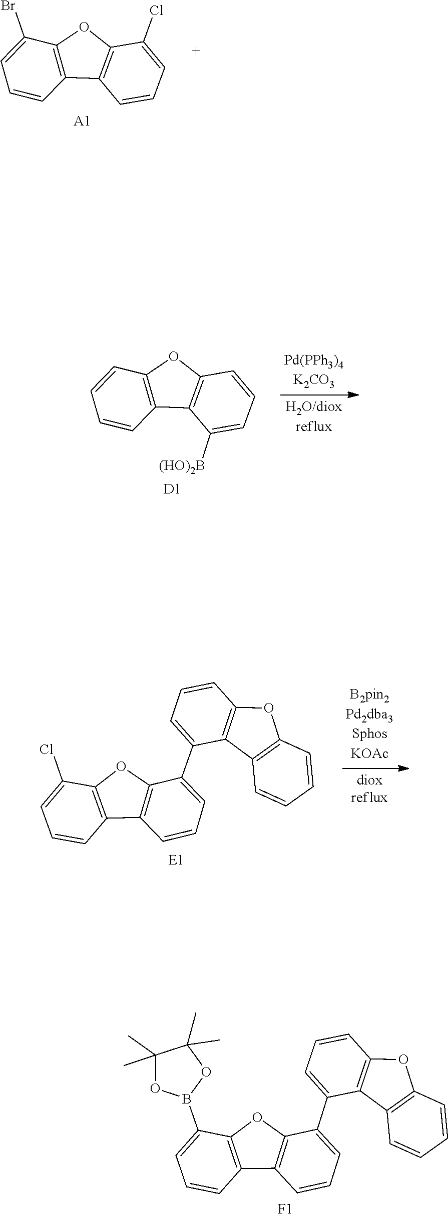

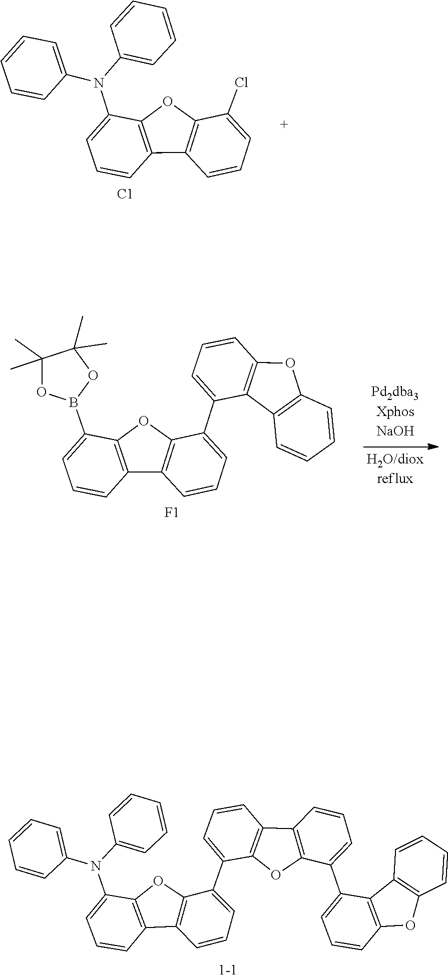

### 1) Preparation of Compound C1

Compound A1 (10.0 g, 35.5 mmol), Compound B1 (6.01 g, 35.5 mmol), Pd(OAc)₂ (0.40 g, 1.78 mmol), Xantphos (2.05 g, 3.55 mmol), NaOtBu (6.82 g, 71.0 mmol) and toluene (100 mL) were introduced to a 250 mL round bottom flask, and the mixture was refluxed for 2 hours. After the reaction was completed, the temperature was lowered to room temperature, and the result was Celite filtered using MC (methylene chloride). The result was concentrated, and then separated using a column under a condition of MC:hexane=1:2 to obtain Compound C1 (8.15 g, 22.0 mmol, yield: 62%).

### 2) Preparation of Compound E1

Compound A1 (20.0 g, 71.0 mmol), Compound D1 (16.6 g, 78.1 mmol), Pd(PPh₃)₄ (4.10 g, 3.55 mmol), K₂CO₃ (19.6 g, 142 mmol), distilled water (60 mL) and dioxane (200 mL) were introduced to a 500 mL round bottom flask, and the mixture was refluxed for 2 hours. After the reaction was completed, the temperature was lowered to room temperature, and the mixture was washed with distilled water and methanol. The solvent was removed using a rotary evaporator, and then the result was recrystallized with toluene to obtain Compound E1 (22.0 g, 59.7 mmol, yield: 84%).

### 3) Preparation of Compound F1

Compound E1 (22.0 g, 59.7 mmol), B₂pin₂ (22.7 g, 89.5 mmol), Pd₂dba₃ (2.73 g, 2.99 mmol), Sphos (2.45 g, 5.97 mmol), KOAc (11.7 g, 119 mmol) and dioxane (220 mL) were introduced to a 1 L round bottom flask, and the mixture was refluxed for 4 hours. After the reaction was completed, the salt was removed through filtering under reduced pressure while hot using MC (methylene chloride). The result was concentrated, and then separated using a column under a condition of MC:hexane=1:3 to obtain Compound F1 (14.3 g, 31.0 mmol, yield: 52%).

### 4) Preparation of Compound 1-1

Compound C1 (8.15 g, 22.0 mmol), Compound F1 (10.7 g, 23.1 mmol), Pd₂dba₃ (1.01 g, 1.10 mmol), Xphos (1.05 g, 2.20 mmol), NaOH (1.76 g, 44.0 mmol), H₂O (24 mL) and dioxane (82 mL) were introduced to a 250 mL round bottom flask, and the mixture was refluxed for 4 hours. After the reaction was completed, the result was extracted with MC (methylene chloride). The result was concentrated, and then separated using a column under a condition of MC:hexane=1:3 to obtain Compound 1-1 (11.9 g, 17.8 mmol, yield: 81%).

### <Preparation Example 2> Preparation of Compounds 1-2, 1-12, 1-13, 1-15, 1-30, 1-32, 1-33, 1-49, 1-57, 1-65, 1-66, 1-76, 1-80, 1-99, 1-109, 1-112, 1-127, 1-139, 1-145, 1-167, 1-169, 1-178, 1-202, 1-226, 1-230, 1-249, 1-265, 1-268, 1-287, 1-327, 1-336, 1-340 and G1

Compounds 1-2, 1-12, 1-13, 1-15, 1-30, 1-32, 1-33, 1-49, 1-57, 1-65, 1-66, 1-76, 1-80, 1-99, 1-109, 1-112, 1-127, 1-139, 1-145, 1-167, 1-169, 1-178, 1-202, 1-226, 1-230, 1-249, 1-265, 1-268, 1-287, 1-327, 1-336, 1-340 and G1 were synthesized in the same manner as in Preparation of Compound 1-1 except that Compounds A1 to F1 of the following Table 1 were used instead of Compounds A1 to F1 described above.

TABLE 1

| Com- | Compound | Compound | Compound | Compound | Compound | Compound | |
|---|---|---|---|---|---|---|---|
| pound | A1 | B1 | C1 | D1 | E1 | F1 | Yield |
| 1-2 | 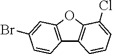 | 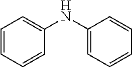 | 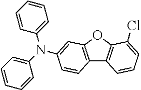 | 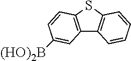 | 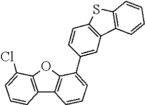 | 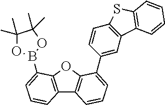 | 23% |
| 1-12 | 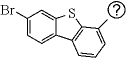 [symbol] | 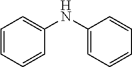 | 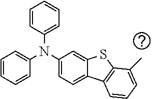 | 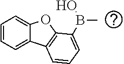 | 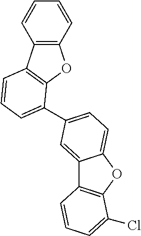 | 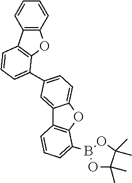 | 31% |
| 1-13 | 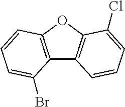 | 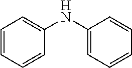 | 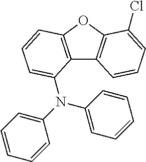 | 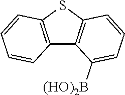 | 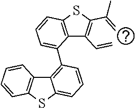 | 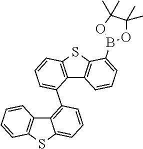 | 27% |
| 1-15 | 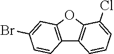 | 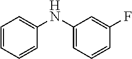 | 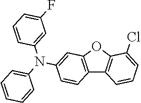 | 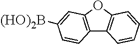 | 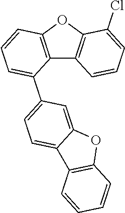 | 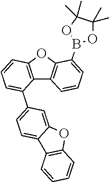 | 25% |
| 1-18 | 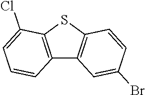 | 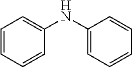 | 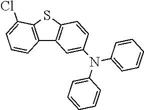 | 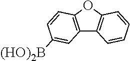 | 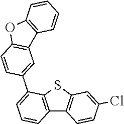 | 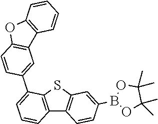 | 26% |
| 1-30 | 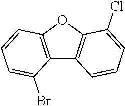 | 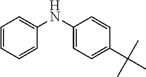 | 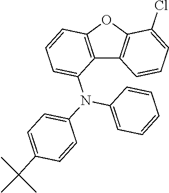 | 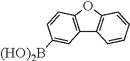 | 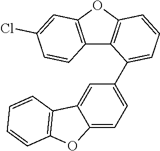 | 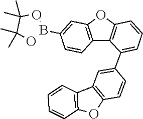 | 23% |
| 1-32 | 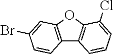 | 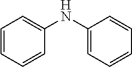 | 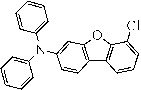 | 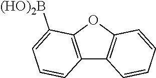 | 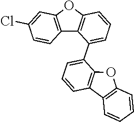 | 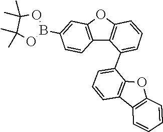 | 19% |
| 1-33 | 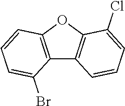 | 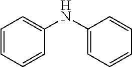 | 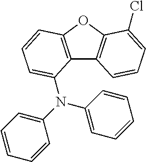 | 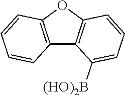 | 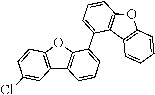 | 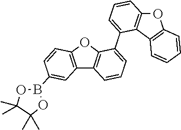 | 20% |
| 1-49 | 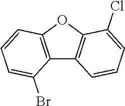 | 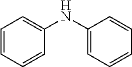 | 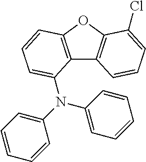 | 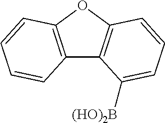 | 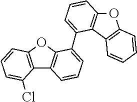 | 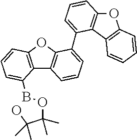 | 24% |
| 1-57 | 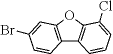 | 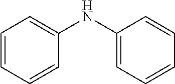 | 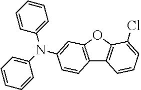 | 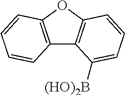 | 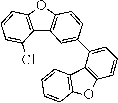 | 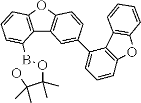 | 22% |
| 1-65 | 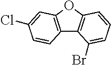 | 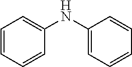 | 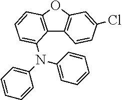 | 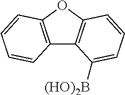 | 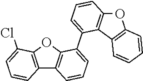 | 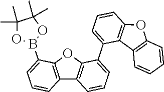 | 23% |
| 1-66 | 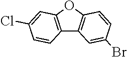 | 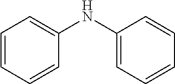 | 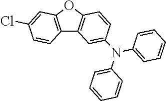 | 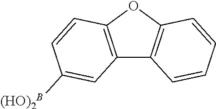 | 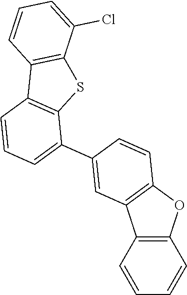 | 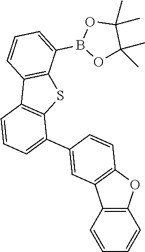 | 31% |
| 1-76 | 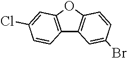 | 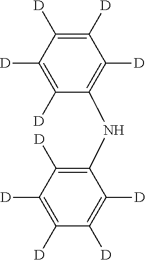 | 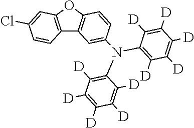 | 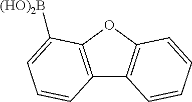 | 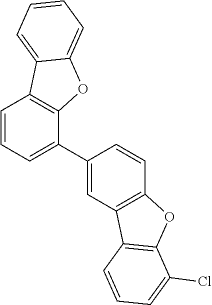 | 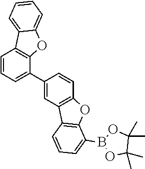 | 34% |
| 1-80 | 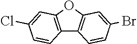 | 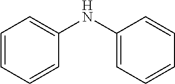 | 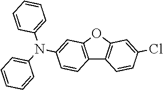 | 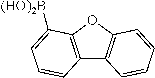 | 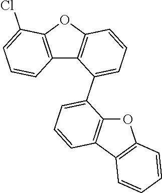 | 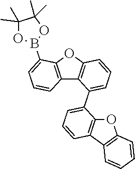 | 20% |
| 1-99 | 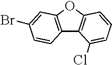 | 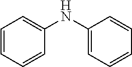 | 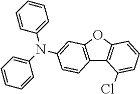 | 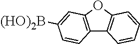 | 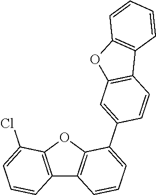 | 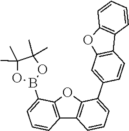 | 21% |
| 1-109 | 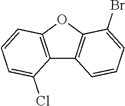 | 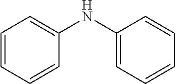 | 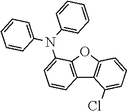 | 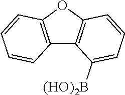 | 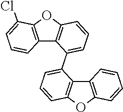 | 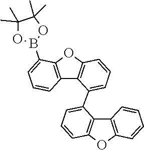 | 19% |
| 1-112 | 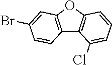 | 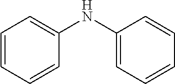 | 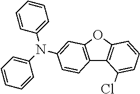 | 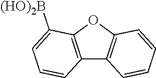 | 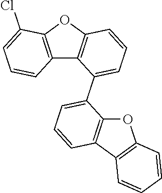 | 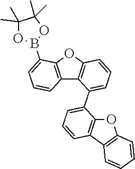 | 25% |
| 1-127 | 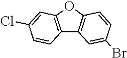 | 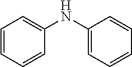 | 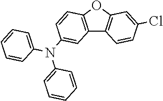 | 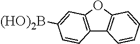 | 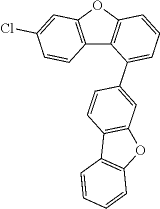 | 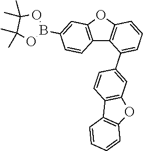 | 23% |
| 1-139 | 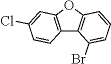 | 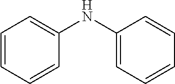 | 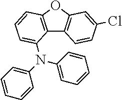 | 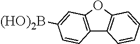 | 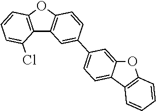 | 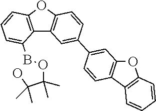 | 22% |
| 1-145 | 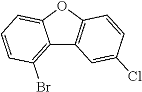 | 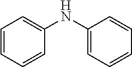 | 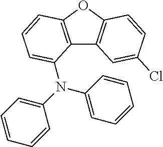 | 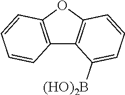 | 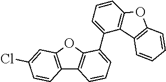 | 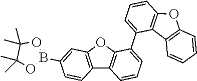 | 18% |
| 1-167 | 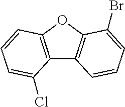 | 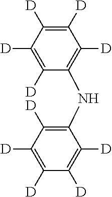 | 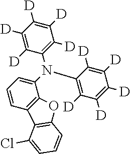 | 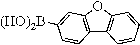 | 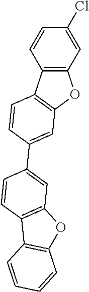 | 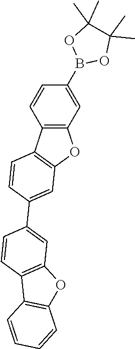 | 31% |
| 1-169 | 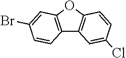 | 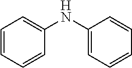 | 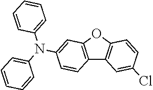 | 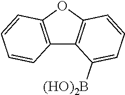 | 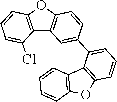 | 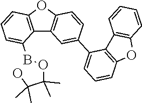 | 23% |
| 1-178 | 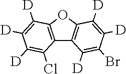 | 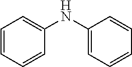 | 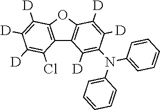 | 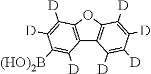 | 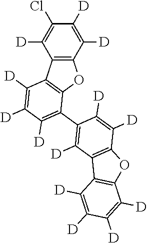 | 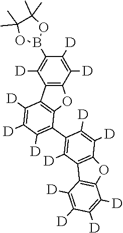 | 25% |
| 1-202 | 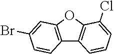 | 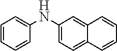 | 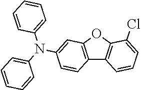 | 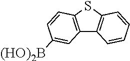 | 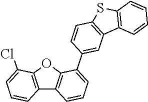 | 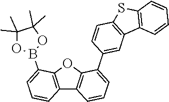 | 21% |
| 1-226 | 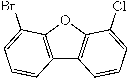 | 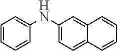 | 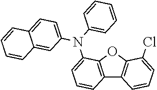 | 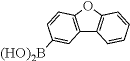 | 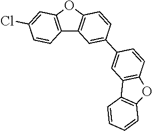 | 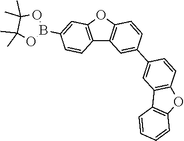 | 25% |
| 1-230 | 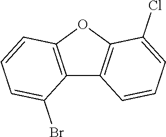 | 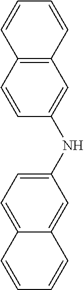 | 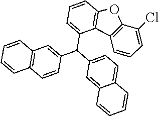 | 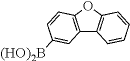 | 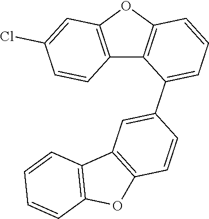 | 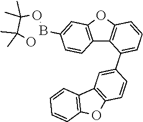 | 33% |
| 1-249 | 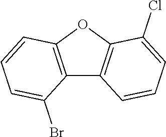 | 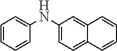 | 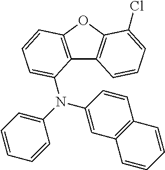 | 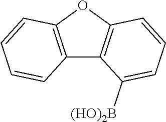 | 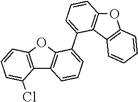 | 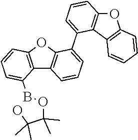 | 19% |
| 1-265 | 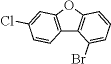 | 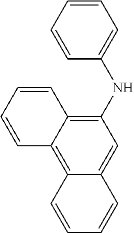 | 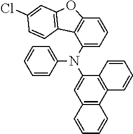 | 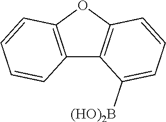 | 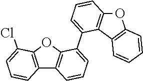 | 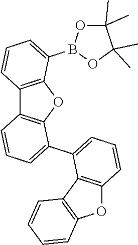 | 27% |
| 1-268 | 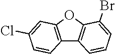 | 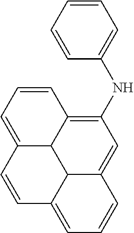 | 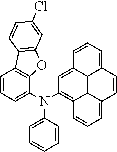 | 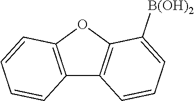 | 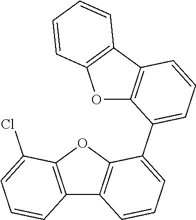 | 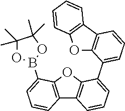 | 34% |
| 1-287 | 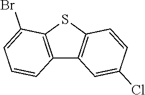 | 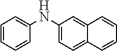 | 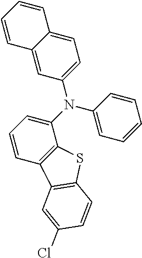 | 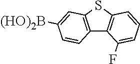 | 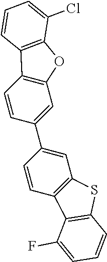 | 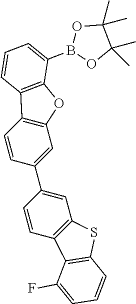 | 36% |
| 1-327 | 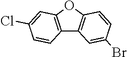 | 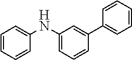 | 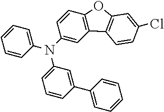 | 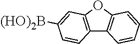 | 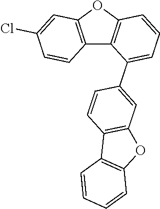 | 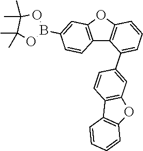 | 24% |
| 1-336 | 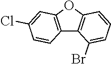 | 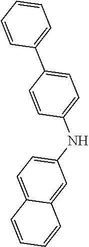 | 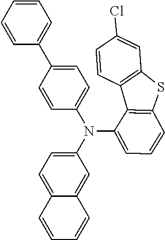 | 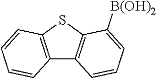 | 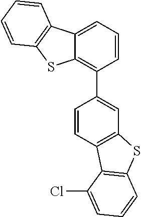 | 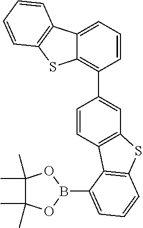 | 33% |
| 1-340 | 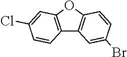 | 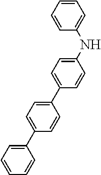 | 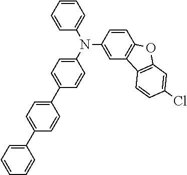 | 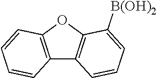 | 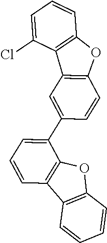 | 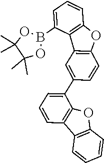 | 29% |
| G1 | 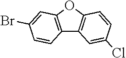 | 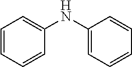 | 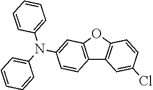 | 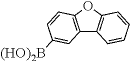 | 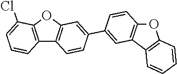 | 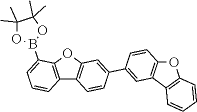 | 22% |

[symbol]

indicates data missing or illegible when filed

### <Preparation Example 3> Preparation of Compound 1-86

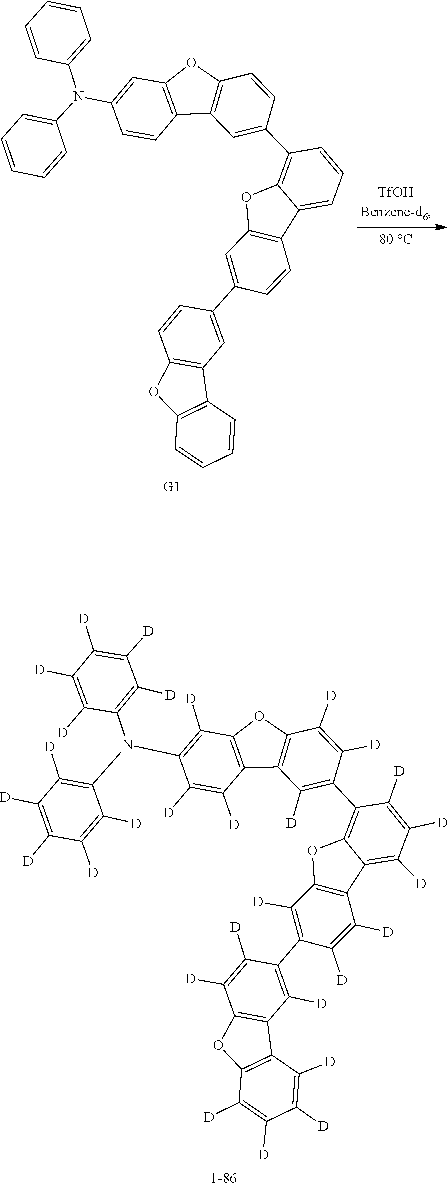 [SMILES: c1ccc(N(c2ccccc2)c2ccc3c(c2)oc2ccc(-c4cccc5c4oc4cc(-c6ccc7oc8ccccc8c7c6)ccc45)cc23)cc1 ; [2H]c1c([2H])c([2H])c(N(c2c([2H])c([2H])c([2H])c([2H])c2[2H])c2c([2H])c([2H])c3c(oc4c([2H])c([2H])c(-c5c([2H])c([2H])c([2H])c6c5oc5c([2H])c(-c7c([2H])c([2H])c8oc9c([2H])c([2H])c([2H])c([2H])c9c8c7[2H])c([2H])c([2H])c56)c([2H])c43)c2[2H])c([2H])c1[2H]]

Compound G1 (10.0 g, 15.0 mmol) and benzene-d6 (100 mL) were introduced to a 1 L round bottom flask, and TfOH (triflic acid) (9 mL) was slowly added dropwise thereto. The mixture was stirred for 1 hour (h) at 80° C. After the reaction was completed, the result was cooled to room temperature, and Celite filtered using methylene chloride (MC). The result was concentrated, and then separated using a column under a condition of MC:hexane=1:3 to obtain Compound 1-86 (8.68 g, 12.5 mmol, 83%).

### <Preparation Example 4> Preparation of Compounds 1-192 and 1-271

Compounds 1-192 and 1-271 were synthesized in the same manner as in Preparation of Compound 1-86 except that Compound G of the following Table 2 was used instead of Compound G described above.

TABLE 2
Target Compound

| Compound | | | |
|---|---|---|---|
| No. | Compound G | Target Compound | Yield |
| 1-192 | 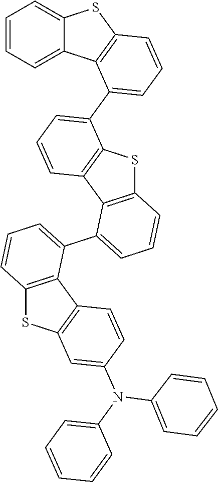 [SMILES: c1ccc(N(c2ccccc2)c2ccc3c(c2)sc2cccc(-c4cccc5sc6c(-c7cccc8sc9ccccc9c78)cccc6c45)c23)cc1] | 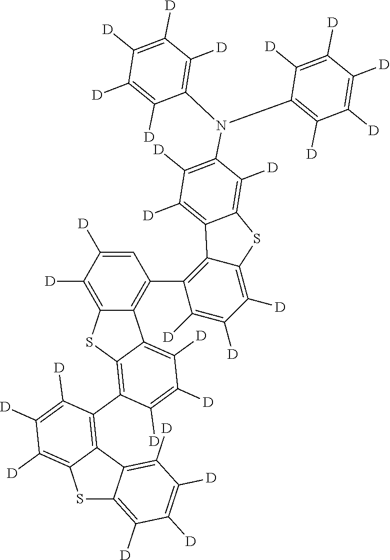 [SMILES: [2H]c1cc(-c2c([2H])c([2H])c([2H])c3sc4c([2H])c(N(c5c([2H])c([2H])c([2H])c([2H])c5[2H])c5c([2H])c([2H])c([2H])c([2H])c5[2H])c([2H])c([2H])c4c23)c2c(sc3c(-c4c([2H])c([2H])c([2H])c5sc6c([2H])c([2H])c([2H])c([2H])c6c45)c([2H])c([2H])c([2H])c32)c1[2H]] | 89% |
| 1-271 | 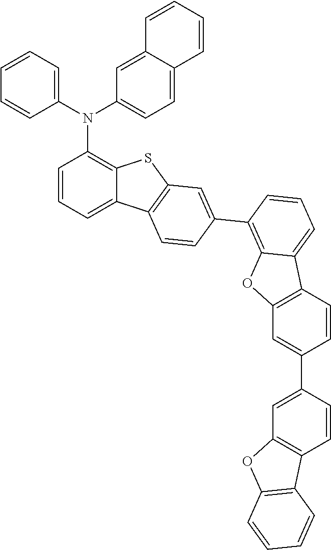 [SMILES: c1ccc(N(c2ccc3ccccc3c2)c2cccc3c2sc2cc(-c4cccc5c4oc4cc(-c6ccc7c(c6)oc6ccccc67)ccc45)ccc23)cc1] | 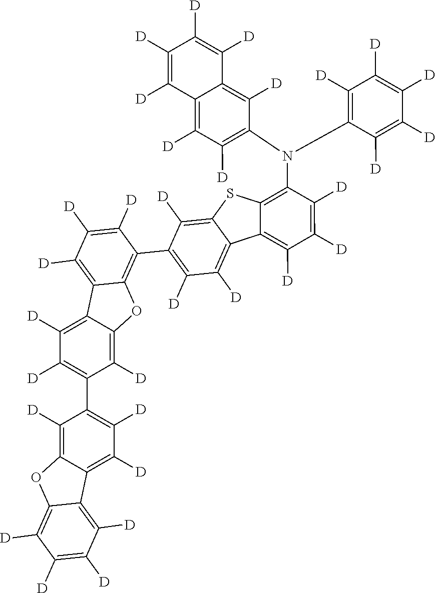 [SMILES: [2H]c1c([2H])c([2H])c(N(c2c([2H])c([2H])c3c([2H])c([2H])c([2H])c([2H])c3c2[2H])c2c([2H])c([2H])c([2H])c3c2sc2c([2H])c(-c4c([2H])c([2H])c([2H])c5c4oc4c([2H])c(-c6c([2H])c([2H])c7c(oc8c([2H])c([2H])c([2H])c([2H])c87)c6[2H])c([2H])c([2H])c45)c([2H])c([2H])c23)c([2H])c1[2H]] | 77% |

### <Preparation Example 5> Preparation of Compound 2-1

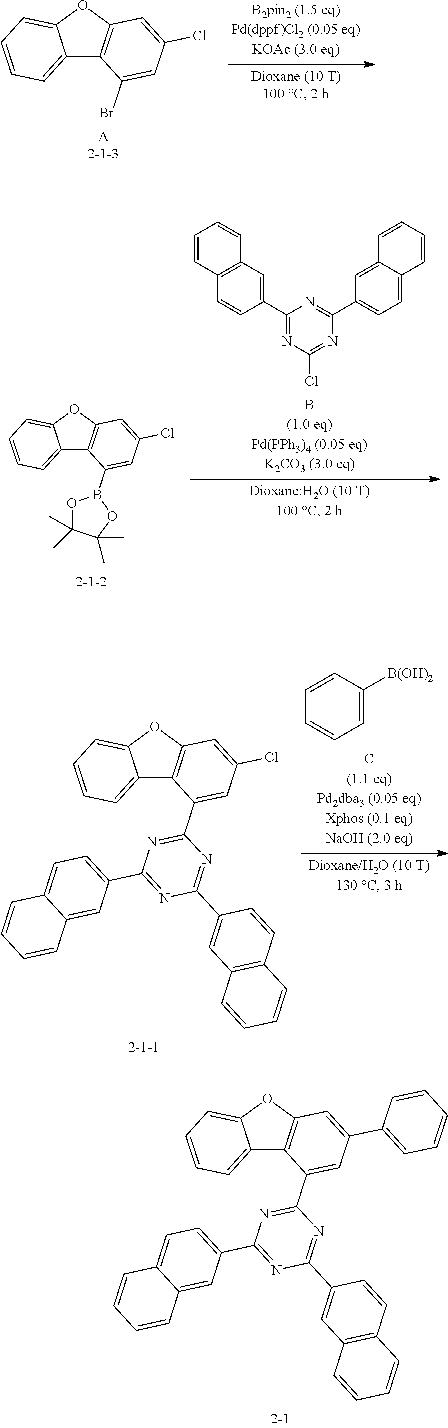 [SMILES: Clc1cc(Br)c2c(c1)oc1ccccc12 ; Clc1nc(-c2ccc3ccccc3c2)nc(-c2ccc3ccccc3c2)n1 ; CC1(C)OB(c2cc(Cl)cc3oc4ccccc4c23)OC1(C)C ; Clc1cc(-c2nc(-c3ccc4ccccc4c3)nc(-c3ccc4ccccc4c3)n2)c2c(c1)oc1ccccc12 ; OB(O)c1ccccc1 ; c1ccc(-c2cc(-c3nc(-c4ccc5ccccc5c4)nc(-c4ccc5ccccc5c4)n3)c3c(c2)oc2ccccc23)cc1]

### 1) Preparation of Intermediate 2-1-2

1-bromo-3-chlorodibenzo[b,d]furan (10.0 g, 35.5 mmol), B₂pin₂ (13.5 g, 53.3 mmol), Pd(dppf)Cl₂ (1.30 g, 1.78 mmol) and KOAc (10.2 g, 107 mmol) were introduced to dioxane (100 mL), and the mixture was stirred for 2 hours at 100° C. The result was filtered while hot, and washed several times with methylene dichloride. The filtrate was concentrated under reduced pressure, and then silica gel filtered. The result was concentrated under reduced pressure to obtain Intermediate 2-1-2 (10.1 g, 87%).

### 2) Preparation of Intermediate 2-1-1

Intermediate 2-1-2 (10.1 g, 30.7 mmol), 2-chloro-4,6-di(naphthalen-2-yl)-1,3,5-triazine (11.3 g, 30.7 mmol), Pd(PPh₃)₄ (1.77 g, 1.54 mmol) and K₂CO₃ (12.7 g, 92.1 mmol) were introduced to dioxane/H₂O (100 mL/30 mL), and the mixture was stirred for 2 hours at 100° C. The reaction solution was cooled to room temperature, and produced solids were filtered to obtain Intermediate 2-1-1 (12.8 g, 80%).

### 3) Preparation of Compound 2-1

Intermediate 2-1-1 (14.8 g, 27.7 mmol), phenylboronic acid (3.72 g, 30.5 mmol), Pd₂dba₃ (1.27 g, 0.05 mmol), Xphos (1.32 g, 2.77 mmol) and NaOH (2.22 g, 55.4 mmol) were introduced to dioxane/H₂O, and the mixture was stirred for 3 hours at 130° C. The result was cooled to room temperature, and produced solids were filtered. The result was silica gel filtered to obtain Compound 2-1 (7.36 g, 74%).

### <Preparation Example 6> Preparation of Compounds 2-3, 2-5, 2-8, 2-21 and 2-30

The following compounds were synthesized in the same manner as in Preparation of Compound 2-1 of Preparation Example 5 except that A, B and C of the following Table 3 were used as the intermediates.

TABLE 3

| Compound | A | B | C | Yield |
|---|---|---|---|---|
| 2-3 | 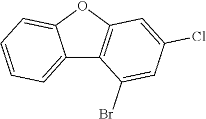 | 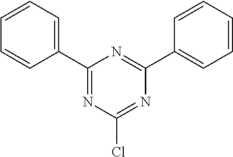 | 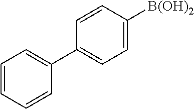 | 80% |
| 2-5 | 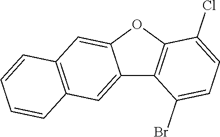 | 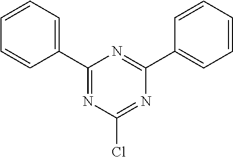 | 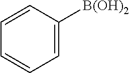 | 77% |
| 2-8 | 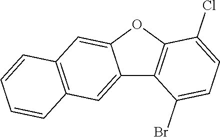 | 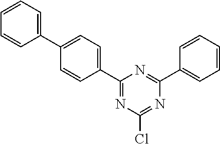 | 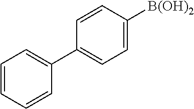 | 87% |
| 2-21 | 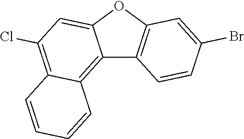 | 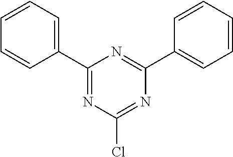 | 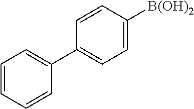 | 82% |
| 2-30 | 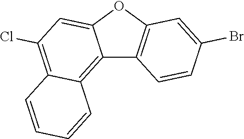 | 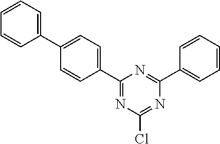 | 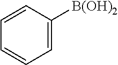 | 83% |

The rest of compounds other than the compounds described in Preparation Examples 1 to 5 and Tables 1 to 3 were also prepared in the same manner as in the preparation examples described above, and the synthesis results are shown in the following Table 4 and Table 5. The following Table 4 shows measurement values of 1H NMR (CDCl₃, 400 MHz), and the following Table 5 shows measurement values of FD-MS spectrometry (FD-MS: field desorption mass spectrometry). ¹H was not present in Compounds 1-86, 1-192 and 1-271, and ¹H NMR (CDCl₃, 400 MHz) measurement was skipped.

TABLE 4

| Com- | |
|---|---|
| pound | ¹H NMR (CDCl₃, 400 MHz) |
| 1-1 | δ = 8.22~8.20 (d, 1H), 8.09~8.07(d, 1H), |
| | 7.77~7.68 (m, 6H), 7.66~7.58 (m, 4H), |
| | 7.54~7.44 (m, 9H), 7.42~7.36 (m, 3H), |
| | 7.29~7.26 (m, 3H), 7.24~7.22 (d, 1H), |
| | 7.17~7.15 (d, 1H) |
| 1-2 | δ = 8.56 (s, 1H), 8.25~8.23 (d, 1H), |
| | 8.11~8.09 (d, 1H), 8.05~8.03 (d, 1H), |
| | 8.01~7.99 (d, 1H), 7.86~7.81 (m, 3H), |
| | 7.75~7.71 (m, 5H), 7.67~7.63 (m, 7H), |
| | 7.54~7.50 (m, 7H), 7.45~7.38 (m, 2H) |
| 1-12 | δ = 8.55 (d, 1H), 8.32 (d, 1H), 8.08~7.98 |
| | (m, 6H), 7.88~7.79 (m, 3H), 7.70 (t, |
| | 1H), 7.64 (s, 1H), 7.54~7.24 (m, 10H), |
| | 7.08~7.00 (m, 6H) |
| 1-13 | δ = 8.55 (d, 1H), 8.45 (d, 1H), 8.32 |
| | (d, 1H), 8.08~7.93 (m, 7H), 7.70~7.68 (m, |
| | 3H), 7.56~7.49 (m, 3H), 7.34 (t, 1H), |
| | 7.25~7.24 (m, 5H), 7.08~6.91 (m, 7H) |
| 1-15 | δ = 8.08~7.98 (m, 7H), 7.82~7.76 (m, 4H), |
| | 7.57~7.51 (m, 4H), 7.39~7.24 (m, 6H), |
| | 7.08~7.00 (m, 3H), 6.91~6.85 (m, 2H), |
| | 6.76 (t, 1H) |
| 1-18 | δ = 8.55 (d, 1H), 8.32~8.17 (m, 5H), |
| | 7.98~7.95 (m, 2H), 7.88~7.70 (m, 5H), 7.54 |
| | (d, 1H), 7.41~7.24 (m, 7H), 7.08~7.00 (m, 6H) |
| 1-30 | δ = 8.08~7.98 (m, 3H), 7.88~7.76 (m, 6H), |
| | 7.69 (d, 1H), 7.57~7.51 (m, 3H), |
| | 7.39~7.24 (m, 6H), 7.10~7.00 (m, 7H), |
| | 6.91 (d, 1H), 1.33 (s, 9H) |
| 1-32 | δ = 8.17~8.15 (d, 1H), 8.12~8.10 (d, 2H), |
| | 8.06~8.04 (d, 1H), 7.79~7.75 (m, |
| | 3H), 7.73~7.52 (m, 13H), 7.43~7.40 |
| | (m, 7H), 7.21~7.19 (d, 2H) |
| 1-33 | δ = 8.12~8.10 (d, 1H), 8.08~8.03 (m, 2H), |
| | 7.94~7.85 (m, 3H), 7.78~7.55 (m, 7H), |
| | 7.52~7.47 (m, 5H), 7.34~7.22 (m, 7H), |
| | 7.19~7.17 (d, 1H), 7.12~6.90 (m, 3H) |
| 1-49 | δ = 8.09~8.01 (m, 3H), 7.83~7.78 (m, 4H), |
| | 7.72-7.67 (m, 4H), 7.58~7.56 (d, 1H), |
| | 7.51~7.48 (m, 6H), 7.33~7.26 (m, 6H), |
| | 7.23~6.92 (m, 5H) |
| 1-57 | δ = 8.28 (s, 1H), 8.14~8.12 (d, 1H), |
| | 7.85~7.81 (m, 5H), 7.70~7.59 (m, 12H), |
| | 7.57~7.40 (m, 5H), 7.27~7.25 (m, 4H), |
| | 7.13~7.11 (t, 1H) |
| 1-65 | δ = 8.15~8.13 (d, 1H), 8.09~8.07(d, 1H), |
| | 7.82-7.80 (d, 1H), 7.78-7.76 (d, 1H), |
| | 7.74~7.72 (m, 4H), 7.70~7.55 (m, 11H), |
| | 7.49~7.41 (m, 6H), 7.16~7.12 (m, 4H) |
| 1-66 | δ = 8.55 (d, 2H), 8.32 (d, 2H), 8.22 (s, 1H), |
| | 8.03~7.98 (m, 2H), 7.88~7.70 (m, |
| | 7H), 7.56~7.54 (m, 2H), 7.39~7.24 |
| | (m, 6H), 7.08~6.97 (m, 7H) |
| 1-76 | δ = 8.22 (s, 1H), 8.08~7.98 (m, 6H), |
| | 7.88~7.76 (m, 5H), 7.56~7.51 (m, 4H), |
| | 7.39 (t, 1H), 7.31 (t, 1H), 6.97 (d, 1H) |
| 1-80 | δ = 8.16~8.14 (d, 1H), 8.10~8.08 (d, 1H), |
| | 7.85~7.82 (d, 2H), 7.77~7.70 (m, 7H), |
| | 7.69~7.60 (m, 3H), 7.58~7.51 (m, 8H), |
| | 7.45~7.43 (m, 6H), 7.21~7.19 (d, 1H) |
| 1-99 | δ = 8.78 (s, 1H), 8.18~8.16 (d, 1H), |
| | 8.13~8.11 (d, 1H), 8.06~8.02 (m, 4H), |
| | 7.82~7.77(m, 3H), 7.73~7.71 (d, 1H), |
| | 7.67~7.62 (m, 8H), 7.55~7.49 (m, 6H), |
| | 7.45~7.34 (m, 4H) |
| 1-109 | δ = 7.80~7.47 (m, 3H), 7.67~7.55 (m, 10H), |
| | 7.50~7.47 (d, 2H), 7.36~7.32 (m, 2H), |
| | 7.25~7.13 (m, 9H), 7.02~7.00 (d, 1H), |
| | 6.97~6.95 (m, 2H) |
| 1-112 | δ = 8.52~8.50 (d, 1H), 8.17~8.15 (m, 3H), |
| | 8.10~8.09 (d, 1H), 8.02~8.00 (d, 1H), |
| | 7.77~7.66 (m, 6H), 7.62~7.51 (m, 9H), |
| | 7.49~7.39 (m, 6H), 7.19~7.17 (m, 2H) |
| 1-127 | δ = 8.15~8.13 (d, 2H), 7.96 (s, 1H), |
| | 7.90~7.81 (m, 3H), 7.70~7.58 (m, 10H), |
| | 7.56~7.50 (m, 5H), 7.47~7.26 (m, 8H) |
| 1-139 | δ = 8.16~8.14 (d, 1H), 8.09~8.07 |
| | (d, 1H), 7.88 (s, 1H) 7.82~7.80 (d, 1H), |
| | 7.73~7.54 (m, 14H), 7.52~7.40 (m, 3H), |
| | 7.38~7.24 (m, 8H) |
| 1-145 | δ = 8.39 (s, 1H), 8.22~8.20 (d, 1H), |
| | 7.91~7.89 (m, 5H), 7.78~7.75 (m, 5H), |
| | 7.65~7.53 (m, 13H), 7.46~7.44 (m, 2H), |
| | 7.19~7.17 (m, 2H) |
| 1-167 | δ = 8.03~7.98 (m, 4H), 7.82~7.54 (m, 11H), |
| | 7.39~7.28 (m, 3H), 6.97 (d, 1H) |
| 1-169 | δ = 8.40~8.38 (d, 1H), 8.26 (s, 1H), |
| | 8.01~7.99 (d, 1H), 7.83~7.81 (m, 2H), |
| | 7.77 (s, 2H), 7.69~7.48 (m, 14H), 7.42~7.38 |
| | (m, 6H), 7.23~7.19 (m, 2H) |
| 1-178 | δ = 7.24 (t, 4H), 7.08 (d, 4H), 7.00 (t, 2H) |
| 1-202 | δ = 8.55 (s, 1H), 8.24~8.22 (d, 1H), |
| | 8.10~8.08 (d, 1H), 8.06~8.04 (d, 1H), |
| | 8.01~7.99 (d, 1H), 7.87~7.82 (m, 3H), |
| | 7.76~7.70 (m, 5H), 7.66~7.60 (m, 8H), |
| | 7.55~7.49 (m, 8H), 7.44~7.38 (m, 2H) |
| 1-226 | δ = 8.65~8.63 (d, 1H), 8.44~8.42 |
| | (d, 1H), 8.21 (s, 1H), 8.20~8.18 (m, 2H), |
| | 8.00~7.98 (m, 4H), 7.84~7.82 (d, 1H), |
| | 7.74-7.72 (d, 1H), 7.70~7.62 (m, 9H), |
| | 7.56~7.49 (m, 5H), 7.42~7.39 (m, 3H), |
| | 7.27~7.25 (d, 1H) |
| 1-230 | δ = 8.08~7.98 (m, 4H), 7.82~7.69 |
| | (m, 11H), 7.57~7.25 (m, 15H), 7.11 |
| | (s, 2H), 6.91 (d, 1H) |
| 1-249 | δ = 8.07~8.00 (m, 3H), 7.82~7.77 (m, 4H), |
| | 7.73~7.68 (m, 4H), 7.59~7.57 (d, 1H), |
| | 7.52~7.49 (m, 7H), 7.40~7.30 (m, 7H), |
| | 7.25~7.16 (m, 5H) |
| 1-265 | δ = 8.98 (d, 1H), 8.84 (d, 1H) 8.08~7.54 |
| | (m, 21H), 7.39~7.24 (m, 6H) |
| | 7.08~6.91 (m, 4H) |
| 1-268 | δ = 8.31 (d, 2H), 8.08~7.98 (m, 8H), |
| | 7.82 (d, 1H), 7.76 (s, 1H), 7.64 (d, 1H), |
| | 7.54~7.49 (m, 5H), 7.39~7.24 (m, 6H), |
| | 7.08~6.97 (m, 4H) |
| 1-287 | δ = 8.24~7.99 (m, 10H), 7.82~7.70 (m, 5H), |
| | 7.54~7.40 (m, 8H), 7.24~7.21 (m, 3H), |
| | 7.11~7.00 (m, 4H) |
| 1-327 | δ = 8.14~8.12 (d, 2H), 7.95 (s, 1H), |
| | 7.89~7.80 (m, 4H), 7.71~7.59 (m, 11H), |
| | 7.57~7.51 (m, 6H), 7.48~7.29 (m, 9H) |
| 1-336 | δ = 8.55 (d, 1H), 8.45 (d, 1H), 8.37~8.17 |
| | (m, 7H), 8.03 (d, 1H), 7.94~7.93 (m, |
| | 2H), 7.78~7.40 (m, 22H), 7.11 (s, 1H) |
| 1-340 | δ = 8.22 (s, 1H), 8.03~7.98 (m, 4H), |
| | 7.88~7.69 (m, 9H), 7.57~7.24 (m, 17H), |
| | 7.08~6.97 (m, 4H) |
| 2-1 | δ = 9.10~9.08 (d, 2H), 8.52~8.50 (d, 2H), |
| | 8.18~7.99 (m, 7H), 7.88~7.73 (m, 4H), |
| | 7.60~7.30 (m, 10H) |
| 2-3 | δ = 8.34~8.32 (d, 4H), 7.97~7.95 (d, 1H), |
| | 7.87~7.73 (m, 4H), 7.54~7.26 (m, 16H) |
| 2-5 | δ = 8.37~8.29 (m, 5H), 7.86~7.73 (m, 4H), |
| | 7.53~7.39 (m, 14H) |
| 2-8 | δ = 8.37~8.23 (m, 5H), 7.86~7.74 (m, 8H), |
| | 7.50~7.39 (m, 12H), 7.26~7.22 (m, 6H) |
| 2-21 | δ = 8.98~8.96 (d, 2H), 8.40~8.38 (d, 4H), |
| | 7.79~7.73 (m, 4H), 7.58~7.40 (m, 12H), |
| | 7.26~7.24 (m, 5H) |
| 2-30 | δ = 8.99~8.97 (d, 2H), 8.35~8.33 (d, 2H), |
| | 8.25~8.23 (d, 2H), 7.84~7.66 (m, 7H), |
| | 7.51~7.40 (m, 11H), 7.33~7.24 (m, 3H) |

TABLE 5

| Compound | FD-MS |
|---|---|
| 1-1 | m/z = 667.19 (C48H29NO3, 667.21) |
| 1-2 | m/z = 683.12 (C48H29NO2S, 683.19) |
| 1-12 | m/z = 683.19 (C48H29NO2S, 683.83) |
| 1-13 | m/z = 699.17 (C48H29NOS2, 699.89) |
| 1-15 | m/z = 685.21 (C48H28FNO3, 685.75) |
| 1-18 | m/z = 699.17 (C48H29NOS2, 699.89) |
| 1-30 | m/z = 723.28 (C52H37NO3, 723.87) |
| 1-32 | m/z = 667.19 (C48H29NO3, 667.21) |
| 1-33 | m/z = 667.23 (C48H29NO3, 667.21) |
| 1-49 | m/z = 667.24 (C48H29NO3, 667.21) |
| 1-57 | m/z = 667.24 (C48H29NO3, 667.21) |
| 1-65 | m/z = 667.21 (C48H29NO3, 667.21) |
| 1-66 | m/z = 683.19 (C48H29NO2S, 683.83) |
| 1-76 | m/z = 677.28 (C48H19D10NO3, 677.83) |
| 1-80 | m/z = 667.21 (C48H29NO3, 667.21) |
| 1-86 | m/z = 687.42 (C48D29NO3, 696.39) |
| 1-99 | m/z = 667.21 (C48H29NO3, 667.21) |
| 1-109 | m/z = 667.24 (C48H29NO3, 667.21) |
| 1-112 | m/z = 667.23 (C48H29NO3, 667.21) |
| 1-127 | m/z = 667.24 (C48H29NO3, 667.21) |
| 1-139 | m/z = 667.18 (C48H29NO3, 667.21) |
| 1-145 | m/z = 667.19 (C48H29NO3, 667.21) |
| 1-167 | m/z = 677.28 (C48H19D10NO3, 677.83) |
| 1-169 | m/z = 667.23 (C48H29NO3, 667.21) |
| 1-178 | m/z = 686.33 (C48H10D19NO3, 686.88) |
| 1-192 | m/z = 744.33 (C48D29NS3, 745.12) |
| 1-202 | m/z = 733.20 (C52H31NO2S, 733.20) |
| 1-226 | m/z = 717.28 (C52H31NO3, 717.23) |
| 1-230 | m/z = 767.25 (C56H33NO3, 767.88) |
| 1-249 | m/z = 717.24 (C52H31NO3, 717.23) |
| 1-265 | m/z = 767.25 (C56H33NO3, 767.88) |
| 1-268 | m/z = 791.25 (C58H33NO3, 791.91) |
| 1-271 | m/z = 764.40 (C52D31NO2S, 765.07) |
| 1-287 | m/z = 767.18 (C52H30FNOS2, 767.94) |
| 1-327 | m/z = 743.29 (C54H33NO3, 743.24) |
| 1-336 | m/z = 841.19 (C58H35NS3, 842.10) |
| 1-340 | m/z = 819.28 (C60H37NO3, 819.96) |
| 2-1 | m/z = 575.20 (C41H25N3O, 575.67) |
| 2-3 | m/z = 551.20 (C39H25N3O, 551.19) |
| 2-5 | m/z = 525.18 (C37H23N3O, 525.18) |
| 2-8 | m/z = 677.25 (C49H31N3O, 677.24) |
| 2-21 | m/z = 601.22 (C43H27N3O, 601.21) |
| 2-30 | m/z = 601.22 (C43H27N3O, 601.21) |

### Experimental Example 1

### (1) Manufacture of Organic Light Emitting Device (Red Host)

A glass substrate on which indium tin oxide (ITO) was coated as a thin film to a thickness of 1,500 Å was ultrasonic cleaned with distilled water. When the cleaning with distilled water was finished, the substrate was ultrasonic cleaned with solvents such as acetone, methanol and isopropyl alcohol, then dried, and then subjected to ultraviolet ozone (UVO) treatment for 5 minutes using ultraviolet (UV) in a UV cleaner. After that, the substrate was transferred to a plasma cleaner (PT), then subjected to plasma treatment under vacuum for ITO work function increase and residual film removal, and transferred to a thermal deposition apparatus for organic deposition.

On the transparent ITO electrode (positive electrode), 2-TNATA (4,4′,4″-tris[2-naphthyl(phenyl)amino]triphenylamine) that is a hole injection layer and NPB (N,N′-di(1-naphthyl)-N, N′-diphenyl-(1,1′-biphenyl)-4, 4′-diamine) that is a hole transport layer, which are common layers, were formed.

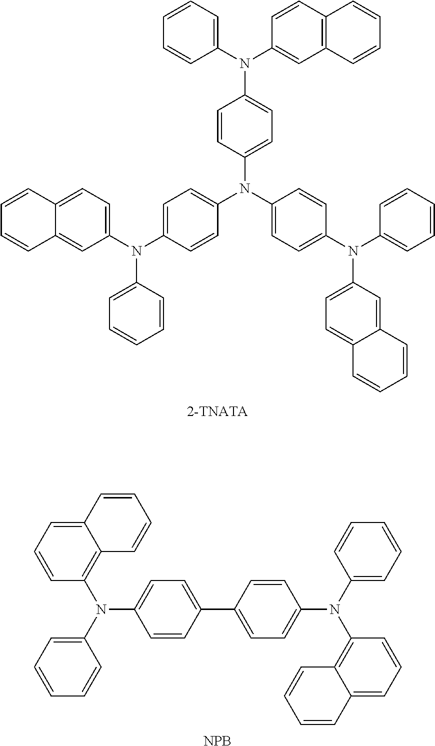 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. As the light emitting layer, a compound described in the following Table 5 was deposited to a thickness of 500 Å as a host, and [(piq)₂(Ir) (acac)], a red phosphorescent dopant, was doped and deposited by 3% by weight with respect to the host weight. After that, bathocuproine (BCP) was deposited to a thickness of 60 Å as a hole blocking layer, and Alq₃ was deposited to a thickness of 200 Å thereon as an electron transport layer. Lastly, lithium fluoride (LiF) was deposited on the electron transport layer to a thickness of 10 Å to form an electron injection layer, and then an aluminum (Al) negative electrode was deposited on the electron injection layer to a thickness of 1,200 Å to form a negative electrode, and as a result, an organic light emitting device was manufactured.

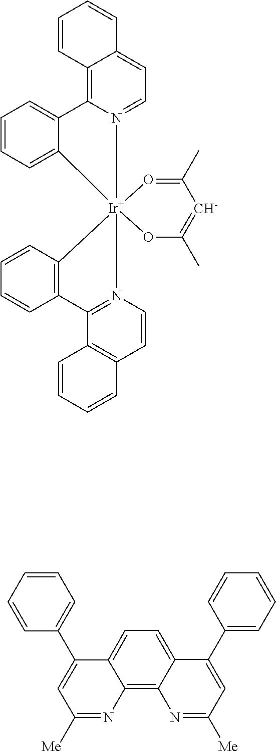

Meanwhile, all the organic compounds required to manufacture the OLED were vacuum sublimation purified under 10-8 torr to 10-6 torr for each material to be used in the manufacture of the OLED.

### (2) Driving Voltage and Light Emission Efficiency of Organic Light Emitting Device

For each of the organic light emitting devices manufactured as above, electroluminescent (EL) properties were measured using M7000 manufactured by McScience Inc., and with the measurement results, Too was measured when standard luminance was 6,000 cd/m² through a lifetime measurement system (M6000) manufactured by McScience Inc. Properties of the organic electroluminescent devices of the present disclosure are as shown in the following Table 6. Herein, T₉₀ means a lifetime (unit: h, hour), a time taken for luminance to become 90% with respect to initial luminance.

TABLE 6

| | | | Driving | | Color | |
|---|---|---|---|---|---|---|
| | | Ratio | Voltage | Efficiency | Coordinate | Lifetime |
| | Compound | (P:N) | (V) | (cd/A) | (x, y) | (T₉₀) |
| Comparative | A | | 4.81 | 11.7 | (0.685, 0.315) | 8 |
| Example 1 | | | | | | |
| Comparative | B | | 4.52 | 10.6 | (0.685, 0.315) | 9 |
| Example 2 | | | | | | |
| Comparative | C | | 4.69 | 11.8 | (0.685, 0.315) | 8 |
| Example 3 | | | | | | |
| Comparative | D | | 4.96 | 10.1 | (0.685, 0.315) | 7 |
| Example 4 | | | | | | |
| Comparative | A:2-1 | 1:1 | 3.65 | 31.7 | (0.685, 0.315) | 49 |
| Example 5 | | | | | | |
| Comparative | D:2-21 | 1:1 | 3.27 | 40.3 | (0.685, 0.315) | 105 |
| Example 6 | | | | | | |
| Example 1 | 1-1 | | 4.26 | 17.9 | (0.685, 0.315) | 10 |
| Example 2 | 1-2 | | 4.17 | 21.5 | (0.685, 0.315) | 10 |
| Example 3 | 1-12 | | 4.22 | 19.8 | (0.684, 0.315) | 12 |
| Example 4 | 1-13 | | 4.28 | 23.1 | (0.683, 0.316) | 11 |
| Example 5 | 1-15 | | 4.26 | 17.6 | (0.685, 0.315) | 30 |
| Example 6 | 1-18 | | 4.19 | 22.4 | (0.685, 0.315) | 13 |
| Example 7 | 1-30 | | 4.21 | 24.1 | (0.684, 0.315) | 12 |
| Example 8 | 1-32 | | 4.30 | 23.5 | (0.685, 0.315) | 21 |
| Example 9 | 1-33 | | 4.28 | 14.9 | (0.685, 0.315) | 20 |
| Example 10 | 1-49 | | 4.21 | 14.2 | (0.685, 0.315) | 15 |
| Example 11 | 1-57 | | 4.32 | 22.8 | (0.685, 0.315) | 23 |
| Example 12 | 1-65 | | 4.33 | 23.2 | (0.685, 0.315) | 10 |
| Example 13 | 1-66 | | 4.19 | 20.7 | (0.683, 0.315) | 13 |
| Example 14 | 1-76 | | 4.29 | 22.7 | (0.685, 0.315) | 22 |
| Example 15 | 1-80 | | 4.26 | 29.8 | (0.685, 0.315) | 10 |
| Example 16 | 1-86 | | 4.18 | 22.6 | (0.685, 0.315) | 27 |
| Example 17 | 1-99 | | 3.71 | 23.5 | (0.685, 0.315) | 11 |
| Example 18 | 1-109 | | 4.12 | 24.7 | (0.685, 0.315) | 10 |
| Example 19 | 1-112 | | 3.89 | 15.1 | (0.685, 0.315) | 11 |
| Example 20 | 1-127 | | 4.05 | 16.4 | (0.685, 0.315) | 10 |
| Example 21 | 1-139 | | 3.98 | 16.5 | (0.685, 0.315) | 11 |
| Example 22 | 1-145 | | 4.16 | 25.6 | (0.685, 0.315) | 11 |
| Example 23 | 1-167 | | 4.03 | 15.9 | (0.685, 0.315) | 24 |
| Example 24 | 1-169 | | 4.02 | 18.0 | (0.685, 0.315) | 10 |
| Example 25 | 1-178 | | 4.18 | 22.6 | (0.685, 0.315) | 17 |
| Example 26 | 1-192 | | 4.28 | 23.3 | (0.684, 0.315) | 28 |
| Example 27 | 1-202 | | 3.93 | 19.8 | (0.685, 0.315) | 13 |
| Example 28 | 1-226 | | 4.18 | 17.9 | (0.685, 0.315) | 10 |
| Example 29 | 1-230 | | 4.12 | 24.5 | (0.685, 0.314) | 13 |
| Example 30 | 1-249 | | 3.86 | 21.3 | (0.685, 0.315) | 11 |
| Example 31 | 1-265 | | 4.12 | 25.5 | (0.684, 0.315) | 12 |
| Example 32 | 1-268 | | 4.22 | 19.7 | (0.685, 0.317) | 14 |
| Example 33 | 1-271 | | 4.14 | 21.2 | (0.685, 0.317) | 27 |
| Example 34 | 1-287 | | 4.23 | 20.9 | (0.685, 0.314) | 23 |
| Example 35 | 1-327 | | 3.94 | 25.5 | (0.685, 0.315) | 12 |
| Example 36 | 1-336 | | 4.30 | 23.1 | (0.684, 0.315) | 10 |
| Example 37 | 1-340 | | 3.98 | 23.8 | (0.685, 0.316) | 16 |
| Example 38 | 1-1:2-1 | 1:2 | 2.55 | 63.7 | (0.685, 0.315) | 177 |
| Example 39 | | 1:1 | 2.40 | 62.1 | (0.685, 0.315) | 178 |
| Example 40 | | 2:1 | 2.45 | 61.5 | (0.685, 0.315) | 180 |
| Example 41 | 1-32:2-3 | 1:1 | 2.99 | 58.5 | (0.685, 0.315) | 231 |
| Example 42 | 1-57:2-3 | 1:2 | 2.55 | 60.8 | (0.685, 0.315) | 229 |
| Example 43 | | 1:1 | 2.58 | 59.3 | (0.685, 0.315) | 235 |
| Example 44 | | 2:1 | 2.75 | 54.1 | (0.685, 0.315) | 228 |
| Example 45 | 1-65:2-5 | 1:1 | 2.45 | 63.0 | (0.685, 0.315) | 198 |
| Example 46 | 1-86:2-5 | 1:1 | 2.80 | 64.1 | (0.685, 0.315) | 229 |
| Example 47 | 1-109:2-8 | 1:1 | 2.43 | 61.1 | (0.685, 0.315) | 354 |
| Example 48 | 1-127:2-8 | 1:1 | 2.40 | 58.3 | (0.685, 0.315) | 383 |
| Example 49 | 1-139:2-8 | 1:2 | 2.40 | 54.1 | (0.685, 0.315) | 251 |
| Example 50 | | 1:1 | 2.35 | 59.5 | (0.685, 0.315) | 270 |
| Example 51 | | 2:1 | 2.36 | 60.2 | (0.685, 0.315) | 298 |
| Example 52 | 1-169:2-21 | 1:1 | 2.38 | 61.0 | (0.685, 0.315) | 243 |
| Example 53 | 1-202:2-21 | 1:1 | 2.40 | 59.6 | (0.685, 0.315) | 234 |
| Example 54 | 1-226:2-30 | 1:1 | 2.51 | 57.7 | (0.685, 0.315) | 243 |
| Example 55 | 1-249:2-30 | 1:2 | 2.41 | 56.0 | (0.685, 0.315) | 185 |
| Example 56 | | 1:1 | 2.60 | 55.4 | (0.685, 0.315) | 234 |
| Example 57 | | 2:1 | 2.45 | 53.9 | (0.685, 0.315) | 194 |

Compounds A to D used in Table 5 are as follows.

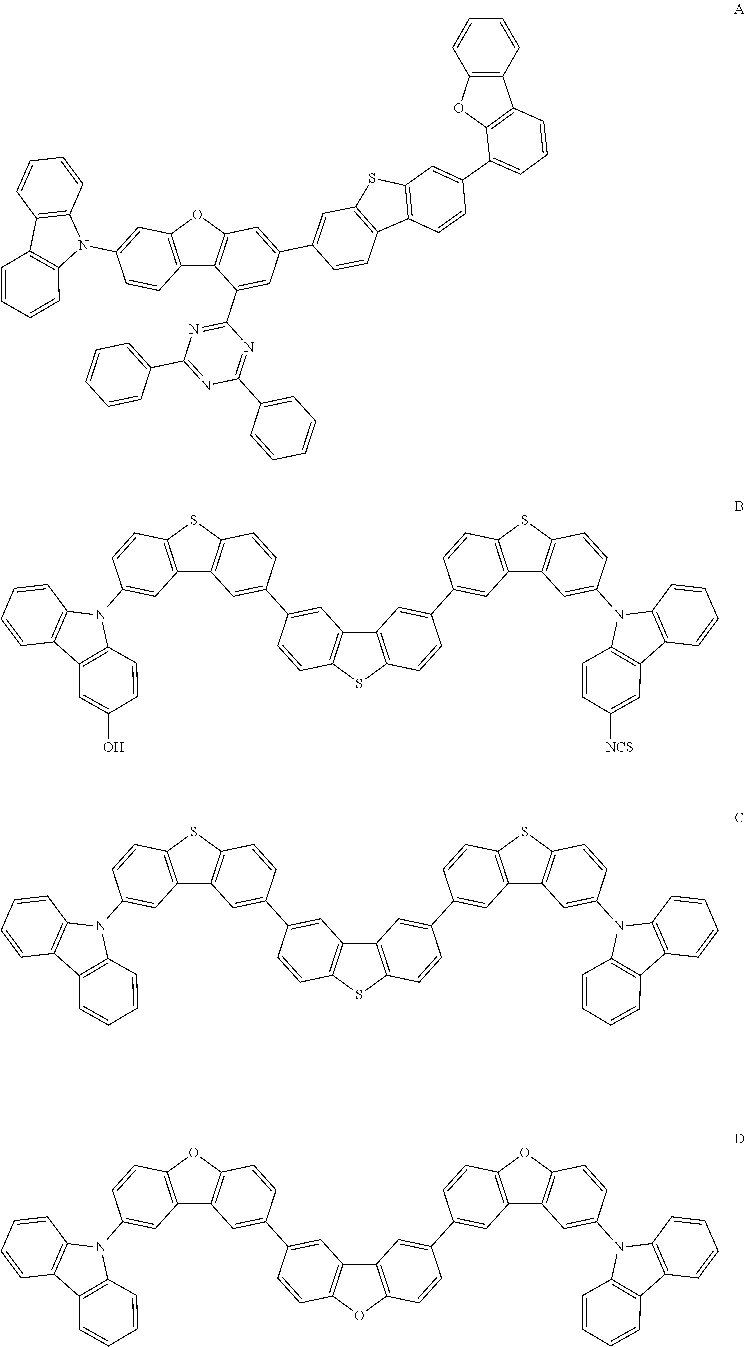

As seen from the results of Table 6, it could be identified that Examples 1 to 53 using the heterocyclic compound represented by Chemical Formula 1 of the present application as an organic material layer, particularly as a host of a light emitting layer, of the organic light emitting device, had improved driving voltage and efficiency compared to the organic light emitting devices of Comparative Examples 1 to 6.

In other words, compared to Compounds A to D used in Comparative Examples 1 to 6, it could be identified that, in the arylamine compound represented by Chemical Formula 1 of the present disclosure used in Examples 1 to 40, strong hole transfer (HT) properties of the arylamine group substituting at a specific position relieve a hole blocking (hole trap) phenomenon occurring in the dopant compared to in the carbazole compounds.

In addition, unlike Compounds A to D, the compound of the present disclosure does not have a linear structure, and has a steric arrangement like Chemical Formula 1, thereby having high thermal stability. In addition, it could be identified that, since strong charge transfer is possible by spatially separating HOMO (highest occupied molecular orbital) and LUMO (lowest unoccupied molecular orbital), the compound is suitable as a red host material, and high efficiency may be expected when used as an organic material in an organic light emitting device.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

1. A heterocyclic compound represented by the following Chemical Formula 1: 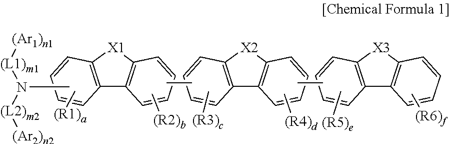
wherein, in Chemical Formula 1,
X1 to X3 are the same as or different from each other, and each independently O or S;
Ar₁ and Ar₂ are the same as or different from each other, and each independently a substituted or unsubstituted C6 to C60 aryl group; or a substituted or unsubstituted C2 to C60 heteroaryl group;
R1 to R6 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102 and —SiR101R102R103, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
L1 and L2 are the same as or different from each other, and each independently a direct bond; a substituted or unsubstituted arylene group having 6 to 60 carbon atoms; or a substituted or unsubstituted heteroarylene group having 2 to 60 carbon atoms;
a to e are the same as or different from each other, and each independently an integer of 0 to 3;
f is an integer of 0 to 4;
m1 and m2 are the same as or different from each other, and each independently an integer of 0 to 4; and
n1 and n2 are the same as or different from each other, and each independently an integer of 1 to 5.

2. The heterocyclic compound of claim 1, wherein Chemical Formula 1 is represented by the following Chemical Formula 1-1 to Chemical Formula 1-8: 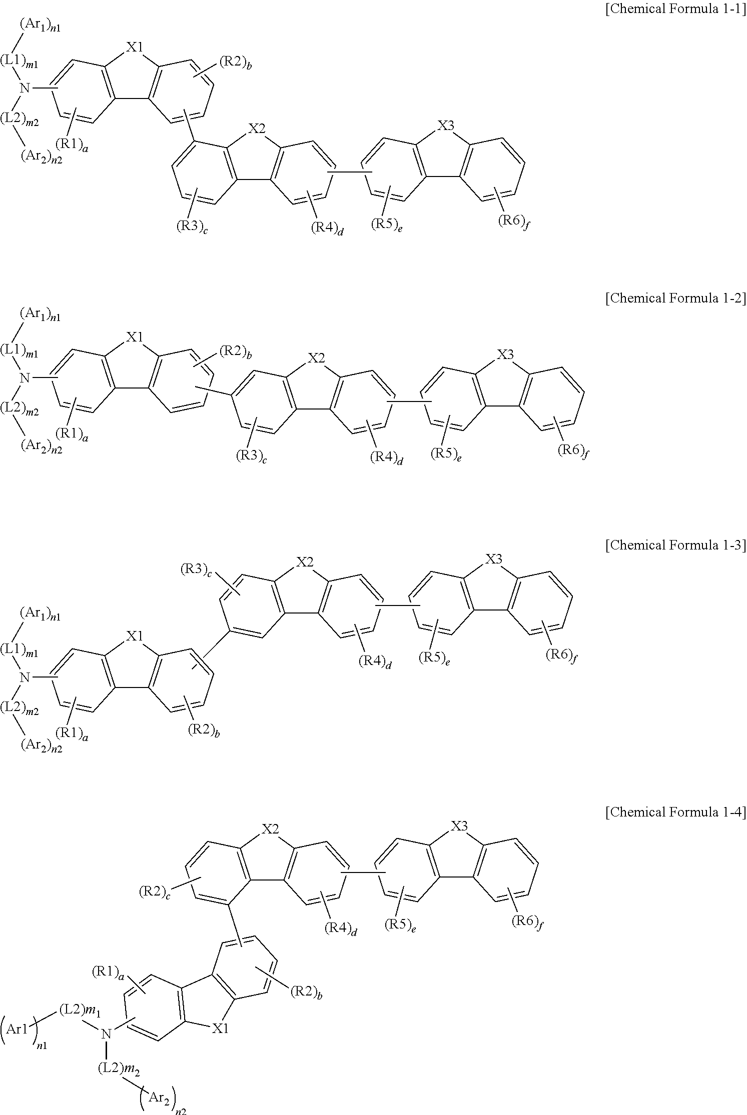 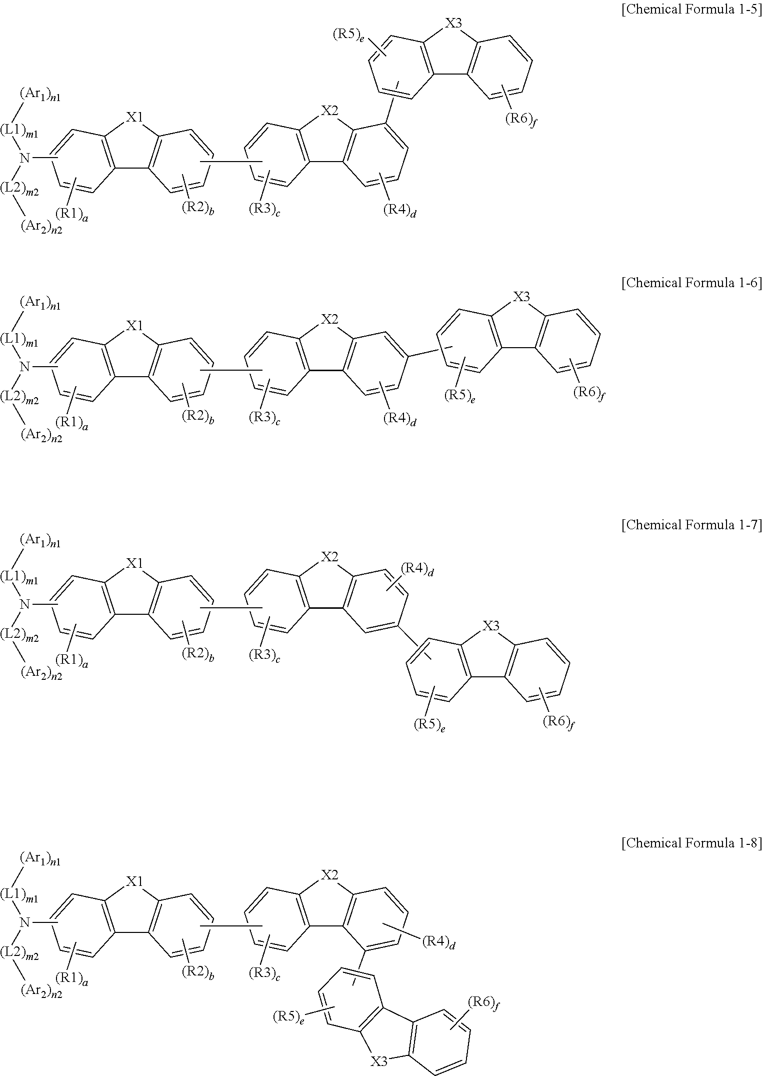
in Chemical Formula 1-1 to Chemical Formula 1-8,
X1 to X3, Ar₁ and Ar₂, R1 to R6, L1 and L2, a to f, m1 and m2 and n1 and n2 have the same definitions as in Chemical Formula 1.

3. The heterocyclic compound of claim 1, wherein Ar₁ and Ar₂ are the same as or different from each other, and each independently a substituted or unsubstituted C6 to C30 aryl group; or a substituted or unsubstituted C2 to C30 heteroaryl group.

4. The heterocyclic compound of claim 1, wherein R1 to R6 are the same as or different from each other, and each independently selected from the group consisting of hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102 and —SiR101R102R103, or two or more groups adjacent to each other bond to each other to form a substituted or unsubstituted C6 to C30 aromatic hydrocarbon ring; or a substituted or unsubstituted C2 to C30 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group.

5. The heterocyclic compound of claim 1, wherein R1 to R6 are the same as or different from each other, and each independently selected from the group consisting of hydrogen; deuterium; halogen; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other bond to each other to form a substituted or unsubstituted C6 to C20 aromatic hydrocarbon ring; or a substituted or unsubstituted C2 to C20 heteroring.

6. The heterocyclic compound of claim 1, wherein R1 to R6 are the same as or different from each other, and each independently selected from the group consisting of hydrogen; deuterium; and halogen.

7. The heterocyclic compound of claim 1, wherein the heterocyclic compound represented by Chemical Formula 1 does not include deuterium as a substituent, or has a deuterium content of 1% to 100% based on a total number of hydrogen atoms and deuterium atoms.

8. The heterocyclic compound of claim 1, wherein the heterocyclic compound represented by Chemical Formula 1 is represented by any one of the following compounds:

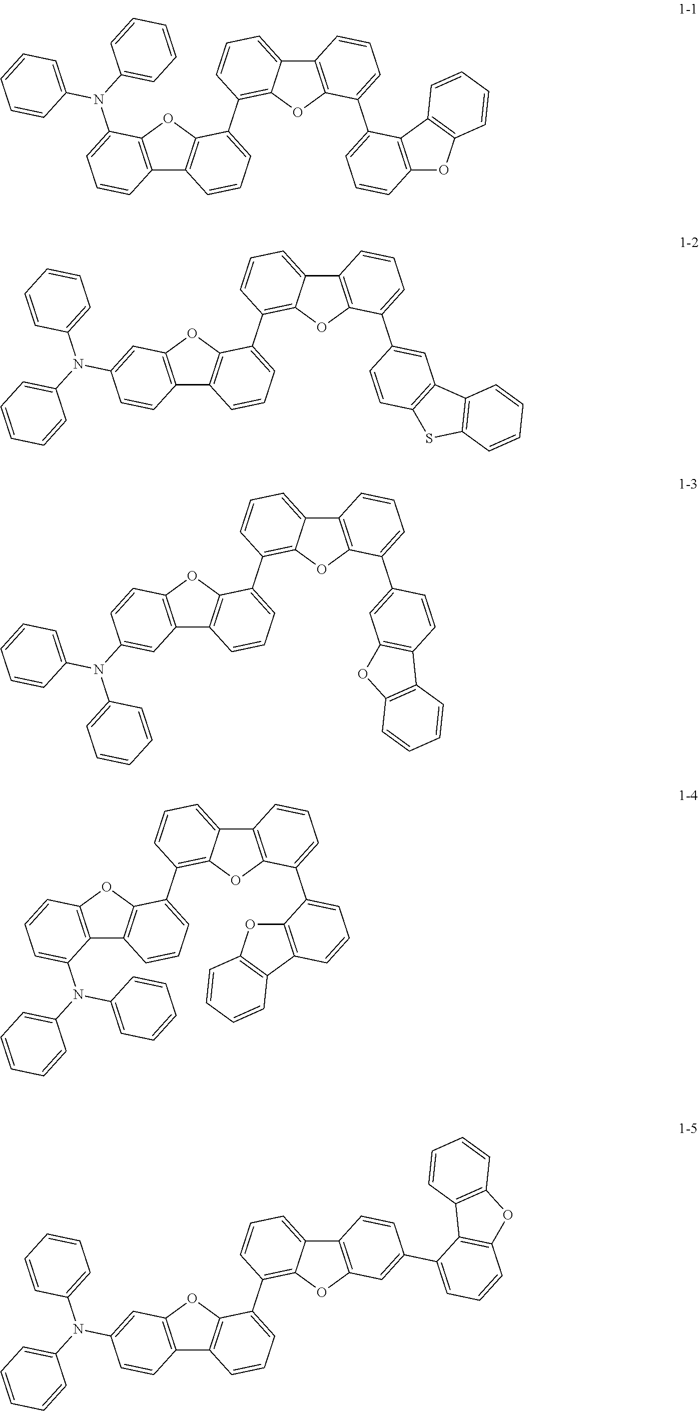 [SMILES: c1ccc(N(c2ccccc2)c2cccc3c2oc2c(-c4cccc5c4oc4c(-c6cccc7oc8ccccc8c67)cccc45)cccc23)cc1 ; c1ccc(N(c2ccccc2)c2ccc3c(c2)oc2c(-c4cccc5c4oc4c(-c6ccc7sc8ccccc8c7c6)cccc45)cccc23)cc1 ; c1ccc(N(c2ccccc2)c2ccc3oc4c(-c5cccc6c5oc5c(-c7ccc8c(c7)oc7ccccc78)cccc56)cccc4c3c2)cc1 ; c1ccc(N(c2ccccc2)c2cccc3oc4c(-c5cccc6c5oc5c(-c7cccc8c7oc7ccccc78)cccc56)cccc4c23)cc1 ; c1ccc(N(c2ccccc2)c2ccc3c(c2)oc2c(-c4cccc5c4oc4cc(-c6cccc7oc8ccccc8c67)ccc45)cccc23)cc1]

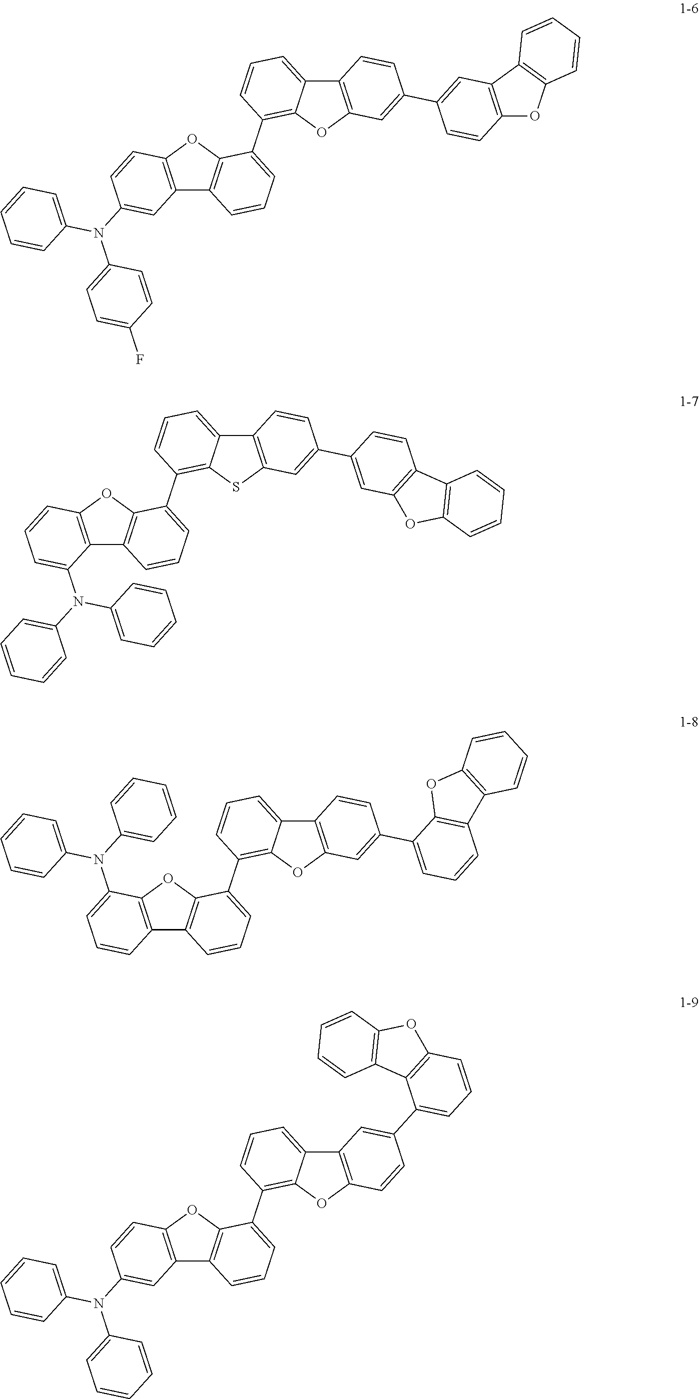 [SMILES: Fc1ccc(N(c2ccccc2)c2ccc3oc4c(-c5cccc6c5oc5cc(-c7ccc8oc9ccccc9c8c7)ccc56)cccc4c3c2)cc1 ; c1ccc(N(c2ccccc2)c2cccc3oc4c(-c5cccc6c5sc5cc(-c7ccc8c(c7)oc7ccccc78)ccc56)cccc4c23)cc1 ; c1ccc(N(c2ccccc2)c2cccc3c2oc2c(-c4cccc5c4oc4cc(-c6cccc7c6oc6ccccc67)ccc45)cccc23)cc1 ; c1ccc(N(c2ccccc2)c2ccc3oc4c(-c5cccc6c5oc5ccc(-c7cccc8oc9ccccc9c78)cc56)cccc4c3c2)cc1]

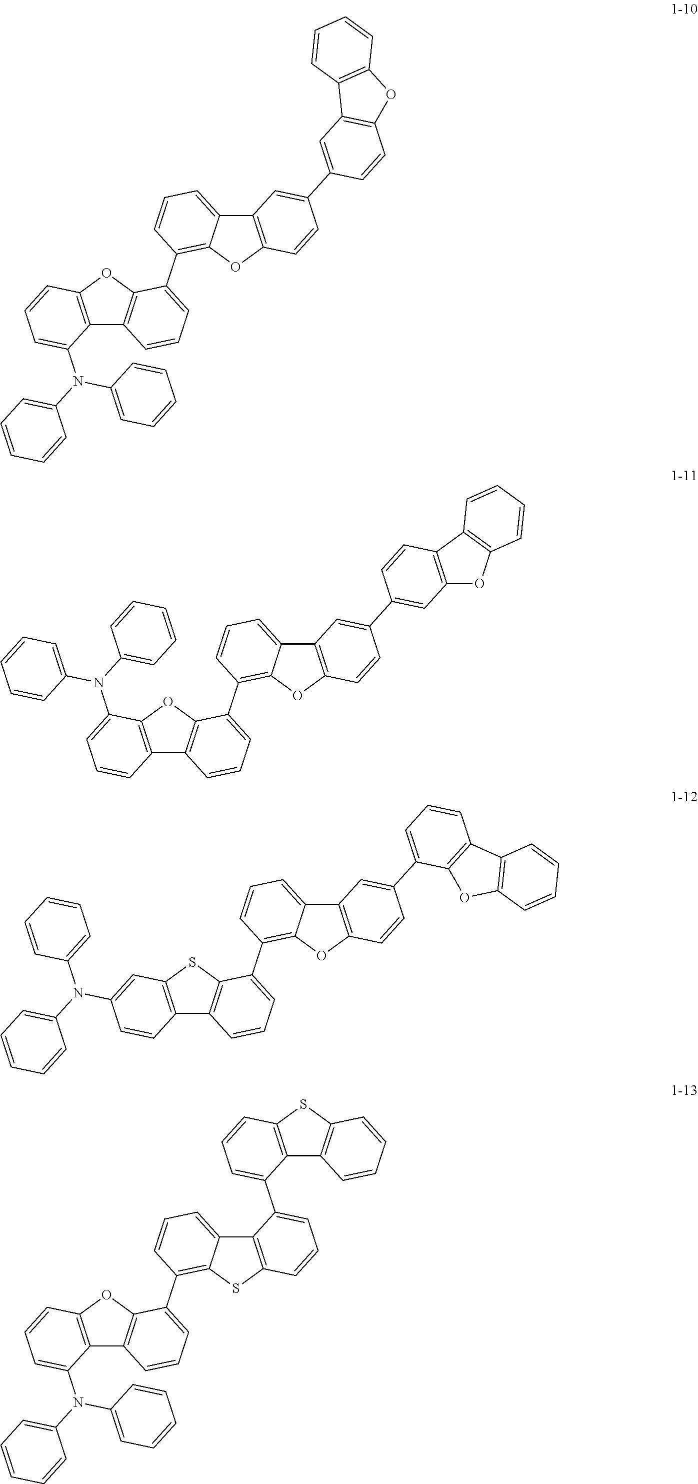 [SMILES: c1ccc(N(c2ccccc2)c2cccc3oc4c(-c5cccc6c5oc5ccc(-c7ccc8oc9ccccc9c8c7)cc56)cccc4c23)cc1 ; c1ccc(N(c2ccccc2)c2cccc3c2oc2c(-c4cccc5c4oc4ccc(-c6ccc7c(c6)oc6ccccc67)cc45)cccc23)cc1 ; c1ccc(N(c2ccccc2)c2ccc3c(c2)sc2c(-c4cccc5c4oc4ccc(-c6cccc7c6oc6ccccc67)cc45)cccc23)cc1 ; c1ccc(N(c2ccccc2)c2cccc3oc4c(-c5cccc6c5sc5cccc(-c7cccc8sc9ccccc9c78)c56)cccc4c23)cc1]

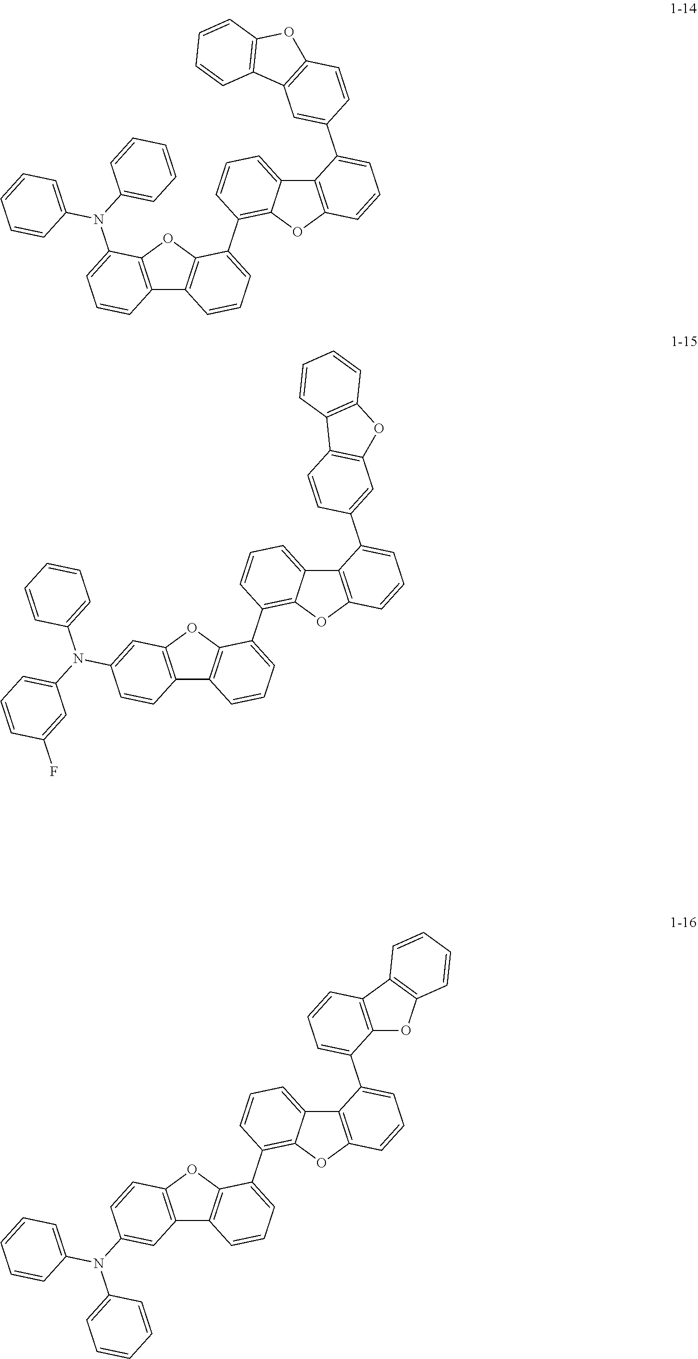 [SMILES: c1ccc(N(c2ccccc2)c2cccc3c2oc2c(-c4cccc5c4oc4cccc(-c6ccc7oc8ccccc8c7c6)c45)cccc23)cc1 ; Fc1cccc(N(c2ccccc2)c2ccc3c(c2)oc2c(-c4cccc5c4oc4cccc(-c6ccc7c(c6)oc6ccccc67)c45)cccc23)c1 ; c1ccc(N(c2ccccc2)c2ccc3oc4c(-c5cccc6c5oc5cccc(-c7cccc8c7oc7ccccc78)c56)cccc4c3c2)cc1]

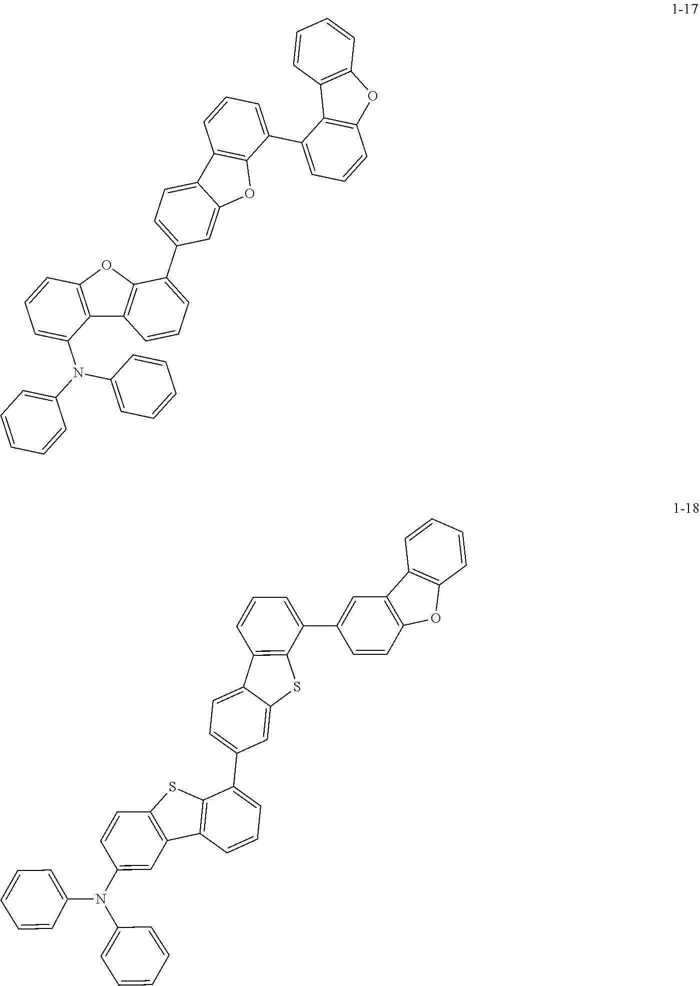 [SMILES: c1ccc(N(c2ccccc2)c2cccc3oc4c(-c5ccc6c(c5)oc5c(-c7cccc8oc9ccccc9c78)cccc56)cccc4c23)cc1 ; c1ccc(N(c2ccccc2)c2ccc3sc4c(-c5ccc6c(c5)sc5c(-c7ccc8oc9ccccc9c8c7)cccc56)cccc4c3c2)cc1]

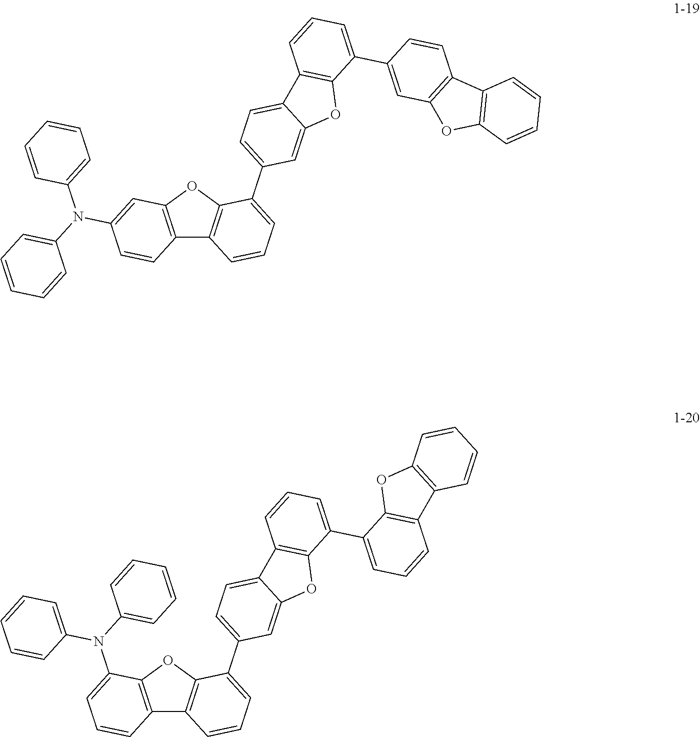 [SMILES: c1ccc(N(c2ccccc2)c2ccc3c(c2)oc2c(-c4ccc5c(c4)oc4c(-c6ccc7c(c6)oc6ccccc67)cccc45)cccc23)cc1 ; c1ccc(N(c2ccccc2)c2cccc3c2oc2c(-c4ccc5c(c4)oc4c(-c6cccc7c6oc6ccccc67)cccc45)cccc23)cc1]

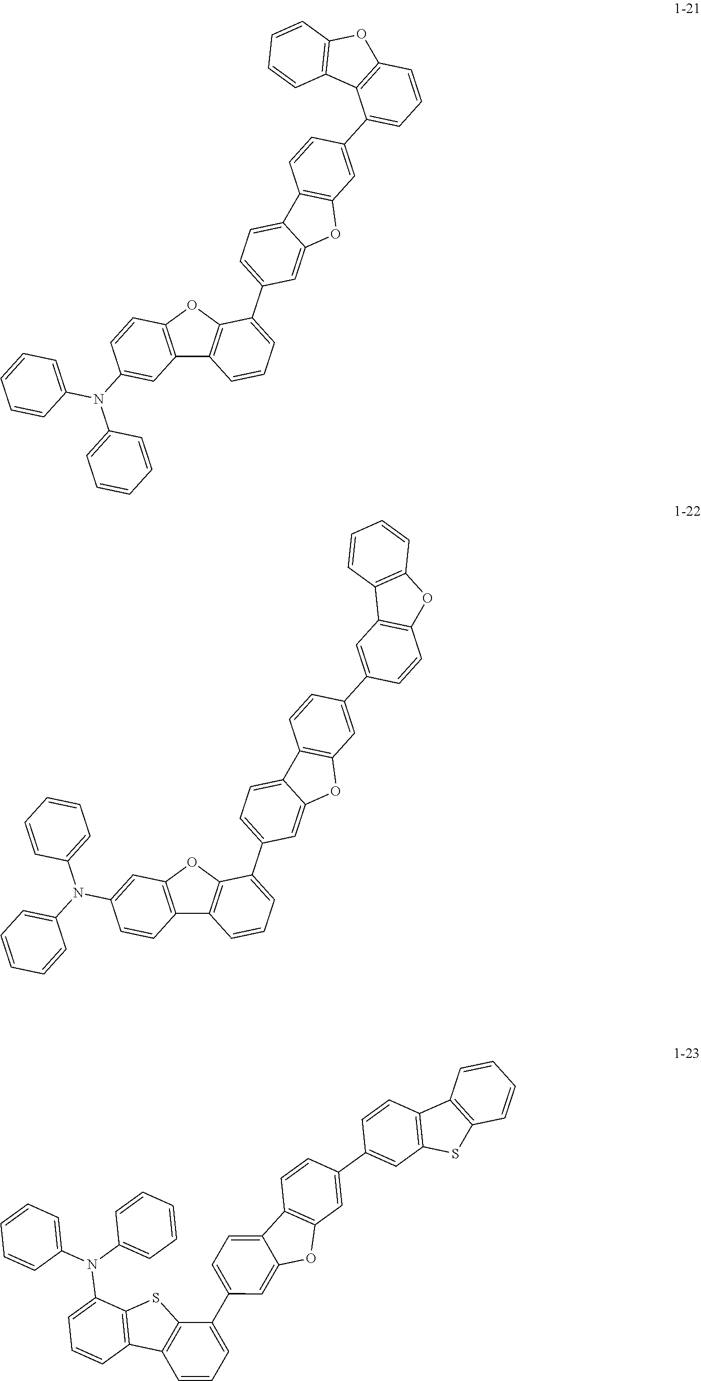 [SMILES: c1ccc(N(c2ccccc2)c2ccc3oc4c(-c5ccc6c(c5)oc5cc(-c7cccc8oc9ccccc9c78)ccc56)cccc4c3c2)cc1 ; c1ccc(N(c2ccccc2)c2ccc3c(c2)oc2c(-c4ccc5c(c4)oc4cc(-c6ccc7oc8ccccc8c7c6)ccc45)cccc23)cc1 ; c1ccc(N(c2ccccc2)c2cccc3c2sc2c(-c4ccc5c(c4)oc4cc(-c6ccc7c(c6)sc6ccccc67)ccc45)cccc23)cc1]

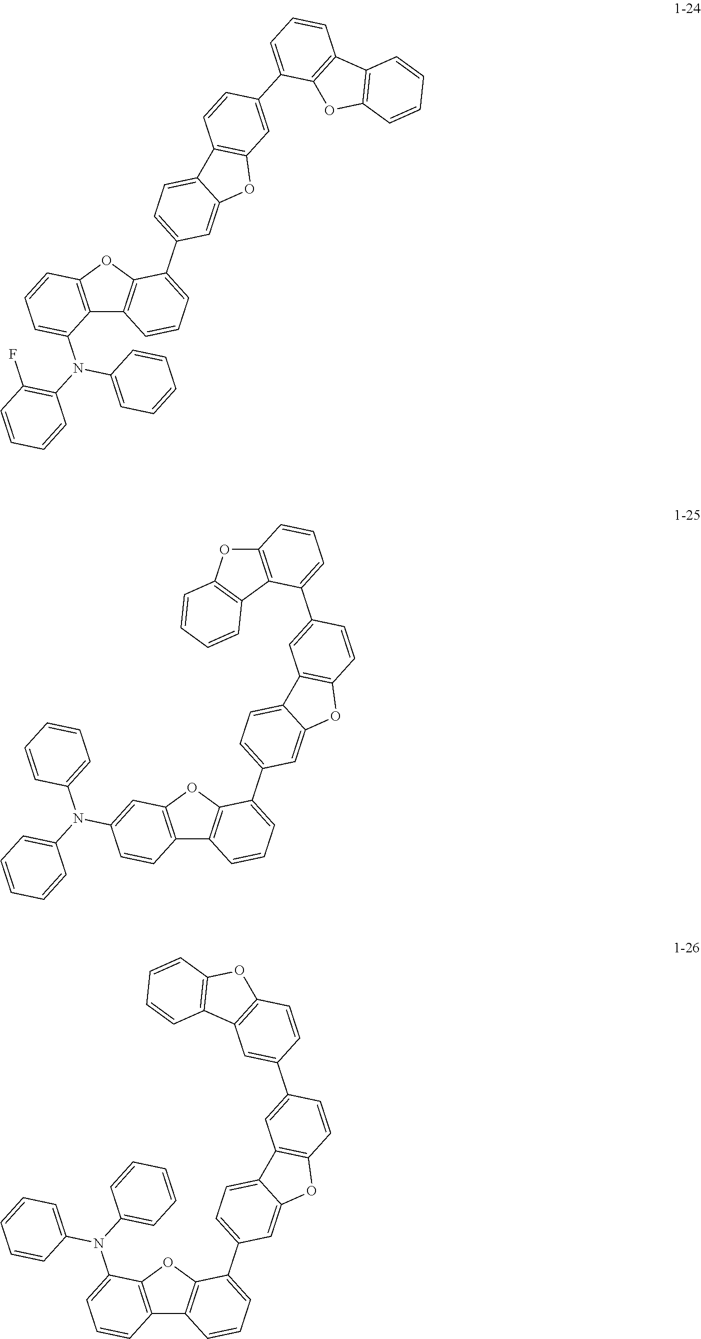 [SMILES: Fc1ccccc1N(c1ccccc1)c1cccc2oc3c(-c4ccc5c(c4)oc4cc(-c6cccc7c6oc6ccccc67)ccc45)cccc3c12 ; c1ccc(N(c2ccccc2)c2ccc3c(c2)oc2c(-c4ccc5c(c4)oc4ccc(-c6cccc7oc8ccccc8c67)cc45)cccc23)cc1 ; c1ccc(N(c2ccccc2)c2cccc3c2oc2c(-c4ccc5c(c4)oc4ccc(-c6ccc7oc8ccccc8c7c6)cc45)cccc23)cc1]

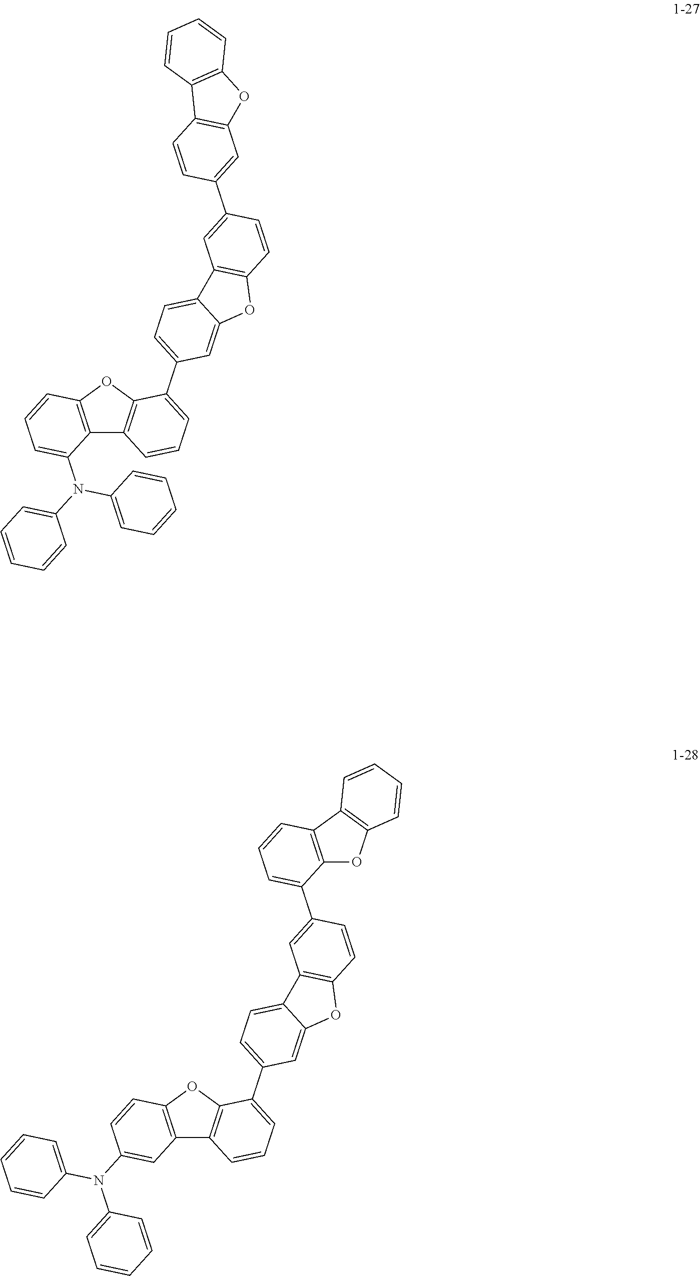 [SMILES: c1ccc(N(c2ccccc2)c2cccc3oc4c(-c5ccc6c(c5)oc5ccc(-c7ccc8c(c7)oc7ccccc78)cc56)cccc4c23)cc1 ; c1ccc(N(c2ccccc2)c2ccc3oc4c(-c5ccc6c(c5)oc5ccc(-c7cccc8c7oc7ccccc78)cc56)cccc4c3c2)cc1]

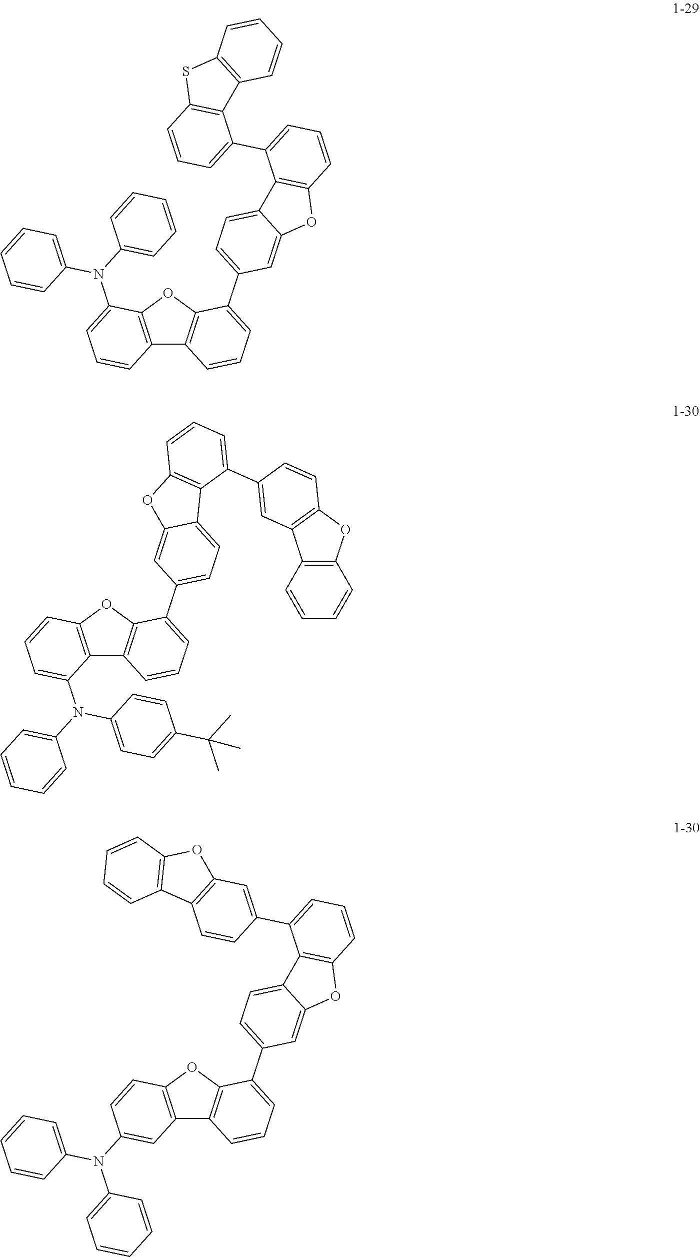 [SMILES: c1ccc(N(c2ccccc2)c2cccc3c2oc2c(-c4ccc5c(c4)oc4cccc(-c6cccc7sc8ccccc8c67)c45)cccc23)cc1 ; CC(C)(C)c1ccc(N(c2ccccc2)c2cccc3oc4c(-c5ccc6c(c5)oc5cccc(-c7ccc8oc9ccccc9c8c7)c56)cccc4c23)cc1 ; c1ccc(N(c2ccccc2)c2ccc3oc4c(-c5ccc6c(c5)oc5cccc(-c7ccc8c(c7)oc7ccccc78)c56)cccc4c3c2)cc1]

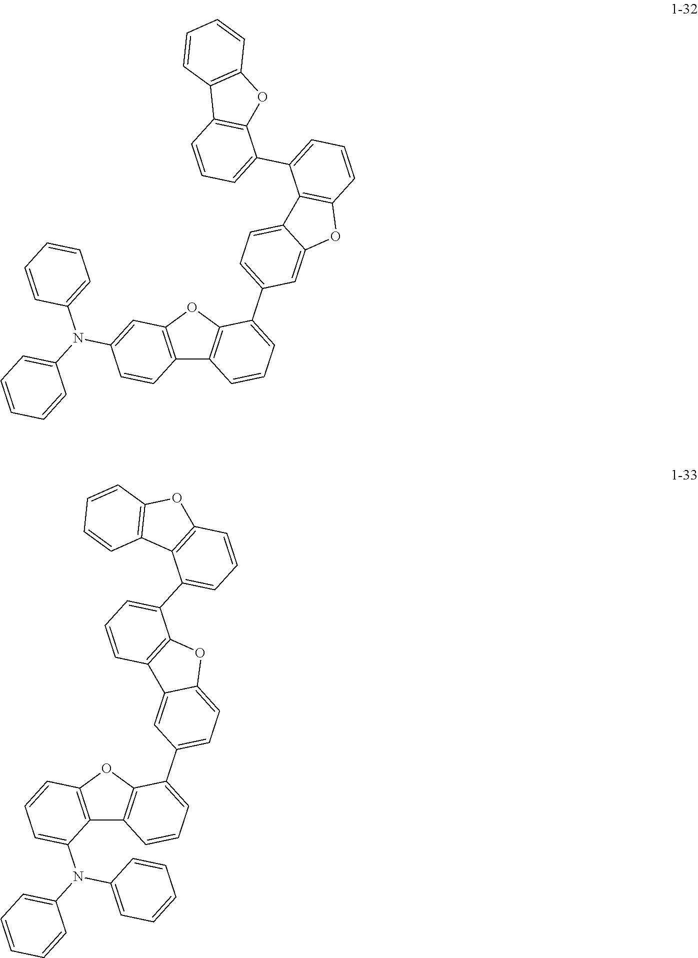 [SMILES: c1ccc(N(c2ccccc2)c2ccc3c(c2)oc2c(-c4ccc5c(c4)oc4cccc(-c6cccc7c6oc6ccccc67)c45)cccc23)cc1 ; c1ccc(N(c2ccccc2)c2cccc3oc4c(-c5ccc6oc7c(-c8cccc9oc%10ccccc%10c89)cccc7c6c5)cccc4c23)cc1]

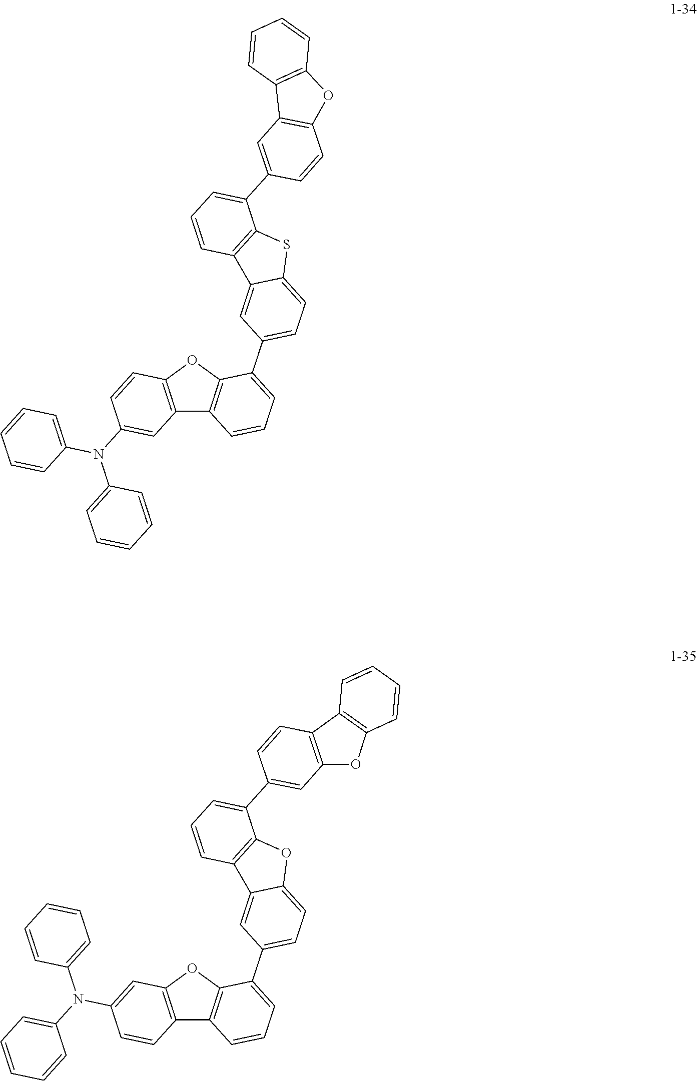 [SMILES: c1ccc(N(c2ccccc2)c2ccc3oc4c(-c5ccc6sc7c(-c8ccc9oc%10ccccc%10c9c8)cccc7c6c5)cccc4c3c2)cc1 ; c1ccc(N(c2ccccc2)c2ccc3c(c2)oc2c(-c4ccc5oc6c(-c7ccc8c(c7)oc7ccccc78)cccc6c5c4)cccc23)cc1]

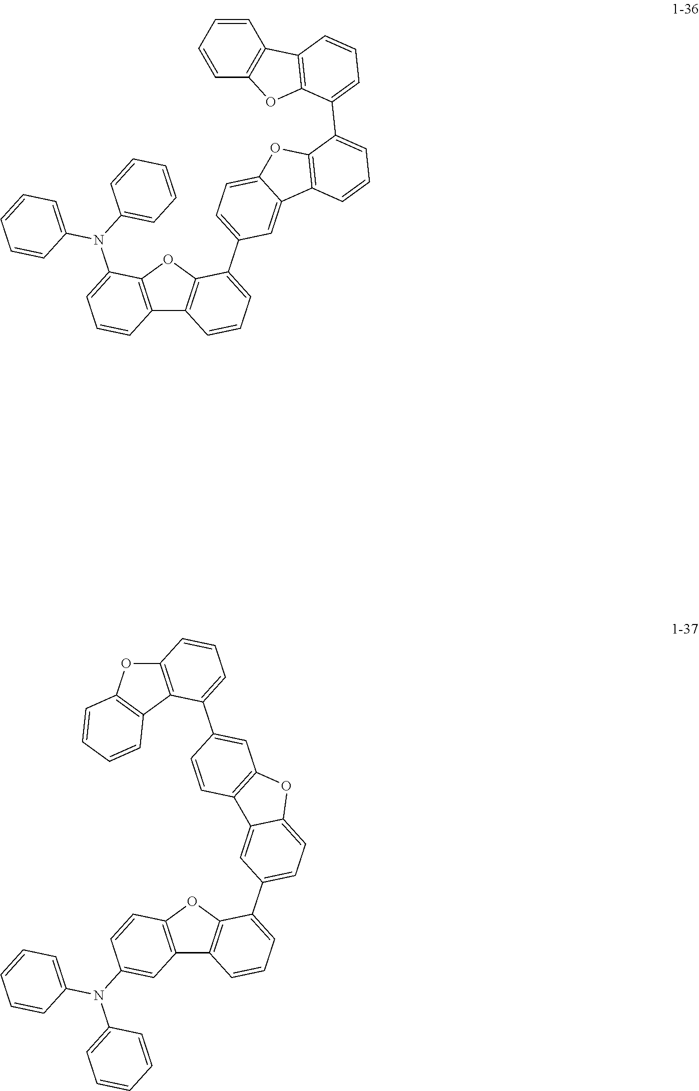 [SMILES: c1ccc(N(c2ccccc2)c2cccc3c2oc2c(-c4ccc5oc6c(-c7cccc8c7oc7ccccc78)cccc6c5c4)cccc23)cc1 ; c1ccc(N(c2ccccc2)c2ccc3oc4c(-c5ccc6oc7cc(-c8cccc9oc%10ccccc%10c89)ccc7c6c5)cccc4c3c2)cc1]

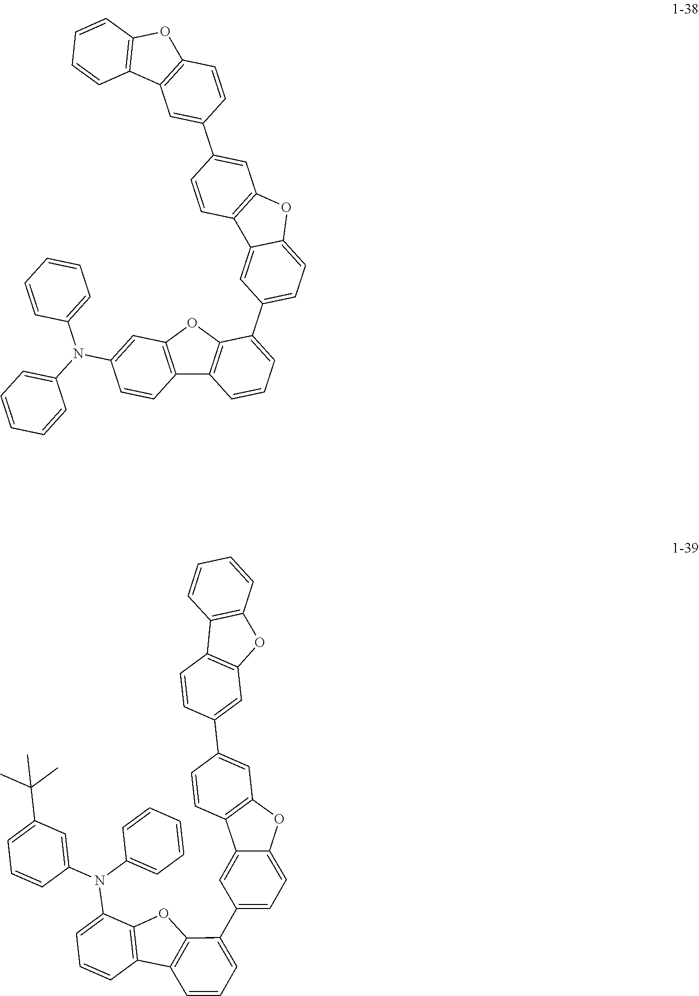 [SMILES: c1ccc(N(c2ccccc2)c2ccc3c(c2)oc2c(-c4ccc5oc6cc(-c7ccc8oc9ccccc9c8c7)ccc6c5c4)cccc23)cc1 ; CC(C)(C)c1cccc(N(c2ccccc2)c2cccc3c2oc2c(-c4ccc5oc6cc(-c7ccc8c(c7)oc7ccccc78)ccc6c5c4)cccc23)c1]

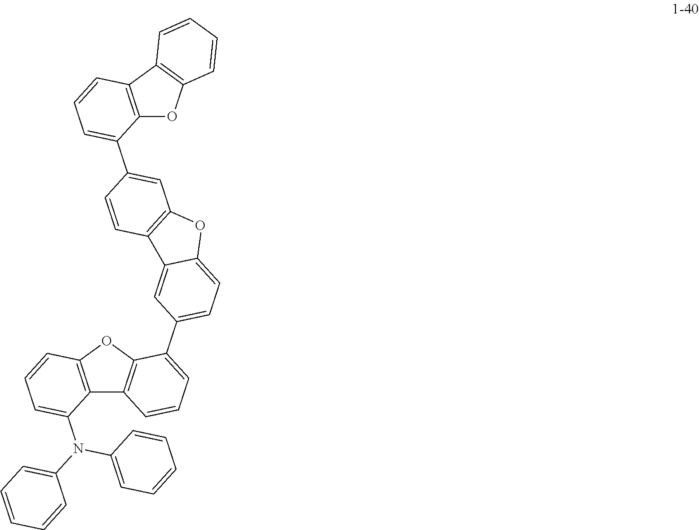 [SMILES: c1ccc(N(c2ccccc2)c2cccc3oc4c(-c5ccc6oc7cc(-c8cccc9c8oc8ccccc89)ccc7c6c5)cccc4c23)cc1]

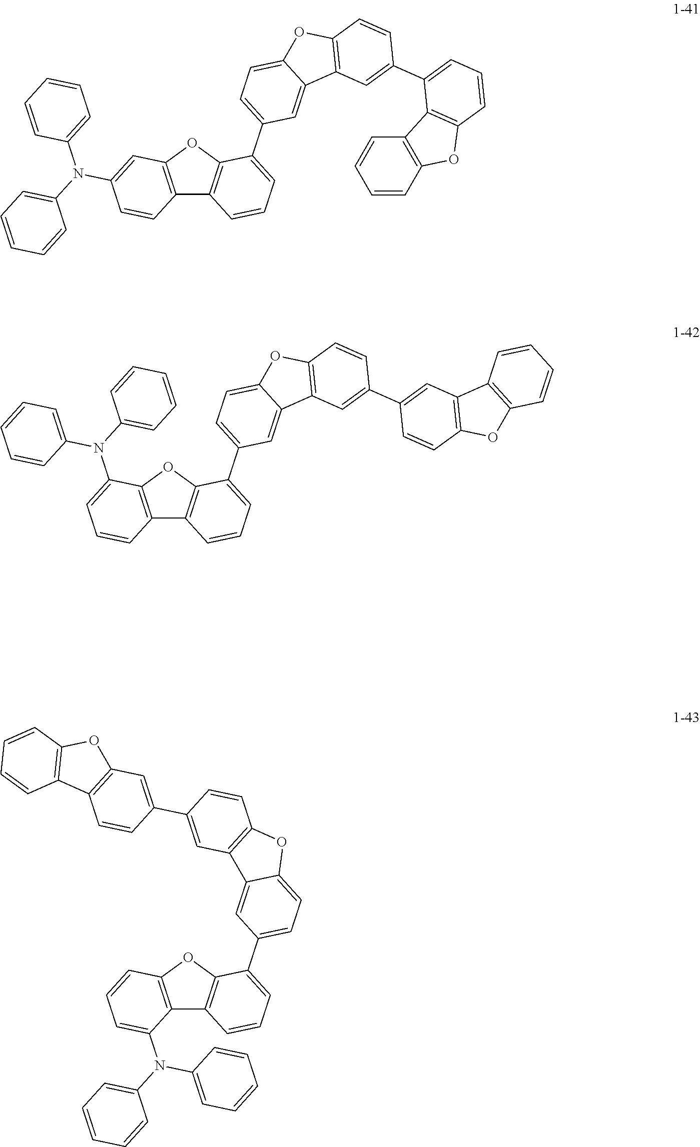 [SMILES: c1ccc(N(c2ccccc2)c2ccc3c(c2)oc2c(-c4ccc5oc6ccc(-c7cccc8oc9ccccc9c78)cc6c5c4)cccc23)cc1 ; c1ccc(N(c2ccccc2)c2cccc3c2oc2c(-c4ccc5oc6ccc(-c7ccc8oc9ccccc9c8c7)cc6c5c4)cccc23)cc1 ; c1ccc(N(c2ccccc2)c2cccc3oc4c(-c5ccc6oc7ccc(-c8ccc9c(c8)oc8ccccc89)cc7c6c5)cccc4c23)cc1]

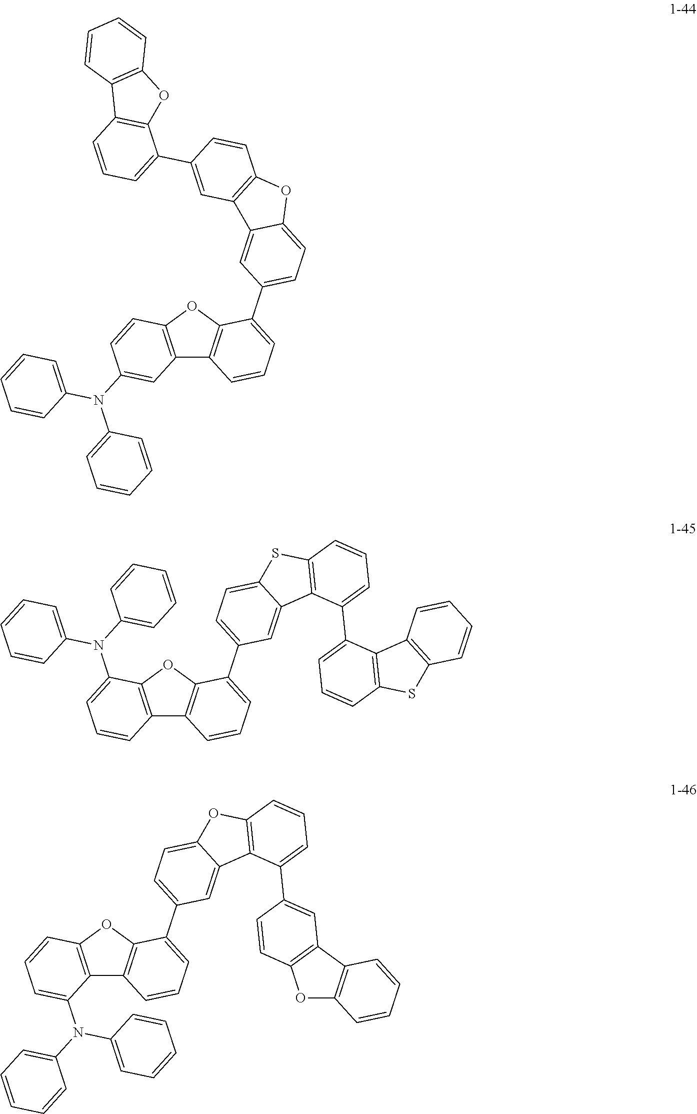 [SMILES: c1ccc(N(c2ccccc2)c2ccc3oc4c(-c5ccc6oc7ccc(-c8cccc9c8oc8ccccc89)cc7c6c5)cccc4c3c2)cc1 ; c1ccc(N(c2ccccc2)c2cccc3c2oc2c(-c4ccc5sc6cccc(-c7cccc8sc9ccccc9c78)c6c5c4)cccc23)cc1 ; c1ccc(N(c2ccccc2)c2cccc3oc4c(-c5ccc6oc7cccc(-c8ccc9oc%10ccccc%10c9c8)c7c6c5)cccc4c23)cc1]

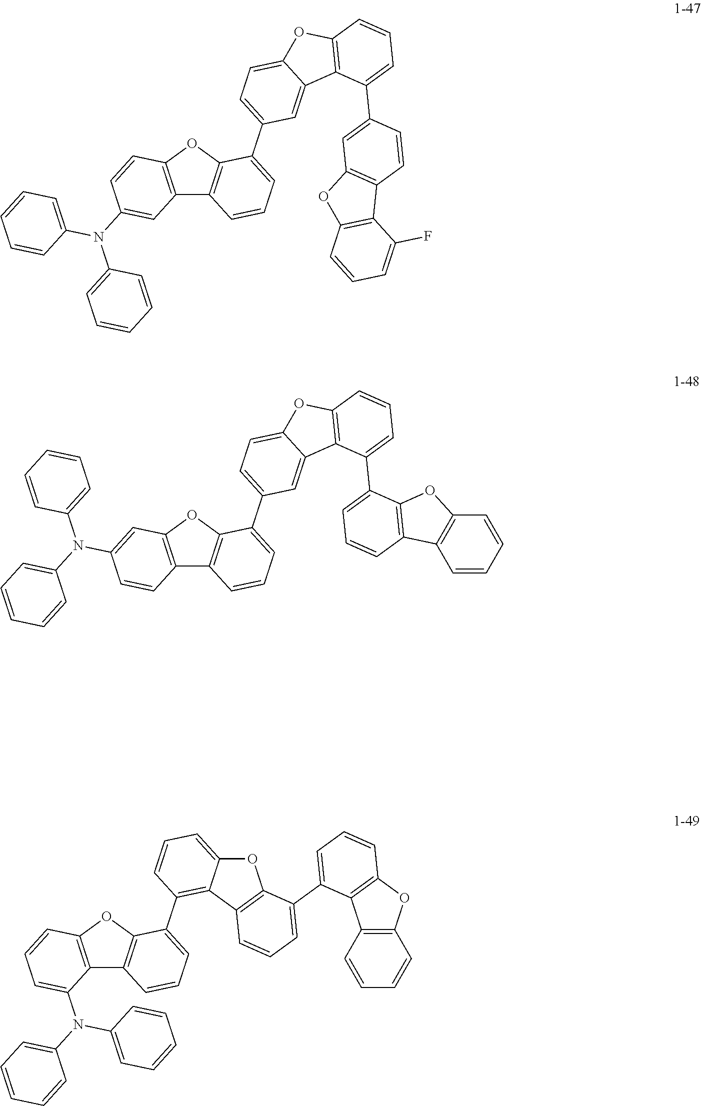 [SMILES: Fc1cccc2oc3cc(-c4cccc5oc6ccc(-c7cccc8c7oc7ccc(N(c9ccccc9)c9ccccc9)cc78)cc6c45)ccc3c12 ; c1ccc(N(c2ccccc2)c2ccc3c(c2)oc2c(-c4ccc5oc6cccc(-c7cccc8c7oc7ccccc78)c6c5c4)cccc23)cc1 ; c1ccc(N(c2ccccc2)c2cccc3oc4c(-c5cccc6oc7c(-c8cccc9oc%10ccccc%10c89)cccc7c56)cccc4c23)cc1]

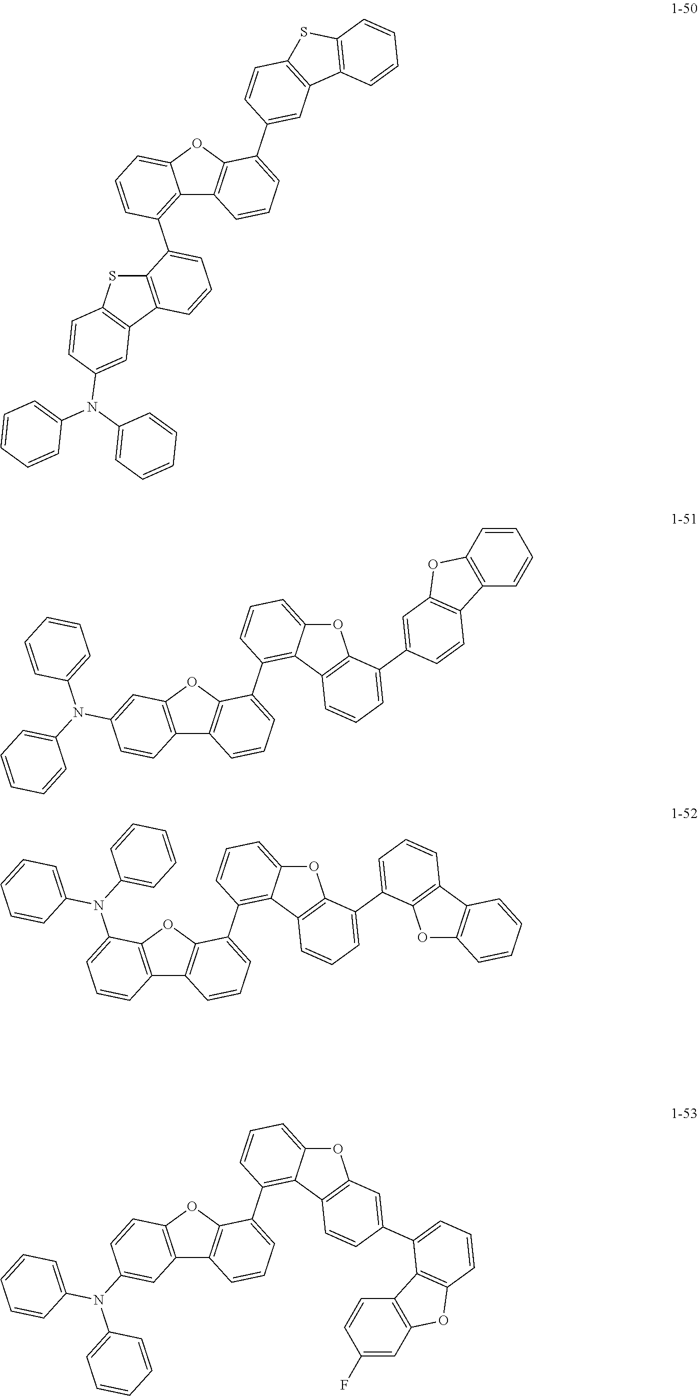 [SMILES: c1ccc(N(c2ccccc2)c2ccc3sc4c(-c5cccc6oc7c(-c8ccc9sc%10ccccc%10c9c8)cccc7c56)cccc4c3c2)cc1 ; c1ccc(N(c2ccccc2)c2ccc3c(c2)oc2c(-c4cccc5oc6c(-c7ccc8c(c7)oc7ccccc78)cccc6c45)cccc23)cc1 ; c1ccc(N(c2ccccc2)c2cccc3c2oc2c(-c4cccc5oc6c(-c7cccc8c7oc7ccccc78)cccc6c45)cccc23)cc1 ; Fc1ccc2c(c1)oc1cccc(-c3ccc4c(c3)oc3cccc(-c5cccc6c5oc5ccc(N(c7ccccc7)c7ccccc7)cc56)c34)c12]

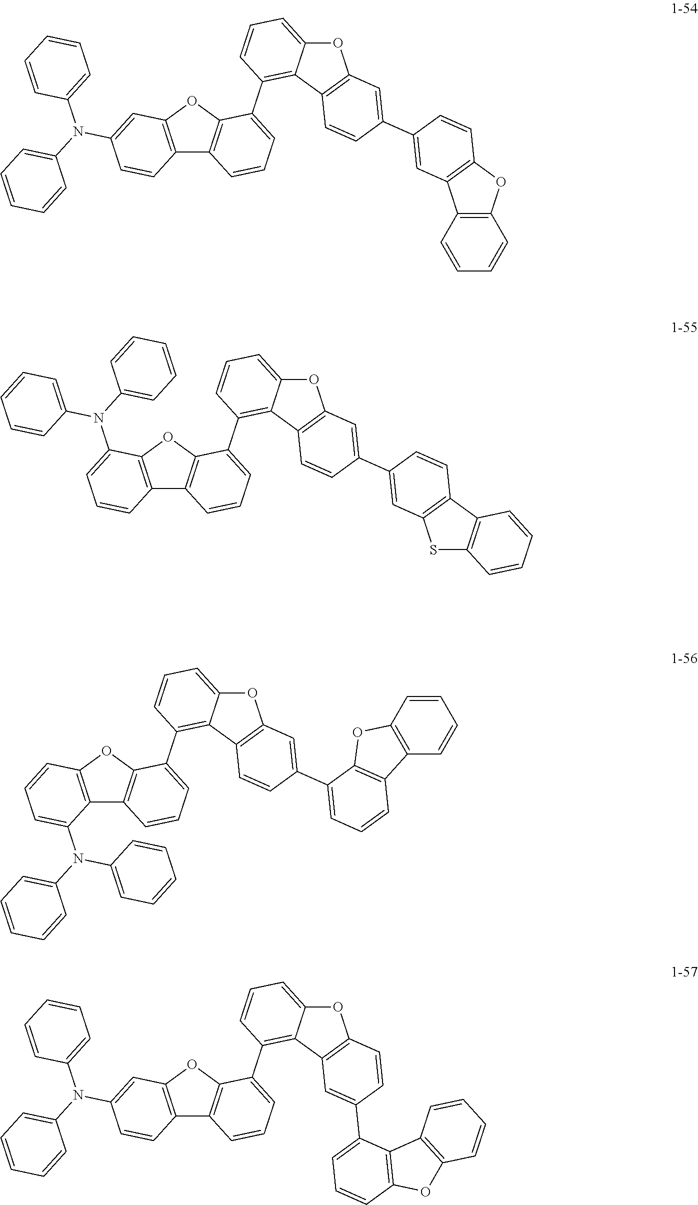 [SMILES: c1ccc(N(c2ccccc2)c2ccc3c(c2)oc2c(-c4cccc5oc6cc(-c7ccc8oc9ccccc9c8c7)ccc6c45)cccc23)cc1 ; c1ccc(N(c2ccccc2)c2cccc3c2oc2c(-c4cccc5oc6cc(-c7ccc8c(c7)sc7ccccc78)ccc6c45)cccc23)cc1 ; c1ccc(N(c2ccccc2)c2cccc3oc4c(-c5cccc6oc7cc(-c8cccc9c8oc8ccccc89)ccc7c56)cccc4c23)cc1 ; c1ccc(N(c2ccccc2)c2ccc3c(c2)oc2c(-c4cccc5oc6ccc(-c7cccc8oc9ccccc9c78)cc6c45)cccc23)cc1]

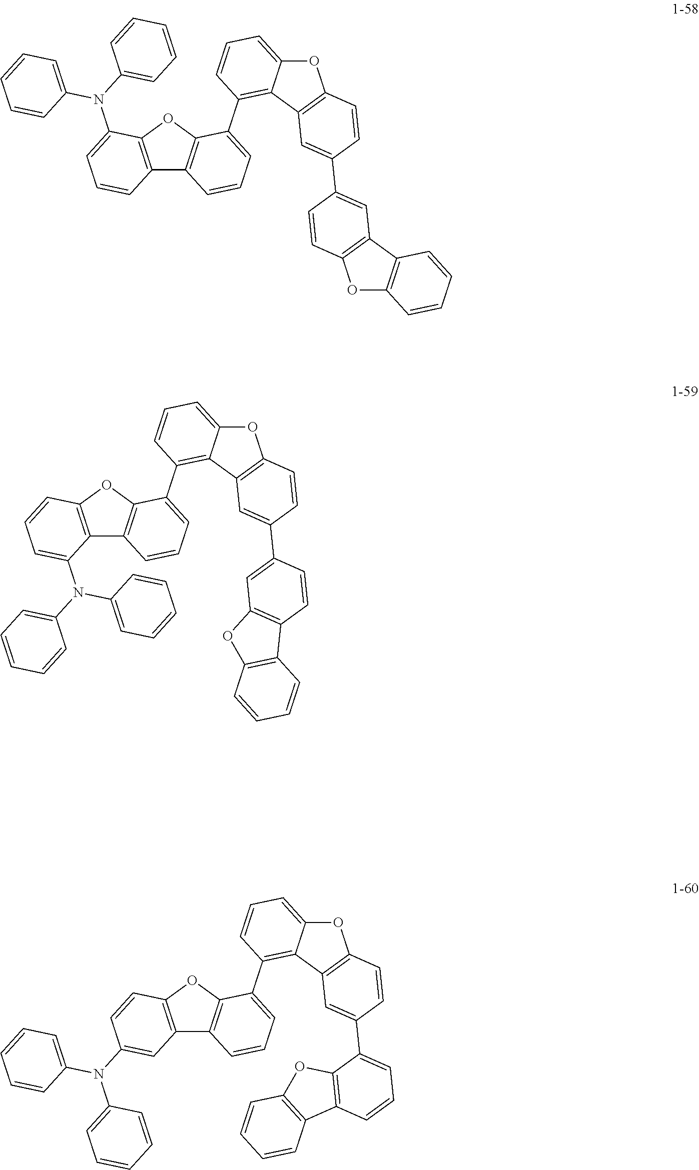 [SMILES: c1ccc(N(c2ccccc2)c2cccc3c2oc2c(-c4cccc5oc6ccc(-c7ccc8oc9ccccc9c8c7)cc6c45)cccc23)cc1 ; c1ccc(N(c2ccccc2)c2cccc3oc4c(-c5cccc6oc7ccc(-c8ccc9c(c8)oc8ccccc89)cc7c56)cccc4c23)cc1 ; c1ccc(N(c2ccccc2)c2ccc3oc4c(-c5cccc6oc7ccc(-c8cccc9c8oc8ccccc89)cc7c56)cccc4c3c2)cc1]

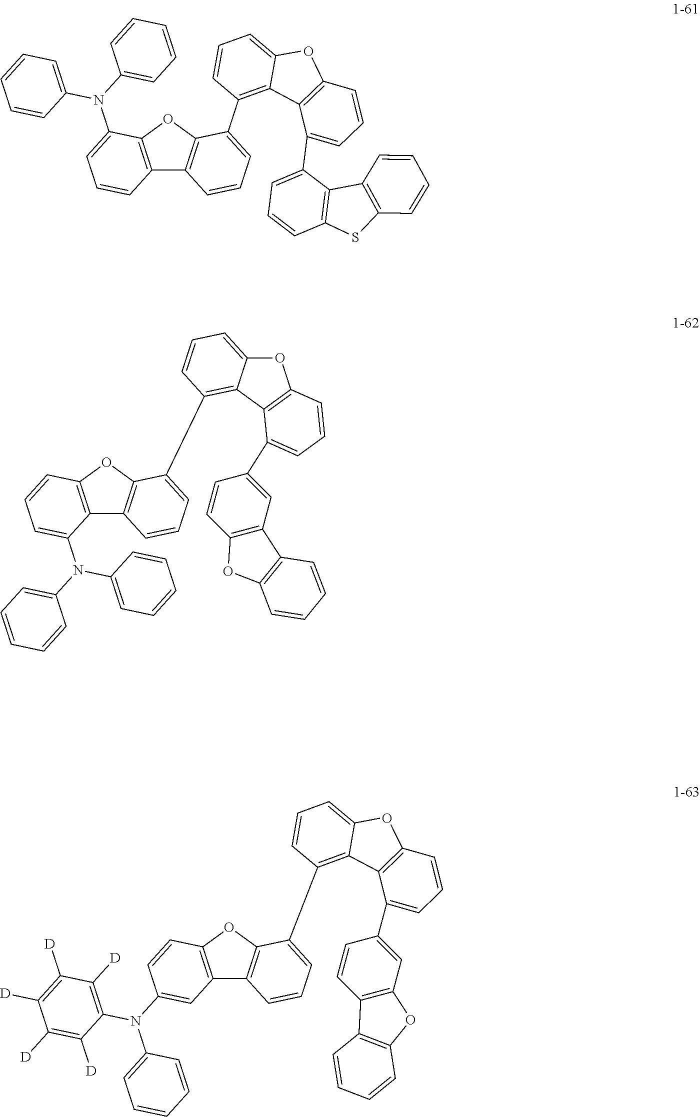 [SMILES: c1ccc(N(c2ccccc2)c2cccc3c2oc2c(-c4cccc5oc6cccc(-c7cccc8sc9ccccc9c78)c6c45)cccc23)cc1 ; c1ccc(N(c2ccccc2)c2cccc3oc4c(-c5cccc6oc7cccc(-c8ccc9oc%10ccccc%10c9c8)c7c56)cccc4c23)cc1 ; [2H]c1c([2H])c([2H])c(N(c2ccccc2)c2ccc3oc4c(-c5cccc6oc7cccc(-c8ccc9c(c8)oc8ccccc89)c7c56)cccc4c3c2)c([2H])c1[2H]]

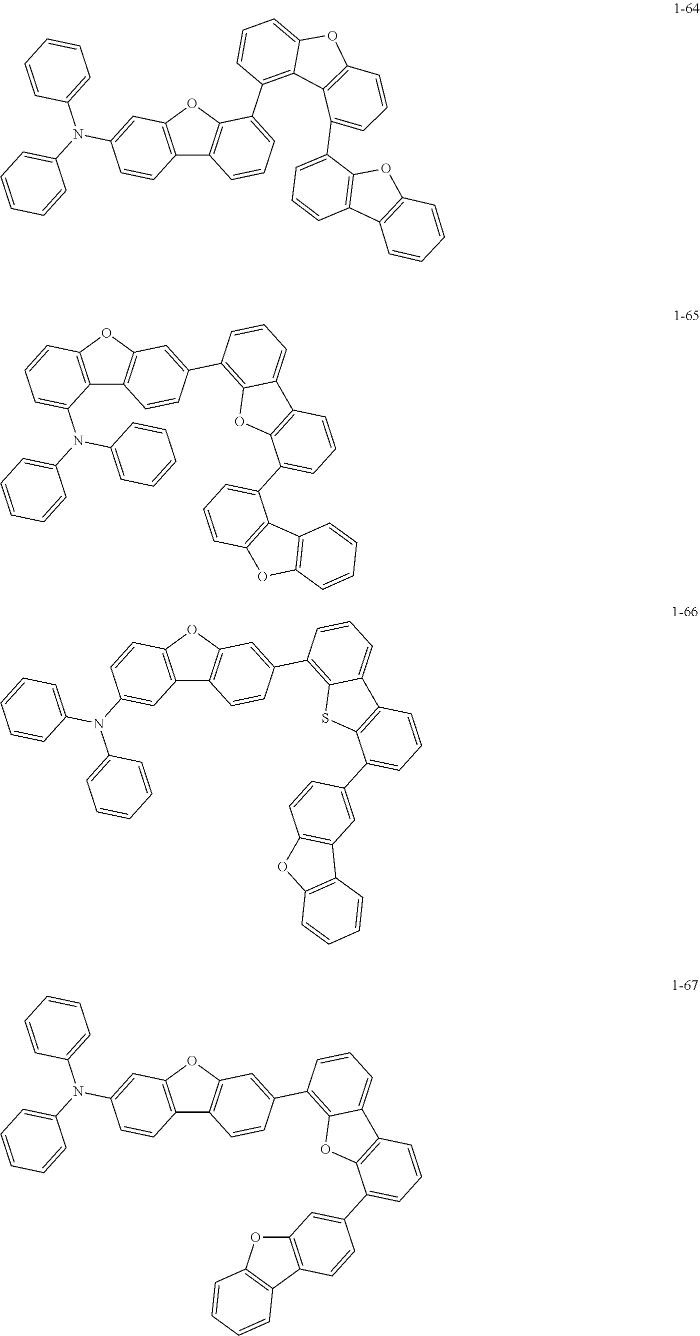

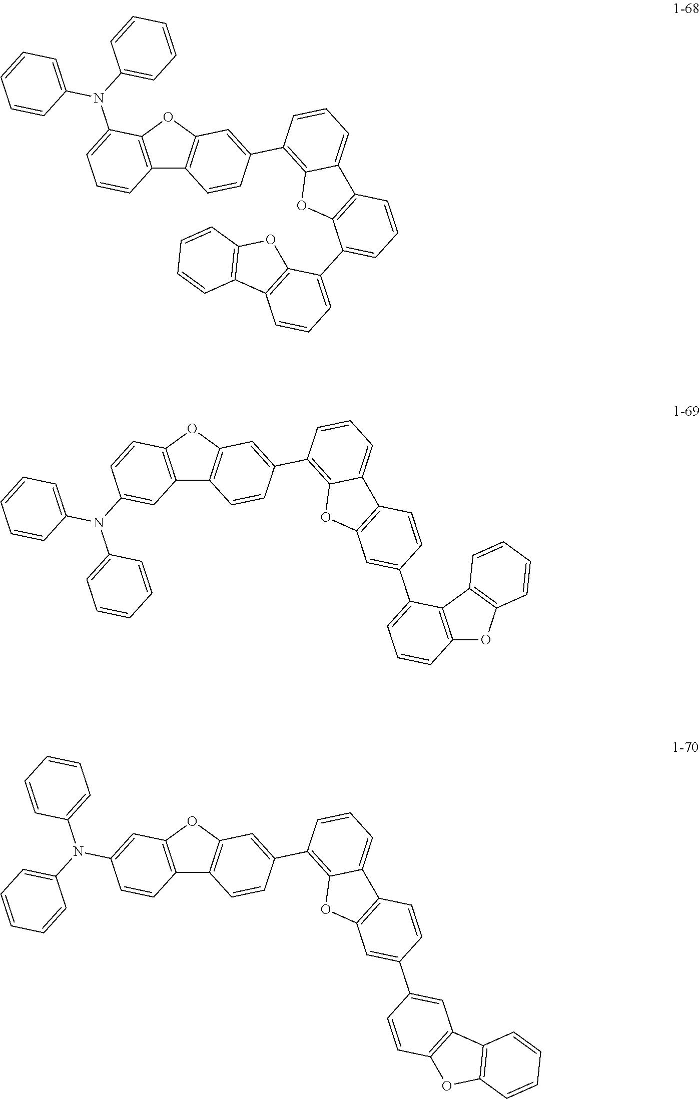 [SMILES: c1ccc(N(c2ccccc2)c2cccc3c2oc2cc(-c4cccc5c4oc4c(-c6cccc7c6oc6ccccc67)cccc45)ccc23)cc1 ; c1ccc(N(c2ccccc2)c2ccc3oc4cc(-c5cccc6c5oc5cc(-c7cccc8oc9ccccc9c78)ccc56)ccc4c3c2)cc1 ; c1ccc(N(c2ccccc2)c2ccc3c(c2)oc2cc(-c4cccc5c4oc4cc(-c6ccc7oc8ccccc8c7c6)ccc45)ccc23)cc1]

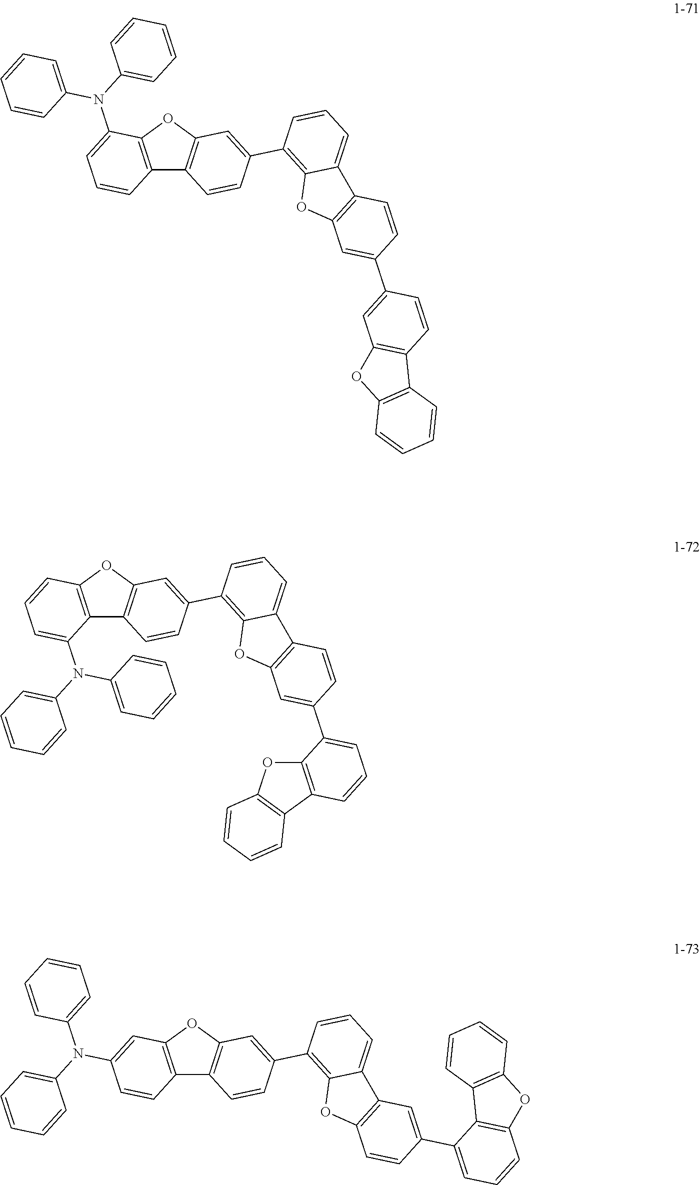 [SMILES: c1ccc(N(c2ccccc2)c2cccc3c2oc2cc(-c4cccc5c4oc4cc(-c6ccc7c(c6)oc6ccccc67)ccc45)ccc23)cc1 ; c1ccc(N(c2ccccc2)c2cccc3oc4cc(-c5cccc6c5oc5cc(-c7cccc8c7oc7ccccc78)ccc56)ccc4c23)cc1 ; c1ccc(N(c2ccccc2)c2ccc3c(c2)oc2cc(-c4cccc5c4oc4ccc(-c6cccc7oc8ccccc8c67)cc45)ccc23)cc1]

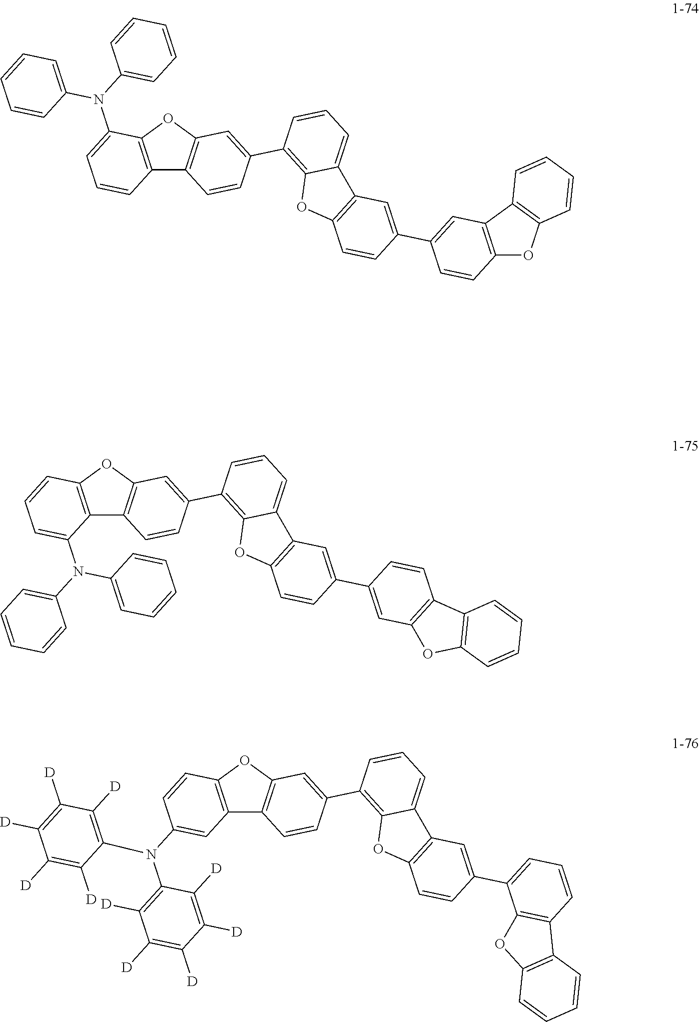 [SMILES: c1ccc(N(c2ccccc2)c2cccc3c2oc2cc(-c4cccc5c4oc4ccc(-c6ccc7oc8ccccc8c7c6)cc45)ccc23)cc1 ; c1ccc(N(c2ccccc2)c2cccc3oc4cc(-c5cccc6c5oc5ccc(-c7ccc8c(c7)oc7ccccc78)cc56)ccc4c23)cc1 ; [2H]c1c([2H])c([2H])c(N(c2ccc3oc4cc(-c5cccc6c5oc5ccc(-c7cccc8c7oc7ccccc78)cc56)ccc4c3c2)c2c([2H])c([2H])c([2H])c([2H])c2[2H])c([2H])c1[2H]]

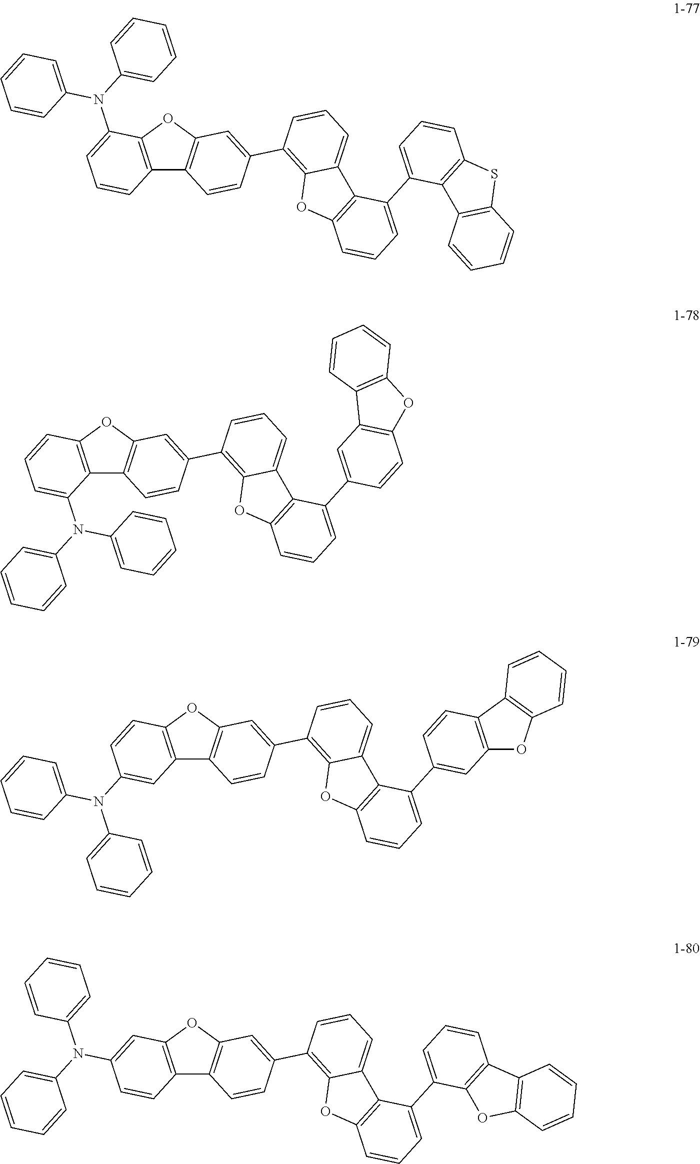 [SMILES: c1ccc(N(c2ccccc2)c2cccc3c2oc2cc(-c4cccc5c4oc4cccc(-c6cccc7sc8ccccc8c67)c45)ccc23)cc1 ; c1ccc(N(c2ccccc2)c2cccc3oc4cc(-c5cccc6c5oc5cccc(-c7ccc8oc9ccccc9c8c7)c56)ccc4c23)cc1 ; c1ccc(N(c2ccccc2)c2ccc3oc4cc(-c5cccc6c5oc5cccc(-c7ccc8c(c7)oc7ccccc78)c56)ccc4c3c2)cc1 ; c1ccc(N(c2ccccc2)c2ccc3c(c2)oc2cc(-c4cccc5c4oc4cccc(-c6cccc7c6oc6ccccc67)c45)ccc23)cc1]

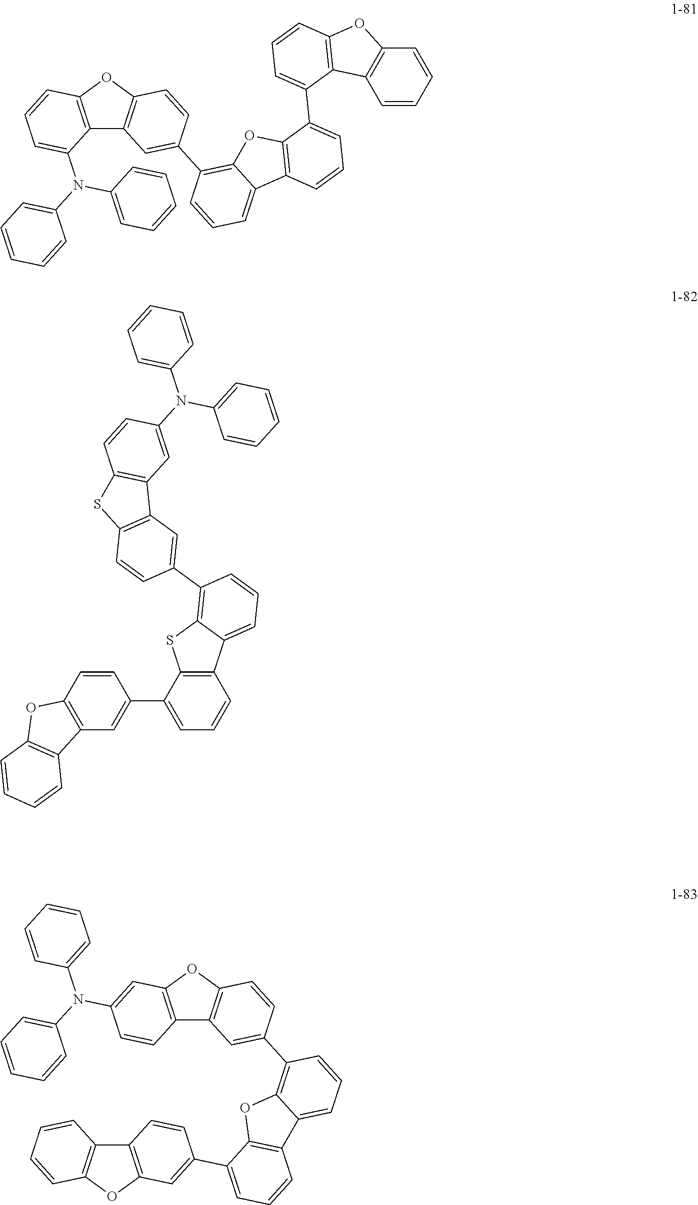 [SMILES: c1ccc(N(c2ccccc2)c2cccc3oc4ccc(-c5cccc6c5oc5c(-c7cccc8oc9ccccc9c78)cccc56)cc4c23)cc1 ; c1ccc(N(c2ccccc2)c2ccc3sc4ccc(-c5cccc6c5sc5c(-c7ccc8oc9ccccc9c8c7)cccc56)cc4c3c2)cc1 ; c1ccc(N(c2ccccc2)c2ccc3c(c2)oc2ccc(-c4cccc5c4oc4c(-c6ccc7c(c6)oc6ccccc67)cccc45)cc23)cc1]

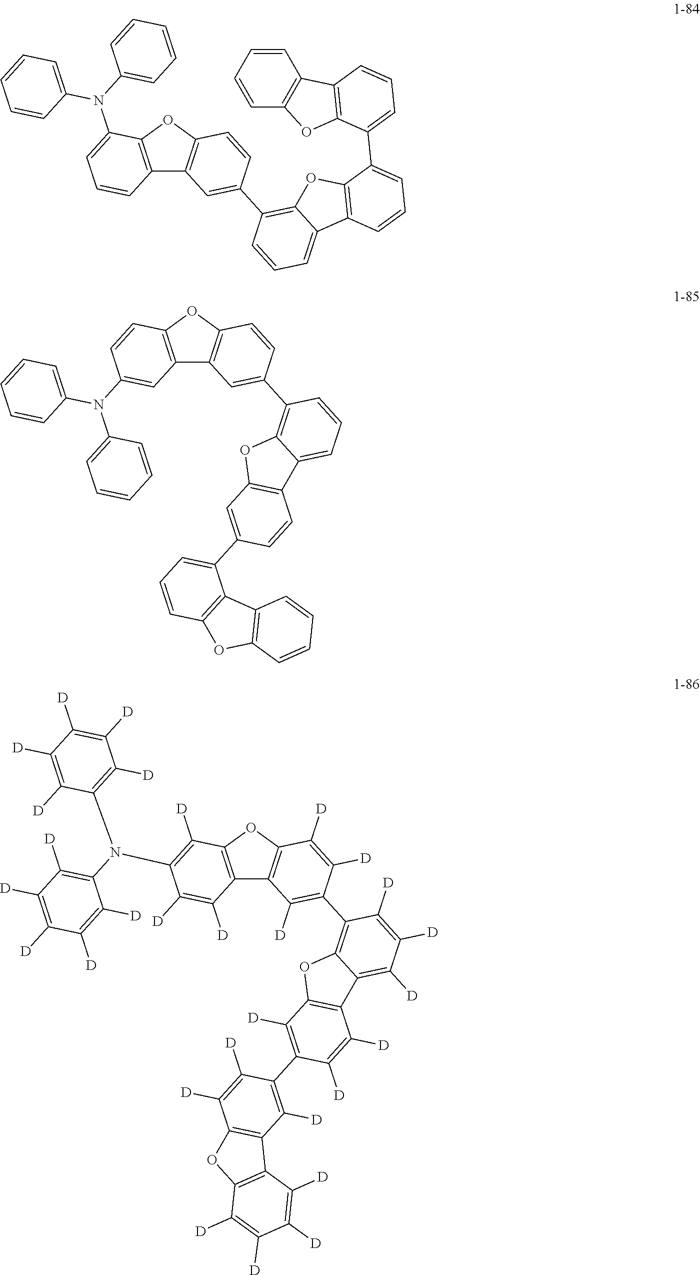 [SMILES: c1ccc(N(c2ccccc2)c2cccc3c2oc2ccc(-c4cccc5c4oc4c(-c6cccc7c6oc6ccccc67)cccc45)cc23)cc1 ; c1ccc(N(c2ccccc2)c2ccc3oc4ccc(-c5cccc6c5oc5cc(-c7cccc8oc9ccccc9c78)ccc56)cc4c3c2)cc1 ; [2H]c1c([2H])c([2H])c(N(c2c([2H])c([2H])c([2H])c([2H])c2[2H])c2c([2H])c([2H])c3c(oc4c([2H])c([2H])c(-c5c([2H])c([2H])c([2H])c6c5oc5c([2H])c(-c7c([2H])c([2H])c8oc9c([2H])c([2H])c([2H])c([2H])c9c8c7[2H])c([2H])c([2H])c56)c([2H])c43)c2[2H])c([2H])c1[2H]]

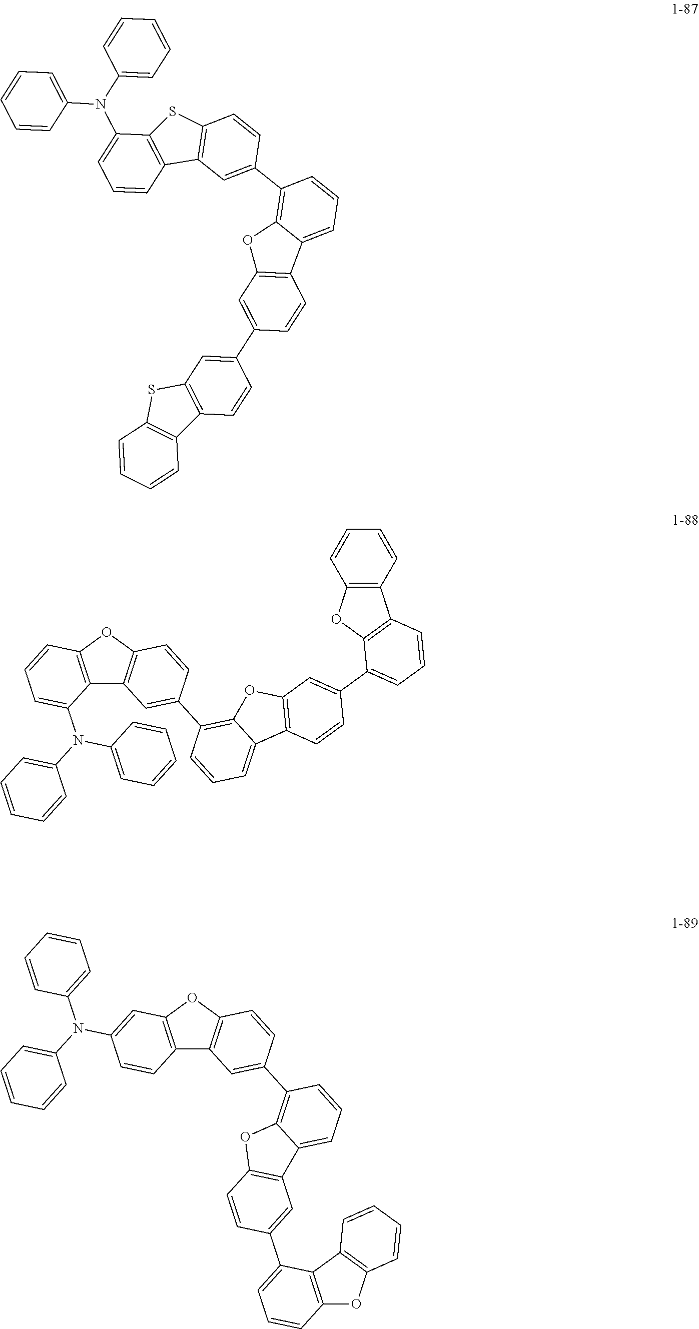 [SMILES: c1ccc(N(c2ccccc2)c2cccc3c2sc2ccc(-c4cccc5c4oc4cc(-c6ccc7c(c6)sc6ccccc67)ccc45)cc23)cc1 ; c1ccc(N(c2ccccc2)c2cccc3oc4ccc(-c5cccc6c5oc5cc(-c7cccc8c7oc7ccccc78)ccc56)cc4c23)cc1 ; c1ccc(N(c2ccccc2)c2ccc3c(c2)oc2ccc(-c4cccc5c4oc4ccc(-c6cccc7oc8ccccc8c67)cc45)cc23)cc1]

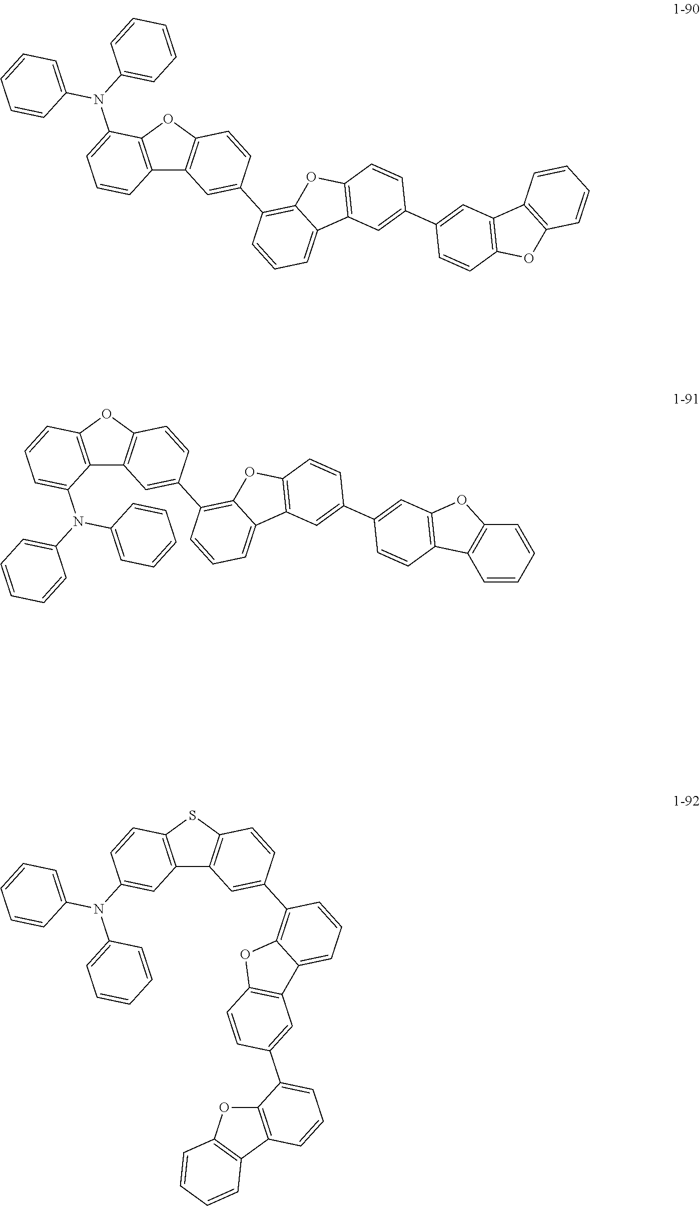 [SMILES: c1ccc(N(c2ccccc2)c2cccc3c2oc2ccc(-c4cccc5c4oc4ccc(-c6ccc7oc8ccccc8c7c6)cc45)cc23)cc1 ; c1ccc(N(c2ccccc2)c2cccc3oc4ccc(-c5cccc6c5oc5ccc(-c7ccc8c(c7)oc7ccccc78)cc56)cc4c23)cc1 ; c1ccc(N(c2ccccc2)c2ccc3sc4ccc(-c5cccc6c5oc5ccc(-c7cccc8c7oc7ccccc78)cc56)cc4c3c2)cc1]

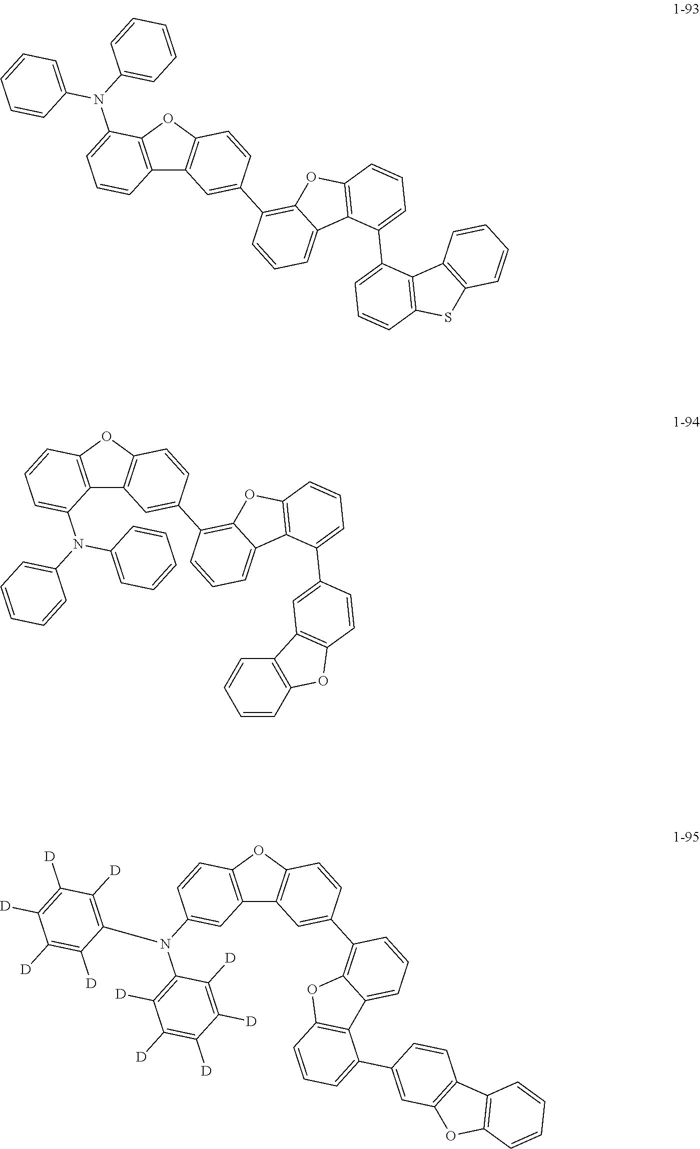 [SMILES: c1ccc(N(c2ccccc2)c2cccc3c2oc2ccc(-c4cccc5c4oc4cccc(-c6cccc7sc8ccccc8c67)c45)cc23)cc1 ; c1ccc(N(c2ccccc2)c2cccc3oc4ccc(-c5cccc6c5oc5cccc(-c7ccc8oc9ccccc9c8c7)c56)cc4c23)cc1 ; [2H]c1c([2H])c([2H])c(N(c2ccc3oc4ccc(-c5cccc6c5oc5cccc(-c7ccc8c(c7)oc7ccccc78)c56)cc4c3c2)c2c([2H])c([2H])c([2H])c([2H])c2[2H])c([2H])c1[2H]]

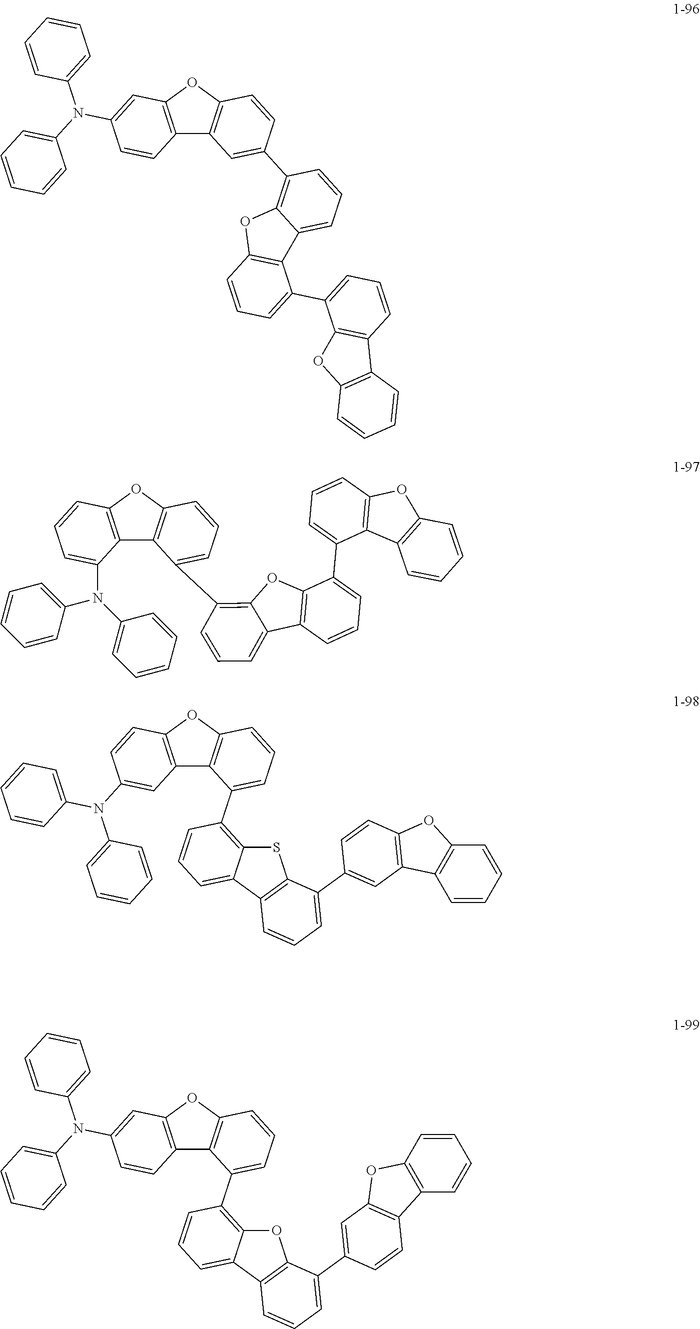

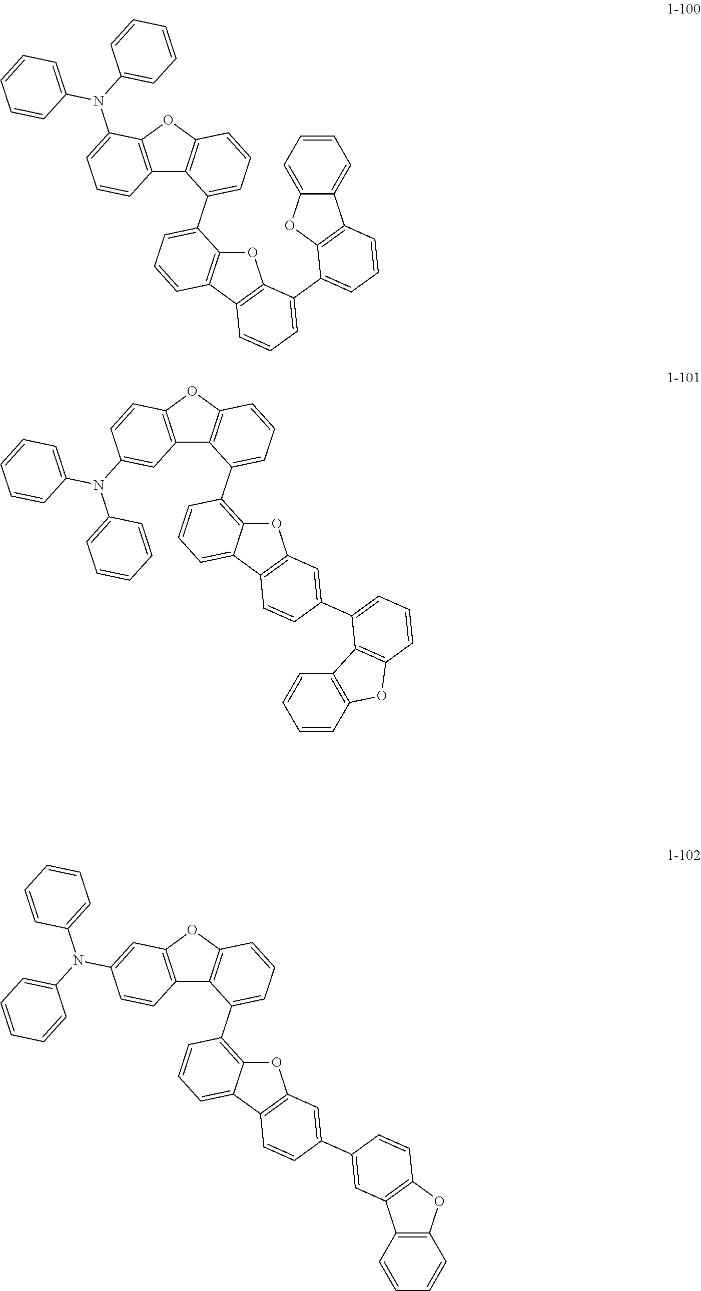 [SMILES: c1ccc(N(c2ccccc2)c2cccc3c2oc2cccc(-c4cccc5c4oc4c(-c6cccc7c6oc6ccccc67)cccc45)c23)cc1 ; c1ccc(N(c2ccccc2)c2ccc3oc4cccc(-c5cccc6c5oc5cc(-c7cccc8oc9ccccc9c78)ccc56)c4c3c2)cc1 ; c1ccc(N(c2ccccc2)c2ccc3c(c2)oc2cccc(-c4cccc5c4oc4cc(-c6ccc7oc8ccccc8c7c6)ccc45)c23)cc1]

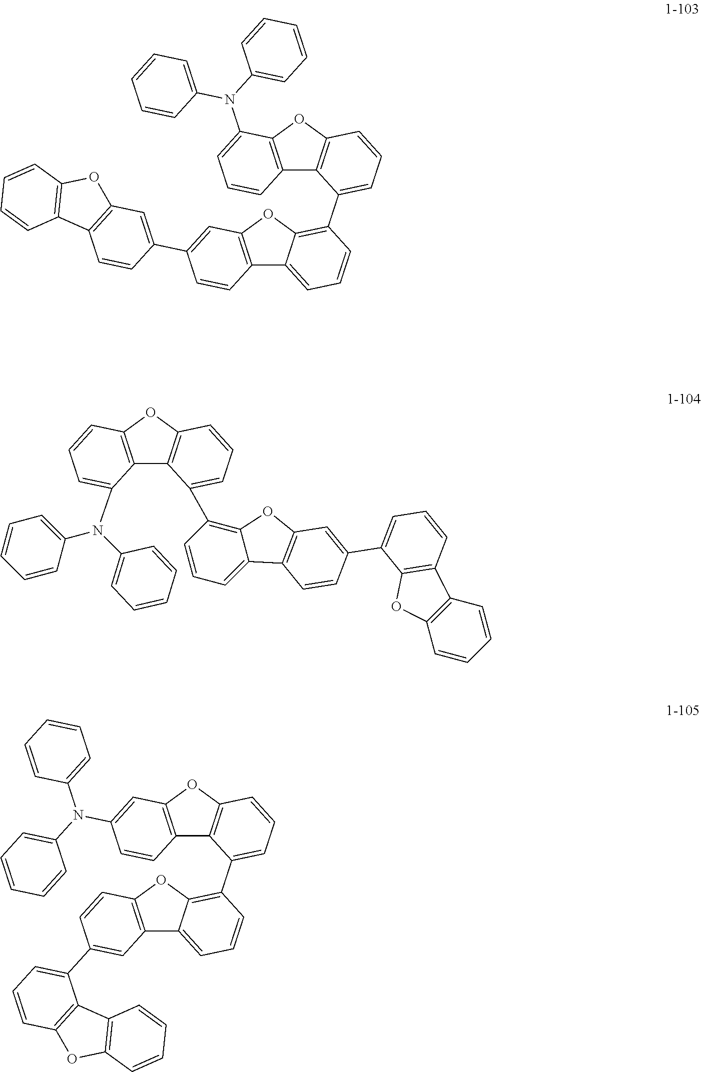 [SMILES: c1ccc(N(c2ccccc2)c2cccc3c2oc2cccc(-c4cccc5c4oc4cc(-c6ccc7c(c6)oc6ccccc67)ccc45)c23)cc1 ; c1ccc(N(c2ccccc2)c2cccc3oc4cccc(-c5cccc6c5oc5cc(-c7cccc8c7oc7ccccc78)ccc56)c4c23)cc1 ; c1ccc(N(c2ccccc2)c2ccc3c(c2)oc2cccc(-c4cccc5c4oc4ccc(-c6cccc7oc8ccccc8c67)cc45)c23)cc1]

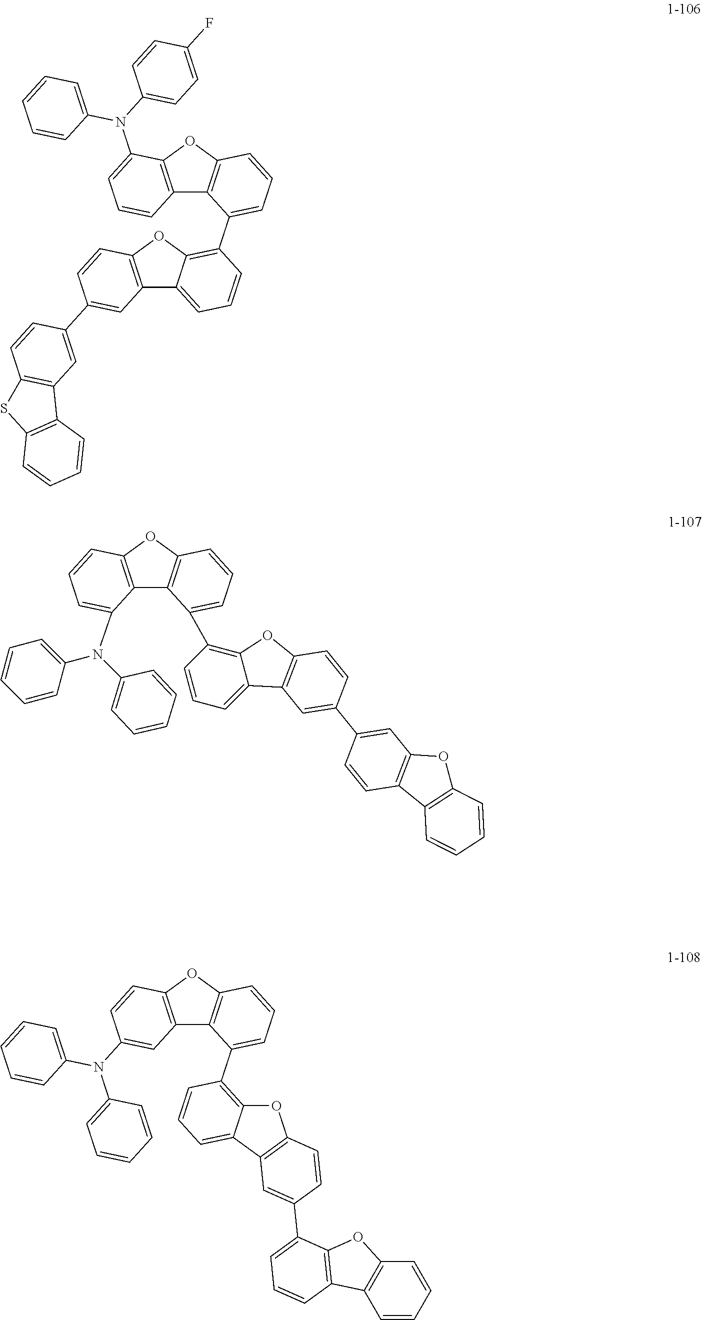 [SMILES: Fc1ccc(N(c2ccccc2)c2cccc3c2oc2cccc(-c4cccc5c4oc4ccc(-c6ccc7sc8ccccc8c7c6)cc45)c23)cc1 ; c1ccc(N(c2ccccc2)c2cccc3oc4cccc(-c5cccc6c5oc5ccc(-c7ccc8c(c7)oc7ccccc78)cc56)c4c23)cc1 ; c1ccc(N(c2ccccc2)c2ccc3oc4cccc(-c5cccc6c5oc5ccc(-c7cccc8c7oc7ccccc78)cc56)c4c3c2)cc1]

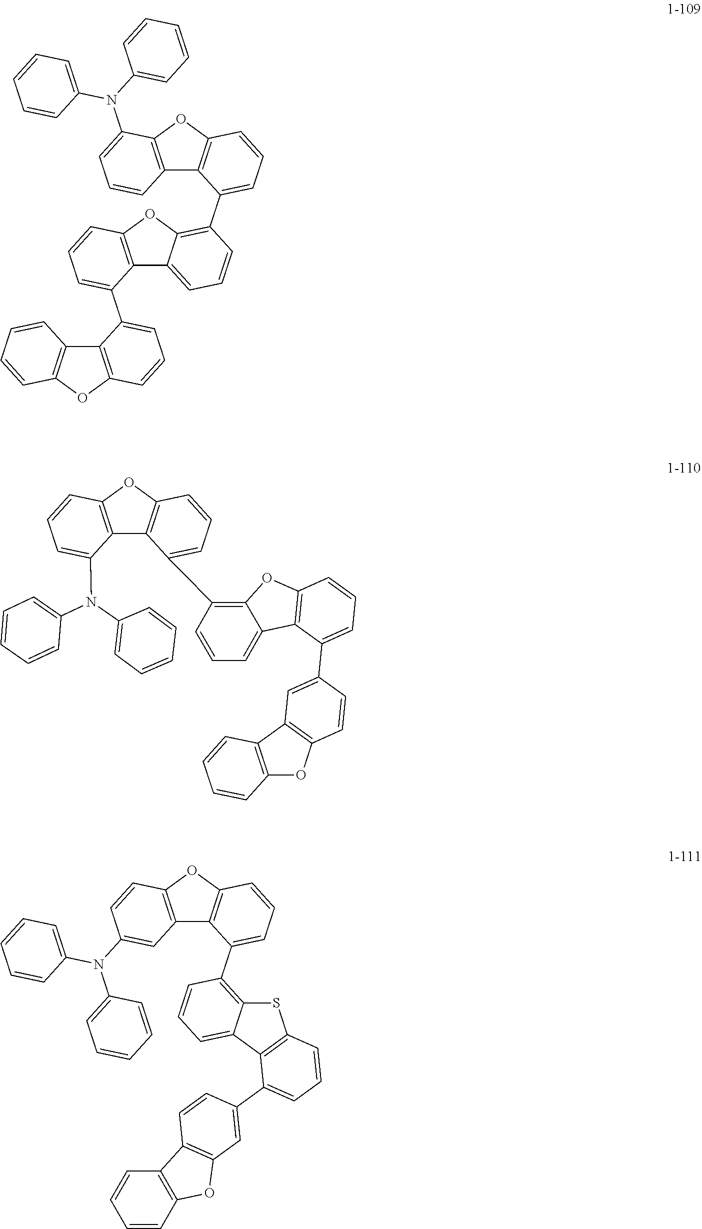 [SMILES: c1ccc(N(c2ccccc2)c2cccc3c2oc2cccc(-c4cccc5c4oc4cccc(-c6cccc7oc8ccccc8c67)c45)c23)cc1 ; c1ccc(N(c2ccccc2)c2cccc3oc4cccc(-c5cccc6c5oc5cccc(-c7ccc8oc9ccccc9c8c7)c56)c4c23)cc1 ; c1ccc(N(c2ccccc2)c2ccc3oc4cccc(-c5cccc6c5sc5cccc(-c7ccc8c(c7)oc7ccccc78)c56)c4c3c2)cc1]

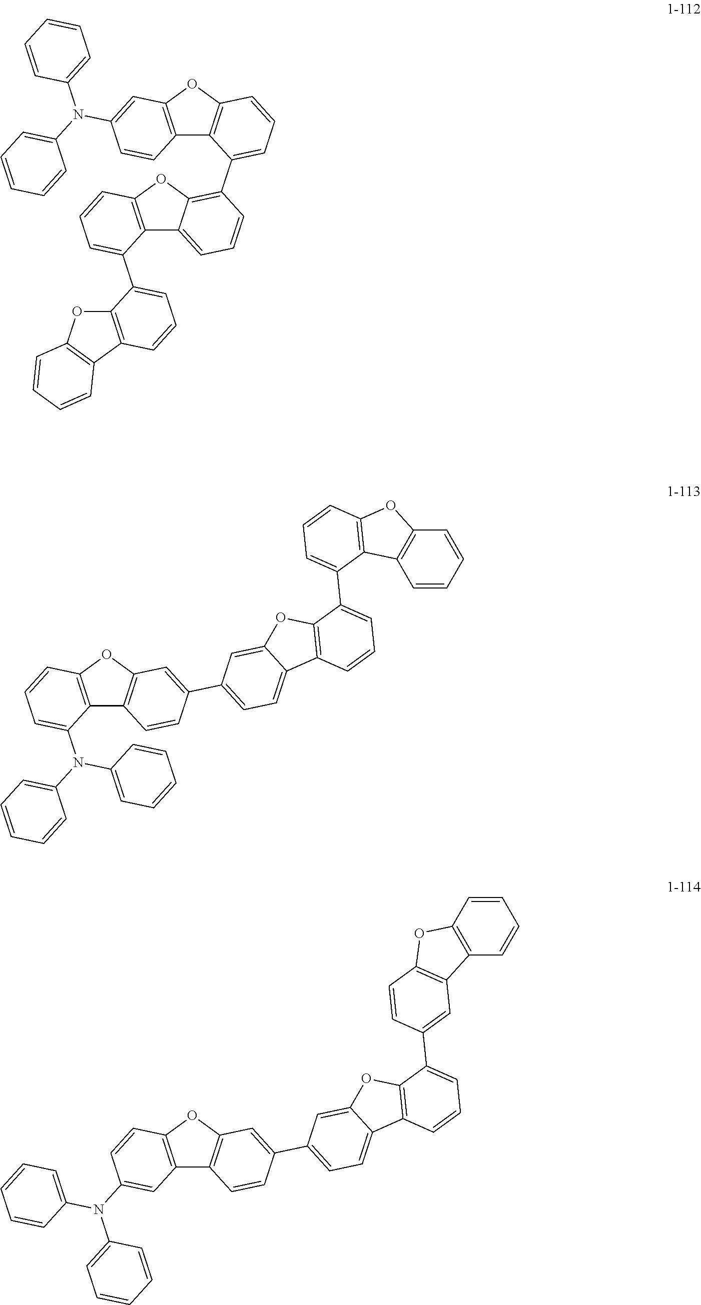 [SMILES: c1ccc(N(c2ccccc2)c2ccc3c(c2)oc2cccc(-c4cccc5c4oc4cccc(-c6cccc7c6oc6ccccc67)c45)c23)cc1 ; c1ccc(N(c2ccccc2)c2cccc3oc4cc(-c5ccc6c(c5)oc5c(-c7cccc8oc9ccccc9c78)cccc56)ccc4c23)cc1 ; c1ccc(N(c2ccccc2)c2ccc3oc4cc(-c5ccc6c(c5)oc5c(-c7ccc8oc9ccccc9c8c7)cccc56)ccc4c3c2)cc1]

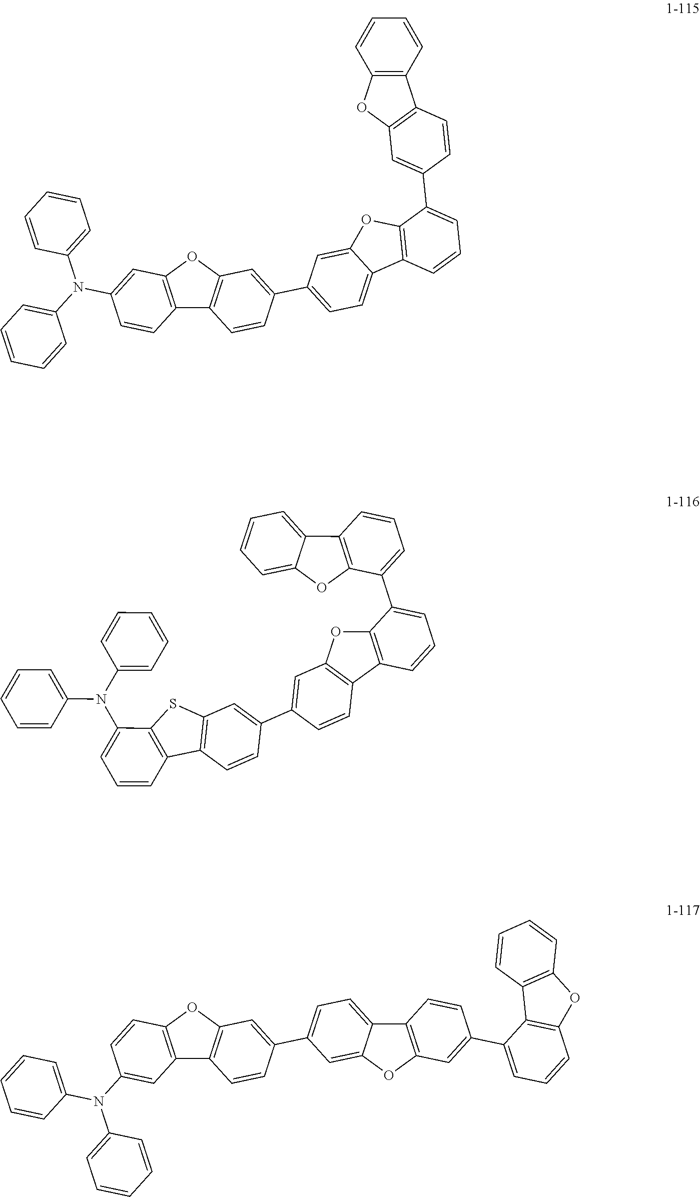 [SMILES: c1ccc(N(c2ccccc2)c2ccc3c(c2)oc2cc(-c4ccc5c(c4)oc4c(-c6ccc7c(c6)oc6ccccc67)cccc45)ccc23)cc1 ; c1ccc(N(c2ccccc2)c2cccc3c2sc2cc(-c4ccc5c(c4)oc4c(-c6cccc7c6oc6ccccc67)cccc45)ccc23)cc1 ; c1ccc(N(c2ccccc2)c2ccc3oc4cc(-c5ccc6c(c5)oc5cc(-c7cccc8oc9ccccc9c78)ccc56)ccc4c3c2)cc1]

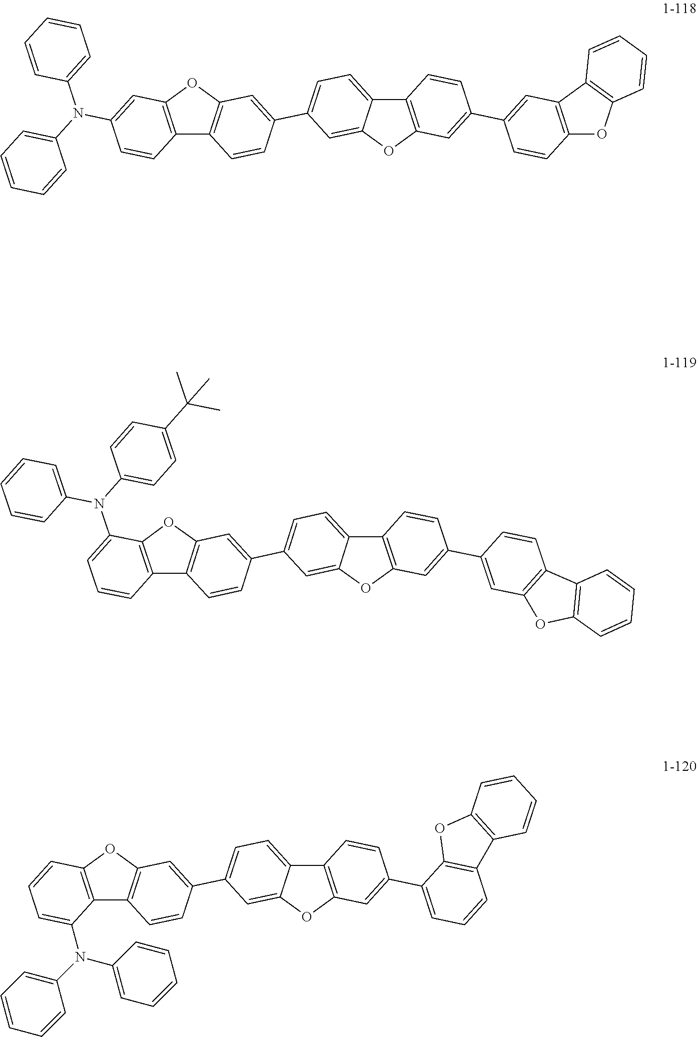 [SMILES: c1ccc(N(c2ccccc2)c2ccc3c(c2)oc2cc(-c4ccc5c(c4)oc4cc(-c6ccc7oc8ccccc8c7c6)ccc45)ccc23)cc1 ; CC(C)(C)c1ccc(N(c2ccccc2)c2cccc3c2oc2cc(-c4ccc5c(c4)oc4cc(-c6ccc7c(c6)oc6ccccc67)ccc45)ccc23)cc1 ; c1ccc(N(c2ccccc2)c2cccc3oc4cc(-c5ccc6c(c5)oc5cc(-c7cccc8c7oc7ccccc78)ccc56)ccc4c23)cc1]

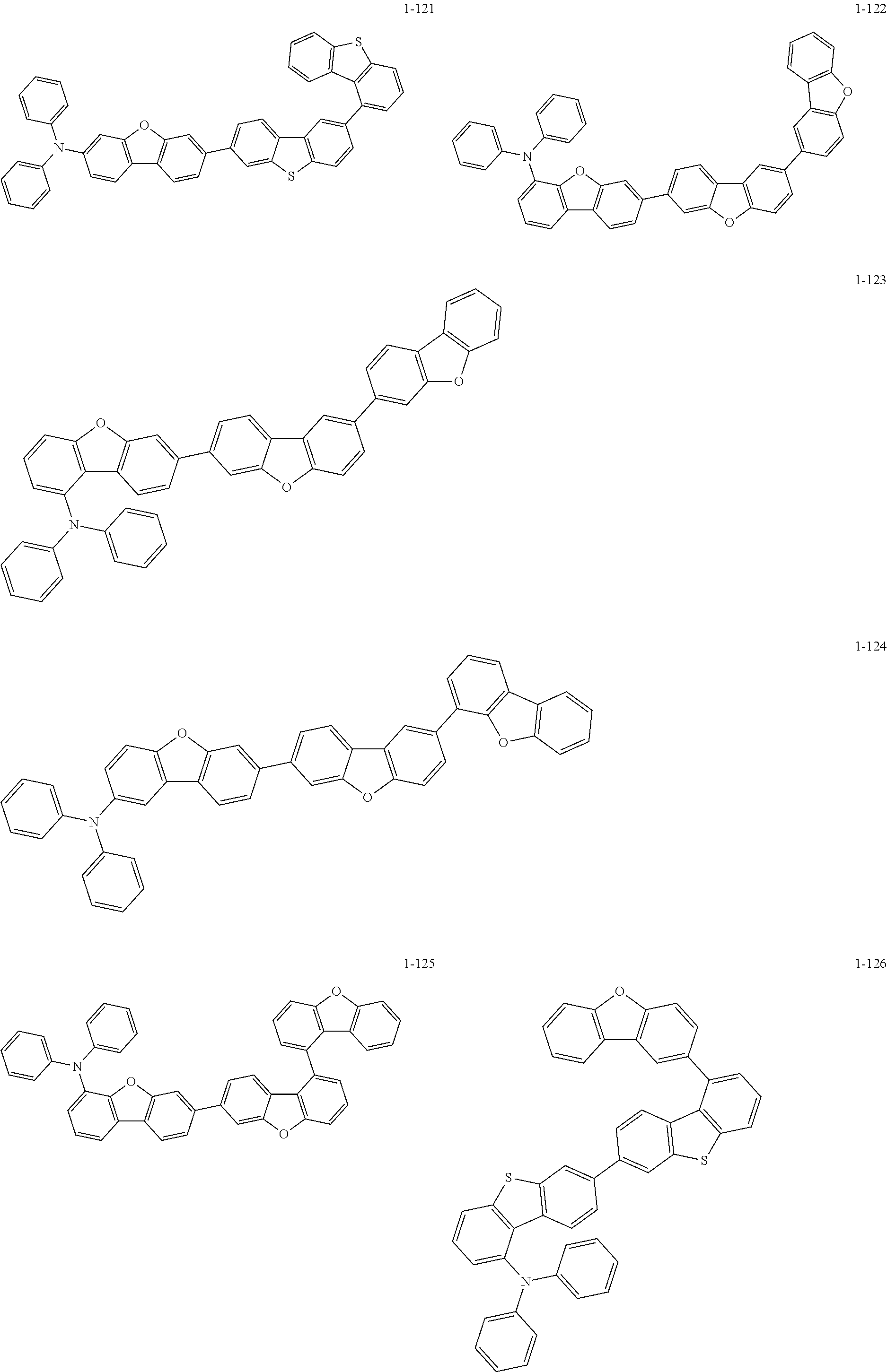

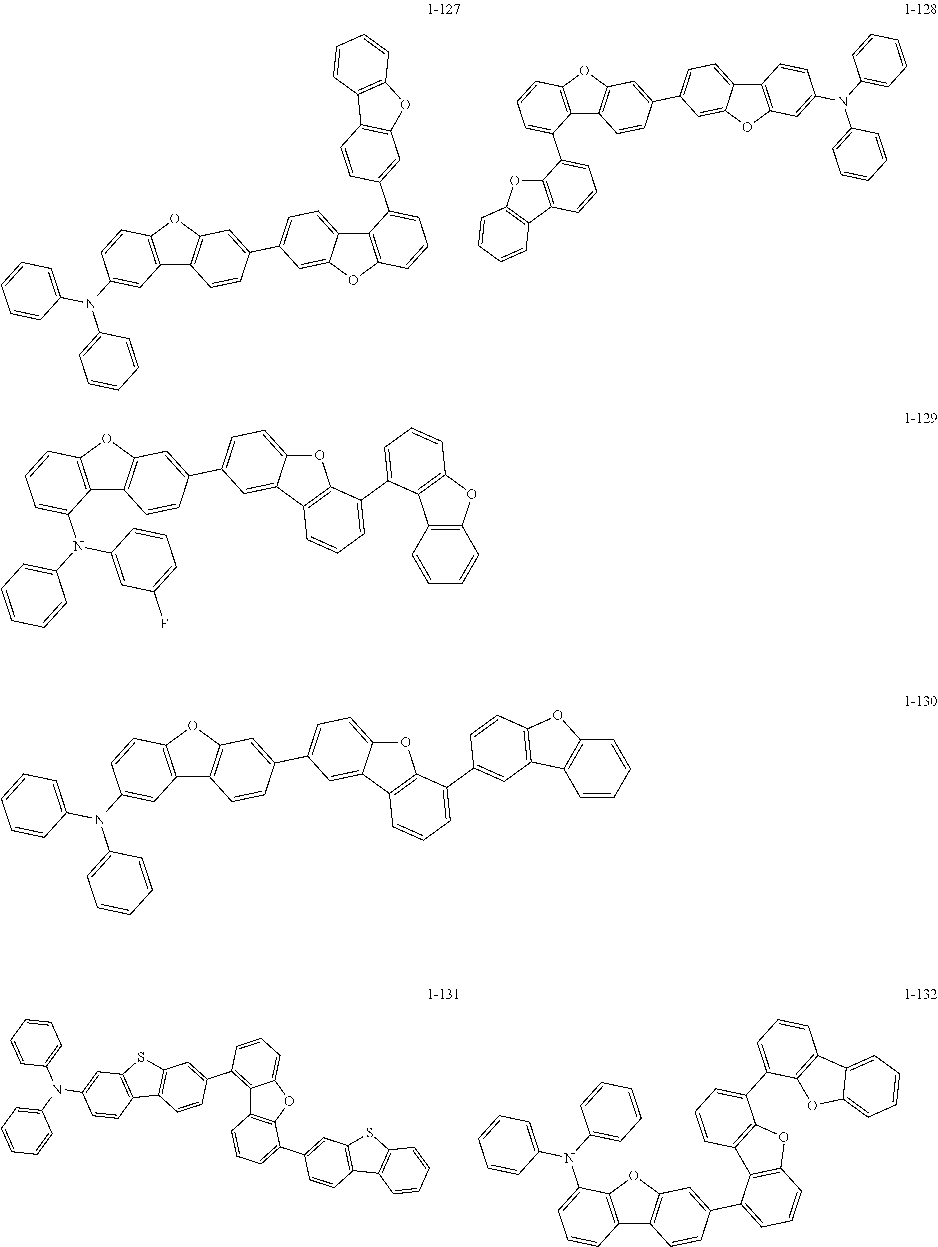

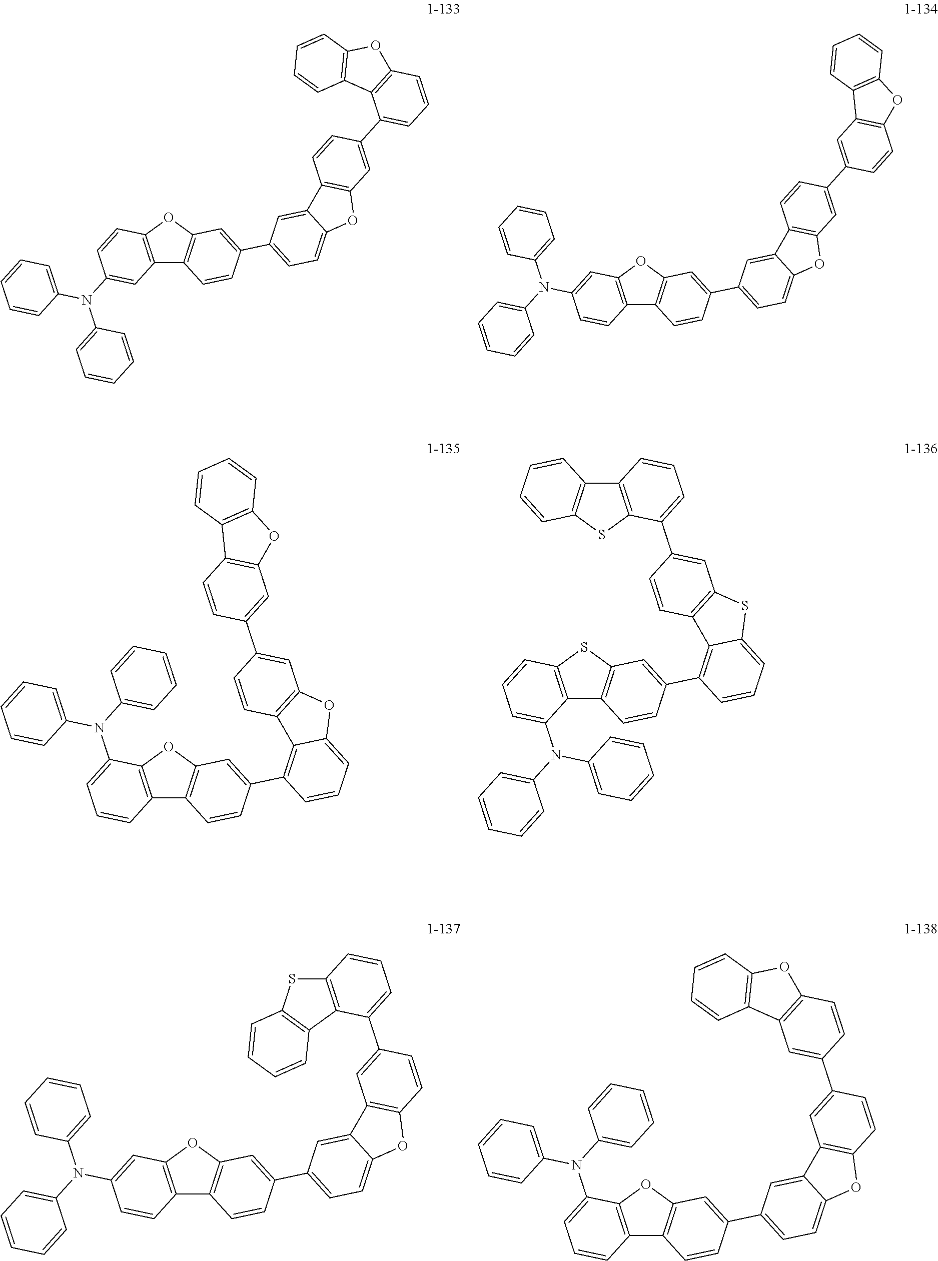

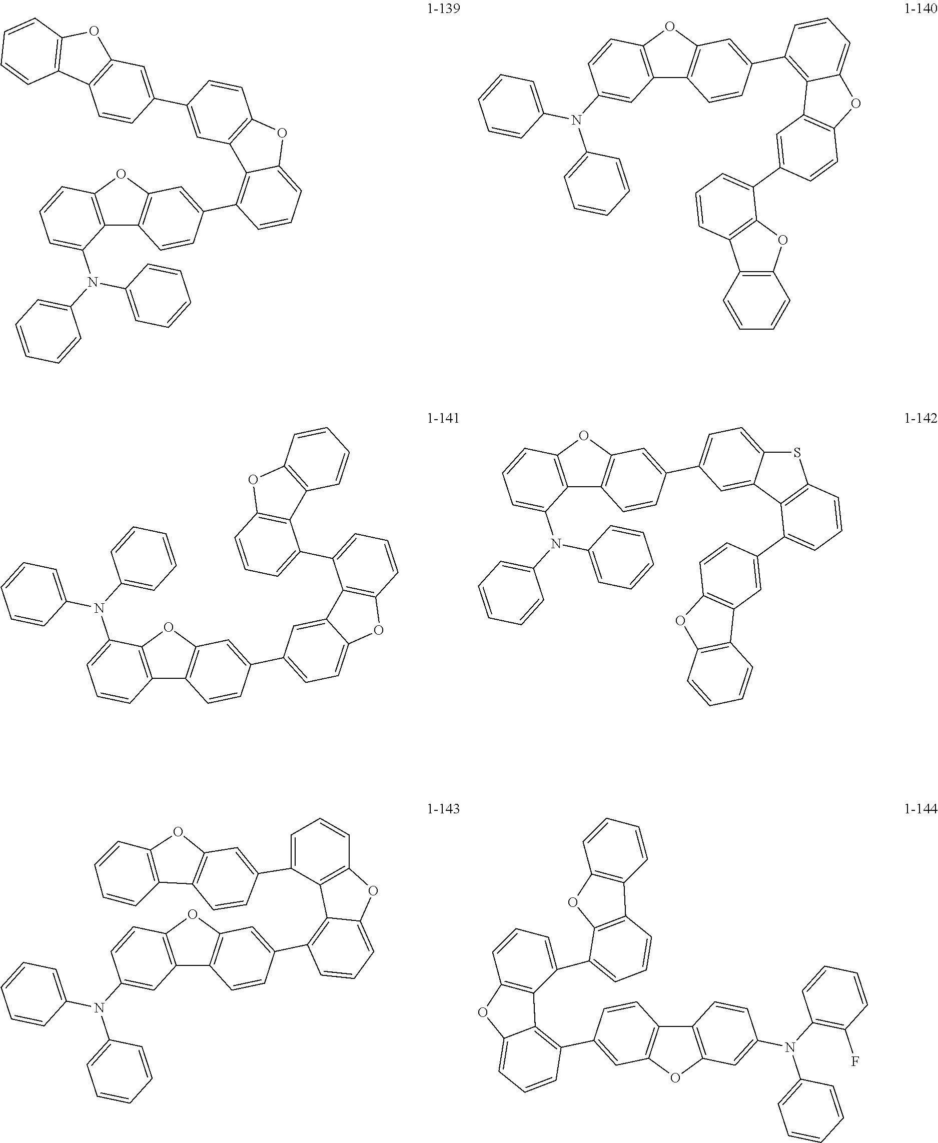

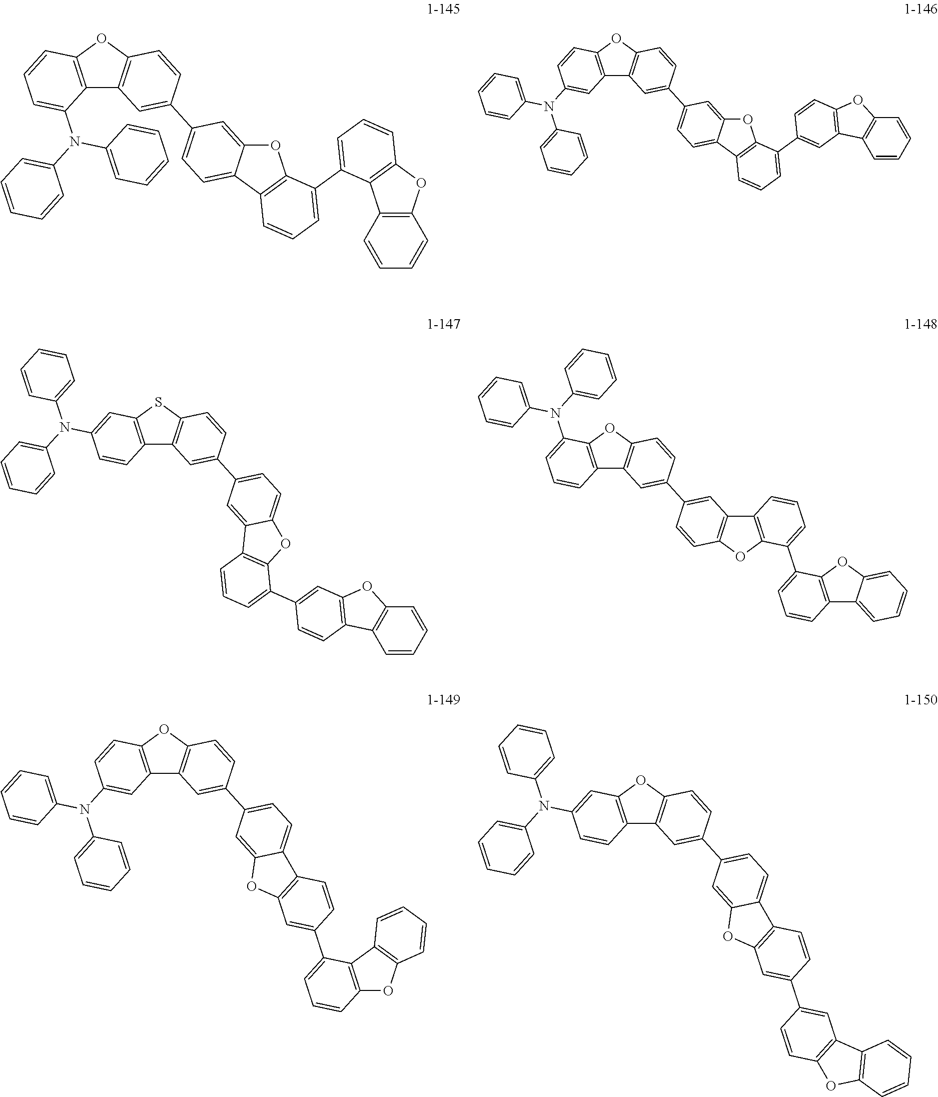

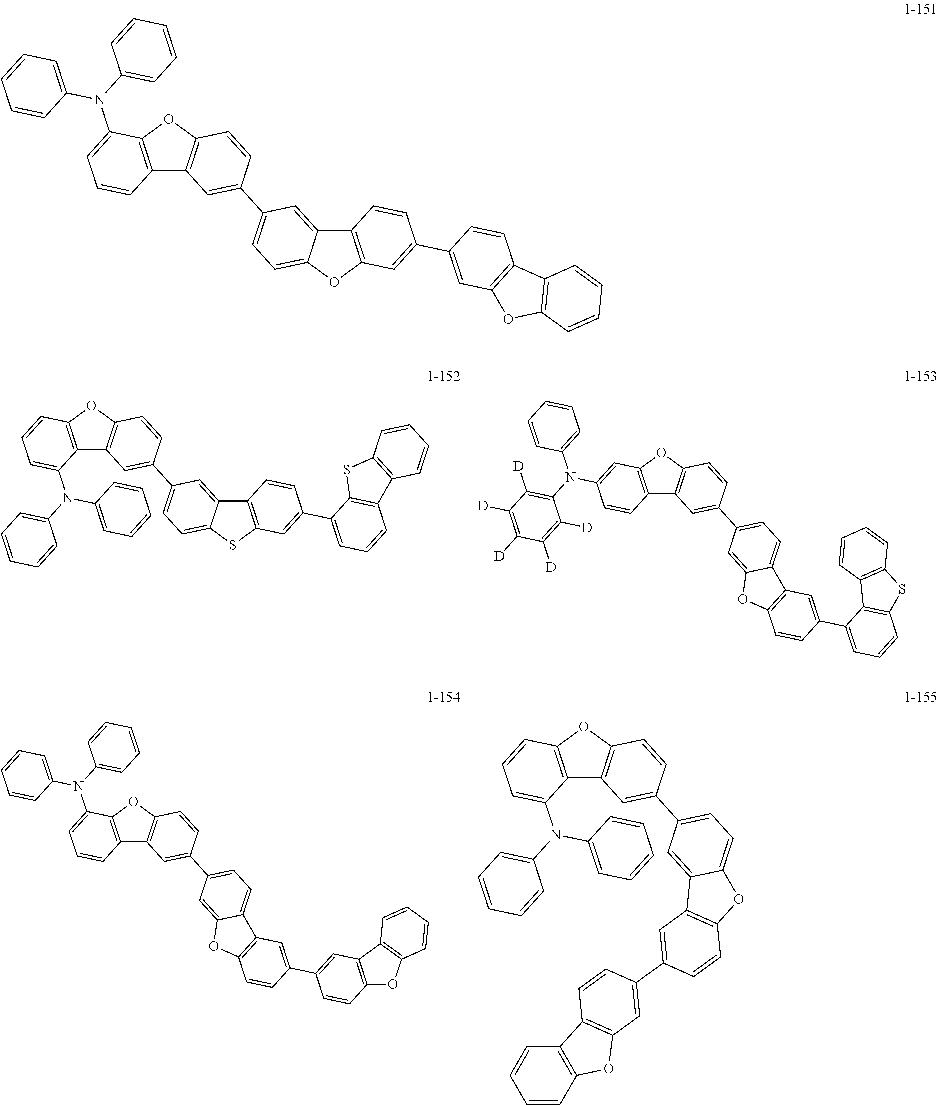 [SMILES: c1ccc(N(c2ccccc2)c2cccc3c2oc2ccc(-c4ccc5oc6cc(-c7ccc8c(c7)oc7ccccc78)ccc6c5c4)cc23)cc1 ; c1ccc(N(c2ccccc2)c2cccc3oc4ccc(-c5ccc6sc7cc(-c8cccc9c8sc8ccccc89)ccc7c6c5)cc4c23)cc1 ; [2H]c1c([2H])c([2H])c(N(c2ccccc2)c2ccc3c(c2)oc2ccc(-c4ccc5c(c4)oc4ccc(-c6cccc7sc8ccccc8c67)cc45)cc23)c([2H])c1[2H] ; c1ccc(N(c2ccccc2)c2cccc3c2oc2ccc(-c4ccc5c(c4)oc4ccc(-c6ccc7oc8ccccc8c7c6)cc45)cc23)cc1 ; c1ccc(N(c2ccccc2)c2cccc3oc4ccc(-c5ccc6oc7ccc(-c8ccc9c(c8)oc8ccccc89)cc7c6c5)cc4c23)cc1]

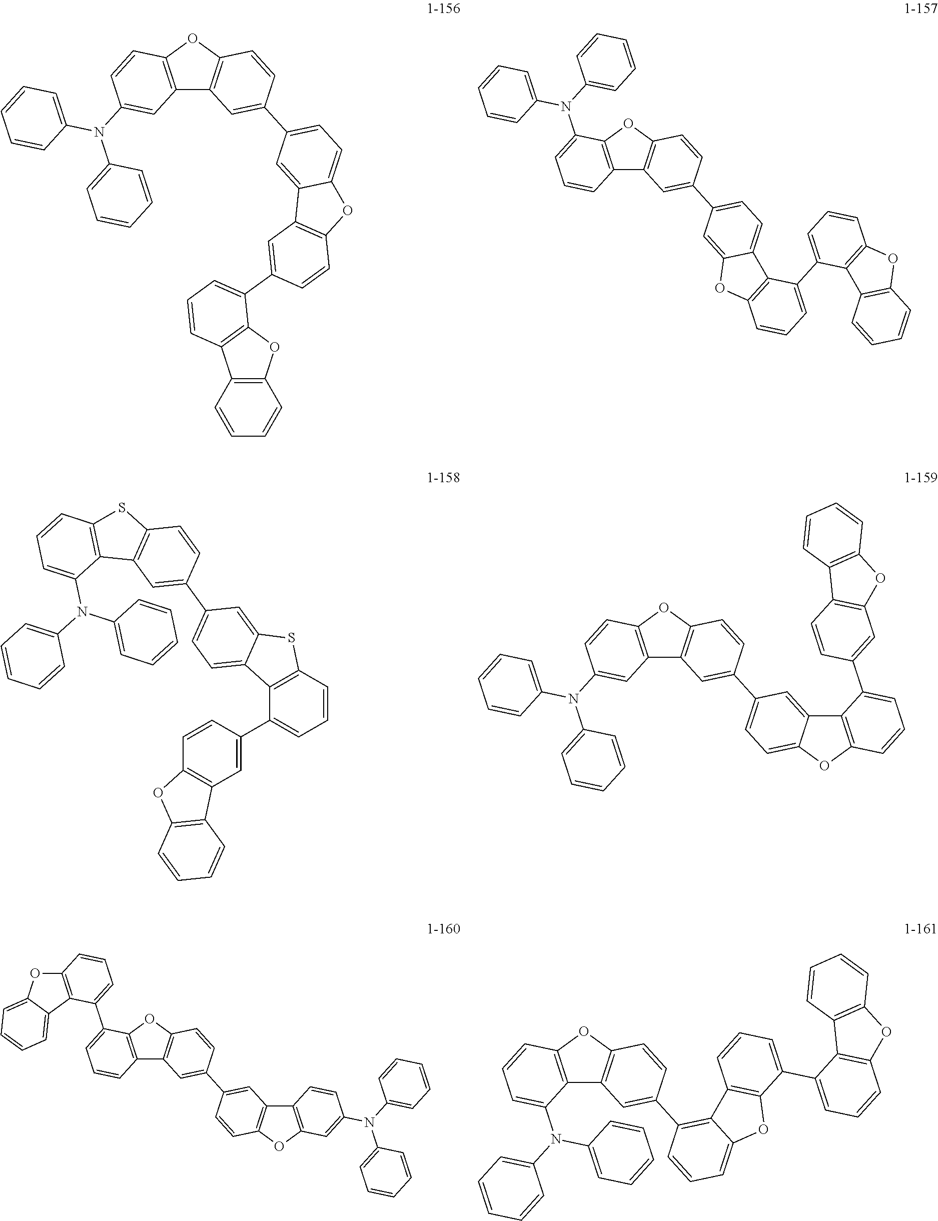

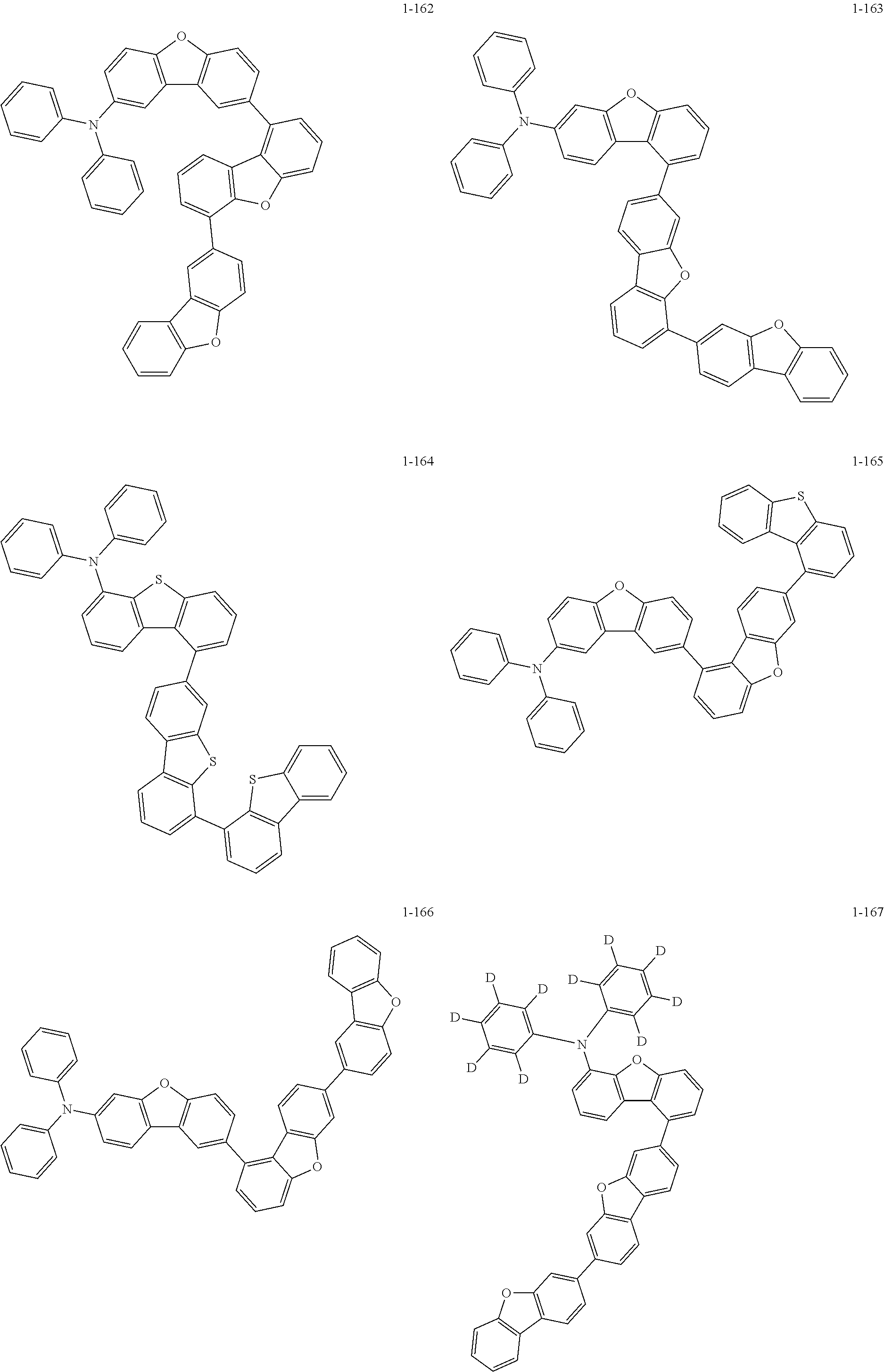

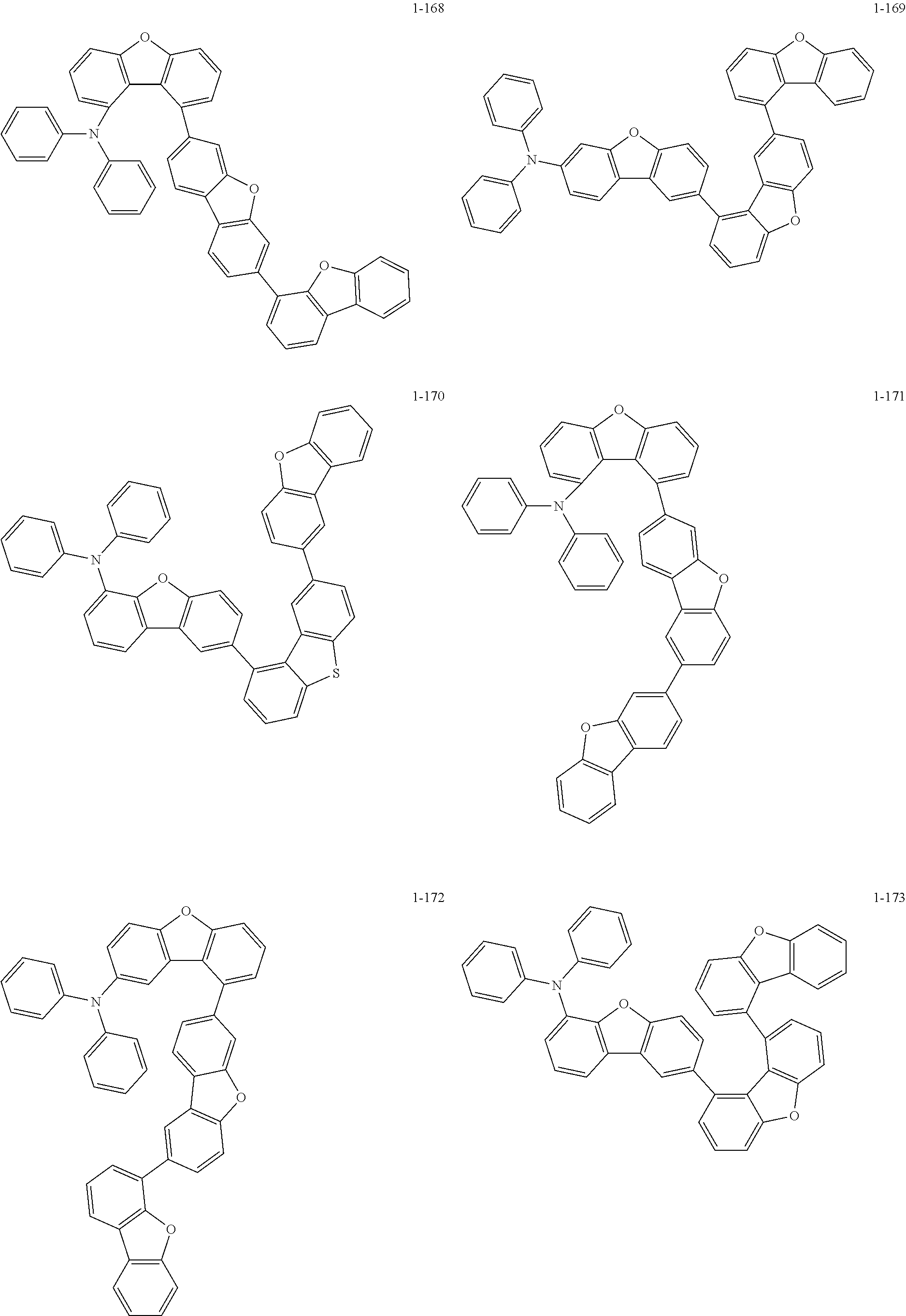

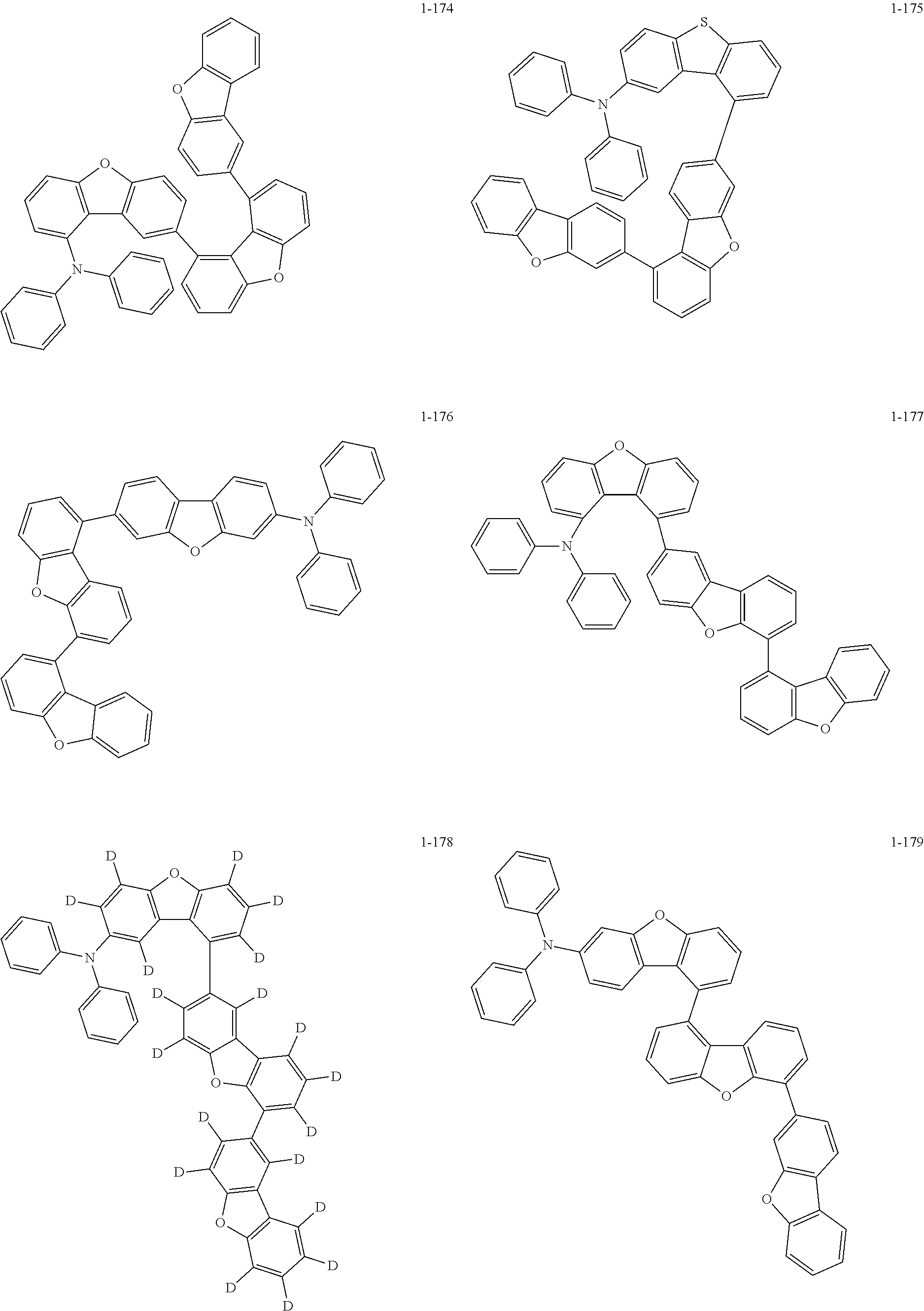

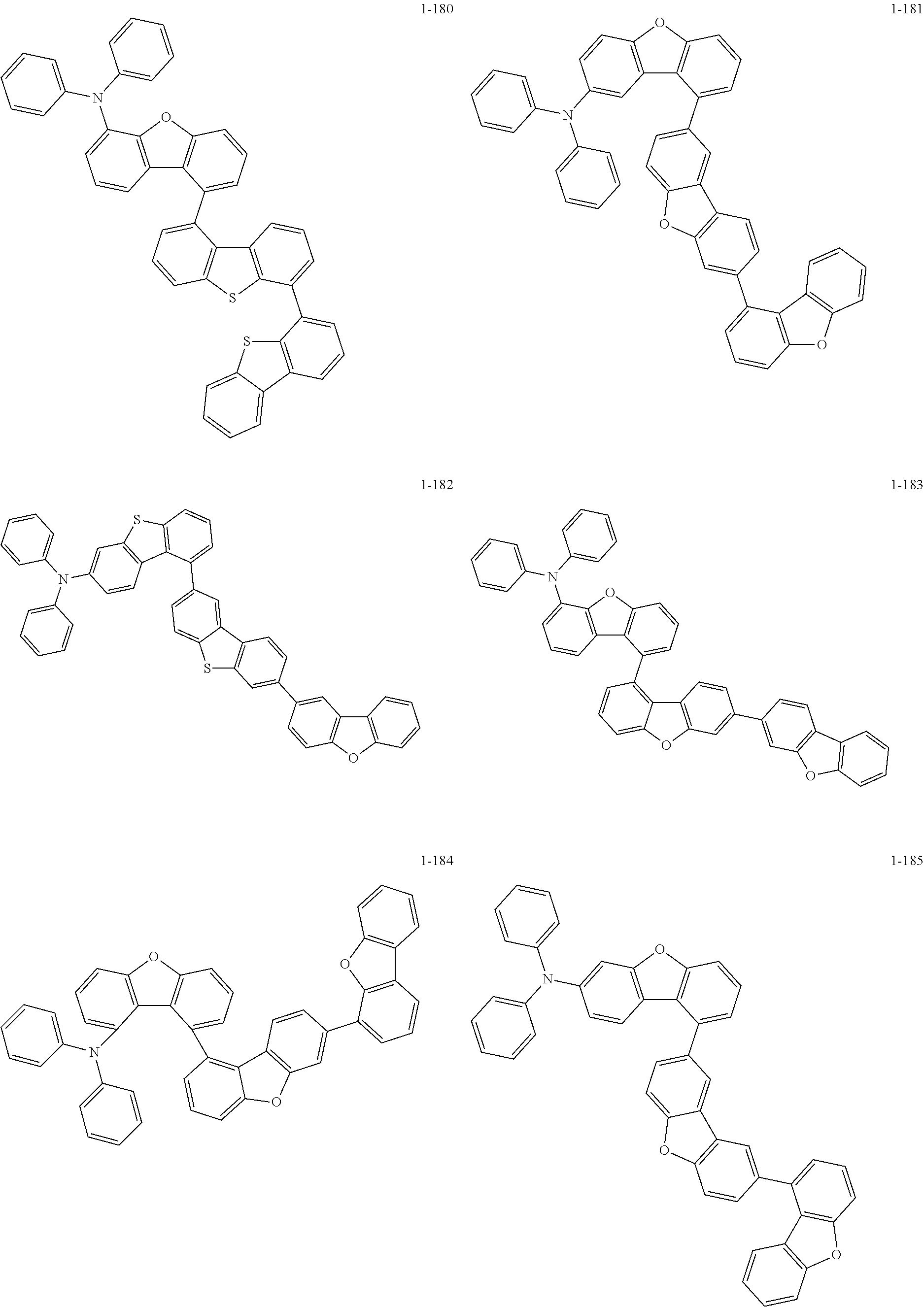

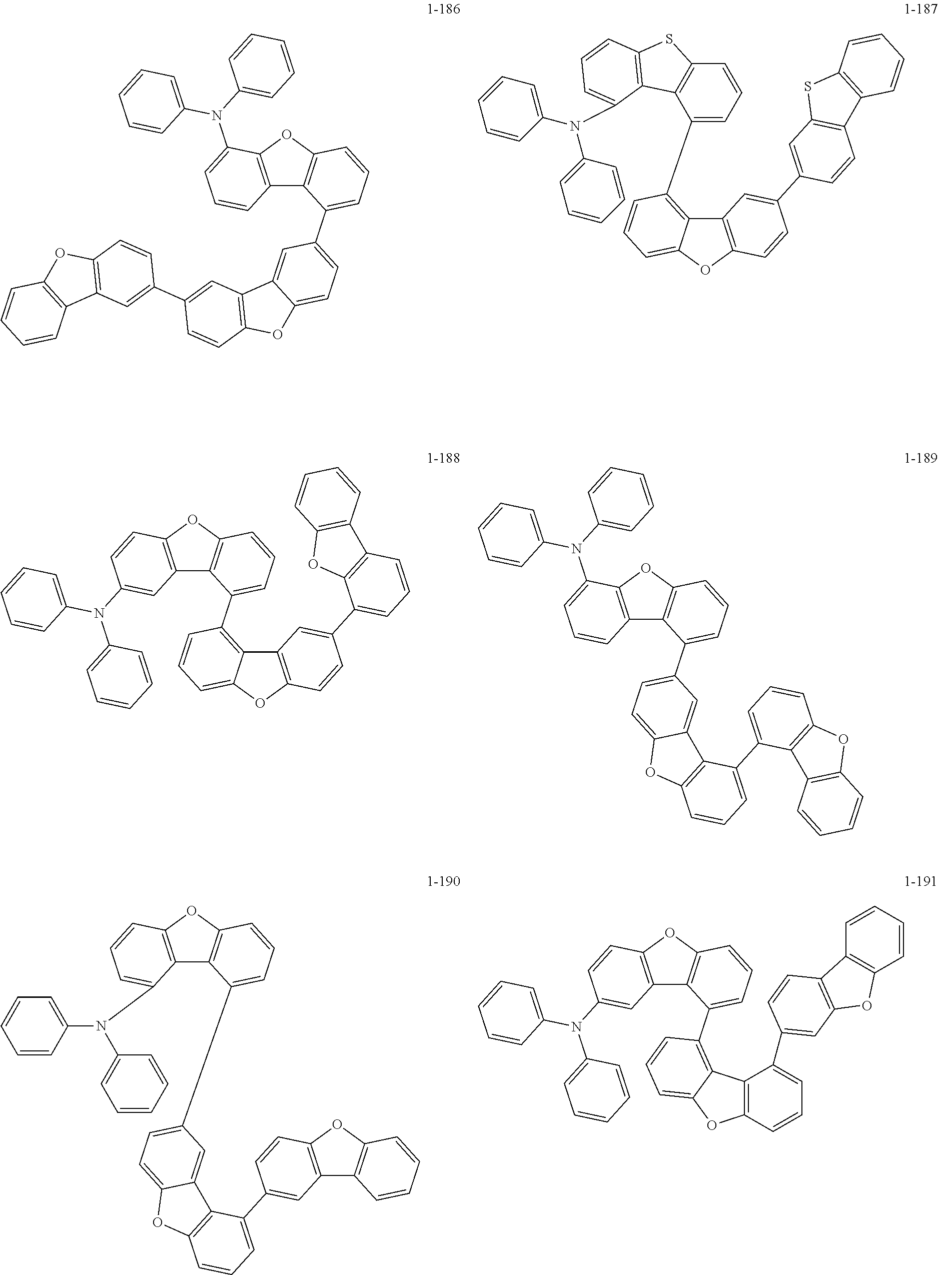

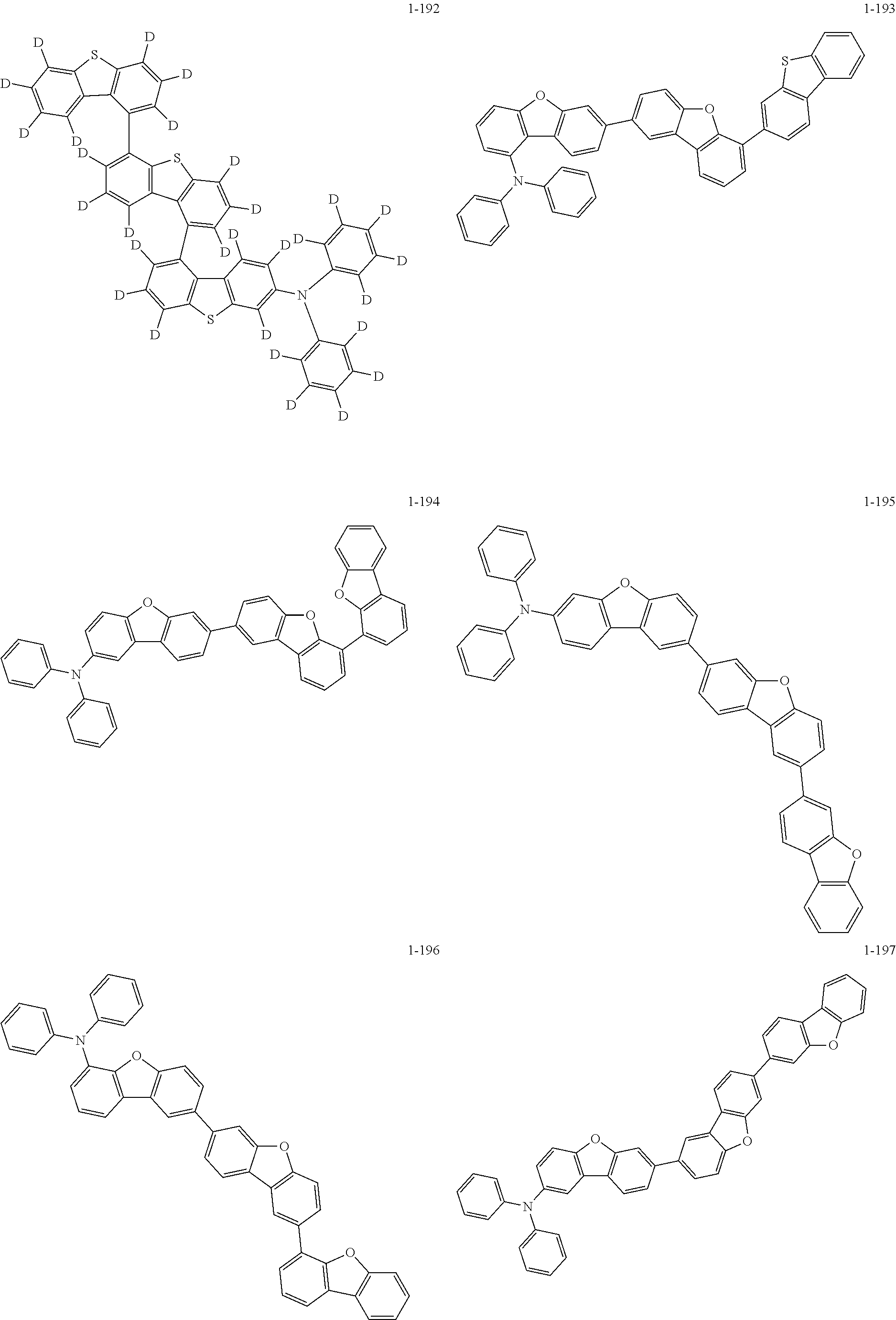

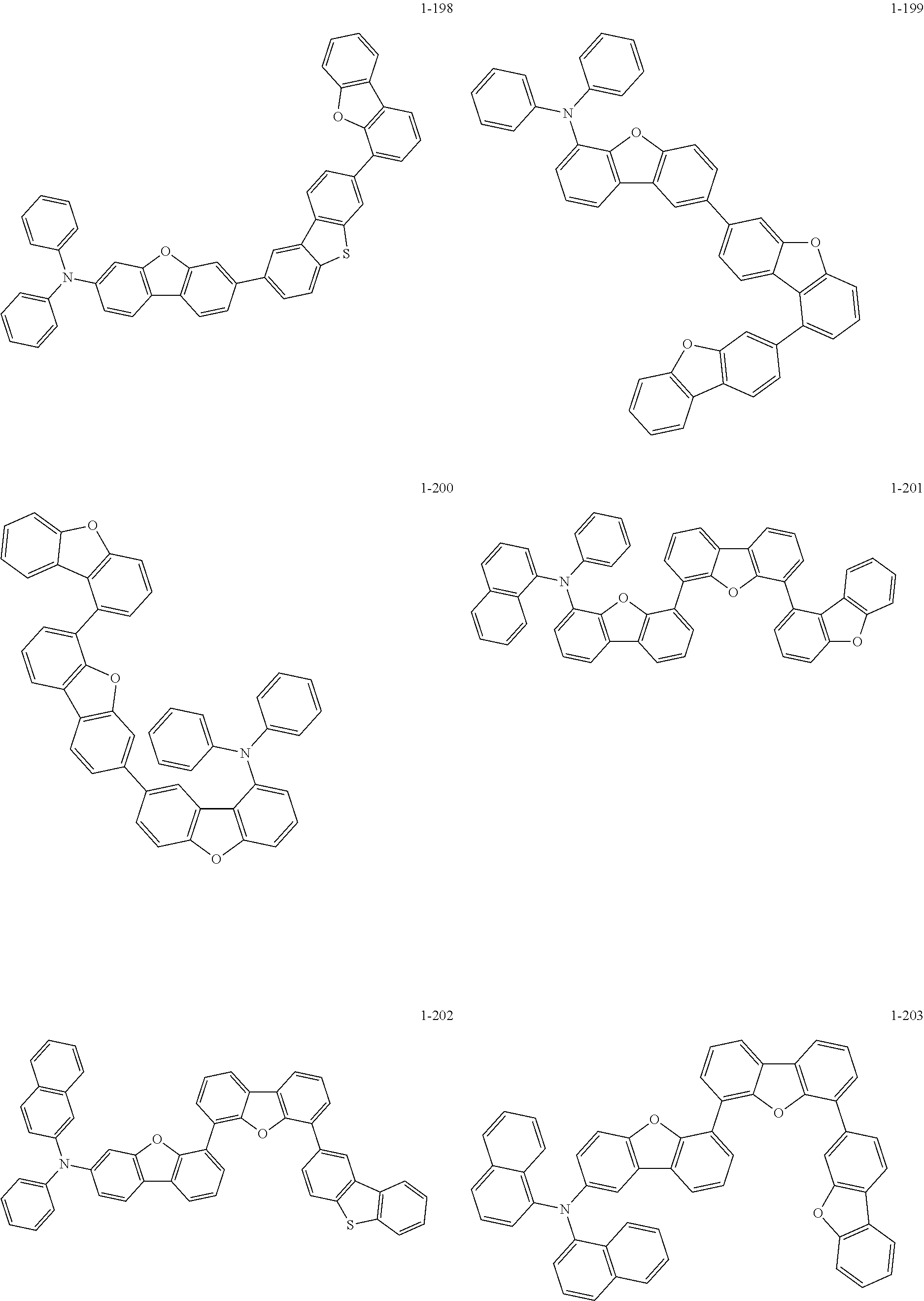

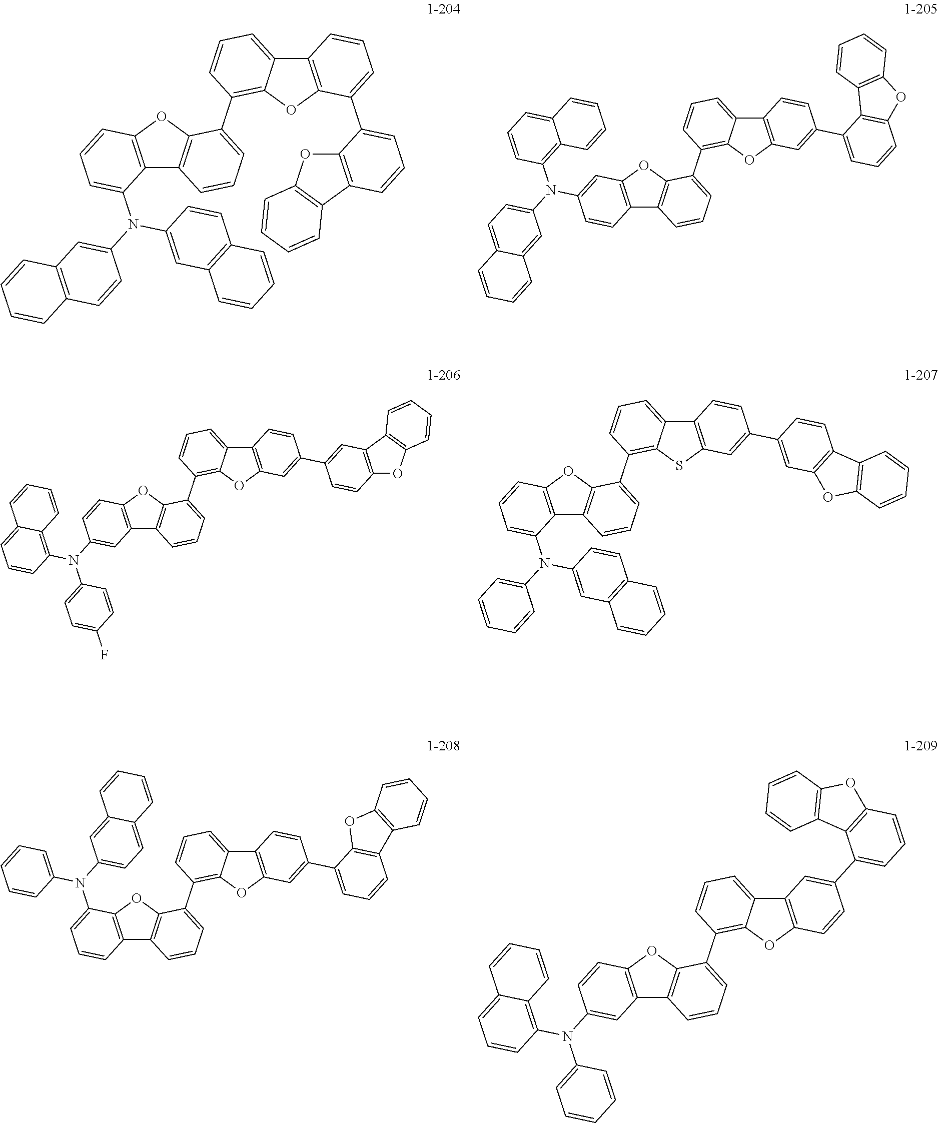

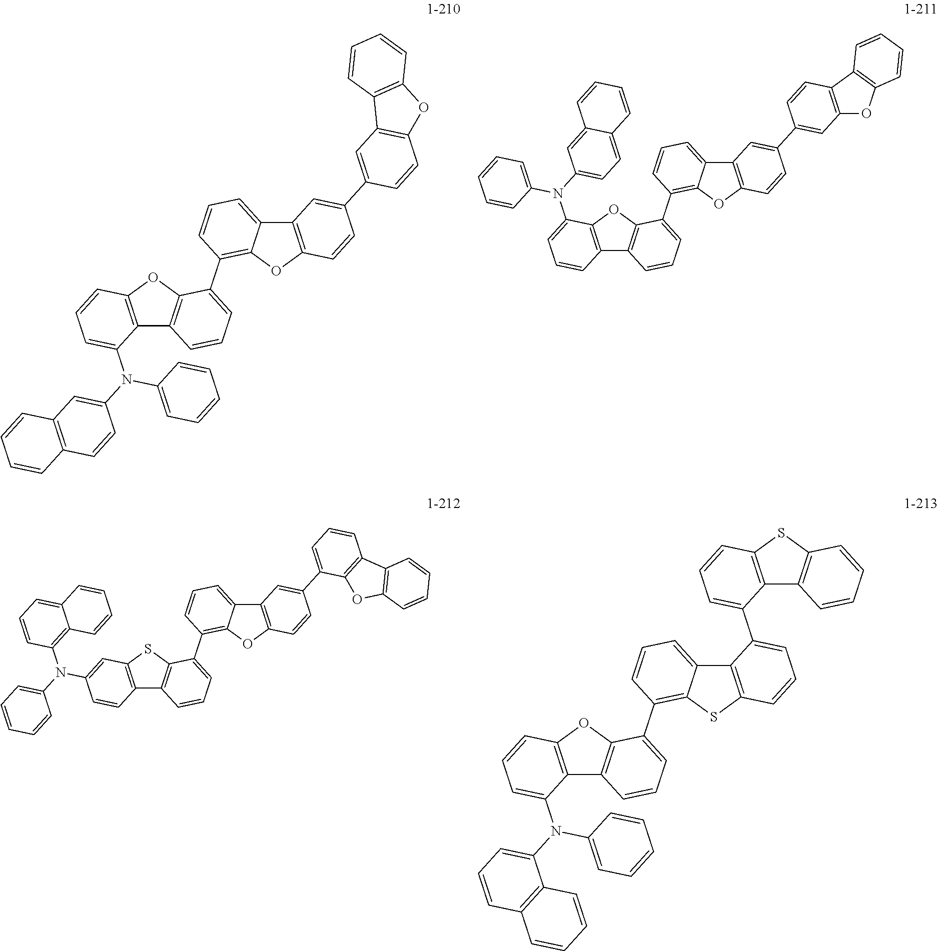 [SMILES: c1ccc(N(c2ccc3ccccc3c2)c2cccc3oc4c(-c5cccc6c5oc5ccc(-c7ccc8oc9ccccc9c8c7)cc56)cccc4c23)cc1 ; c1ccc(N(c2ccc3ccccc3c2)c2cccc3c2oc2c(-c4cccc5c4oc4ccc(-c6ccc7c(c6)oc6ccccc67)cc45)cccc23)cc1 ; c1ccc(N(c2ccc3c(c2)sc2c(-c4cccc5c4oc4ccc(-c6cccc7c6oc6ccccc67)cc45)cccc23)c2cccc3ccccc23)cc1 ; c1ccc(N(c2cccc3ccccc23)c2cccc3oc4c(-c5cccc6c5sc5cccc(-c7cccc8sc9ccccc9c78)c56)cccc4c23)cc1]

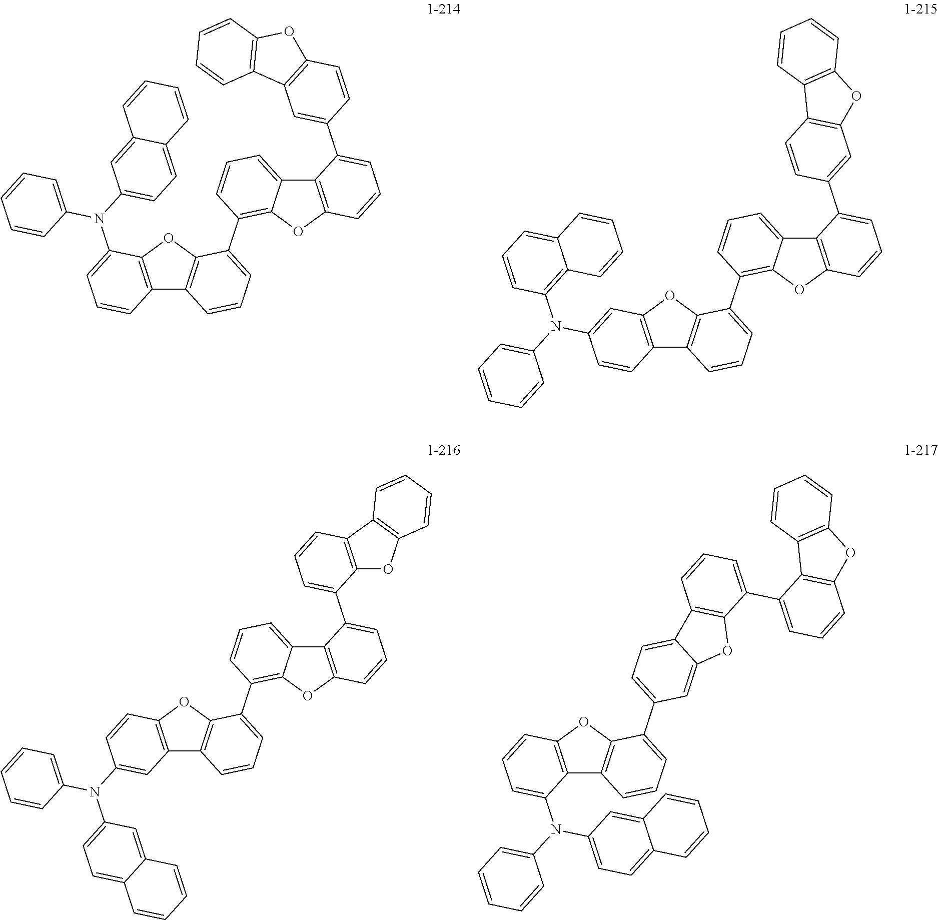 [SMILES: c1ccc(N(c2ccc3ccccc3c2)c2cccc3c2oc2c(-c4cccc5c4oc4cccc(-c6ccc7oc8ccccc8c7c6)c45)cccc23)cc1 ; c1ccc(N(c2ccc3c(c2)oc2c(-c4cccc5c4oc4cccc(-c6ccc7c(c6)oc6ccccc67)c45)cccc23)c2cccc3ccccc23)cc1 ; c1ccc(N(c2ccc3ccccc3c2)c2ccc3oc4c(-c5cccc6c5oc5cccc(-c7cccc8c7oc7ccccc78)c56)cccc4c3c2)cc1 ; c1ccc(N(c2ccc3ccccc3c2)c2cccc3oc4c(-c5ccc6c(c5)oc5c(-c7cccc8oc9ccccc9c78)cccc56)cccc4c23)cc1]

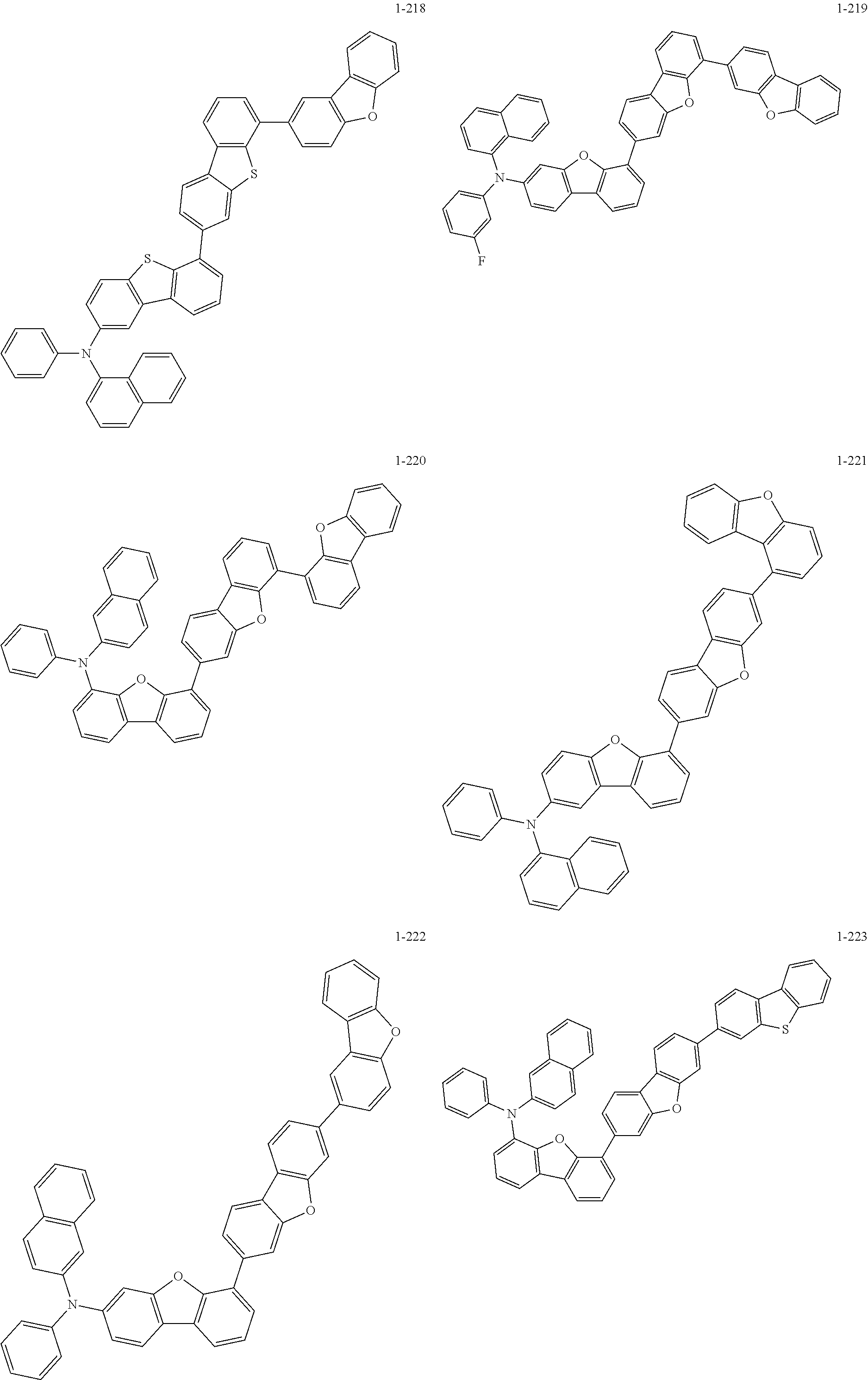

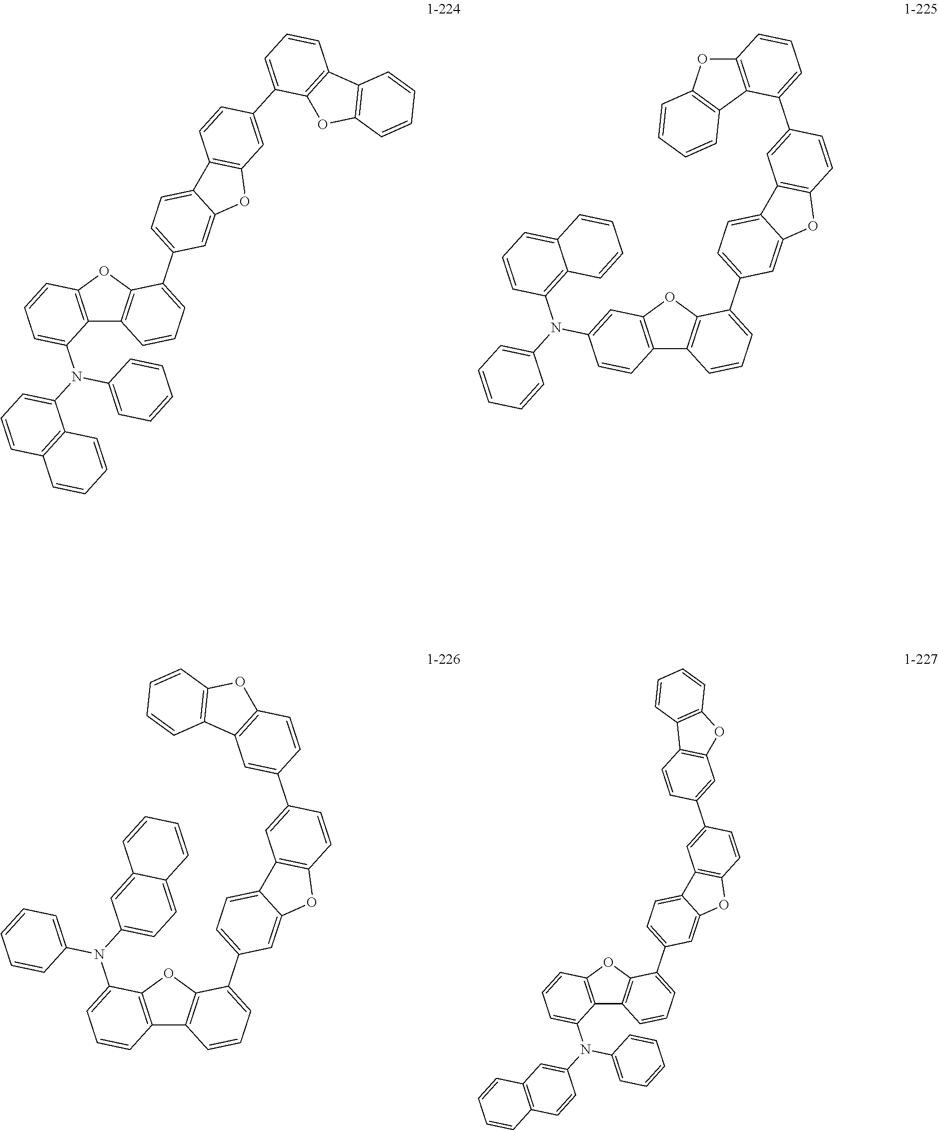 [SMILES: c1ccc(N(c2cccc3ccccc23)c2cccc3oc4c(-c5ccc6c(c5)oc5cc(-c7cccc8c7oc7ccccc78)ccc56)cccc4c23)cc1 ; c1ccc(N(c2ccc3c(c2)oc2c(-c4ccc5c(c4)oc4ccc(-c6cccc7oc8ccccc8c67)cc45)cccc23)c2cccc3ccccc23)cc1 ; c1ccc(N(c2ccc3ccccc3c2)c2cccc3c2oc2c(-c4ccc5c(c4)oc4ccc(-c6ccc7oc8ccccc8c7c6)cc45)cccc23)cc1 ; c1ccc(N(c2ccc3ccccc3c2)c2cccc3oc4c(-c5ccc6c(c5)oc5ccc(-c7ccc8c(c7)oc7ccccc78)cc56)cccc4c23)cc1]

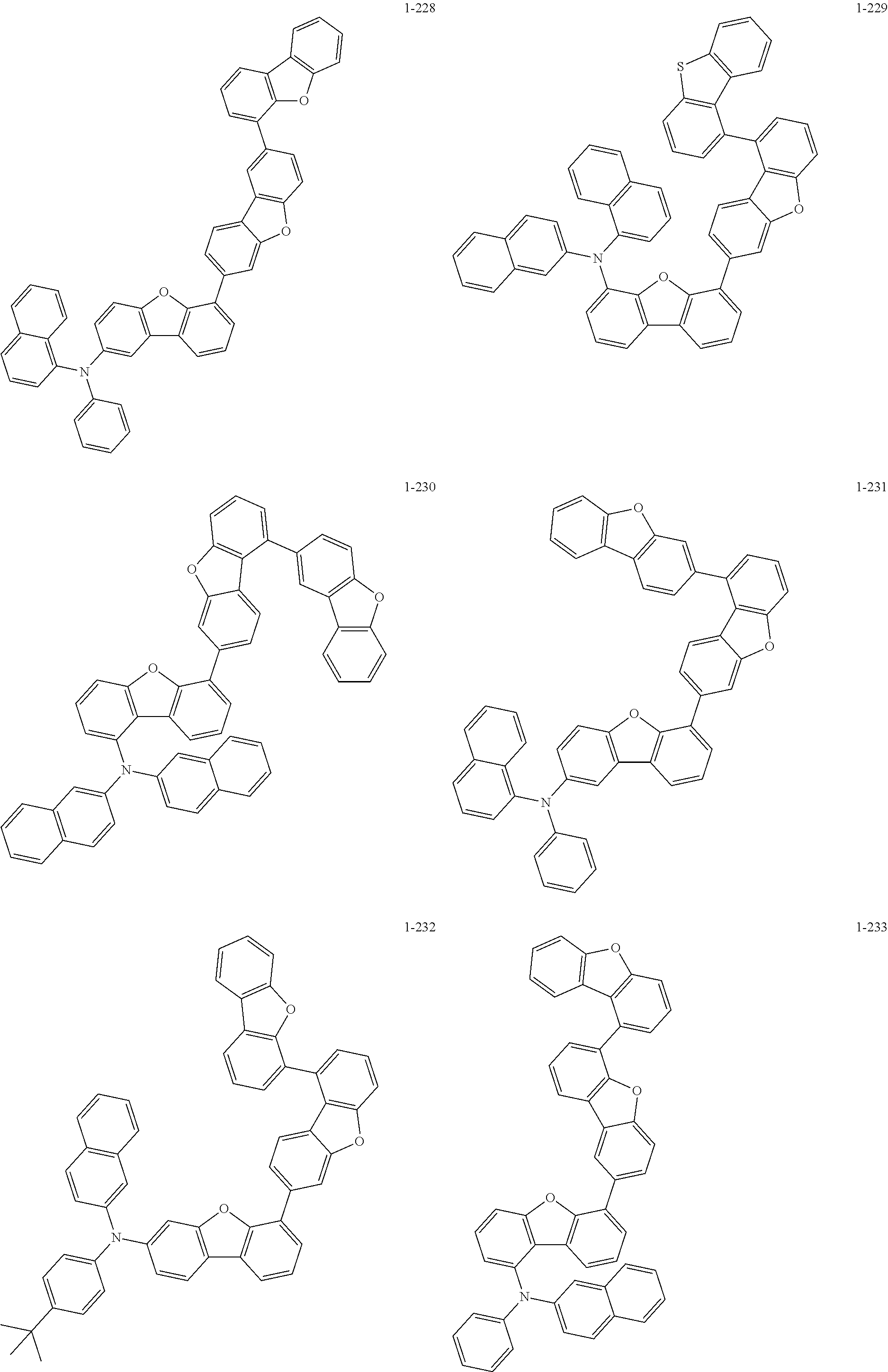

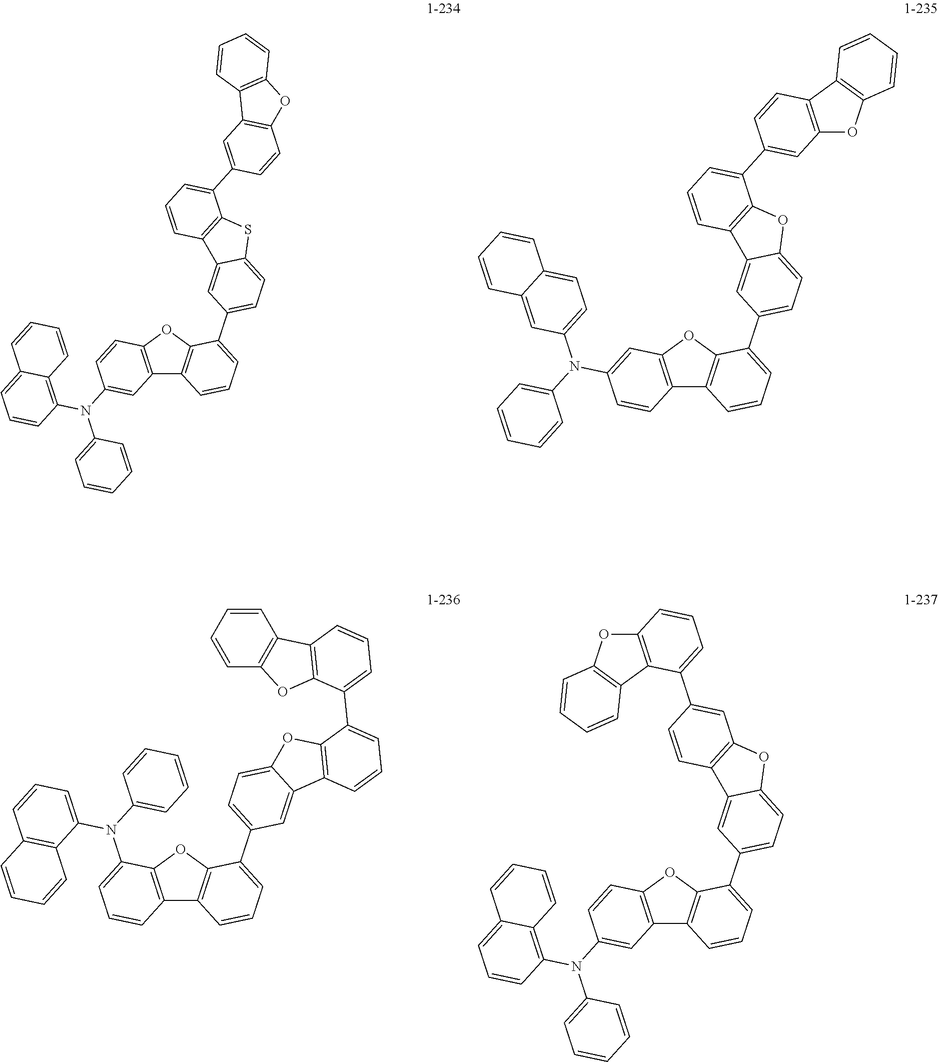 [SMILES: c1ccc(N(c2ccc3oc4c(-c5ccc6sc7c(-c8ccc9oc%10ccccc%10c9c8)cccc7c6c5)cccc4c3c2)c2cccc3ccccc23)cc1 ; c1ccc(N(c2ccc3ccccc3c2)c2ccc3c(c2)oc2c(-c4ccc5oc6c(-c7ccc8c(c7)oc7ccccc78)cccc6c5c4)cccc23)cc1 ; c1ccc(N(c2cccc3ccccc23)c2cccc3c2oc2c(-c4ccc5oc6c(-c7cccc8c7oc7ccccc78)cccc6c5c4)cccc23)cc1 ; c1ccc(N(c2ccc3oc4c(-c5ccc6oc7cc(-c8cccc9oc%10ccccc%10c89)ccc7c6c5)cccc4c3c2)c2cccc3ccccc23)cc1]

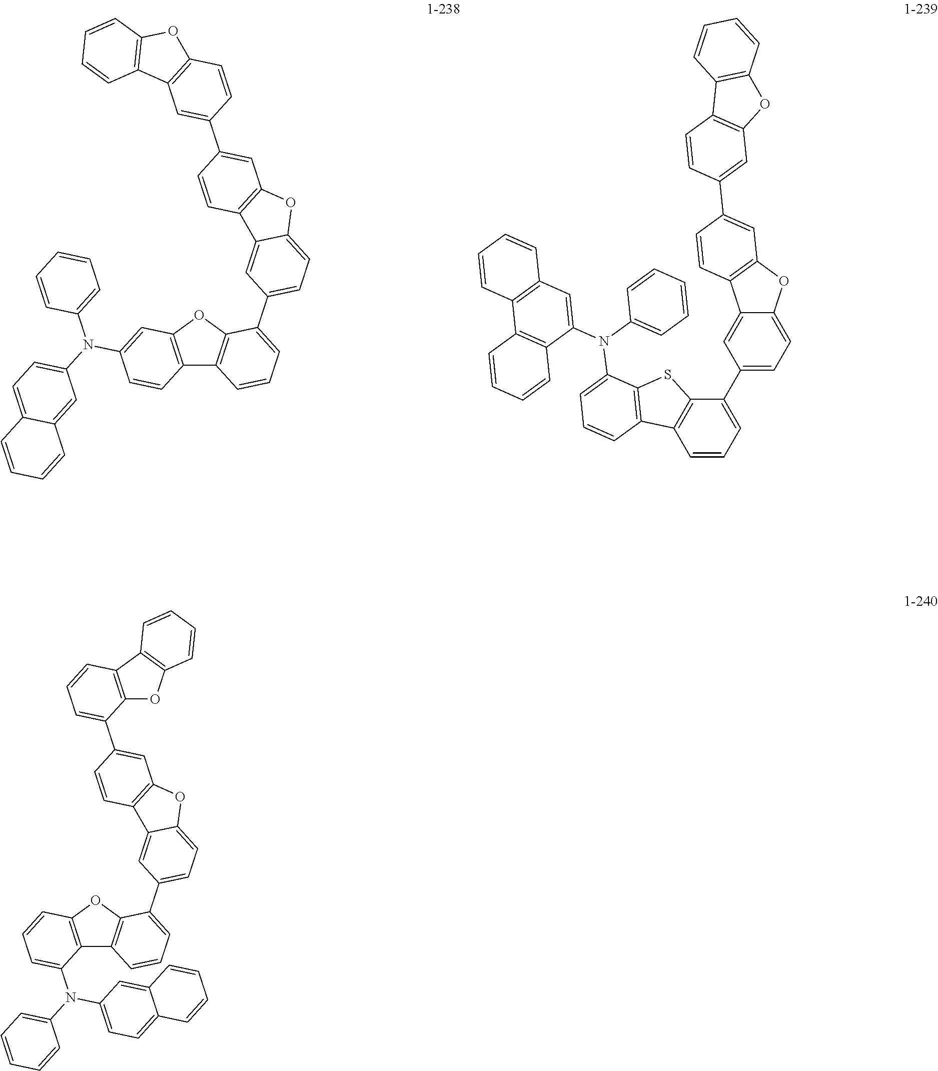 [SMILES: c1ccc(N(c2ccc3ccccc3c2)c2ccc3c(c2)oc2c(-c4ccc5oc6cc(-c7ccc8oc9ccccc9c8c7)ccc6c5c4)cccc23)cc1 ; c1ccc(N(c2cc3ccccc3c3ccccc23)c2cccc3c2sc2c(-c4ccc5oc6cc(-c7ccc8c(c7)oc7ccccc78)ccc6c5c4)cccc23)cc1 ; c1ccc(N(c2ccc3ccccc3c2)c2cccc3oc4c(-c5ccc6oc7cc(-c8cccc9c8oc8ccccc89)ccc7c6c5)cccc4c23)cc1]

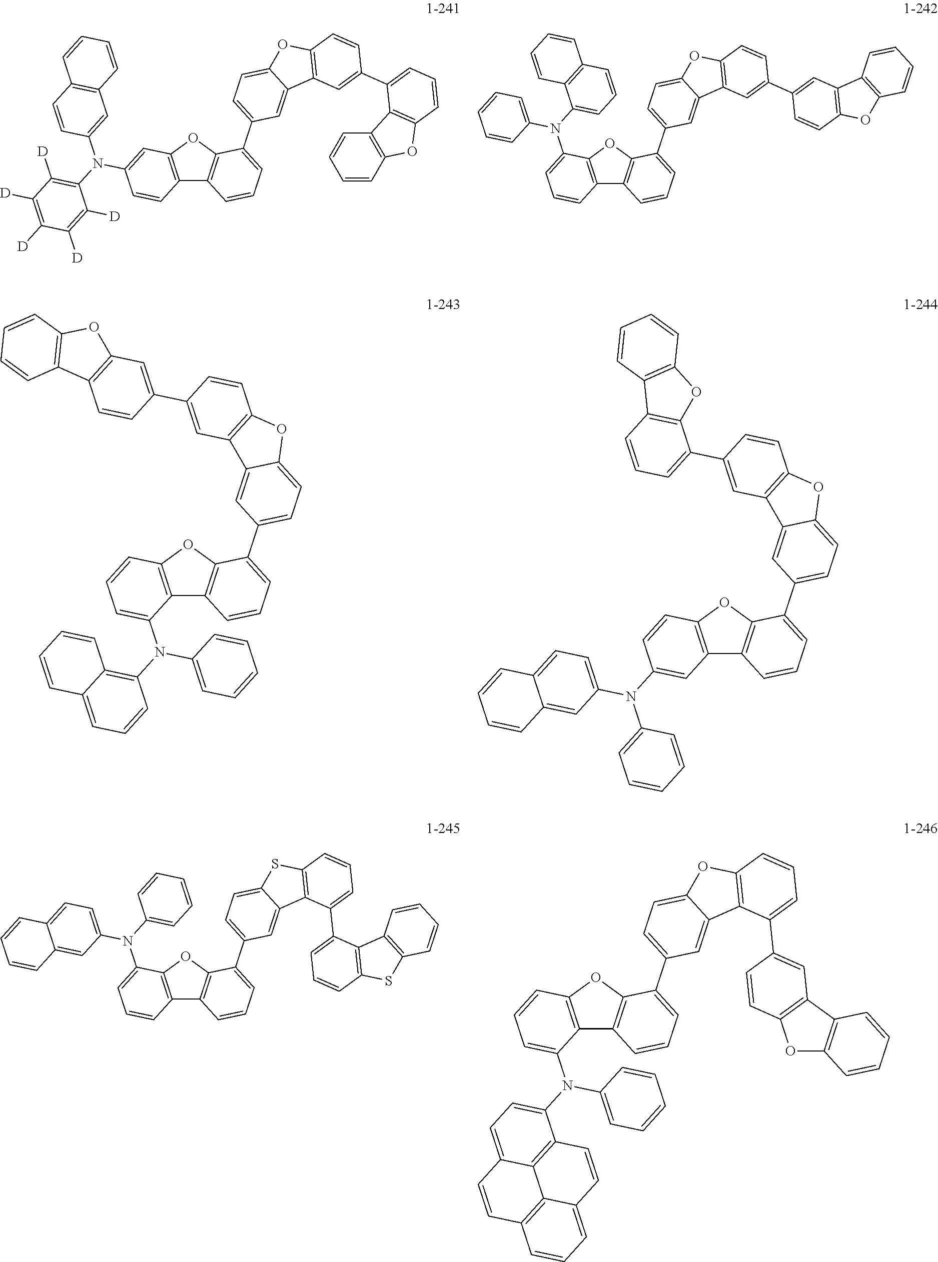

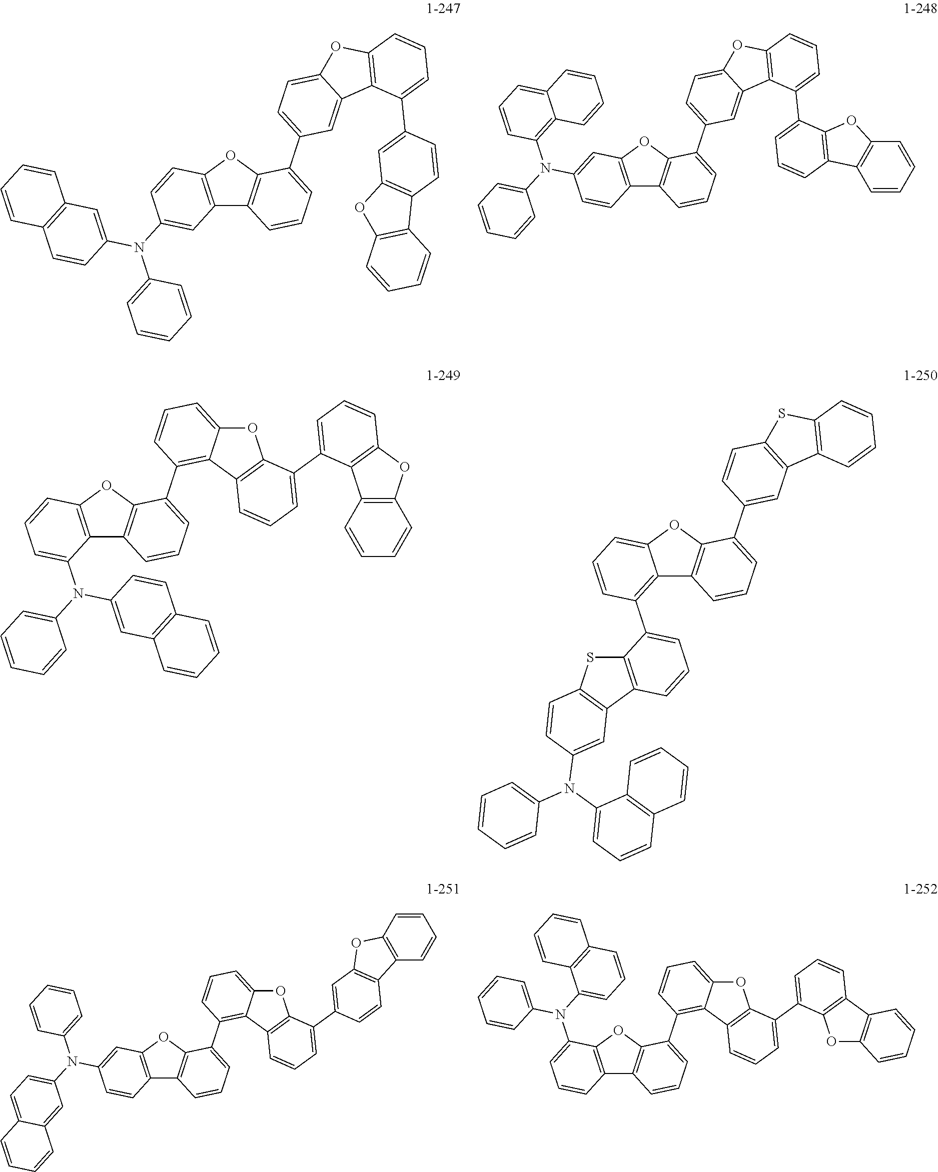

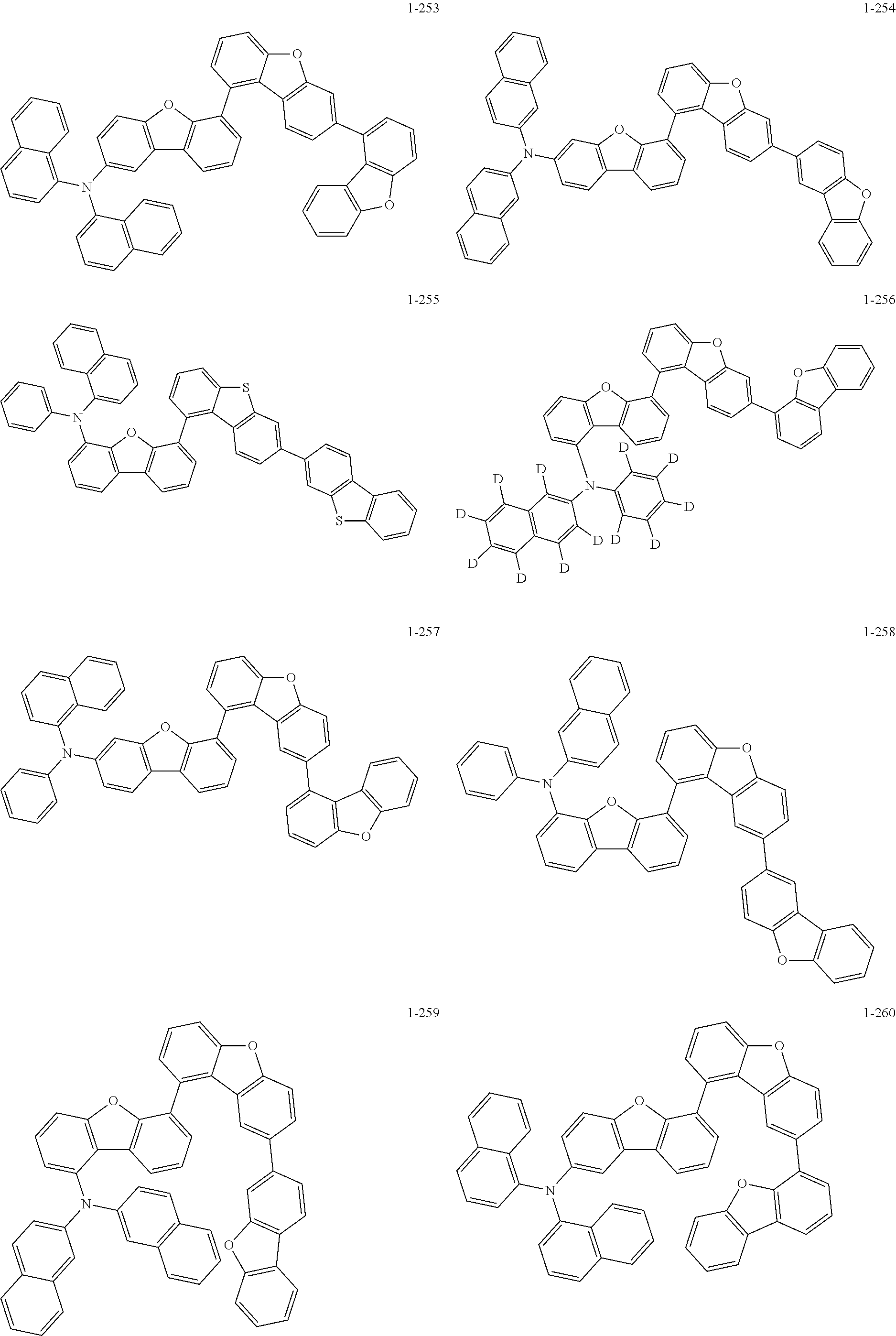

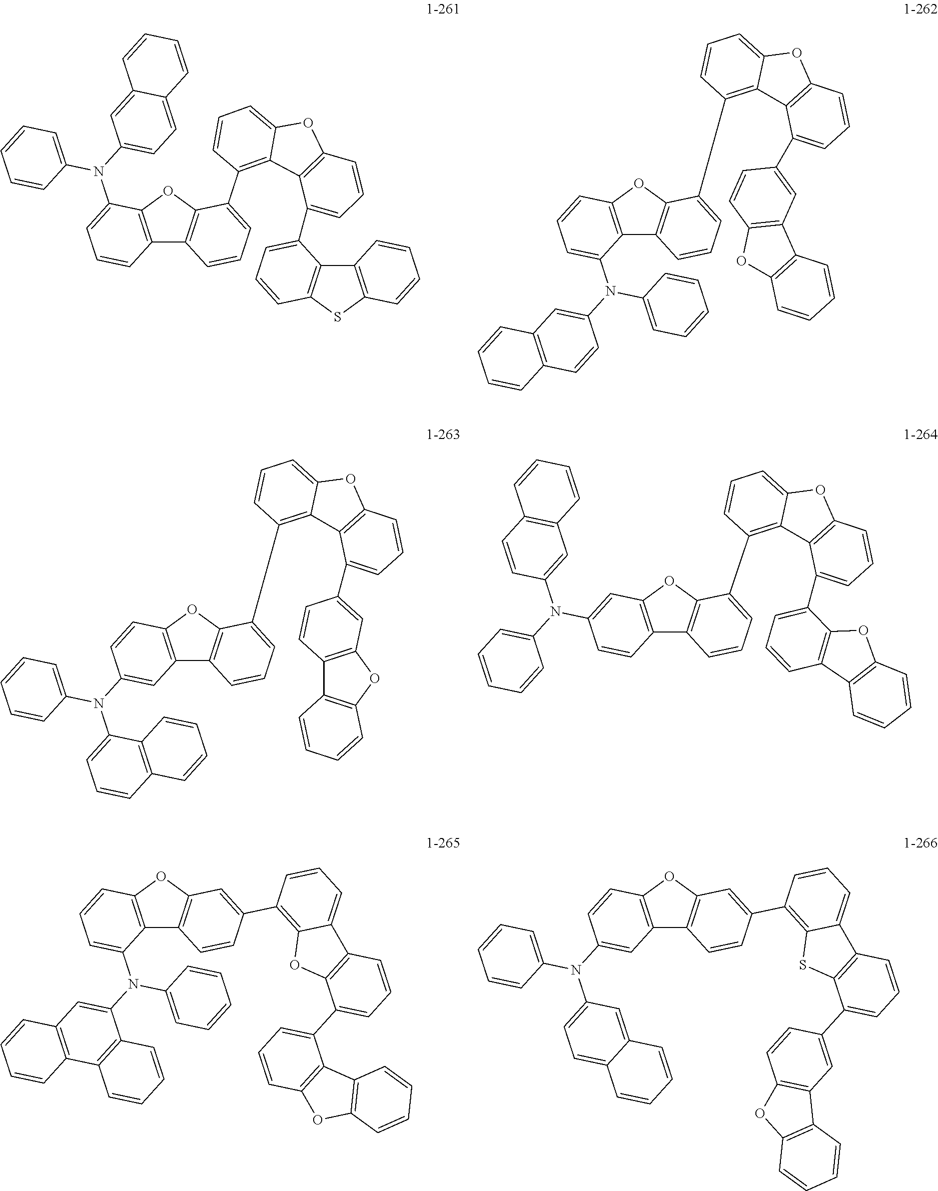

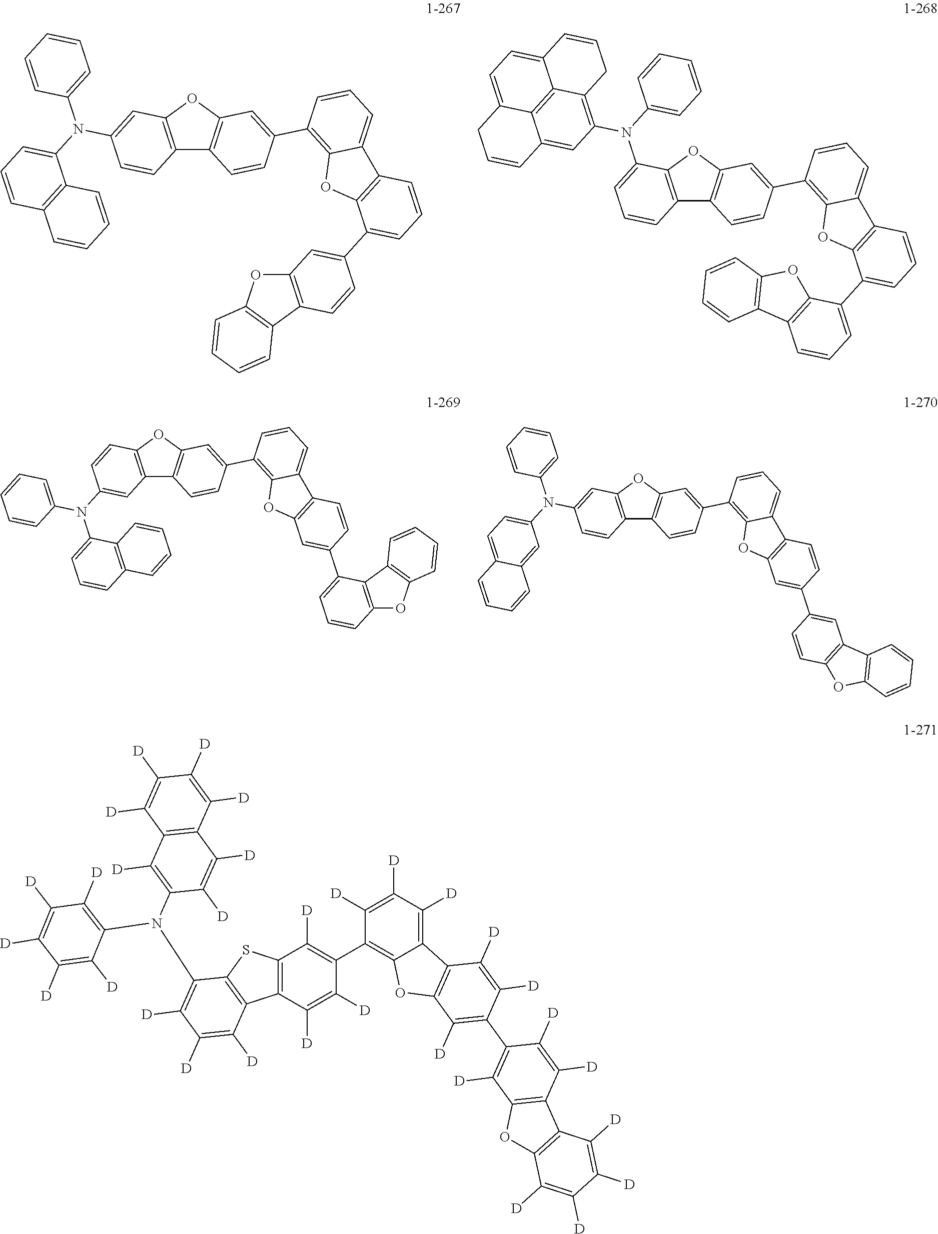

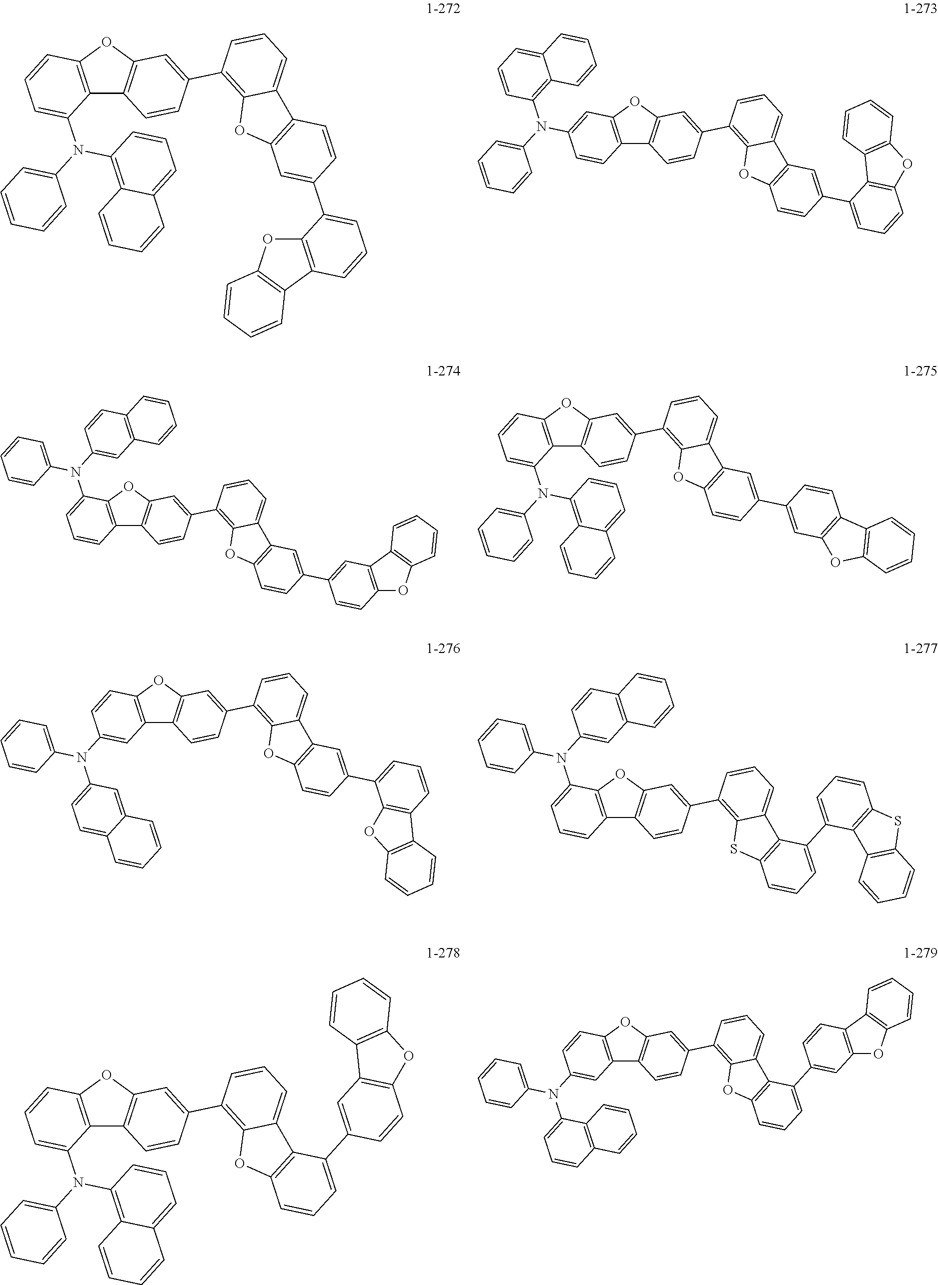

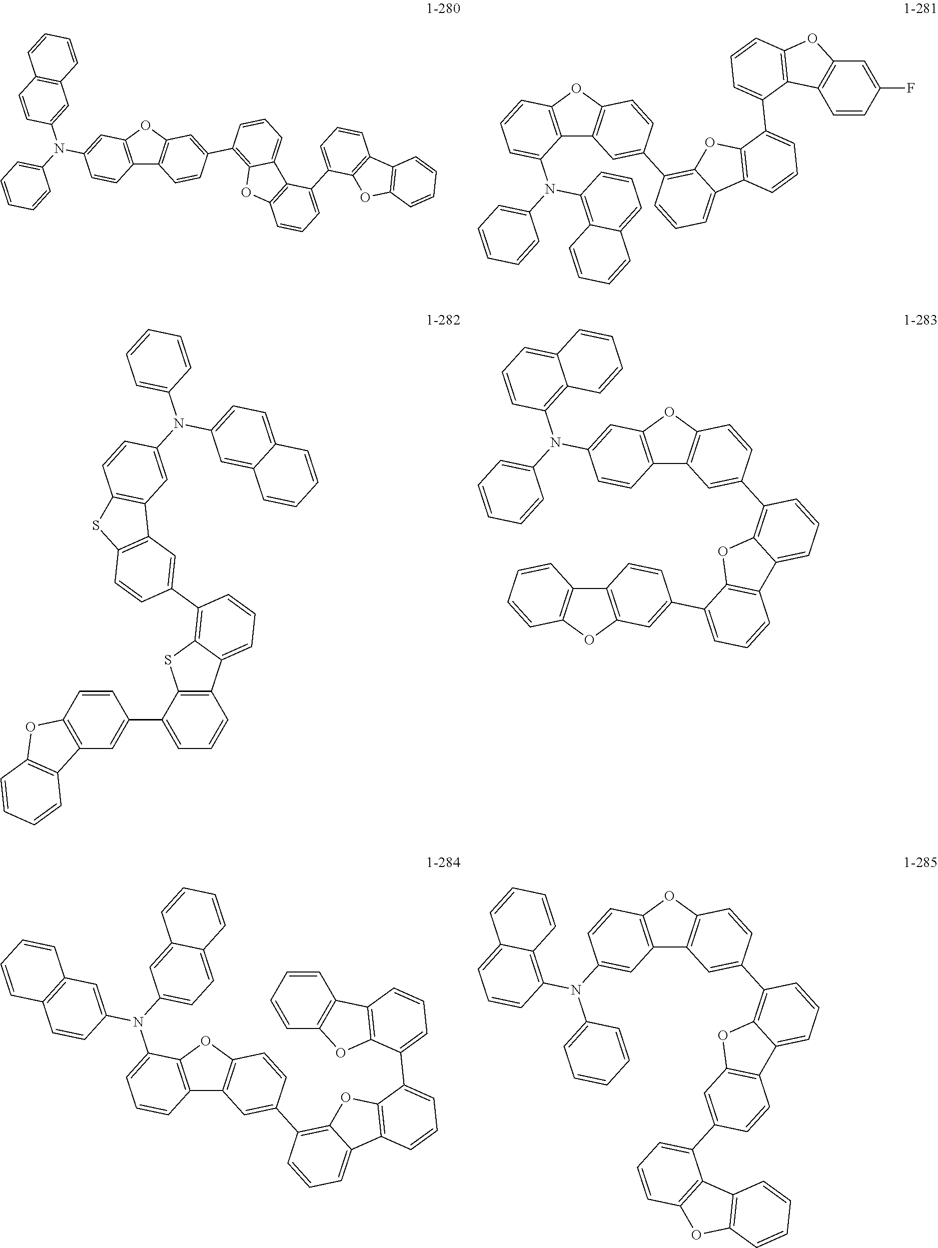

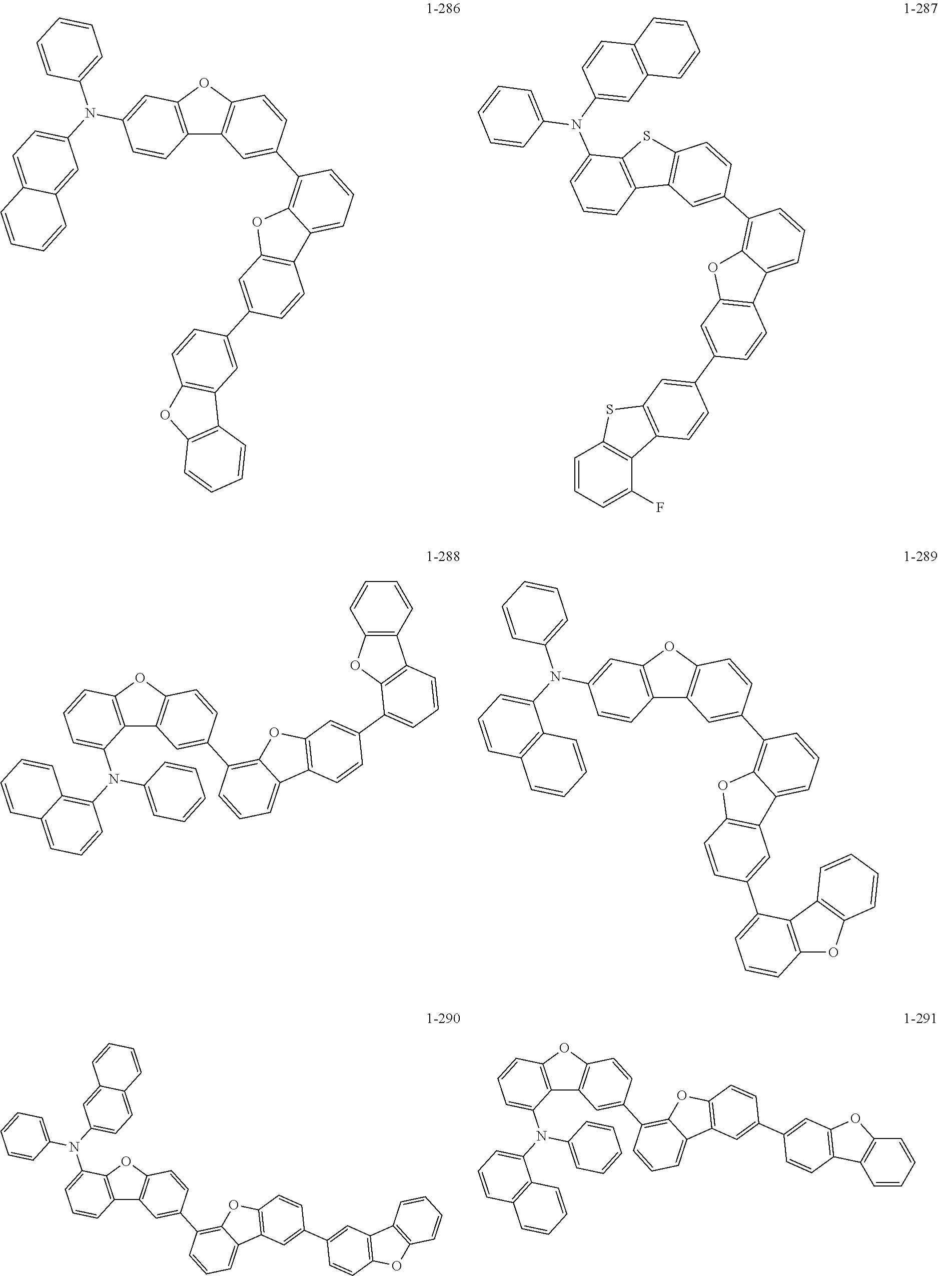

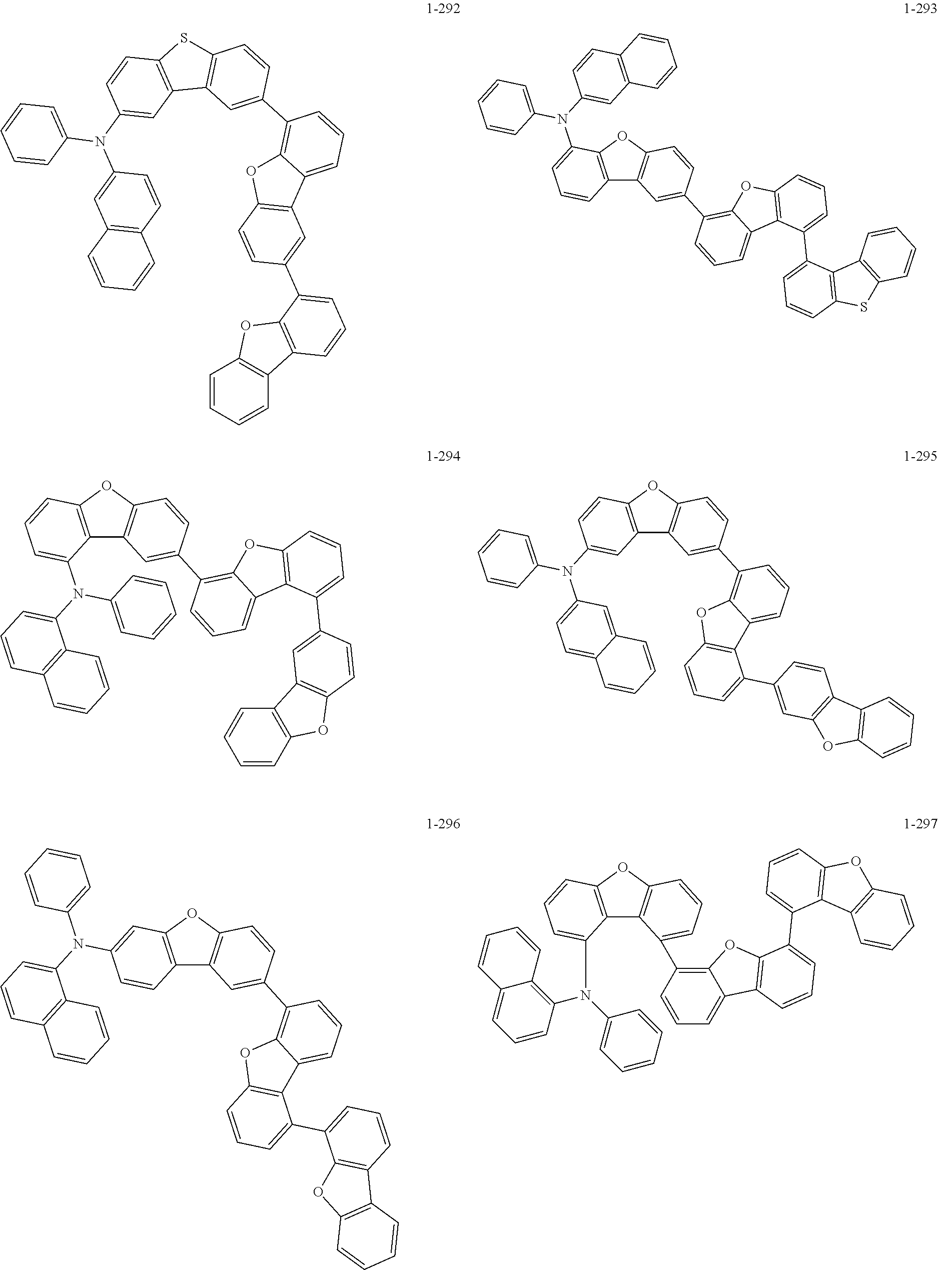

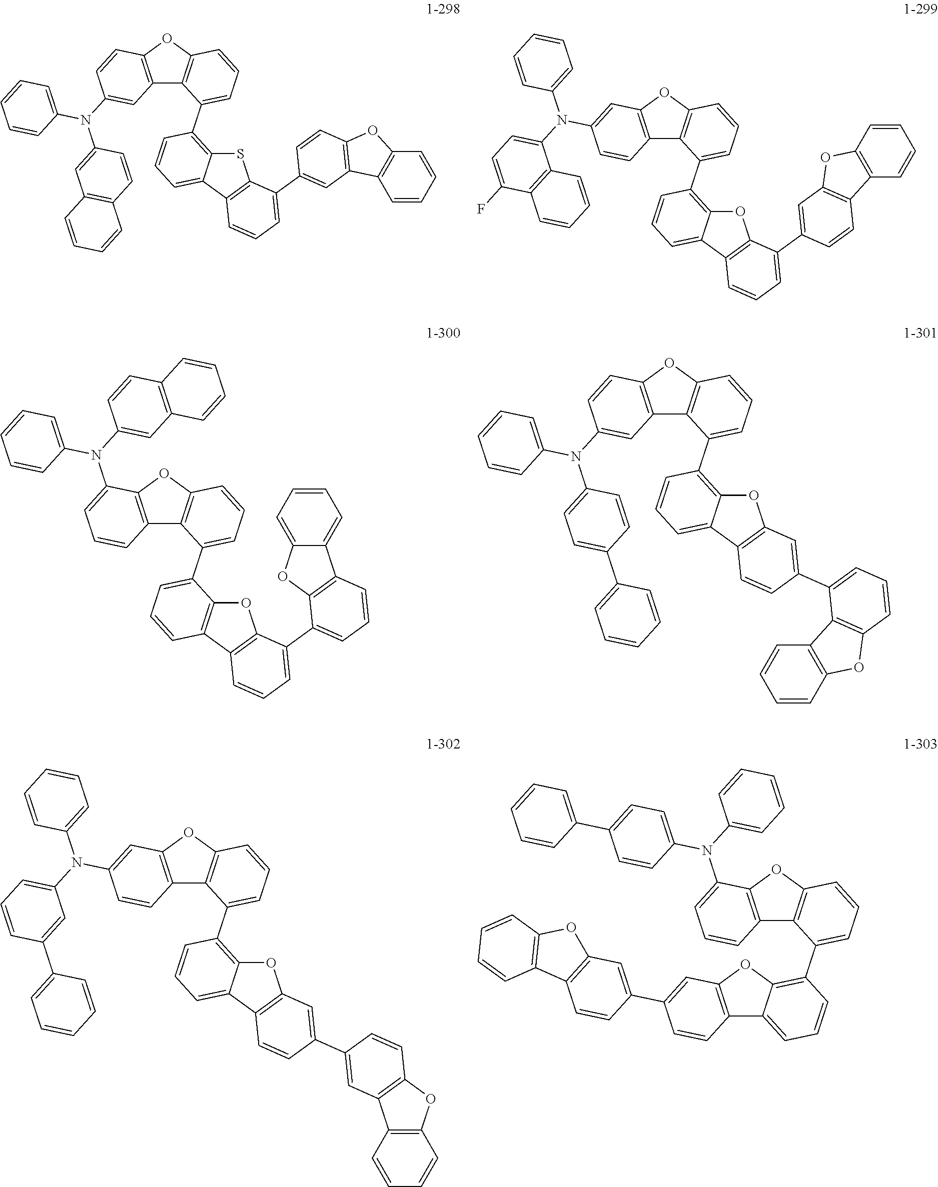

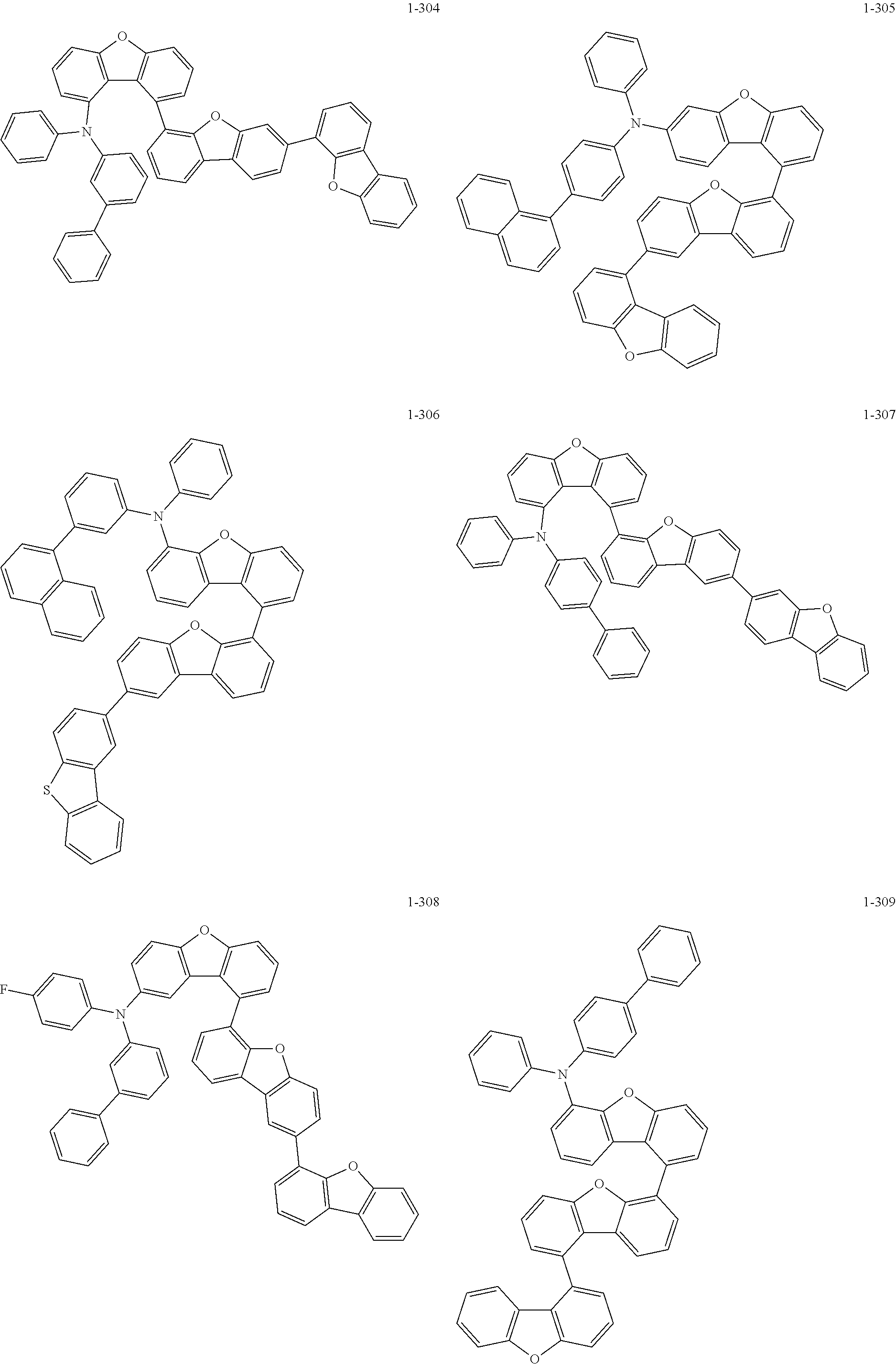

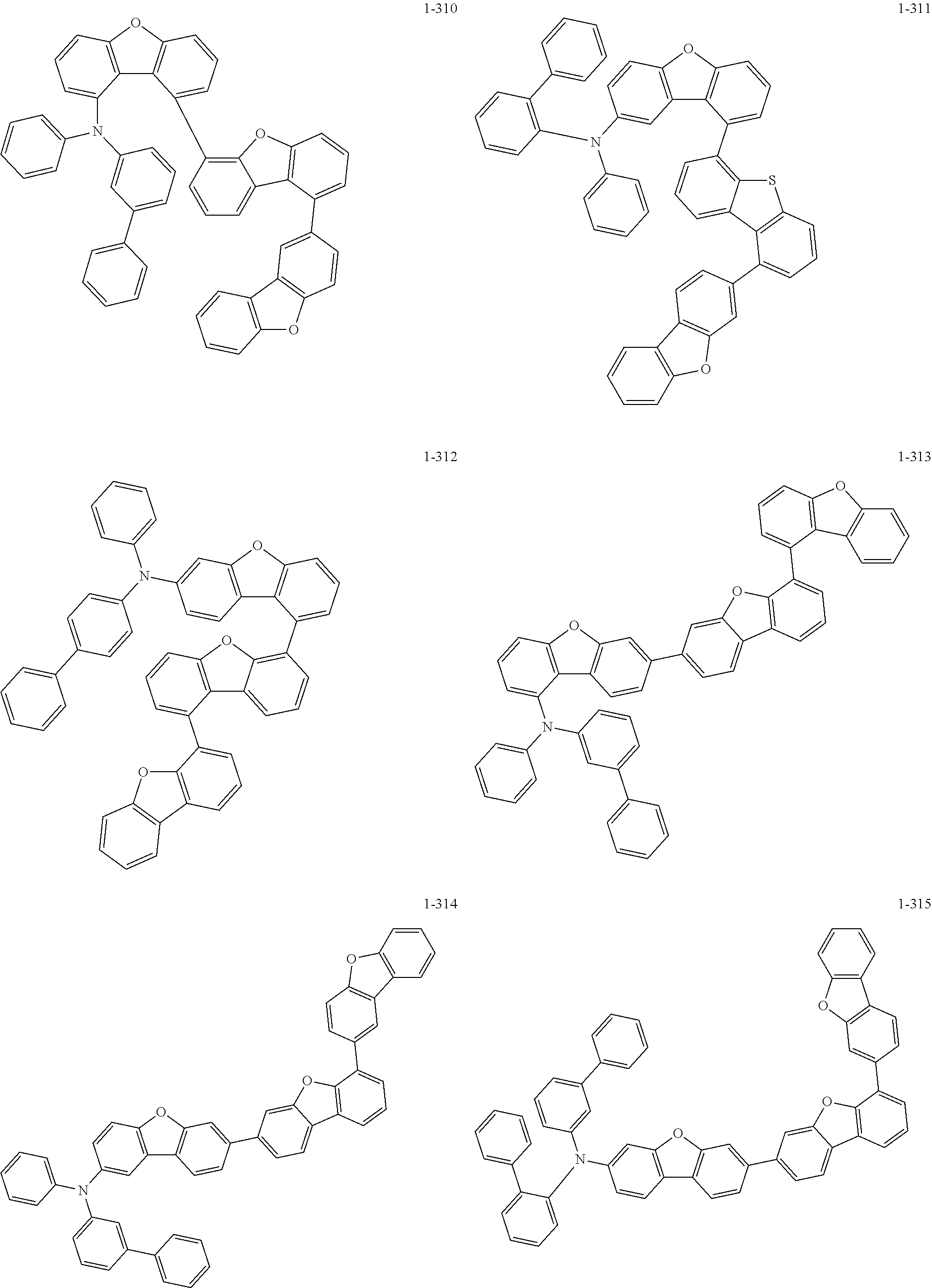

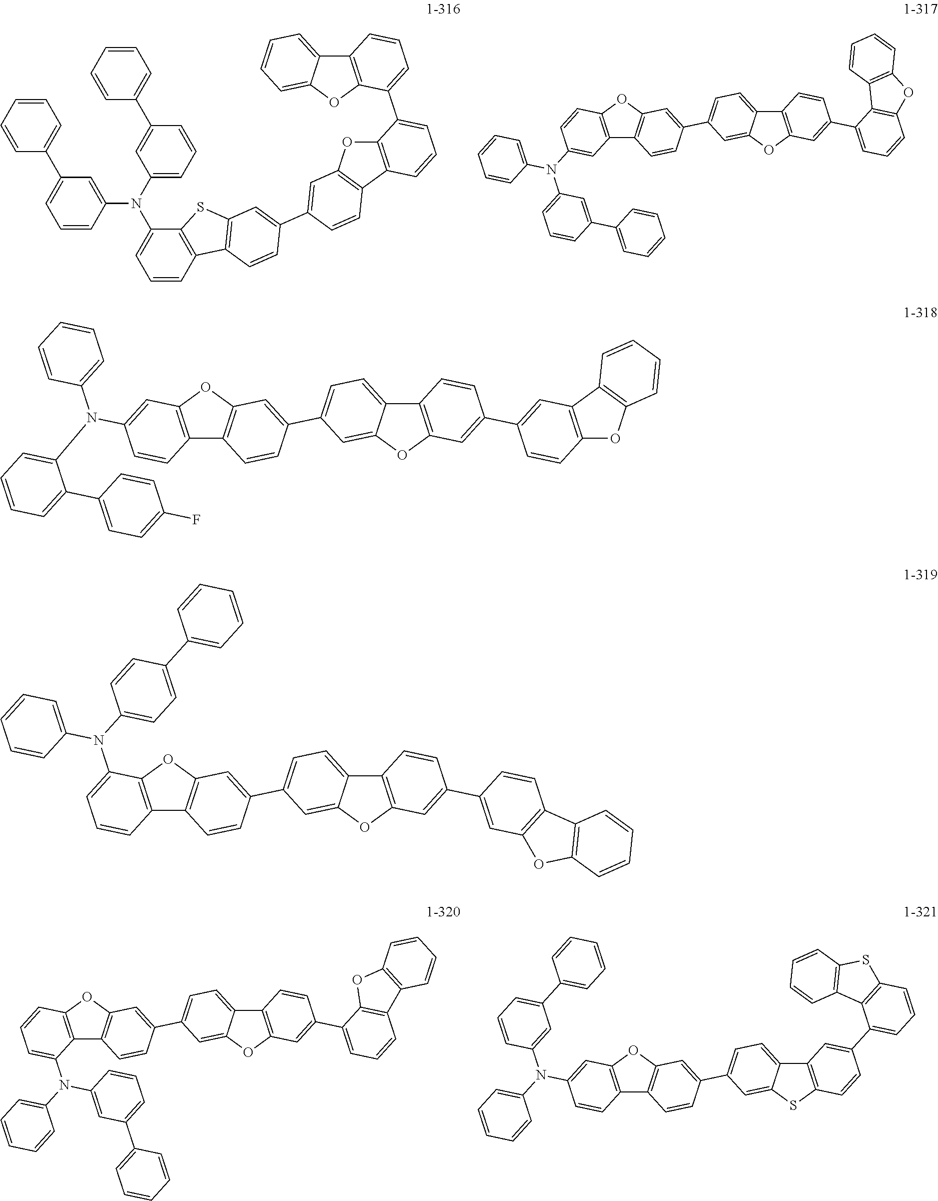

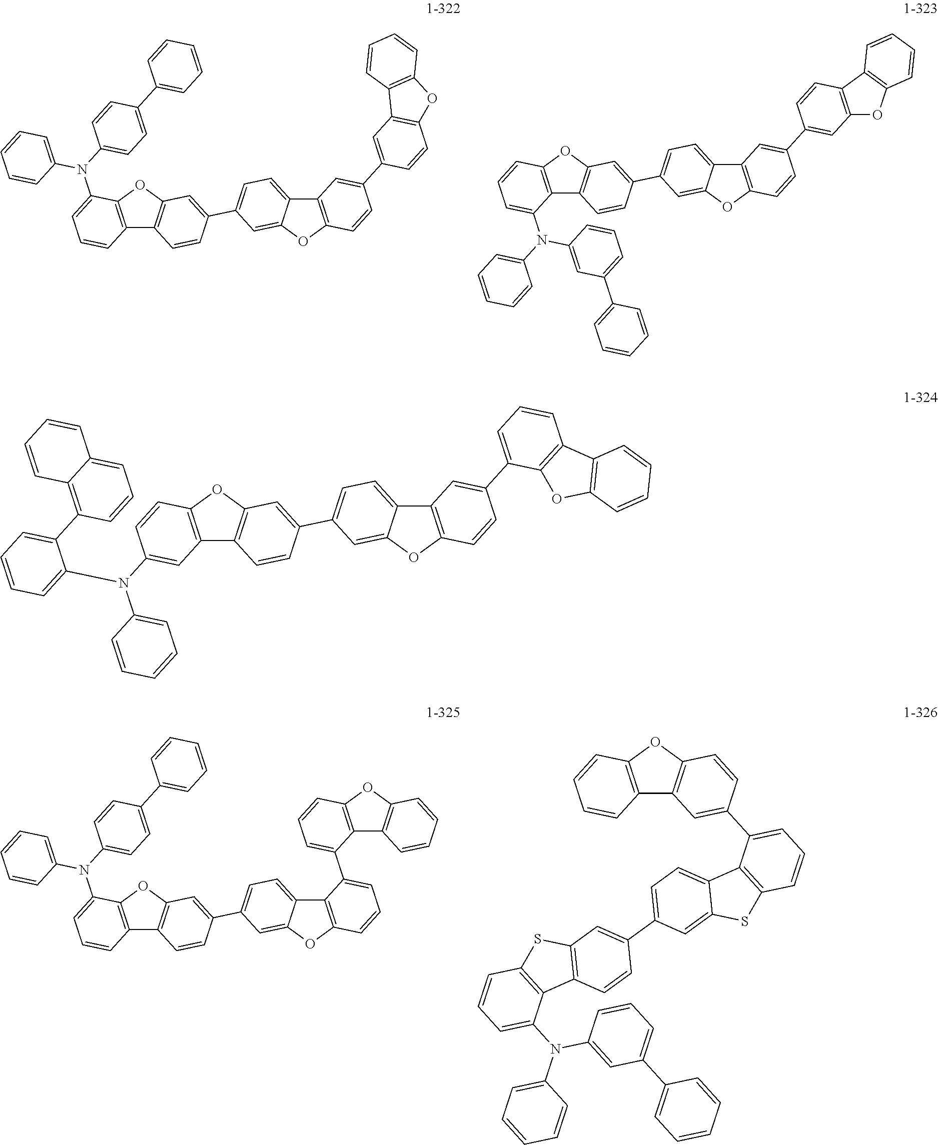

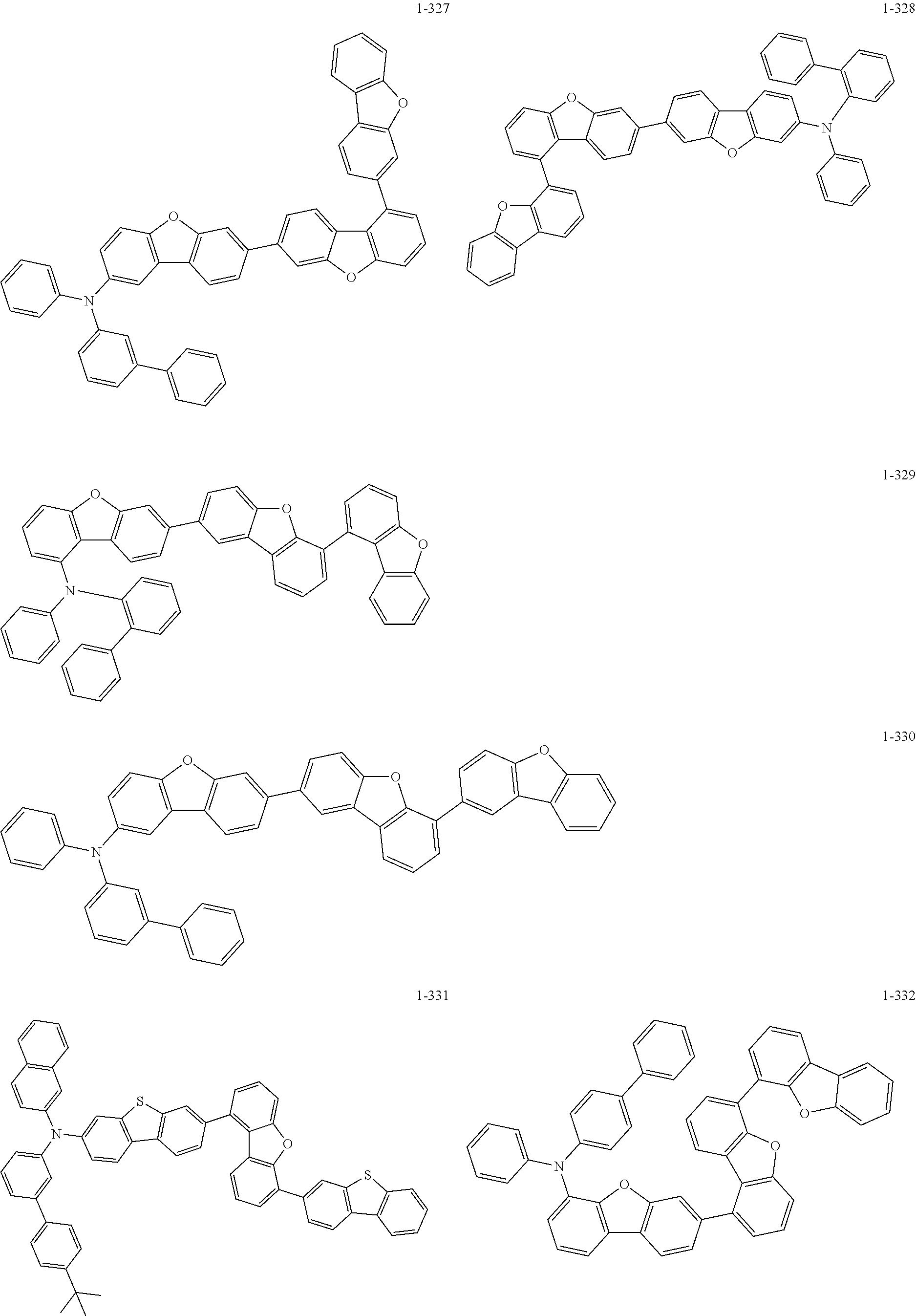

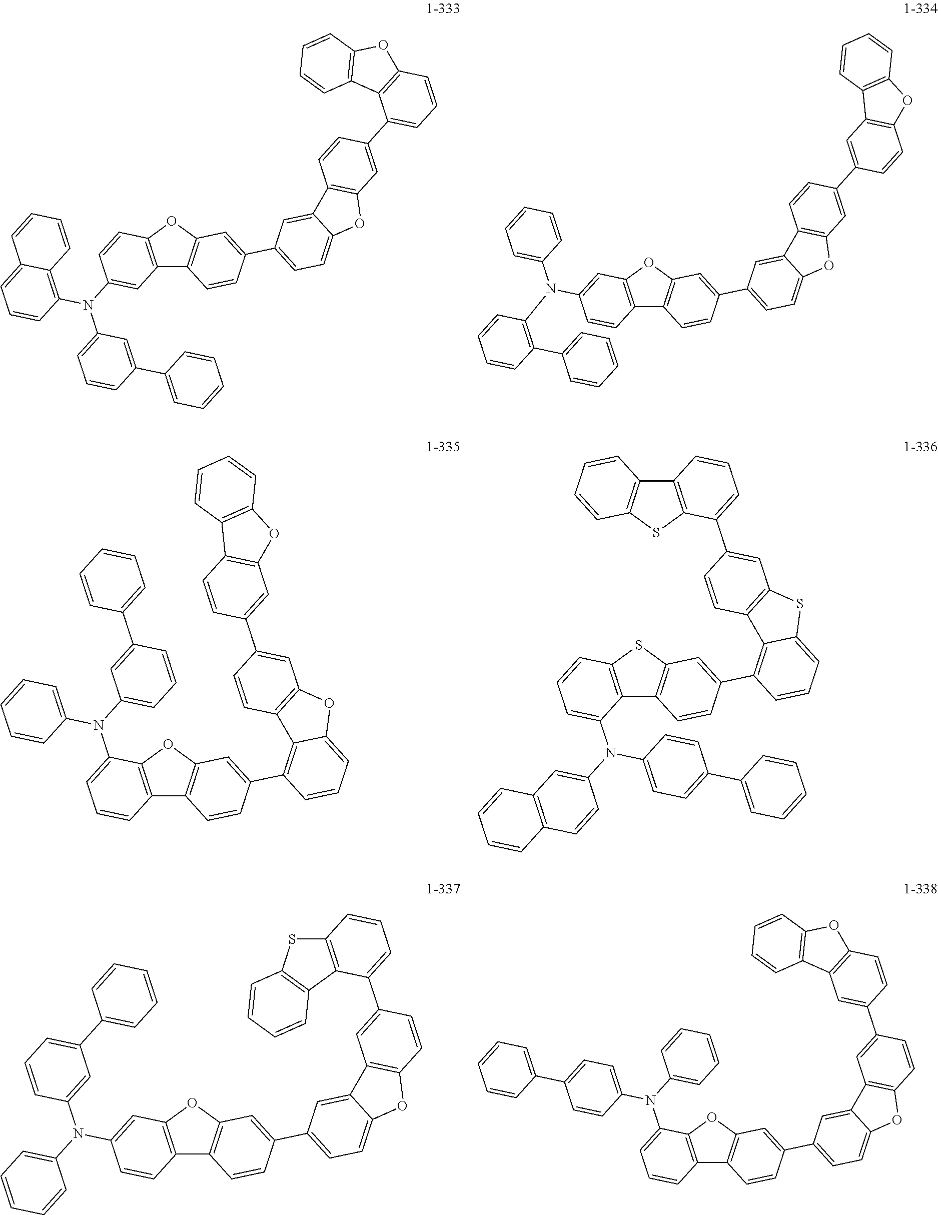

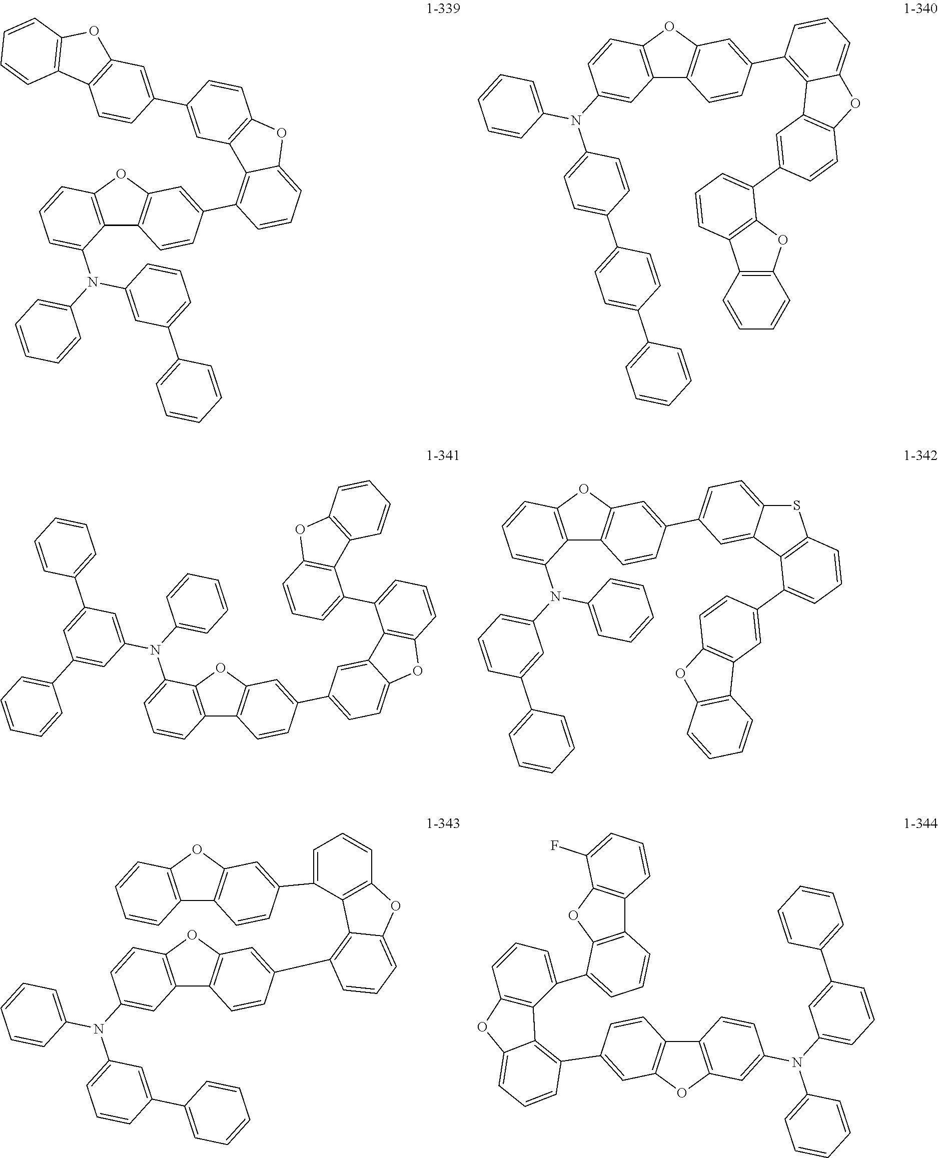

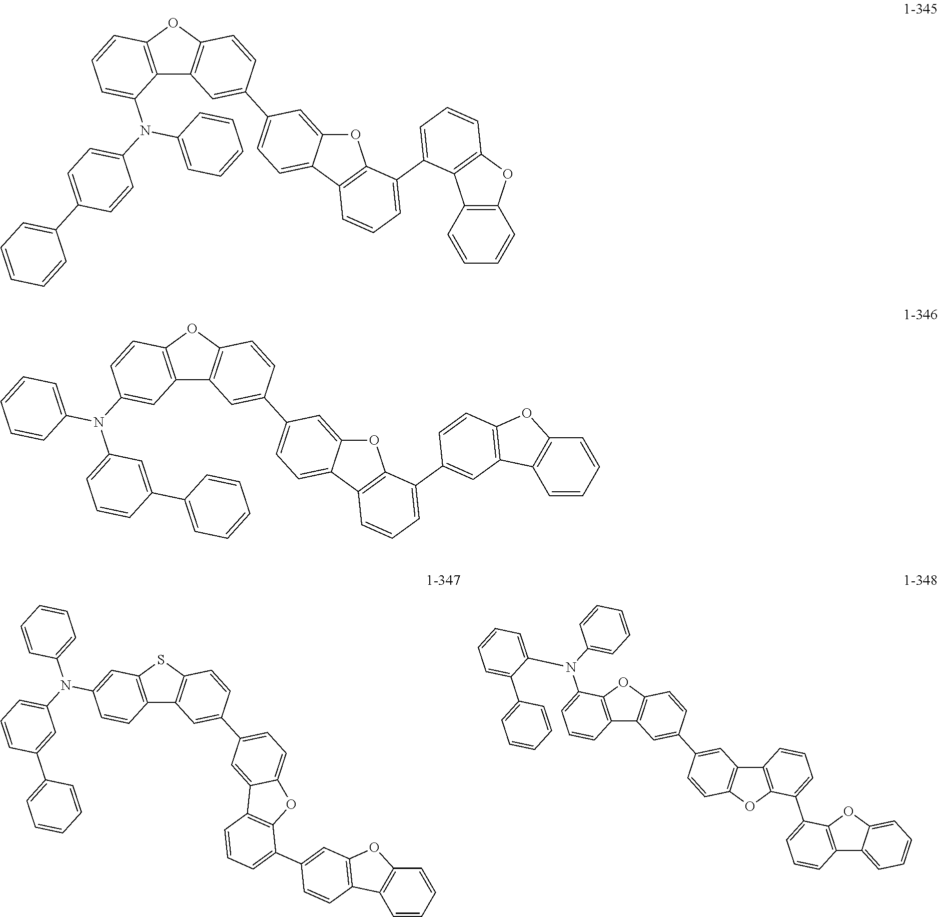 [SMILES: c1ccc(-c2ccc(N(c3ccccc3)c3cccc4oc5ccc(-c6ccc7c(c6)oc6c(-c8cccc9oc%10ccccc%10c89)cccc67)cc5c34)cc2)cc1 ; c1ccc(-c2cccc(N(c3ccccc3)c3ccc4oc5ccc(-c6ccc7c(c6)oc6c(-c8ccc9oc%10ccccc%10c9c8)cccc67)cc5c4c3)c2)cc1 ; c1ccc(-c2cccc(N(c3ccccc3)c3ccc4c(c3)sc3ccc(-c5ccc6oc7c(-c8ccc9c(c8)oc8ccccc89)cccc7c6c5)cc34)c2)cc1 ; c1ccc(-c2ccccc2N(c2ccccc2)c2cccc3c2oc2ccc(-c4ccc5oc6c(-c7cccc8c7oc7ccccc78)cccc6c5c4)cc23)cc1]

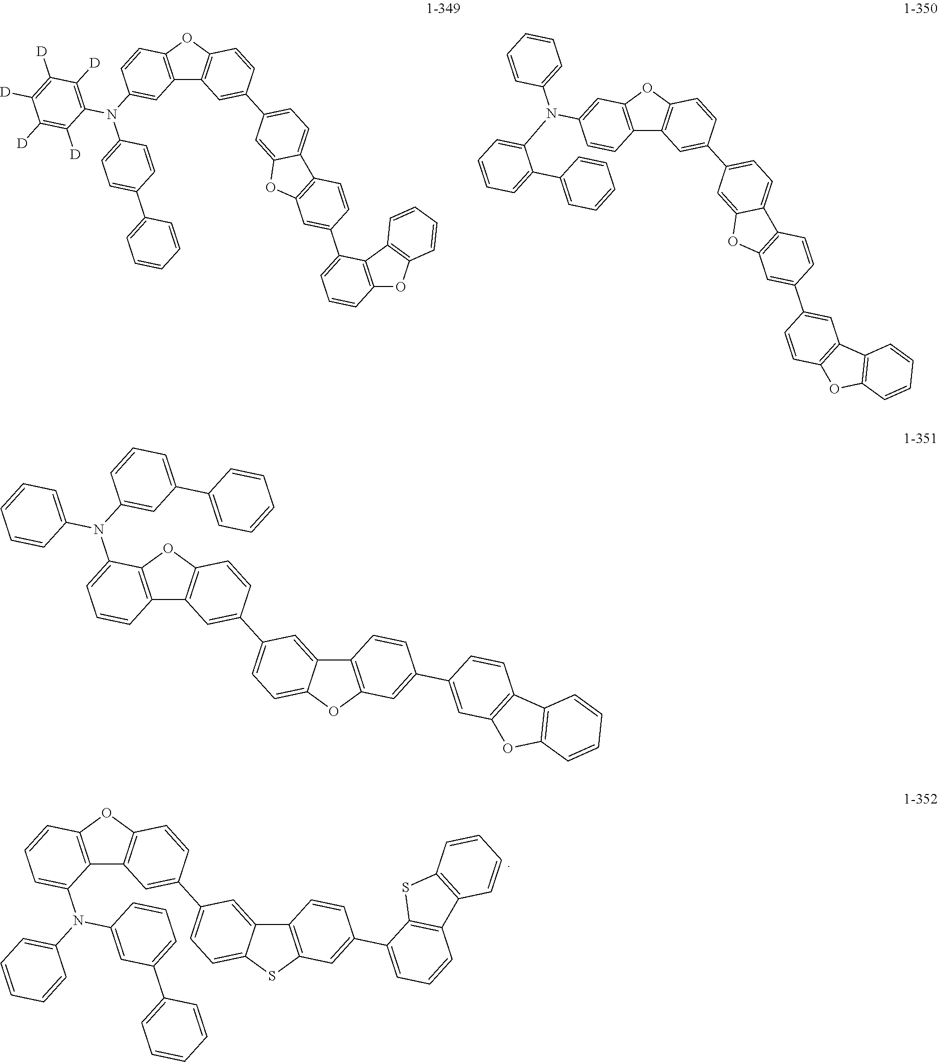 [SMILES: [2H]c1c([2H])c([2H])c(N(c2ccc(-c3ccccc3)cc2)c2ccc3oc4ccc(-c5ccc6c(c5)oc5cc(-c7cccc8oc9ccccc9c78)ccc56)cc4c3c2)c([2H])c1[2H] ; c1ccc(-c2ccccc2N(c2ccccc2)c2ccc3c(c2)oc2ccc(-c4ccc5c(c4)oc4cc(-c6ccc7oc8ccccc8c7c6)ccc45)cc23)cc1 ; c1ccc(-c2cccc(N(c3ccccc3)c3cccc4c3oc3ccc(-c5ccc6oc7cc(-c8ccc9c(c8)oc8ccccc89)ccc7c6c5)cc34)c2)cc1 ; c1ccc(-c2cccc(N(c3ccccc3)c3cccc4oc5ccc(-c6ccc7sc8cc(-c9cccc%10c9sc9ccccc9%10)ccc8c7c6)cc5c34)c2)cc1]

9. An organic light emitting device comprising:
a first electrode;
a second electrode provided opposite to the first electrode; and
one or more organic material layers provided between the first electrode and the second electrode,
wherein the one or more organic material layers include the heterocyclic compound of claim 1.

10. The organic light emitting device of claim 9, wherein the one or more organic material layer include a light emitting layer, and the light emitting layer includes the heterocyclic compound represented by Chemical Formula 1.

11. The organic light emitting device of claim 9, wherein the one or more organic material layer include a light emitting layer, the light emitting layer includes a host material, and the host material includes the heterocyclic compound represented by Chemical Formula 1.

12. The organic light emitting device of claim 9, wherein the one or more organic material layer further include a heterocyclic compound represented by the following Chemical Formula A: 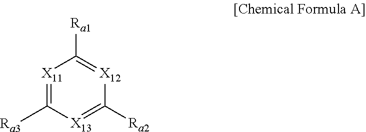
in Chemical Formula A,
X₁₁ to X₁₃ are the same as or different from each other, and each independently N or CR′;
at least one of X₁₁ to X₁₃ is N; and
Rₐ₁ to Rₐ₃ and R′ are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

13. The organic light emitting device of claim 12, wherein the heterocyclic compound represented by Chemical Formula A is represented by any one of the following compounds:

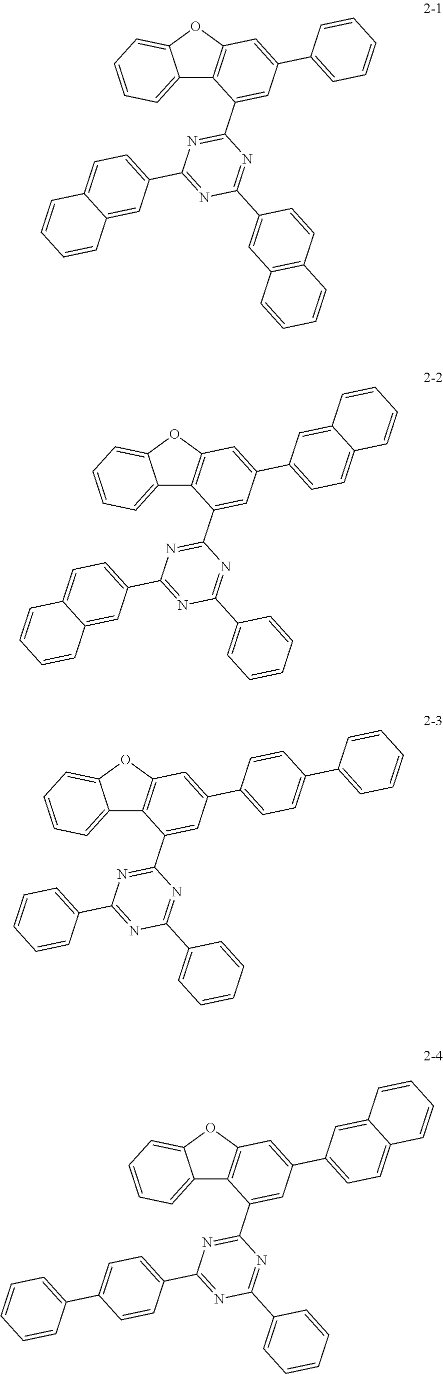 [SMILES: c1ccc(-c2cc(-c3nc(-c4ccc5ccccc5c4)nc(-c4ccc5ccccc5c4)n3)c3c(c2)oc2ccccc23)cc1 ; c1ccc(-c2nc(-c3ccc4ccccc4c3)nc(-c3cc(-c4ccc5ccccc5c4)cc4oc5ccccc5c34)n2)cc1 ; c1ccc(-c2ccc(-c3cc(-c4nc(-c5ccccc5)nc(-c5ccccc5)n4)c4c(c3)oc3ccccc34)cc2)cc1 ; c1ccc(-c2ccc(-c3nc(-c4ccccc4)nc(-c4cc(-c5ccc6ccccc6c5)cc5oc6ccccc6c45)n3)cc2)cc1]

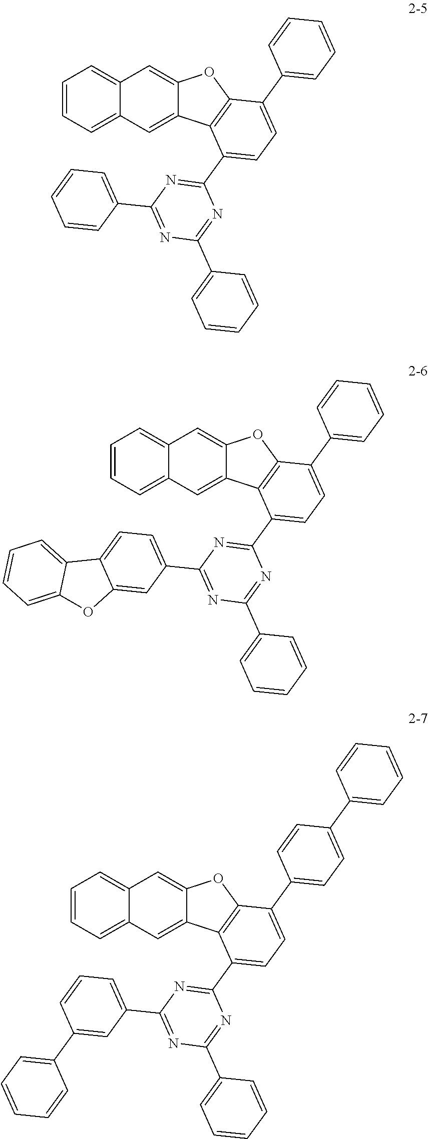 [SMILES: c1ccc(-c2nc(-c3ccccc3)nc(-c3ccc(-c4ccccc4)c4oc5cc6ccccc6cc5c34)n2)cc1 ; c1ccc(-c2nc(-c3ccc4c(c3)oc3ccccc34)nc(-c3ccc(-c4ccccc4)c4oc5cc6ccccc6cc5c34)n2)cc1 ; c1ccc(-c2ccc(-c3ccc(-c4nc(-c5ccccc5)nc(-c5cccc(-c6ccccc6)c5)n4)c4c3oc3cc5ccccc5cc34)cc2)cc1]

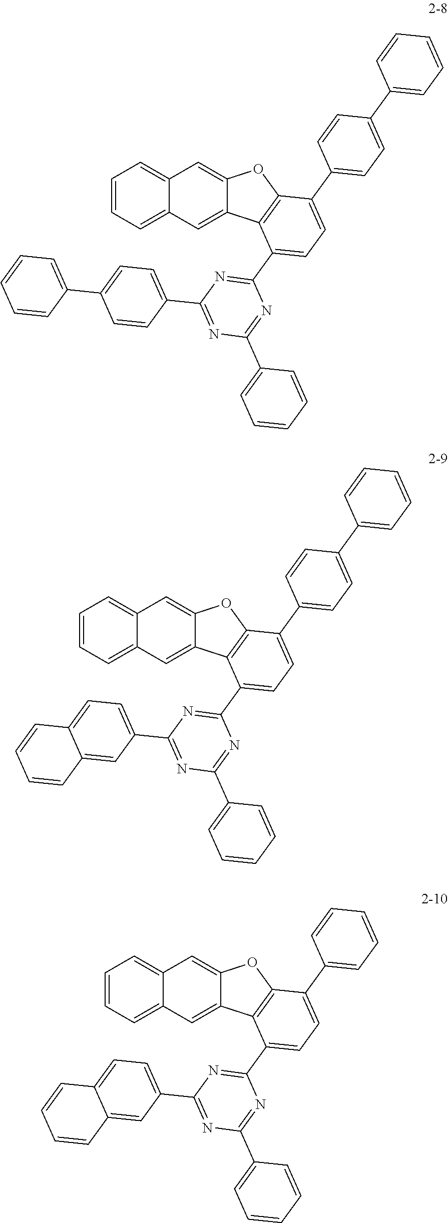 [SMILES: c1ccc(-c2ccc(-c3nc(-c4ccccc4)nc(-c4ccc(-c5ccc(-c6ccccc6)cc5)c5oc6cc7ccccc7cc6c45)n3)cc2)cc1 ; c1ccc(-c2ccc(-c3ccc(-c4nc(-c5ccccc5)nc(-c5ccc6ccccc6c5)n4)c4c3oc3cc5ccccc5cc34)cc2)cc1 ; c1ccc(-c2nc(-c3ccc4ccccc4c3)nc(-c3ccc(-c4ccccc4)c4oc5cc6ccccc6cc5c34)n2)cc1]

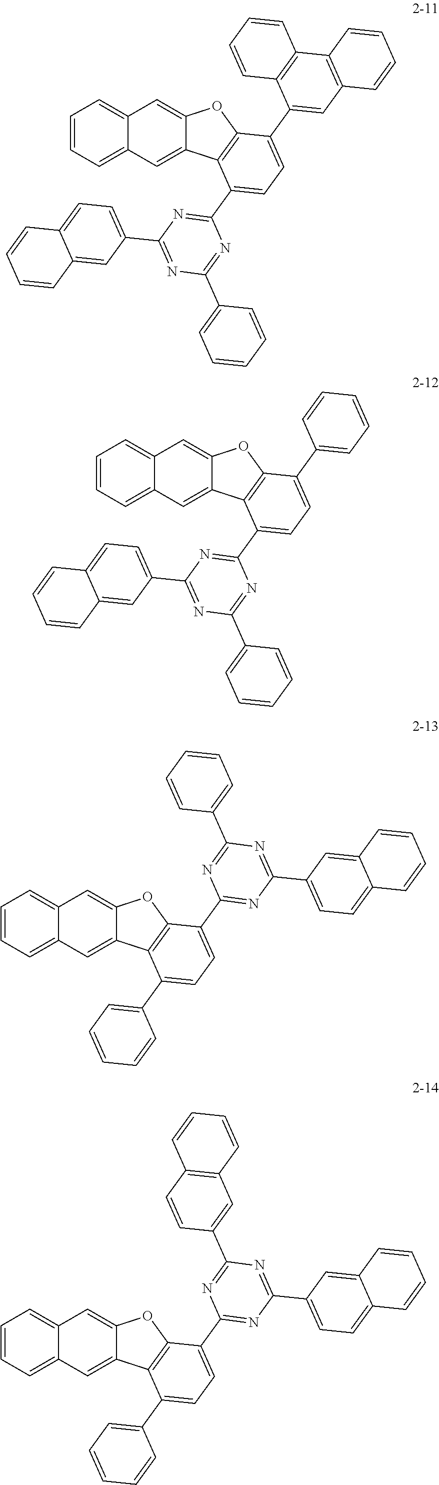 [SMILES: c1ccc(-c2nc(-c3ccc4ccccc4c3)nc(-c3ccc(-c4cc5ccccc5c5ccccc45)c4oc5cc6ccccc6cc5c34)n2)cc1 ; c1ccc(-c2nc(-c3ccc4ccccc4c3)nc(-c3ccc(-c4ccccc4)c4oc5cc6ccccc6cc5c34)n2)cc1 ; c1ccc(-c2nc(-c3ccc4ccccc4c3)nc(-c3ccc(-c4ccccc4)c4c3oc3cc5ccccc5cc34)n2)cc1 ; c1ccc(-c2ccc(-c3nc(-c4ccc5ccccc5c4)nc(-c4ccc5ccccc5c4)n3)c3oc4cc5ccccc5cc4c23)cc1]

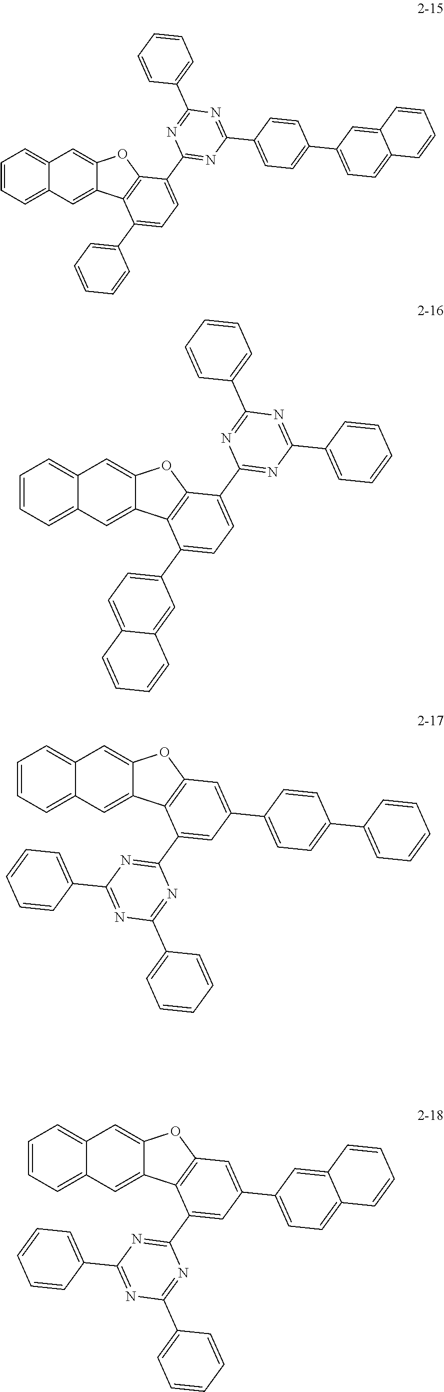 [SMILES: c1ccc(-c2nc(-c3ccc(-c4ccc5ccccc5c4)cc3)nc(-c3ccc(-c4ccccc4)c4c3oc3cc5ccccc5cc34)n2)cc1 ; c1ccc(-c2nc(-c3ccccc3)nc(-c3ccc(-c4ccc5ccccc5c4)c4c3oc3cc5ccccc5cc34)n2)cc1 ; c1ccc(-c2ccc(-c3cc(-c4nc(-c5ccccc5)nc(-c5ccccc5)n4)c4c(c3)oc3cc5ccccc5cc34)cc2)cc1 ; c1ccc(-c2nc(-c3ccccc3)nc(-c3cc(-c4ccc5ccccc5c4)cc4oc5cc6ccccc6cc5c34)n2)cc1]

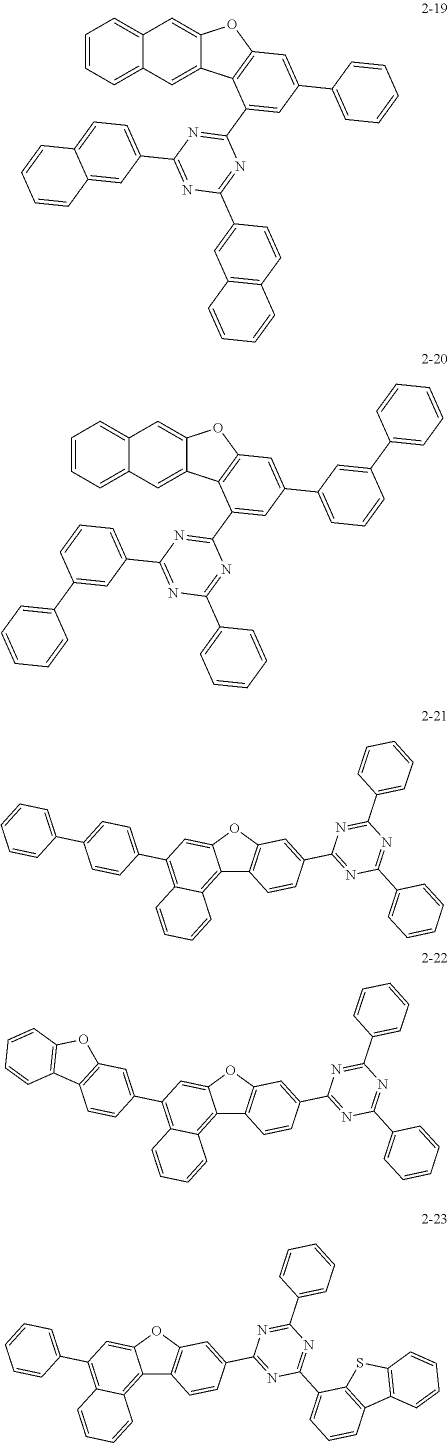 [SMILES: c1ccc(-c2cc(-c3nc(-c4ccc5ccccc5c4)nc(-c4ccc5ccccc5c4)n3)c3c(c2)oc2cc4ccccc4cc23)cc1 ; c1ccc(-c2cccc(-c3cc(-c4nc(-c5ccccc5)nc(-c5cccc(-c6ccccc6)c5)n4)c4c(c3)oc3cc5ccccc5cc34)c2)cc1 ; c1ccc(-c2ccc(-c3cc4oc5cc(-c6nc(-c7ccccc7)nc(-c7ccccc7)n6)ccc5c4c4ccccc34)cc2)cc1 ; c1ccc(-c2nc(-c3ccccc3)nc(-c3ccc4c(c3)oc3cc(-c5ccc6c(c5)oc5ccccc56)c5ccccc5c34)n2)cc1 ; c1ccc(-c2nc(-c3ccc4c(c3)oc3cc(-c5ccccc5)c5ccccc5c34)nc(-c3cccc4c3sc3ccccc34)n2)cc1]

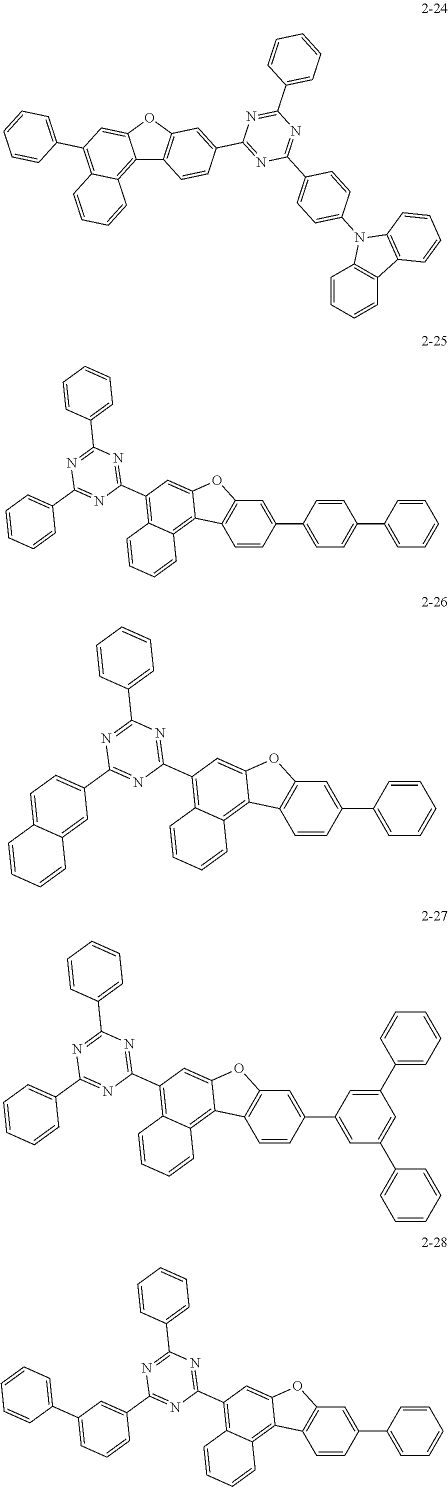 [SMILES: c1ccc(-c2nc(-c3ccc(-n4c5ccccc5c5ccccc54)cc3)nc(-c3ccc4c(c3)oc3cc(-c5ccccc5)c5ccccc5c34)n2)cc1 ; c1ccc(-c2ccc(-c3ccc4c(c3)oc3cc(-c5nc(-c6ccccc6)nc(-c6ccccc6)n5)c5ccccc5c34)cc2)cc1 ; c1ccc(-c2ccc3c(c2)oc2cc(-c4nc(-c5ccccc5)nc(-c5ccc6ccccc6c5)n4)c4ccccc4c23)cc1 ; c1ccc(-c2cc(-c3ccccc3)cc(-c3ccc4c(c3)oc3cc(-c5nc(-c6ccccc6)nc(-c6ccccc6)n5)c5ccccc5c34)c2)cc1 ; c1ccc(-c2cccc(-c3nc(-c4ccccc4)nc(-c4cc5oc6cc(-c7ccccc7)ccc6c5c5ccccc45)n3)c2)cc1]

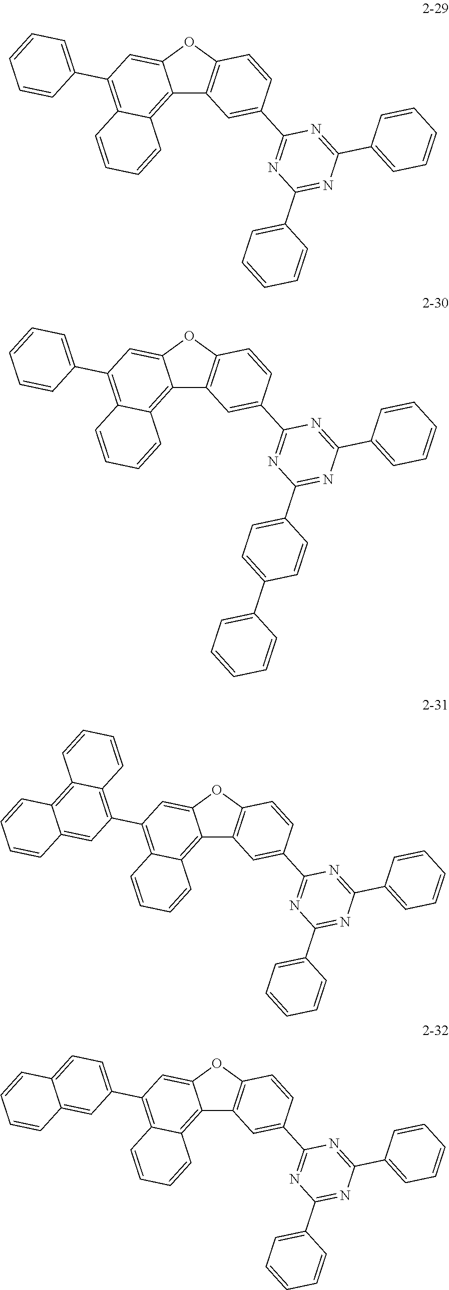 [SMILES: c1ccc(-c2nc(-c3ccccc3)nc(-c3ccc4oc5cc(-c6ccccc6)c6ccccc6c5c4c3)n2)cc1 ; c1ccc(-c2ccc(-c3nc(-c4ccccc4)nc(-c4ccc5oc6cc(-c7ccccc7)c7ccccc7c6c5c4)n3)cc2)cc1 ; c1ccc(-c2nc(-c3ccccc3)nc(-c3ccc4oc5cc(-c6cc7ccccc7c7ccccc67)c6ccccc6c5c4c3)n2)cc1 ; c1ccc(-c2nc(-c3ccccc3)nc(-c3ccc4oc5cc(-c6ccc7ccccc7c6)c6ccccc6c5c4c3)n2)cc1]

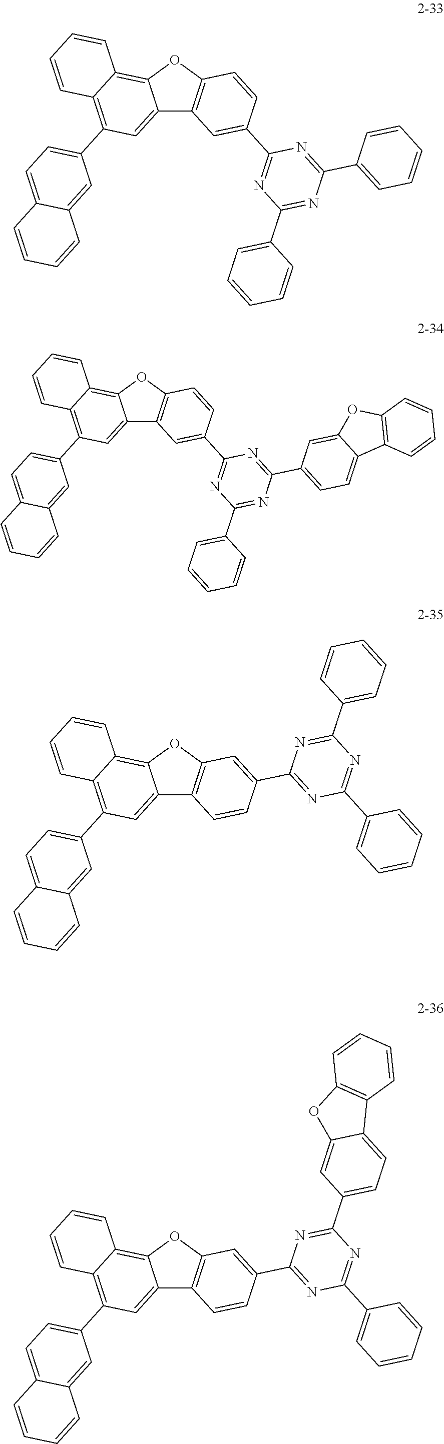 [SMILES: c1ccc(-c2nc(-c3ccccc3)nc(-c3ccc4oc5c6ccccc6c(-c6ccc7ccccc7c6)cc5c4c3)n2)cc1 ; c1ccc(-c2nc(-c3ccc4c(c3)oc3ccccc34)nc(-c3ccc4oc5c6ccccc6c(-c6ccc7ccccc7c6)cc5c4c3)n2)cc1 ; c1ccc(-c2nc(-c3ccccc3)nc(-c3ccc4c(c3)oc3c5ccccc5c(-c5ccc6ccccc6c5)cc43)n2)cc1 ; c1ccc(-c2nc(-c3ccc4c(c3)oc3ccccc34)nc(-c3ccc4c(c3)oc3c5ccccc5c(-c5ccc6ccccc6c5)cc43)n2)cc1]

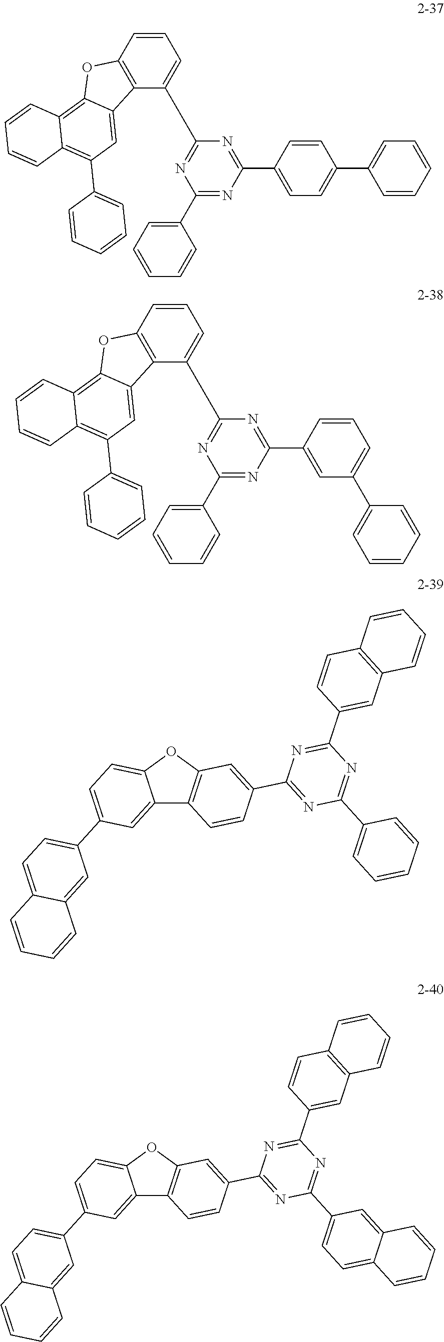

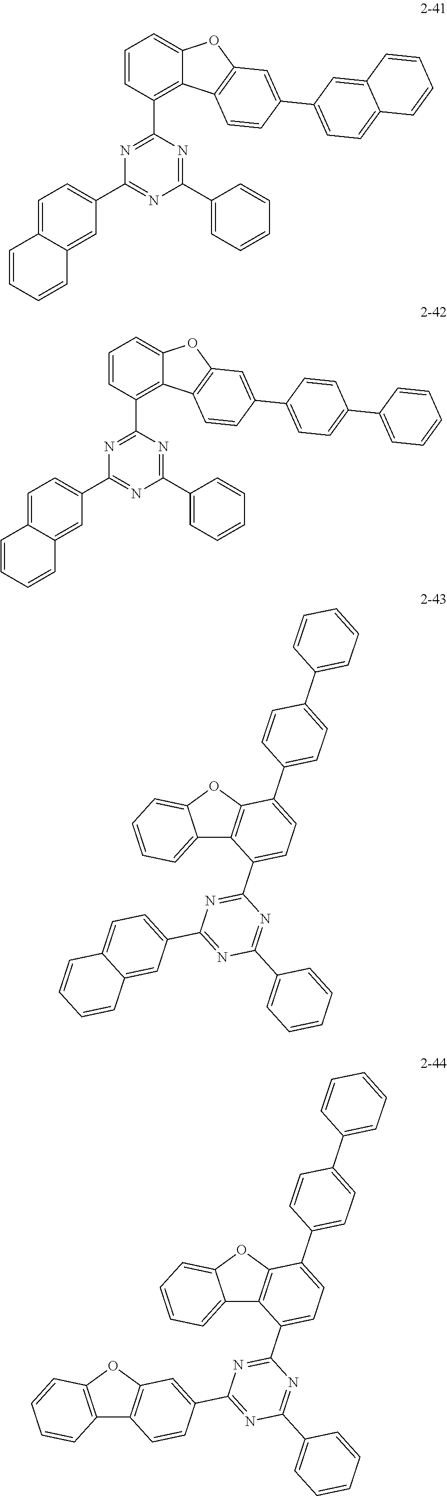 [SMILES: c1ccc(-c2nc(-c3ccc4ccccc4c3)nc(-c3cccc4oc5cc(-c6ccc7ccccc7c6)ccc5c34)n2)cc1 ; c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1 ; c1ccc(-c2ccc(-c3ccc(-c4nc(-c5ccccc5)nc(-c5ccc6c(c5)oc5ccccc56)n4)c4c3oc3ccccc34)cc2)cc1]

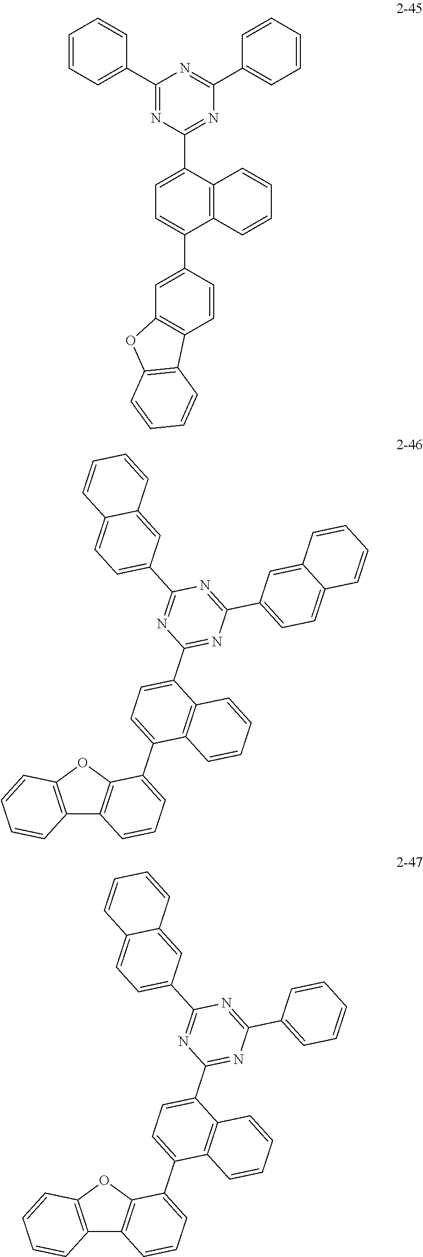

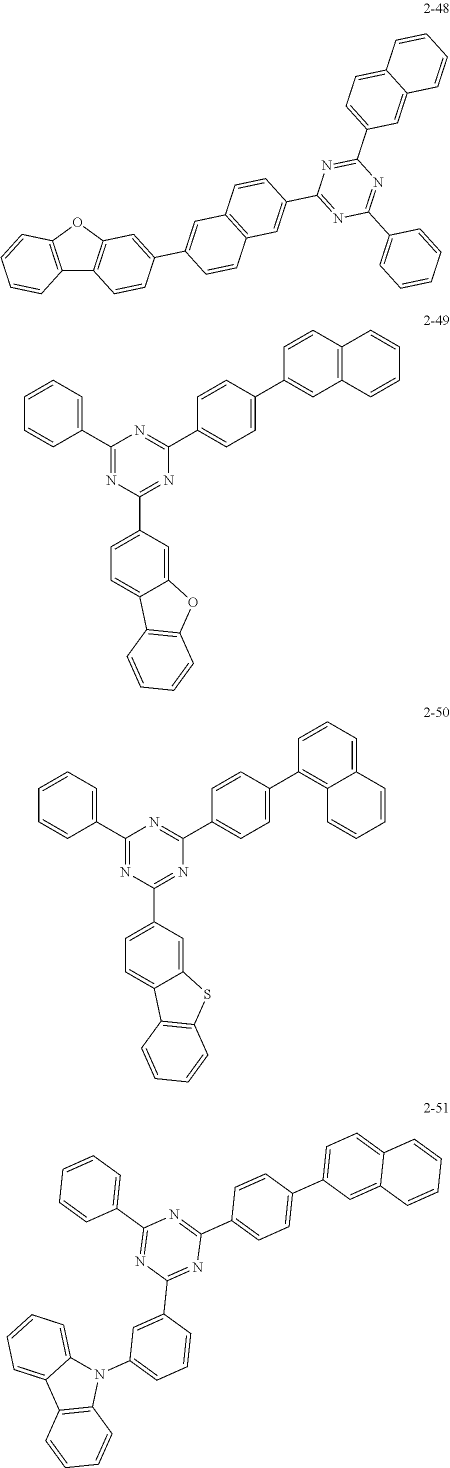 [SMILES: c1ccc(-c2nc(-c3ccc4ccccc4c3)nc(-c3ccc4cc(-c5ccc6c(c5)oc5ccccc56)ccc4c3)n2)cc1 ; c1ccc(-c2nc(-c3ccc(-c4ccc5ccccc5c4)cc3)nc(-c3ccc4c(c3)oc3ccccc34)n2)cc1 ; c1ccc(-c2nc(-c3ccc(-c4cccc5ccccc45)cc3)nc(-c3ccc4c(c3)sc3ccccc34)n2)cc1 ; c1ccc(-c2nc(-c3ccc(-c4ccc5ccccc5c4)cc3)nc(-c3cccc(-n4c5ccccc5c5ccccc54)c3)n2)cc1]

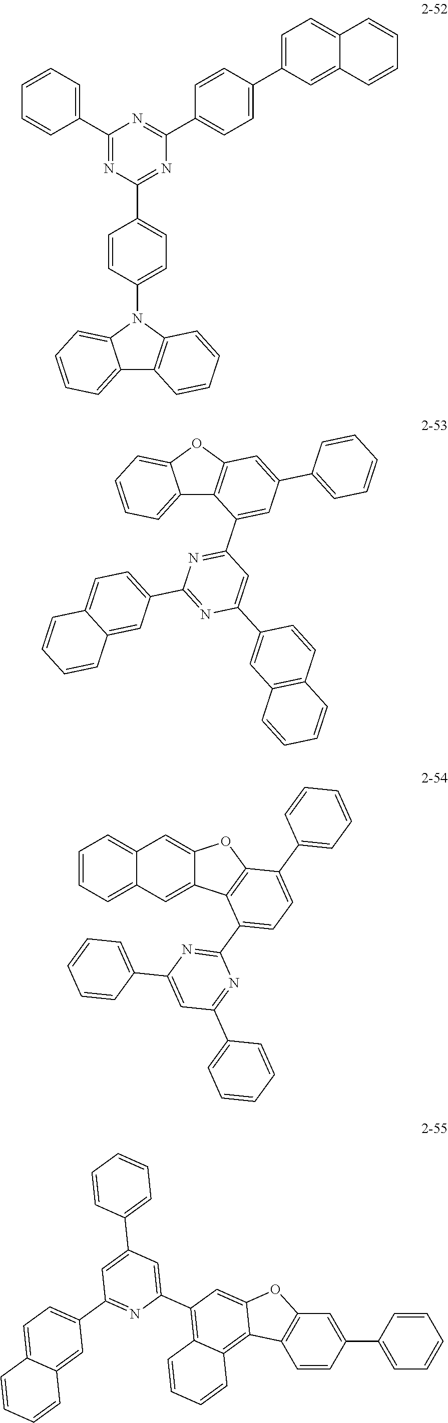 [SMILES: c1ccc(-c2nc(-c3ccc(-c4ccc5ccccc5c4)cc3)nc(-c3ccc(-n4c5ccccc5c5ccccc54)cc3)n2)cc1 ; c1ccc(-c2cc(-c3cc(-c4ccc5ccccc5c4)nc(-c4ccc5ccccc5c4)n3)c3c(c2)oc2ccccc23)cc1 ; c1ccc(-c2cc(-c3ccccc3)nc(-c3ccc(-c4ccccc4)c4oc5cc6ccccc6cc5c34)n2)cc1 ; c1ccc(-c2cc(-c3ccc4ccccc4c3)nc(-c3cc4oc5cc(-c6ccccc6)ccc5c4c4ccccc34)c2)cc1]

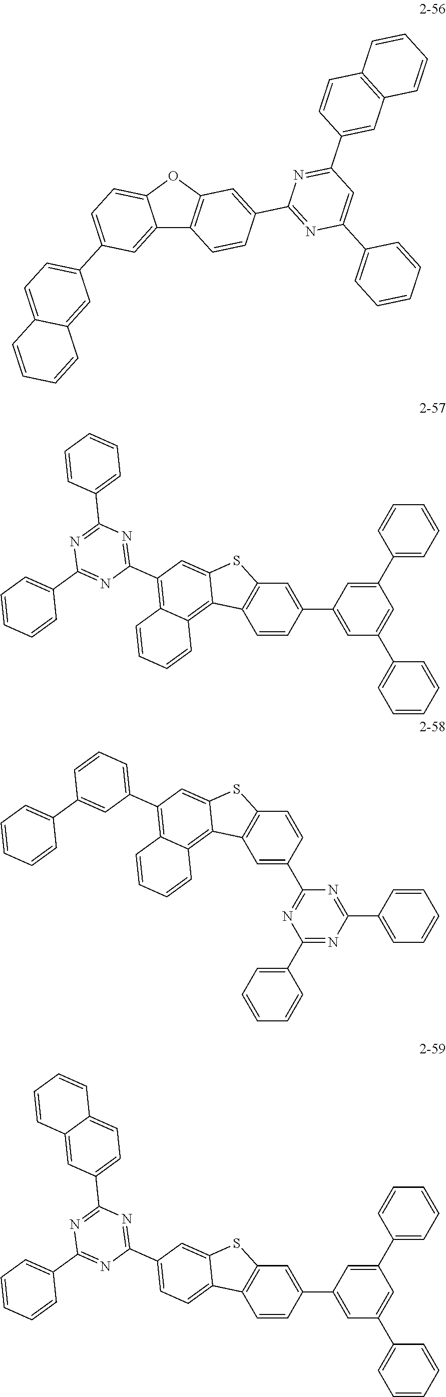 [SMILES: c1ccc(-c2cc(-c3ccc4ccccc4c3)nc(-c3ccc4c(c3)oc3ccc(-c5ccc6ccccc6c5)cc34)n2)cc1 ; c1ccc(-c2cc(-c3ccccc3)cc(-c3ccc4c(c3)sc3cc(-c5nc(-c6ccccc6)nc(-c6ccccc6)n5)c5ccccc5c34)c2)cc1 ; c1ccc(-c2cccc(-c3cc4sc5ccc(-c6nc(-c7ccccc7)nc(-c7ccccc7)n6)cc5c4c4ccccc34)c2)cc1 ; c1ccc(-c2cc(-c3ccccc3)cc(-c3ccc4c(c3)sc3cc(-c5nc(-c6ccccc6)nc(-c6ccc7ccccc7c6)n5)ccc34)c2)cc1]

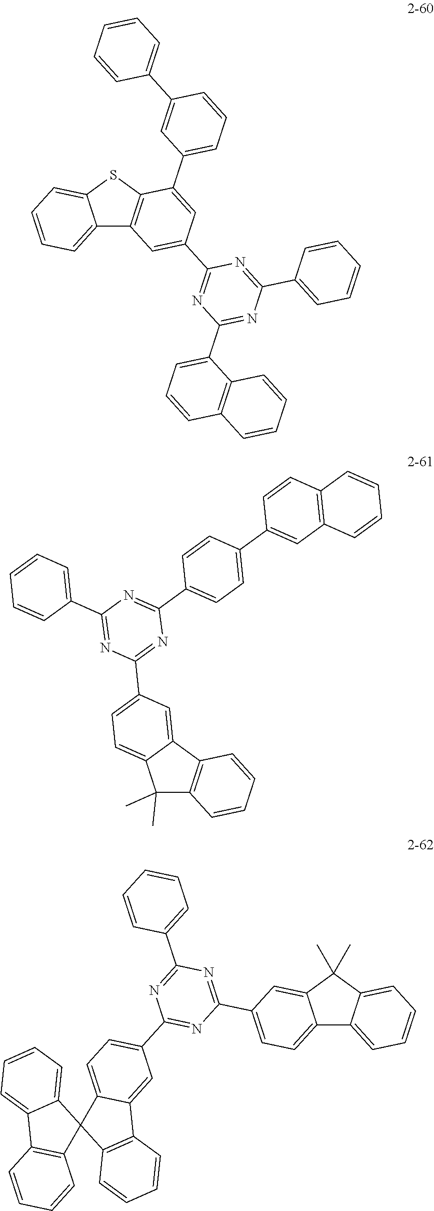

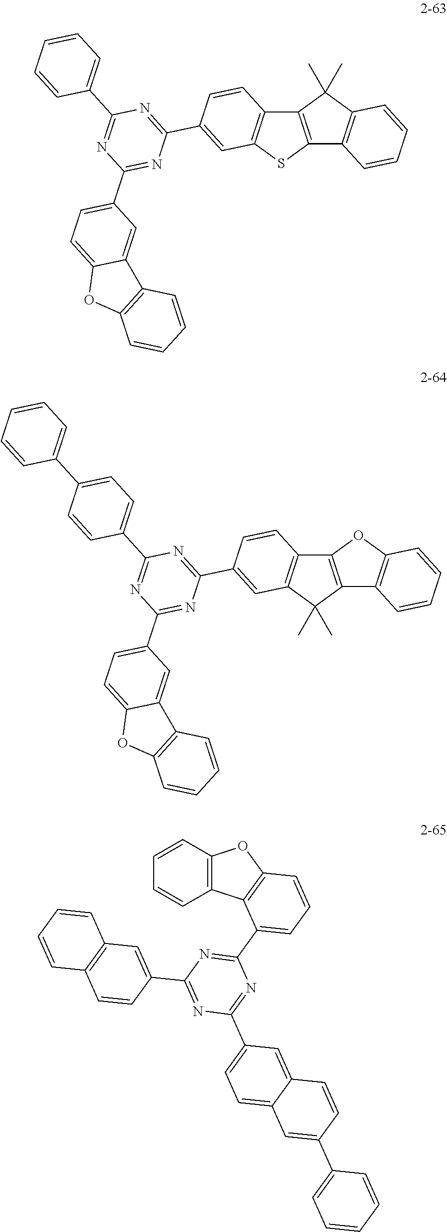 [SMILES: CC1(C)c2ccccc2-c2sc3cc(-c4nc(-c5ccccc5)nc(-c5ccc6oc7ccccc7c6c5)n4)ccc3c21 ; CC1(C)c2cc(-c3nc(-c4ccc(-c5ccccc5)cc4)nc(-c4ccc5oc6ccccc6c5c4)n3)ccc2-c2oc3ccccc3c21 ; c1ccc(-c2ccc3cc(-c4nc(-c5ccc6ccccc6c5)nc(-c5cccc6oc7ccccc7c56)n4)ccc3c2)cc1]

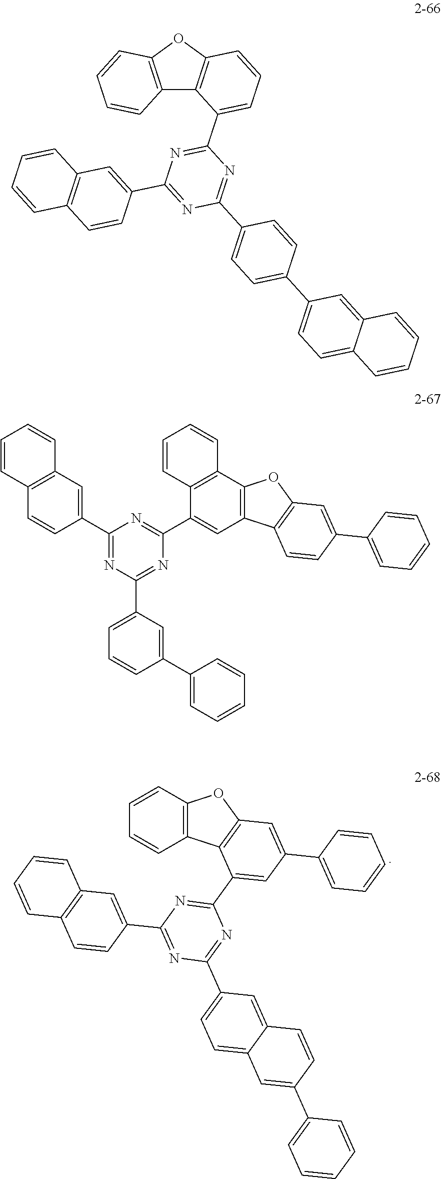 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2cccc(-c3nc(-c4ccc5ccccc5c4)nc(-c4cc5c6ccc(-c7ccccc7)cc6oc5c5ccccc45)n3)c2)cc1 ; c1ccc(-c2ccc3cc(-c4nc(-c5ccc6ccccc6c5)nc(-c5cc(-c6ccccc6)cc6oc7ccccc7c56)n4)ccc3c2)cc1]

14. The organic light emitting device of claim 9, further comprising one or more layers selected from the group consisting of a light emitting layer, a hole injection layer, a hole transport layer, a hole transport auxiliary layer, an electron injection layer, an electron transport layer, an electron blocking layer and a hole blocking layer.

15. A composition for an organic material layer, the composition comprising:
the heterocyclic compound of claim 1; and
a heterocyclic compound represented by the following Chemical Formula A: 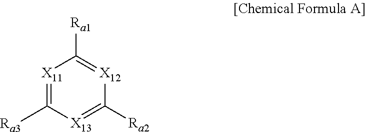
wherein, in Chemical Formula A,
X₁₁ to X₁₃ are the same as or different from each other, and each independently N or CR′;
at least one of X₁₁ to X₁₃ is N; and
Rₐ₁ to Rₐ₃ and R′ are the same as or different from each other, and each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group.

16. The composition of claim 15, wherein, in the composition, the heterocyclic compound represented by Chemical Formula 1 and the heterocyclic compound represented by Chemical Formula A have a weight ratio of 1:10 to 10:1.
